(19) United States
(12) Patent Application Publication
(10) Pub. No.: US 2012/0065126 A1
(43) Pub. Date: Mar. 15, 2012

(54) Pharmaceutical Compositions Containing Antifungal Peptides

(75) Inventors: Heike Brüser, Speyer (DE); Daniel Hümmerich, Frankenthal (DE); Claus Bollschweiler, Hedelberg (DE); Carsten Schwalb, Mannnheim (DE)

(73) Assignee: BASF SE, Ludwigshafen (DE)

(21) Appl. No.: 13/320,467

(22) PCT Filed: May 12, 2010
(86) PCT No.: PCT/EP2010/056536

§ 371 Date: Nov. 14, 2011

(30) Foreign Application Priority Data
May 15, 2009 (EP) 09160404.1

(51) Int. Cl. A61K 38/16; A61K 38/10; A61P 17/08; A61P 29/00; A61P 31/04; A61K 38/03; A61P 31/10

(52) U.S. Cl. 514/3.3

(57) ABSTRACT

The invention relates to a pharmaceutical composition containing, in a pharmaceutical carrier, a peptide comprising at least one sequence motif of the following general formula (I) Hel1-HB-Hel2. The invention also relates to the use and production of said pharmaceutical compositions.

The present invention relates to the use of specific peptides in pharmaceutical compositions which find use especially in topical form as antifungal and antibacterial compositions. The invention further relates to the production of such compositions, to the production thereof and to nucleotide sequences coding for such peptides.

### BACKGROUND OF THE INVENTION

Various antimicrobial peptides have already been described in the literature and summarized in reviews (Hancock, R. E. W. and Lehrer, R. 1998 in Trends in Biotechnology, 16: 82-88; Hancock, R. E. W. and Sahl, H. G. 2006 in Nature Biotechnology, 24: 1551-1557).

Fusion peptides, which combine two active peptides in one, are likewise described in the literature. Wade et al., report the antibacterial action of various fusions of cecropin A from Hyalophora cecropia and the bee toxin melittin (Wade, D. et al., 1992, International Journal of Peptide and Protein Research, 40: 429-436). Shin et al. describe the antibacterial action of a fusion peptide of cecropin A from Hyalophora cecropia and magainin 2 from Xenopus laevis, consisting of 20 amino acids. Cecropin A consists of 37 amino acids and exhibits activity toward Gram-negative bacteria, but lower activity toward Gram-positive bacteria. Magainin 2 consists of 23 amino acids and is active toward bacteria, but also tumor cell lines. Compared to the fusion of cecropin A and melittin, the fusion of cecropin and magainin exhibits much lower hemolytic activity and antimicrobial activity against Escherichia coli and Bacillus subtilis (Shin, S. Y. L Kang, J. H., Lee, M. K., Kim, S. Y., Kim, Y., Hahm, K. S., 1998, Biochemistry and Molecular Biology International, 44: 1119-1126).

US 2003/0096745 A1 and U.S. Pat. No. 6,800,727 B2 claim these fusion peptides consisting of 20 amino acids and variants of this fusion which have stronger positive charges and are more hydrophobic as a result of the exchange of amino acids, especially of positively charged amino acids and hydrophobic amino acids.

Further developments of this cecropin A-magainin 2 fusion peptide were described by Shin et al. in 1999. It was found here that the P18 construct (HT2, SEQ ID NO: 3) had a lower hemolytic activity compared to the starting fusion, but the antibacterial activity toward Escherichia coli and Bacillus subtilis was not impaired (Shin et al. 1999 Journal of Peptide Research, 53: 82-90).

In addition, Shin et al. in 2001 also demonstrated the activity of the P18 construct and analogous constructs on Pseudomonas aeruginosa, Proteus vulgaris, Staphylococcus aureus and Bacillus megaterium (Shin et al., 2001, Journal of Peptide Research, 58: 504-514).

First studies of the efficacy of P18 against fungi were published in January 2002 by Shin et al. A better efficacy of the P18 peptide against Candida albicans as compared with magainin 2 was found here. In addition, antimicrobial action of P18 against Salmonella typhimurium, Staphylococcus epidermidis, Enterococcus faecalis, Enterococcus faecium and Stenotrophomonas maltophilia was demonstrated (Shin et al., 2002, Biochemical and Biophysical Research Communications, 290: 558-562).

Lee et al. in 2002 reported the action of P18 and two reverse peptides against Trichopsoron beigelii, Aspergillus flavus, Fusarium oxysporum, and also against Streptococcus pyogenes and Serratia marcescens (Lee et al., 2002, Protein and Peptide Letters, 9: 395-402)

In addition to Candida albicans, the ascomycetes Aspergillus flavus, Fusarium oxysporum and the basidiomycete Trichosporon beigelii were inhibited by P18 and variants of the peptide. However, the most effective inhibition was achieved with P18 (Lee et al., 2004, Biotechnology Letters, 26: 337-341). There is no teaching of inhibition of lipophilic fungi, especially of the Malassezia genus. There is also no description of any experiments which demonstrate more effective action of cecropin-magainin fusions as compared with known commercial antifungal substances.

WO-A-00/032220 describes the use of a fungal polypeptide as an antifungal active for treatment of dandruff. There is also no internal comparison here with prior art antifungal actives.

US 2003/0096745 describes a polypeptide of the sequence KWKKLLKKPPPLLKKLLKKL with antibacterial and antifungal activity against particular microorganisms. Antifungal activity was shown against Candida albicans and Tricosphoron beigelii.

Shin et al. describe the efficacy of a peptide with the sequence KWKKLPKKLLKLL-NH₂ against Escherichia coli, Pseudomonas aeruginosa, Salmonella typhimurium, Bacillus subtilis, Staphylococcus aureus and Staphylococcus epidermidis. Efficacy against fungi is not studied (Shin et al., 2004, Biotechnology Letters, 26:735-739). Antibacterial and antifungal action of the active ZPT (zinc pyrithione) for commercial treatment of dandruff is known (WO 01/00151, U.S. Pat. No. 3,236,733, Khattar, M. M. et al., 1988, Journal of Applied Biotechnology, 64:265-272, Carol, J. C. et al., 1978, Antimicrobial Agents and Chemotherapy, 14:60-68). However, the action occurs only after a relatively long contact time.

However, antimicrobial substances in use, particularly in regular use, can lead to intolerance in humans, or even to damaged health. Intolerances may be reddened skin, irritations or sensitizations. Systemic uptake into the human body can lead to impairment of body functions. In particular, regular use of some antimicrobial substances can lead to enrichment. A known example is parabens (Dabre et al., 2004, Journal of Applied Toxicology, 24: 5-13). Depending on the application, there may also be accumulation in the human body or in the environment.

In addition, excessive and inappropriate use of antimicrobial substances is constantly causing resistances in the target organisms.

There is therefore a need to provide novel pharmaceutical antimicrobial compositions which firstly have a high antifungal efficacy and secondly a low general cytotoxicity, such that safe therapeutic use of these compositions becomes possible.

It was therefore an object of the present invention to provide a novel, effective active with a low level of side effects for treatment of mycoses, especially dermatomycoses, which is also suitable for the treatment of external bacterial infections, for example of the skin, nails, hair and mucous membranes.

### SUMMARY OF THE INVENTION

This object was achieved by pharmaceutical compositions as defined in the appended claims.

### DETAILED DESCRIPTION OF THE INVENTION

### 1. General Definitions

A “helix breaker” means a section within an inventive peptide which inhibits the formation of a helical secondary structure in the region of this section of the peptide chain. However, the formation of a helix structure at a relatively great distance from the helix breaker is not suppressed. Typical helix breakers are known to those skilled in the art. More particularly, the amino acid proline is a peptide unit with the property of a helix breaker. The same applies to proline-containing peptide fragments.

“Hydrophobic amino acids” in the context of the invention are alanina, valine, leucine, isoleucine, phenylalanine, methionine and tryptophan.

“Hydrophilic amino acids” in the context of the invention are especially amino acids with polar side chains, such as serine, threonine, cysteine, tyrosine, asparagine and glutamine; acidic amino acids such as aspartic acid and glutamic acid; and especially basic amino acids such as lysine, arginine and histidine.

A sequence “capable of forming an alpha-helix arm” is one which promotes the formation of a helical structure under suitable conditions. Artificial suitable conditions for formation of helix structures are, for example, the solvent systems based on trifluoroethanol and SDS which promote alpha-helix formation.

“Percentage alpha-helicity” is understood to mean a measurement obtained with the aid of circular dichroism (CD) analysis, wherein the sample to be analyzed is obtained under standard conditions, such as especially 50% (v/v) trifluoroethanol in 10 mM sodium phosphate buffer, pH 7.0, or 30 mM SDS in 10 mM sodium phosphate buffer, pH 7.0, using an analysis cell with path length 1 mm at a peptide concentration of 100 μg/ml. The calculation is effected according to the following formula:

% helicity=100([θ]−[θ]⁰)/[θ]¹⁰⁰
in which
[θ] is the experimentally determined ellipticity at 222 nm;
[θ]⁰ is the ellipticity at 222 nm and 0% helicity and
[θ]¹⁰⁰ is the ellipticity at 222 nm and 100% helicity.

Suitable analysis conditions are described, for example, by Shin et al., 1999, Journal of Peptide Research, 53:82-90, which is hereby explicitly incorporated by reference.

A “repetitive sequence motif” is understood to mean the linear arrangement of preferably identical peptide sequences, which are joined to one another directly or indirectly, i.e. via “linker groups” as defined herein.

The terms “mutants” and “variants” are used synonymously. These are especially understood to mean “functional” or “functionally equivalent” modifications, as will be explained in more detail later, which still exhibit the desired activity and hence usability as an antimycotic.

A “fusion product” is understood to mean the covalent or noncovalent linkage of peptides and proteins (“fusion peptides”) and the covalent or noncovalent linkage of peptides and polymers (“fusion polymers”). The mutually linked constituents are bonded to one another either irreversibly or reversibly, i.e. are cleavable biologically, especially enzymatically.

### 2. Preferred Embodiments

The invention firstly relates to a pharmaceutical composition comprising, in a pharmaceutical carrier, a peptide comprising at least one sequence motif of the following general formula I

Hel1-HB-Hel2 (I)

in which
“HB” comprises 1 to 5, especially 1, 2 or 3, consecutive amino acid residues and represents a subsequence motif with the function of a helix breaker, and “Hel1” and “Hel2” are identical or different subsequence motifs each comprising 5 to 15, for example 6 to 12, especially 8, 9 or 10, consecutive amino acid residues which are selected essentially from hydrophilic, especially basic, residues and hydrophobic residues other than proline, and are each capable of forming an alpha-helix arm, at least one of the helix arms in the axial projection thereof, i.e. in the top view corresponding to a “helical wheel” diagram, having an incomplete separation into a hydrophobic, especially basic, and hydrophilic helix half. It is possible, for example, for 1, 2, 3 or 4 positions of one half of one type (hydrophobic or hydrophilic) to be occupied by amino acid residues of the other type (hydrophilic or hydrophobic).

In contrast, completely separated hydrophobic and hydrophilic helix halves would consist exclusively of hydrophobic and hydrophilic amino acid residues as defined above. One example of a helix with complete hydrophilic/hydrophobic separation is the sequence motif KLKKLLKK.

One “helix half” should not necessarily be understood to mean the numerical half, i.e. half of the total number of amino acids in a helix. The numerical size of two halves may differ, for example, by 1 to 3 amino acids.

The invention relates secondly to a pharmaceutical composition comprising, in a pharmaceutical carrier, a peptide comprising at least one sequence motif of the following general formula I

Hel1-HB-Hel2 (I)

in which
“HB” comprises 1 to 5, especially 1, 2 or 3, consecutive amino acid residues and represents a subsequence motif with the function of a helix breaker, and
“Hel1” and “Hel2” are identical or different subsequence motifs each comprising 5 to 15, for example 6 to 12, especially 8, 9 or 10, consecutive amino acid residues which are selected essentially from hydrophilic, especially basic, residues and hydrophobic residues other than proline, and are each capable of forming an alpha-helix arm,
the peptide having a percentage alpha-helicity (% helicity) of about 7 to 98%, for example 30 to 80% or 30 to 60%, in 50% (v/v) trifluoroethanol, pH 7.0; or a % helicity value of about 8 to 60%, or 12 to 55%, or 12 to 40%, in 30 mM SDS, pH 7.0, in each case determined by CD spectrometry.

The invention relates thirdly to a pharmaceutical composition comprising, in a pharmaceutical carrier, at least one peptide with a sequence or a repetitive sequence motif according to SEQ ID NO: 1:

(SEQ ID NO: 1)

X₁ X₂K X₃ X₄ X₅KIP X₁₀ KFX₆X₇ X₈ AX₉KF

in which
X₁₀ is a peptide bond or any one or two basic or hydrophobic amino acid residues or one or two proline residues and
X₁ to X₉ are any basic or hydrophobic amino acid residues other than proline; where the repetitive sequence motifs may be the same or different;
and/or mutants or derivatives thereof.

More particularly, the invention relates to compositions as defined above, comprising at least one peptide with a sequence or a repetitive sequence motif according to SEQ ID NO: 2:

(SEQ ID NO: 2)

X₁ X₂K X₃ X₄ X₅KIP X₁₁ X₁₂ KFX₆X₇ X₈ AX₉KF

in which
X₁ is lysine, arginine or phenylalanine,
X₂ is lysine or tryptophan,
X₃ is leucine or lysine,
X₄ is phenylalanine or leucine,
X₅ is leucine or lysine,
X₆ is leucine or lysine,
X₇ is histidine or lysine,
X₈ is alanine, leucine, valine or serine,
X₉ is leucine or lysine,
X₁₁ is proline or a chemical bond, and
X₁₂ is proline or a chemical bond,
where the repetitive sequence motifs are the same or different;
and/or mutants or derivatives thereof.

Nonlimiting examples of above sequences or repetitive sequence motifs according to SEQ ID NO: 3 are:

| | | |
|---|---|---|
| P18 | KWKLFKKIPKFLHLAKKF-NH₂ | (SEQ ID NO: 3) |
| RP18 | RWKLFKKIPKFLHLAKKF | (SEQ ID NO: 4) |
| KKFP18 | FKKLFKKIPKFLHAAKKF | (SEQ ID NO: 5) |
| KKLP18 | KWKLLKKIPKFKKLALKF | (SEQ ID NO: 6) |
| AP18 | KWKLFKKIPKFLHAAKKF | (SEQ ID NO: 7) |
| KFLP18 | KWKKFLKIPKFLHAAKKF | (SEQ ID NO: 8) |
| KLLP18 | KWKKLLKIPKFLHAAKKF | (SEQ ID NO: 9) |

and/or a mutant or derivative thereof.

Inventive compositions may especially comprise peptides with a repetitive sequence motif wherein a multitude, such as especially 2 to 10 or 3 to 5, of peptides of the general formula I or according to SEQ ID NO: 1 to 9 or mutants or derivatives thereof are peptide-bonded to one another via linker groups.

These “linker groups” may comprise 1 to 10 identical or different consecutive amino acid residues, preferably selected from alanine, glycine, threonine and serine, for example GGSGGT, GGSGGS, or polyalanine linkers and polyglycine linkers, where “poly” represents 2 to 10; or selected from Asp, Pro, Asn and Gly, for example Asp-Pro and Asn-Gly.

It is additionally possible to use peptides whose C-terminal carboxyl group has been amidated.

The invention also provides compositions comprising an optionally cleavable fusion polypeptide of at least one pharmaceutical, preferably peptidic, excipient or active and at least one peptide as defined above. Examples of such actives include: hydrophobins, keratin binding domains, albumin, lactoferrin, avidin, antibodies, preferably keratin-binding antibodies, binding peptides for surfaces, preferably keratin-binding peptides, silk proteins, spider silk proteins, preferably C16, collagen, fibronectin, keratin, elastin, other structural proteins, preferably hair and skin structure proteins, binding proteins for skin or hair structure proteins, enamel-building proteins, amelogenin, binding proteins of the enamel-building proteins, binding proteins of amelogenin; where these fusions may be permanent or else cleavable.

The invention also provides fusion polymers of at least one pharmaceutical polymer and at least one peptide as defined above. Examples of such polymers include: polyhydroxyalkanoates, hyaluronic acid, glucan, spheroglucan, cellulose, xanthan, polyethylene glycol, polyglycerol, polylysine and silicones, which are present in the form of covalent or noncovalent linkages.

It is also conceivable that the abovementioned peptides may also be present in the compositions in the form of a covalent linkage to pharmaceutically active ingredients such as panthenol, bisabolol, retinol, carotenoids, protein hydrolyzates.

The invention also provides compositions as defined above, additionally comprising at least one further pharmaceutical active, for example at least one anti-inflammatory active, an antimicrobial active for inhibition of the growth and/or of the pathophysiological activity of unwanted bacteria, such as especially Malasezzia furfur, and/or a sebum-regulating active.

Examples of anti-inflammatory actives include: corticoids (e.g. cortisone), azathioprin, bisabolol, cyclosporin A, acetylsalicylic acid, ibuprofen, panthenol, chamomile extract or aloe extracts, antiphlogistics, cytostatics, etc.

Examples of antimicrobial agents include: typical preservatives known to those skilled in the art, such as alcohols, p-hydroxybenzoic esters, imidazolidinyl urea, formaldehyde, sorbic acid, benzoic acid, salicylic acid, etc. Such deodorizing substances are, for example, zinc ricinoleate, triclosan, undecylenoic acid alkylolamides, triethyl citrate, chlorhexidine, etc (cf. also section 3.5 below). In addition, they include azoles (ketoconazole, climbazole), zinc pyrithione, selenium sulfides, etc.

Examples of sebum-regulating actives include: azelaic acid, potassium azelaoyl diglycinate, sebacic acid, 10-hydroxydecanoic acid, 1,10-decanediol, aluminum salts, for example aluminum chloride.

The invention further provides the above-described peptides in the use as a medicament, especially as a medicament for treatment of mycoses, particularly dermatomycoses, and also for treatment of bacterial infections, especially external infections such as infections of the skin, nails, hair or mucous membranes.

These medicaments may, as well as the antimicrobially active peptides, also comprise further pharmaceutically active substances, for example antibiotics, which can be administered simultaneously with the antimicrobially active peptides or at time intervals.

The inventive peptidic actives have antimicrobial and antimycotic effects. They have a very broad spectrum of antimycotic action, especially against dermatophytes and yeast-like fungi, and also biphasic fungi, for example against Candida species such as Candida albicans, Candida dubliniensis, Candida famata, Candida glabrata, Candida guilliermondii, Candida kefyr, Candida krusei, Candida lusitaniae, Candida parapsilosis, Candida tropicalis, Epidermophyton species such as Epidermophyton floccosum, Aspergillus species such as Aspergillus niger, Aspergillus flavus and Aspergillus fumigatus, Trichophyton species such as Trichophyton rubrum, Trichophyton tonsurans, Trichophyton ajelloi, Trichophyton equinum, Trichophyton erinacei, Trichophyton interdigitale, Trichophyton megninii, Trichophyton mentagrophytes, Trichophyton quinckeanum, Trichophyton schoenleinii, Trichophyton soudanense, Trichophyton terrestre, Trichophyton verrucosum, Trichophyton violaceum, Microsporum species such as Microsporum felineum, Microsporum audouinii, Microsporum canis, Microsporum cookei, Microsporum distortum, Microsporum ferrugineum, Microsporum gallinae, Microsporum gypseum, Microsporum nanum and Microsporum racemosum, and Torulopsis species such as Torulopsis glabrata, and also against Malassezia species such as Malassezia furfur, Malassezia globosa, Malassezia obtusa, Malassezia pachydermatis, Malassezia restricta, Malassezia slooffiae and Malassezia sympodialis.

Mention should also be made of Trichosporon ssp., Piedraia hortae, and also of various black fungi. The enumeration of these microorganisms in no way constitutes a restriction of the bacteria which can be controlled, but is merely of illustrative character.

Consequently, the inventive pharmaceutical compositions can be assumed to be effective against the following disorders associated with the fungal pathogens enumerated: aspergillosis, bronchopulmonary aspergillosis (ABPA), candidid, candidiasis, extrinsic allergic alveolitis (EAA), genital mycosis (vulva mycosis, vaginal mycosis), microsporosis, mucositis/thrush, onychial and paronychial candidiasis, onychomycosis, piedra, Pityriasis versicolor, Pityriasis folliculitis, Tinea capitis, Tinea corporis, Tinea gladiatorum, Tinea ungium, trichophytia, zygomycosis, Tinea manuum, Tinea pedis.

The inventive peptidic actives have antibacterial action against Staphylococci such as Staphylococcus epidermidis and Staphylococcus aureus, Streptococci such as Streptococcus mutans and Streptococcus pyogenes, Propionibacteria such as Propionibacterium acnes and Propionibacterium granulosum, but also Pseudomonas aeruginosa and Enterobacteriaceae such as Escherichia coli, Shigella ssp., Enterococcus ssp and Klebsiella ssp. The enumeration of these microorganisms in no way constitutes a restriction of the bacteria which can be controlled, but is merely of illustrative character.

Consequently, the inventive pharmaceutical compositions can be assumed to be effective against the following disorders associated with the bacterial pathogens enumerated: comedones, abscesses, Acne vulgaris, purulent discharges, boils, pustules, pus-forming infections of the skin and mucous membranes, exfoliative dermatitis, staphylococcal scalded skin syndrome, caries, excrescences and dermatitis.

In addition, it can be assumed that patients with seborrheaic eczema, allergic contact eczema, atopic dermatitis, Psoriasis vulgaris, cystic fibrosis (mucoviscidosis), open wounds or chronic wounds can profit from treatment with the inventive pharmaceutical compositions, since these disorders are often additionally associated with infections by the abovementioned organisms, for example in biofilms.

The enumeration of these disorders in no way constitutes a restriction, but is merely of illustrative character.

The inventive peptidic actives have rapid efficacy.

Indication examples in human medicine may include, for example: dermatomycoses and systemic mycoses caused by Trichophyton rubrum, Trichophyton mentagrophytes and other Trichophyton species, Microsporum species and Epidermophyton floccosum, yeast-like fungi and biphasic fungi, and also molds.

Indication areas in animal medicine may include, for example: all dermatomycoses and systemic mycoses, especially those which are caused by the abovementioned pathogens.

The present invention includes pharmaceutical formulations which, as well as nontoxic inert pharmaceutically suitable carriers, comprise one or more inventive actives, or which consist of one or more inventive actives.

The present invention also includes pharmaceutical formulations in dosage units. This means that the formulations are in the form of individual parts, for example tablets, coated tablets, capsules, pills, suppositories and ampoules, the active content of which corresponds to a fraction or a multiple of a single dose. The dosage units may comprise, for example, 1, 2, 3 or 4 single doses or ½, ⅓ or ¼ of a single dose. A single dose preferably comprises the amount of active which is administered in one administration, and which usually corresponds to a whole daily dose, or to half or a third or a quarter of a daily dose.

Nontoxic inert pharmaceutically suitable carriers are understood to mean solid, semisolid or liquid diluents, fillers or formulation aids of any kind.

Preferred pharmaceutical formulations include tablets, coated tablets, capsules, pills, granules, suppositories, solutions, suspensions and emulsions, pastes, ointments, gels, creams, lotions, powder or sprays.

Tablets, coated tablets, capsules, pills and granules may comprise the active(s) as well as the customary carriers, such as (a) fillers and extenders, for example starches, milk sugar, cane sugar, glucose, mannitol and silica, (b) binders, for example carboxymethylcellulose, alginates, gelatins, polyvinylpyrrolidone, (c) humectants, for example glycerol, (d) disintegrants, for example agar-agar, calcium carbonate and sodium carbonate, (e) dissolution retardants, for example paraffin and (f) absorption accelerators, for example quaternary ammonium compounds, (g) wetting agents, for example cetyl alcohol, glyceryl monostearate, (h) adsorbents, for example kaolin and bentonite, and (i) lubricants, for example talc, calcium stearate and magnesium stearate and solid polyethylene glycols, or mixtures of the substances listed under (a) to (i).

The tablets, coated tablets, capsules, pills and granules may be provided with the customary coatings and shells optionally comprising opacifiers, and have such a composition that they release the active(s) only or preferentially within a particular part of the intestinal tract, optionally in a retarded manner, for which polymer substances and waxes, for example, can be used as embedding compositions.

The active(s) may also be in microencapsulated form, optionally with one or more of the carriers specified above.

Suppositories may, as well as the active(s), comprise the customary water-soluble or water-insoluble carriers, for example polyethylene glycols, fat, for example cocoa fat, and higher esters (e.g. C14 alcohol with C16 fatty acid) or mixtures of these substances.

Ointments, pastes, creams and gels may, as well as the active(s), comprise the customary carriers, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may, as well as the active(s), comprise the customary carriers, for example milk sugar, talc, silica, aluminum hydroxide, calcium silicate and polyamide powder, or mixtures of these substances; sprays may additionally comprise the customary propellants, for example hydrochlorofluorocarbons.

Solutions and emulsions may, as well as the active(s), comprise the customary carriers such as solvents, dissolution retardants and emulsifiers, for example water, ethyl alcohol, isopropyl alcohol, ethyl carbonate, ethyl acetate, benzyl alcohol, benzyl benzoate, propylene glycol, 1,3-butylene glycol, dimethylformamide, oils, especially cottonseed oil, peanut oil, maize kernel oil, olive oil, castor oil and sesame oil, glycerol, glycerol formal, tetrahydrofurfuryl alcohol, polyethylene glycols and fatty acid esters of sorbitan, or mixtures of these substances.

For parenteral administration, the solutions and emulsions may also be in sterile and isotonic form.

Suspensions may, as well as the active(s), comprise the customary carriers, such as liquid diluents, for example water, ethyl alcohol, propyl alcohol, suspension media, for example ethoxylated isostearyl alcohols, polyoxyethylene sorbitol and sorbitan esters, microcrystalline cellulose, aluminum metahydroxide, bentonite, agar-agar and tragacanth, or mixtures of these substances.

The formulation forms mentioned may also comprise colorants, preservatives and odor- and taste-improving additives, for example peppermint oil and eucalyptus oil, and sweeteners, for example saccharin.

The therapeutically active compounds should be present in the above-listed pharmaceutical formulations preferably in a concentration of about 0.0001 to 99.5% and preferably of 0.01 to 95% by weight of the overall mixture.

The pharmaceutical formulations detailed above may, apart from the inventive actives, also comprise further pharmaceutical actives.

Positive effects or even synergisms of the inventive peptidic actives are to be expected in combination with the following actives: cortisone, dithranol, zinc, vitamin C, folic acid, biotin, cyclosporin, voriconazole, clotrimazole, pentamidine, potassium iodide, essential oils, saturated fatty acids, capric acid, lauric acid, propolis, tea tree oil, eucalyptus oil, dolastatin 10, auristatin PHE, N-chlorotaurine, prostaglandin inhibitors such as aspirin, indomethacin, amphotericin B, candins, nikkomycins, azoles, allylamines, strobilurins, echinocandin, pneumocandin, pradimicin, benanomicin, oligopeptides, imidazoles, triazoles, polyenes, ciclopirox olamine, sordarins, atovaquone, 5-FC, griseofulvin, caspofungin, flucytosine, fluconazole, itraconazole, ciclopirox, terbinafine, griseofulvin, nystatin, urea, salicylic acid, hydrocortisone, prednisolone, fluocortin butyl ester, triamcinolone acetonide, dexamethasone, clocortolone pivalate, clobetasone butyrate, hydrocortisone aceponate, dexamethasone sulfobenzoate, alclomethasone dipropionate, flumethasone pivalate, triamcinolone acetonide, fluprednidene acetate, flurandrenolone, hydrocortisone butyrate, hydrocortisone buteprate, betamethasone benzoate, fluocortolone, mometasone furoate, betamethasone valerate, fluticasone propionate, halomethasone, betametasone dipropionate, fluocortolone hexanoate, fluocinolone acetonide, nonsteroidal antirheumatics, antiphlogistics, cytostatics, bufexamacum, comfrey root, bromelain, diclofenac, flurbiprofen, ibuprofen, evening primrose seed oil, paracetamol, phenazone, piroxicam, propyphenazone, salicylic acid, devil's claw root, chamomile, Hamamelis, marigold, St John's wort, tannins, zinc oxide, bismuth complexes, aluminum sulfate, sulfonated shale oils, aminoglycosides, chloramphenicol, cephalosporins, diaminobenzyl pyrimidines, phosphomycin, macrolides, penems, sulfonamides, tetracyclines, quinolones, ethambutol, fusidinic acid, glycopeptides, isonicotinamide, lincomycins, monobactams, nitrofurans, nitroimidazoles, oxalactams, paraminosalicylic acid, penicillins, polypeptides, polymyxin B, colistin, rifamycins, sulfones.

The pharmaceutical formulations detailed above are produced in a customary manner by known methods, for example by mixing the active(s) with the carrier(s).

The present invention also includes the use of the inventive actives, and of pharmaceutical formulations comprising one or more inventive actives, in human and veterinary medicine for prevention, improvement and/or healing of the disorders detailed above.

The actives or the pharmaceutical formulations can be administered in a local, oral, parenteral, intraperitoneal, intravenous and/or rectal manner, preferably in a topical local manner.

In general, it has been found to be advantageous both in human and in veterinary medicine to administer the inventive active(s) in total amounts of about 2.5 to about 200 and preferably 5 to 150 mg/kg of body weight per 24 hours, optionally in the form of several individual doses, to achieve the desired results.

In the case of oral administrations, the inventive actives are administered in total amounts of about 2.5 to about 200 and preferably 5 to 150 mg/kg of body weight per 24 hours and, in the case of parenteral administration, in total amounts of about 2.5 to about 50 and preferably 1 to 25 mg/kg of body weight per 24 hours.

It may, however, be necessary to depart from the dosages mentioned, specifically depending on the nature and the body weight of the object to be treated, the nature and severity of the disorder, the nature of the formulation and the administration of the medicament, and also the period or interval over which the administration is effected. For instance, less than the abovementioned amount of active may be sufficient in some cases, while the amount of active stated above must be exceeded in other cases. The optimal dosage and administration method of the actives required in each case can be determined easily by any person skilled in the art on the basis of his or her specialist knowledge.

Advantageously, the pharmaceutical composition comprises the peptide of SEQ ID NO: 1 or SEQ ID NO: 2 in a concentration of 0.0001-50% by weight, preferably 0.001-25% by weight, especially 0.01-5% by weight and more preferably 0.1-1% by weight, based on the total weight of the pharmaceutical composition.

The invention further relates to compositions comprising at least one peptide as defined above, which has a minimum inhibitory concentration with respect to Malassezia furfur in the range from about 1500 to 0.1 μM, for example 500 to 1 μM, 100 to 5 μM or 50 to 10 μM, determined under standard conditions. Standard conditions relate to the determination of the minimum inhibitory concentration of a Malassezia furfur culture which has an initial optical density of 0.02 at 600 nm, and, after incubation with the peptide which is present in the culture medium in this minimum concentration for 24 hours, has less than 1 colony forming unit (CFU) of the microorganism per μl of culture medium.

The present invention further relates to a process for producing a pharmaceutical composition as defined above, wherein a peptide as defined above is formulated to the desired administration form together with at least one customary pharmaceutical excipient and optionally further cosmetic or pharmaceutical actives.

P18 (SEQ ID NO: 3) is a peptide with a chain length of 18 amino acids, which derives from a fusion peptide of fragments of cecropin A from Hyalaphora cecropia and magainin from Xenopus laevis. Fungicidal activity has been found in experiments for Candida albicans, Trichosporon beigelii, Aspergillus flavus and Fusarium oxysporum (Lee et al., (2004) Biotechnology Letters, 26:337-341). Nevertheless, it is known to the person skilled in the art that the action of fungicidal substances can be very different on different organisms. Particularly the effect on the lipophilic fungus Malassezia furfur and the lipophilic species of the Malassezia genus can differ distinctly, for example, from the effect on Candida albicans (Hanson et al., (1989) Antimicrobial Agents and Chemotherapy, 33:1391-1392; Nenhoff et al., (2002) Acta Derm Venereol., 82:170-173). The effect observed in accordance with the invention for P18 and structurally and functionally related peptides of the type described herein is therefore completely surprising to the person skilled in the art. The same applies to the antibacterial effect. The person skilled in the art is aware that the effect of antibacterial substances on different organism can likewise be very different.

In a further particular embodiment, the secondary structure of the inventive peptides is a helix divided in the middle by a helix-breaking amino acid into two helices. In a representation as a “helical wheel”, hydrophobic amino acids predominate on one side (i.e. one half of the helix), especially leucine radicals, and positively charged amino acids on the other side, especially lysine radicals.

The inventive peptides are composed especially of D- and/or L-amino acids, especially L-amino acids.

Peptides and/or derivatives thereof described herein can be prepared in a manner per se, such as by chemical solid phase synthesis, liquid synthesis, or by biotechnological means using recombinant production strains or cell cultures.

### 3. Further Configurations of the Invention

### 3.1 Examples of Further Suitable Sequence Motifs

3.1.1 Sequence motif KX₂KX₃X₄X₅KIPX₁₁X₁₂KFLHX₈AKKF

| | | |
|---|---|---|
| variant_0 | KLKLLLKIPPPKFLHAAKKF | seqid_4612 |
| variant_1 | KWKLLLKIPPPKFLHAAKKF | seqid_2329 |
| variant_2 | KLKKLLKIPPPKFLHAAKKF | seqid_4613 |
| variant_3 | KWKKLLKIPPPKFLHAAKKF | seqid_2335 |
| variant_4 | KLKLFLKIPPPKFLHAAKKF | seqid_4614 |
| variant_5 | KWKLFLKIPPPKFLHAAKKF | seqid_2317 |
| variant_6 | [symbol] | [symbol] |
| variant_7 | [symbol] | [symbol] |
| variant_8 | [symbol] | [symbol] |
| variant_9 | KWKLLKKIPPPKFLHAAKKF | seqid_2353 |
| variant_10 | KLKKLKKIPPPKFLHAAKKF | seqid_4617 |
| variant_11 | KWKKLKKIPPPKFLHAAKKF | seqid_2359 |
| variant_12 | KLKLFKKIPPPKFLHAAKKF | seqid_4618 |
| variant_13 | KWKLFKKIPPPKFLHAAKKF | seqid_2341 |
| variant_14 | KLKKFKKIPPPKFLHAAKKF | seqid_4619 |
| variant_15 | KWKKFKKIPPPKFLHAAKKF | seqid_2347 |
| variant_16 | KLKLLLKIPPKFLHAAKKF | seqid_4620 |
| variant_17 | KWKLLLKIPPKFLHAAKKF | seqid_2377 |
| variant_18 | KLKKLLKIPPKFLHAAKKF | seqid_4621 |
| variant_19 | KWKKLLKIPPKFLHAAKKF | seqid_2383 |
| variant_20 | KLKLFLKIPPKFLHAAKKF | seqid_4622 |
| variant_21 | KWKLFLKIPPKFLHAAKKF | seqid_2365 |
| variant_22 | KLKKFLKIPPKFLHAAKKF | seqid_4623 |
| variant_23 | KWKKFLKIPPKFLHAAKKF | seqid_2371 |
| variant_24 | KLKLLKKIPPKFLHAAKKF | seqid_4624 |
| variant_25 | KWKLLKKIPPKFLHAAKKF | seqid_2401 |
| variant_26 | KLKKLKKIPPKFLHAAKKF | seqid_4625 |
| variant_27 | KWKKLKKIPPKFLHAAKKF | seqid_2407 |
| variant_28 | KLKLFKKIPPKFLHAAKKF | seqid_4626 |
| variant_29 | KWKLFKKIPPKFLHAAKKF | seqid_2389 |
| variant_30 | KLKKFKKIPPKFLHAAKKF | seqid_4627 |
| variant_31 | KWKKFKKIPPKFLHAAKKF | seqid_2395 |
| variant_48 | KLKLLLKIPKFLHAAKKF | seqid_4628 |
| variant_49 | KWKLLLKIPKFLHAAKKF | seqid_2424 |
| variant_50 | KLKKLLKIPKFLHAAKKF | seqid_4629 |
| variant_51 | KWKKLLKIPKFLHAAKKF | seqid_9 |
| variant_52 | KLKLFLKIPKFLHAAKKF | seqid_4630 |
| variant_53 | KWKLFLKIPKFLHAAKKF | seqid_2413 |
| variant_54 | KLKKFLKIPKFLHAAKKF | seqid_4631 |
| variant_55 | KWKKFLKIPKFLHAAKKF | seqid_8 |
| variant_56 | KLKLLKKIPKFLHAAKKF | seqid_4632 |
| variant_57 | KWKLLKKIPKFLHAAKKF | seqid_2445 |
| variant_58 | KLKKLKKIPKFLHAAKKF | seqid_4633 |
| variant_59 | KWKKLKKIPKFLHAAKKF | seqid_2451 |
| variant_60 | [symbol] | [symbol] 634 |
| variant_61 | [symbol] | [symbol] |
| variant_62 | [symbol] | [symbol] 635 |
| variant_63 | KWKKFKKIPKFLHAAKKF | seqid_2439 |
| variant_64 | KLKLLLKIPPPKFLHVAKKF | seqid_4636 |
| variant_65 | KWKLLLKIPPPKFLHVAKKF | seqid_3475 |
| variant_66 | KLKKLLKIPPPKFLHVAKKF | seqid_4637 |
| variant_67 | KWKKLLKIPPPKFLHVAKKF | seqid_3481 |
| variant_68 | KLKLFLKIPPPKFLHVAKKF | seqid_4638 |
| variant_69 | KWKLFLKIPPPKFLHVAKKF | seqid_3463 |
| variant_70 | KLKKFLKIPPPKFLHVAKKF | seqid_4639 |
| variant_71 | KWKKFLKIPPPKFLHVAKKF | seqid_3469 |
| variant_72 | KLKLLKKIPPPKFLHVAKKF | seqid_4640 |
| variant_73 | KWKLLKKIPPPKFLHVAKKF | seqid_3499 |
| variant_74 | KLKKLKKIPPPKFLHVAKKF | seqid_4641 |
| variant_75 | KWKKLKKIPPPKFLHVAKKF | seqid_3505 |
| variant_76 | KLKLFKKIPPPKFLHVAKKF | seqid_4642 |
| variant_77 | KWKLFKKIPPPKFLHVAKKF | seqid_3487 |
| variant_78 | KLKKFKKIPPPKFLHVAKKF | seqid_4643 |
| variant_79 | KWKKFKKIPPPKFLHVAKKF | seqid_3493 |
| variant_80 | KLKLLLKIPPKFLHVAKKF | seqid_4644 |
| variant_81 | KWKLLLKIPPKFLHVAKKF | seqid_3523 |
| variant_82 | KLKKLLKIPPKFLHVAKKF | seqid_4645 |
| variant_83 | KWKKLLKIPPKFLHVAKKF | seqid_3529 |
| variant_84 | KLKLFLKIPPKFLHVAKKF | seqid_4646 |
| variant_85 | KWKLFLKIPPKFLHVAKKF | seqid_3511 |
| variant_86 | KLKKFLKIPPKFLHVAKKF | seqid_4647 |
| variant_87 | KWKKFLKIPPKFLHVAKKF | seqid_3517 |
| variant_88 | KLKLLKKIPPKFLHVAKKF | seqid_4648 |
| variant_89 | KWKLLKKIPPKFLHVAKKF | seqid_3547 |
| variant_90 | KLKKLKKIPPKFLHVAKKF | seqid_4649 |
| variant_91 | KWKKLKKIPPKFLHVAKKF | seqid_3553 |
| variant_92 | KLKLFKKIPPKFLHVAKKF | seqid_4650 |
| variant_93 | KWKLFKKIPPKFLHVAKKF | seqid_3535 |
| variant_94 | KLKKFKKIPPKFLHVAKKF | seqid_4651 |
| variant_95 | KWKKFKKIPPKFLHVAKKF | seqid_3541 |
| variant_112 | KLKLLLKIPKFLHVAKKF | seqid_4652 |
| variant_113 | KWKLLLKIPKFLHVAKKF | seqid_3571 |
| variant_114 | [symbol] | [symbol] 4653 |
| variant_115 | [symbol] | [symbol] 77 |
| variant_116 | [symbol] | [symbol] 54 |
| variant_117 | KWKLFLKIPKFLHVAKKF | seqid_3559 |
| variant_118 | KLKKFLKIPKFLHVAKKF | seqid_4655 |
| variant_119 | KWKKFLKIPKFLHVAKKF | seqid_3565 |
| variant_120 | KLKLLKKIPKFLHVAKKF | seqid_4656 |
| variant_121 | KWKLLKKIPKFLHVAKKF | seqid_3595 |
| variant_122 | KLKKLKKIPKFLHVAKKF | seqid_4657 |
| variant_123 | KWKKLKKIPKFLHVAKKF | seqid_3601 |
| variant_124 | KLKLFKKIPKFLHVAKKF | seqid_4658 |
| variant_125 | KWKLFKKIPKFLHVAKKF | seqid_3583 |
| variant_126 | KLKKFKKIPKFLHVAKKF | seqid_4659 |
| variant_127 | KWKKFKKIPKFLHVAKKF | seqid_3589 |
| variant_128 | KLKLLLKIPPPKFLHLAKKF | seqid_4660 |
| variant_129 | KWKLLLKIPPPKFLHLAKKF | seqid_2901 |
| variant_130 | KLKKLLKIPPPKFLHLAKKF | seqid_4661 |
| variant_131 | KWKKLLKIPPPKFLHLAKKF | seqid_2907 |
| variant_132 | KLKLFLKIPPPKFLHLAKKF | seqid_4662 |
| variant_133 | KWKLFLKIPPPKFLHLAKKF | seqid_2889 |
| variant_134 | KLKKFLKIPPPKFLHLAKKF | seqid_4663 |
| variant_135 | KWKKFLKIPPPKFLHLAKKF | seqid_2895 |
| variant_136 | KLKLLKKIPPPKFLHLAKKF | seqid_4664 |
| variant_137 | KWKLLKKIPPPKFLHLAKKF | seqid_2925 |
| variant_138 | KLKKLKKIPPPKFLHLAKKF | seqid_4665 |
| variant_139 | KWKKLKKIPPPKFLHLAKKF | seqid_2931 |
| variant_140 | KLKLFKKIPPPKFLHLAKKF | seqid_4666 |
| variant_141 | KWKLFKKIPPPKFLHLAKKF | seqid_2913 |
| variant_142 | KLKKFKKIPPPKFLHLAKKF | seqid_4667 |
| variant_143 | KWKKFKKIPPPKFLHLAKKF | seqid_2919 |
| variant_144 | KLKLLLKIPPKFLHLAKKF | seqid_4668 |
| variant_145 | KWKLLLKIPPKFLHLAKKF | seqid_2949 |
| variant_146 | KLKKLLKIPPKFLHLAKKF | seqid_4669 |
| variant_147 | KWKKLLKIPPKFLHLAKKF | seqid_2955 |
| variant_148 | KLKLFLKIPPKFLHLAKKF | seqid_4670 |
| variant_149 | KWKLFLKIPPKFLHLAKKF | seqid_2937 |
| variant_150 | KLKKFLKIPPKFLHLAKKF | seqid_4671 |
| variant_151 | KWKKFLKIPPKFLHLAKKF | seqid_2943 |
| variant_152 | [symbol] | [symbol] 4672 |
| variant_153 | [symbol] | [symbol] 2973 |
| variant_154 | [symbol] | [symbol] 4673 |
| variant_155 | KWKKLKKIPPKFLHLAKKF | seqid_2979 |
| variant_156 | KLKLFKKIPPKFLHLAKKF | seqid_4674 |
| variant_157 | KWKLFKKIPPKFLHLAKKF | seqid_2961 |
| variant_158 | KLKKFKKIPPKFLHLAKKF | seqid_4675 |
| variant_159 | KWKKFKKIPPKFLHLAKKF | seqid_2967 |
| variant_176 | KLKLLLKIPKFLHLAKKF | seqid_4676 |
| variant_177 | KWKLLLKIPKFLHLAKKF | seqid_2997 |
| variant_178 | KLKKLLKIPKFLHLAKKF | seqid_4677 |
| variant_179 | KWKKLLKIPKFLHLAKKF | seqid_3003 |
| variant_180 | KLKLFLKIPKFLHLAKKF | seqid_4678 |
| variant_181 | KWKLFLKIPKFLHLAKKF | seqid_2985 |
| variant_182 | KLKKFLKIPKFLHLAKKF | seqid_4679 |
| variant_183 | KWKKFLKIPKFLHLAKKF | seqid_2991 |
| variant_184 | KLKLLKKIPKFLHLAKKF | seqid_4680 |
| variant_185 | KWKLLKKIPKFLHLAKKF | seqid_3019 |
| variant_186 | KLKKLKKIPKFLHLAKKF | seqid_4681 |
| variant_187 | KWKKLKKIPKFLHLAKKF | seqid_3025 |
| variant_188 | KLKLFKKIPKFLHLAKKF | seqid_4682 |
| variant_189 | KWKLFKKIPKFLHLAKKF | seqid_3 |
| variant_190 | KLKKFKKIPKFLHLAKKF | seqid_4683 |
| variant_191 | KWKKFKKIPKFLHLAKKF | seqid_3013 |
| variant_192 | KLKLLLKIPPPKFLHSAKKF | seqid_4684 |
| variant_193 | KWKLLLKIPPPKFLHSAKKF | seqid_4051 |
| variant_194 | KLKKLLKIPPPKFLHSAKKF | seqid_4685 |
| variant_195 | KWKKLLKIPPPKFLHSAKKF | seqid_4057 |
| variant_196 | KLKLFLKIPPPKFLHSAKKF | seqid_4686 |
| variant_197 | KWKLFLKIPPPKFLHSAKKF | seqid_4039 |
| variant_198 | KLKKFLKIPPPKFLHSAKKF | seqid_4687 |
| variant_199 | KWKKFLKIPPPKFLHSAKKF | seqid_4045 |
| variant_200 | KLKLLKKIPPPKFLHSAKKF | seqid_4688 |
| variant_201 | KWKLLKKIPPPKFLHSAKKF | seqid_4075 |
| variant_202 | KLKKLKKIPPPKFLHSAKKF | seqid_4689 |
| variant_203 | KWKKLKKIPPPKFLHSAKKF | seqid_4081 |
| variant_204 | KLKLFKKIPPPKFLHSAKKF | seqid_4690 |
| variant_205 | KWKLFKKIPPPKFLHSAKKF | seqid_4063 |
| variant_206 | [symbol] | [symbol] id_4691 |
| variant_207 | [symbol] | [symbol] id_4069 |
| variant_208 | [symbol] | [symbol] id_4692 |
| variant_209 | KWKLLLKIPPKFLHSAKKF | seqid_4099 |
| variant_210 | KLKKLLKIPPKFLHSAKKF | seqid_4693 |
| variant_211 | KWKKLLKIPPKFLHSAKKF | seqid_4105 |
| variant_212 | KLKLFLKIPPKFLHSAKKF | seqid_4694 |
| variant_213 | KWKLFLKIPPKFLHSAKKF | seqid_4087 |
| variant_214 | KLKKFLKIPPKFLHSAKKF | seqid_4695 |
| variant_215 | KWKKFLKIPPKFLHSAKKF | seqid_4093 |
| variant_216 | KLKLLKKIPPKFLHSAKKF | seqid_4696 |
| variant_217 | KWKLLKKIPPKFLHSAKKF | seqid_4123 |
| variant_218 | KLKKLKKIPPKFLHSAKKF | seqid_4697 |
| variant_219 | KWKKLKKIPPKFLHSAKKF | seqid_4129 |
| variant_220 | KLKLFKKIPPKFLHSAKKF | seqid_4698 |
| variant_221 | KWKLFKKIPPKFLHSAKKF | seqid_4111 |
| variant_222 | KLKKFKKIPPKFLHSAKKF | seqid_4699 |
| variant_223 | KWKKFKKIPPKFLHSAKKF | seqid_4117 |
| variant_240 | KLKLLLKIPKFLHSAKKF | seqid_4700 |
| variant_241 | KWKLLLKIPKFLHSAKKF | seqid_4147 |
| variant_242 | KLKKLLKIPKFLHSAKKF | seqid_4701 |
| variant_243 | KWKKLLKIPKFLHSAKKF | seqid_4153 |
| variant_244 | KLKLFLKIPKFLHSAKKF | seqid_4702 |
| variant_245 | KWKLFLKIPKFLHSAKKF | seqid_4135 |
| variant_246 | KLKKFLKIPKFLHSAKKF | seqid_4703 |
| variant_247 | KWKKFLKIPKFLHSAKKF | seqid_4141 |
| variant_248 | KLKLLKKIPKFLHSAKKF | seqid_4704 |
| variant_249 | KWKLLKKIPKFLHSAKKF | seqid_4171 |
| variant_250 | KLKKLKKIPKFLHSAKKF | seqid_4705 |
| variant_251 | KWKKLKKIPKFLHSAKKF | seqid_4177 |
| variant_252 | KLKLFKKIPKFLHSAKKF | seqid_4706 |
| variant_253 | KWKLFKKIPKFLHSAKKF | seqid_4708 |
| variant_254 | KLKKFKKIPKFLHSAKKF | seqid_4707 |
| variant_255 | KWKKFKKIPKFLHSAKKF | seqid_4165 |

[symbol]

indicates data missing or illegible when filed

3.1.2 Sequence Motif X₁X₂KX₃X₄X₅KIPX₁₁X₁₂KFX₆X₇X₈AX₉KF (SEQ ID NO: 2)

| | | |
|---|---|---|
| variant_0 | KKKLFLKIPPPKFLHAALKF | seqid_11 |
| variant_1 | RKKLFLKIPPPKFLHAALKF | seqid_12 |
| variant_2 | FKKLFLKIPPPKFLHAALKF | seqid_13 |
| variant_3 | KWKLFLKIPPPKFLHAALKF | seqid_14 |
| variant_4 | RWKLFLKIPPPKFLHAALKF | seqid_15 |
| variant_5 | FWKLFLKIPPPKFLHAALKF | seqid_16 |
| variant_6 | KKKKFLKIPPPKFLHAALKF | seqid_17 |
| variant_7 | RKKKFLKIPPPKFLHAALKF | seqid_18 |
| variant_8 | FKKKFLKIPPPKFLHAALKF | seqid_19 |
| variant_9 | KWKKFLKIPPPKFLHAALKF | seqid_20 |
| variant_10 | RWKKFLKIPPPKFLHAALKF | seqid_21 |
| variant_11 | FWKKFLKIPPPKFLHAALKF | seqid_22 |
| variant_12 | KKKLLLKIPPPKFLHAALKF | seqid_23 |
| variant_13 | RKKLLLKIPPPKFLHAALKF | seqid_24 |
| variant_14 | FKKLLLKIPPPKFLHAALKF | seqid_25 |
| variant_15 | KWKLLLKIPPPKFLHAALKF | seqid_26 |
| variant_16 | RWKLLLKIPPPKFLHAALKF | seqid_27 |
| variant_17 | FWKLLLKIPPPKFLHAALKF | seqid_28 |
| variant_18 | KKKKLLKIPPPKFLHAALKF | seqid_29 |
| variant_19 | RKKKLLKIPPPKFLHAALKF | seqid_30 |
| variant_20 | FKKKLLKIPPPKFLHAALKF | seqid_31 |
| variant_21 | KWKKLLKIPPPKFLHAALKF | seqid_32 |
| variant_22 | RWKKLLKIPPPKFLHAALKF | seqid_33 |
| variant_23 | FWKKLLKIPPPKFLHAALKF | seqid_34 |
| variant_24 | KKKLFKKIPPPKFLHAALKF | seqid_35 |
| variant_25 | RKKLFKKIPPPKFLHAALKF | seqid_36 |
| variant_26 | FKKLFKKIPPPKFLHAALKF | seqid_37 |
| variant_27 | KWKLFKKIPPPKFLHAALKF | seqid_38 |
| variant_28 | RWKLFKKIPPPKFLHAALKF | seqid_39 |
| variant_29 | FWKLFKKIPPPKFLHAALKF | seqid_40 |
| variant_30 | KKKKFKKIPPPKFLHAALKF | seqid_41 |
| variant_31 | RKKKFKKIPPPKFLHAALKF | seqid_42 |
| variant_32 | FKKKFKKIPPPKFLHAALKF | seqid_43 |
| variant_33 | KWKKFKKIPPPKFLHAALKF | seqid_44 |
| variant_34 | RWKKFKKIPPPKFLHAALKF | seqid_45 |
| variant_35 | FWKKFKKIPPPKFLHAALKF | seqid_46 |
| variant_36 | KKKLLKKIPPPKFLHAALKF | seqid_47 |
| variant_37 | RKKLLKKIPPPKFLHAALKF | seqid_48 |
| variant_38 | FKKLLKKIPPPKFLHAALKF | seqid_49 |
| variant_39 | KWKLLKKIPPPKFLHAALKF | seqid_50 |
| variant_40 | RWKLLKKIPPPKFLHAALKF | seqid_51 |
| variant_41 | FWKLLKKIPPPKFLHAALKF | seqid_52 |
| variant_42 | KKKKLKKIPPPKFLHAALKF | seqid_53 |
| variant_43 | RKKKLKKIPPPKFLHAALKF | seqid_54 |
| variant_44 | FKKKLKKIPPPKFLHAALKF | seqid_55 |
| variant_45 | KWKKLKKIPPPKFLHAALKF | seqid_56 |
| variant_46 | RWKKLKKIPPPKFLHAALKF | seqid_57 |
| variant_47 | FWKKLKKIPPPKFLHAALKF | seqid_58 |
| variant_48 | KKKLFLKIPPKFLHAALKF | seqid_59 |
| variant_49 | RKKLFLKIPPKFLHAALKF | seqid_60 |
| variant_50 | FKKLFLKIPPKFLHAALKF | seqid_61 |
| variant_51 | KWKLFLKIPPKFLHAALKF | seqid_62 |
| variant_52 | RWKLFLKIPPKFLHAALKF | seqid_63 |
| variant_53 | FWKLFLKIPPKFLHAALKF | seqid_64 |
| variant_54 | KKKKFLKIPPKFLHAALKF | seqid_65 |
| variant_55 | RKKKFLKIPPKFLHAALKF | seqid_66 |
| variant_56 | FKKKFLKIPPKFLHAALKF | seqid_67 |
| variant_57 | KWKKFLKIPPKFLHAALKF | seqid_68 |
| variant_58 | RWKKFLKIPPKFLHAALKF | seqid_69 |
| variant_59 | FWKKFLKIPPKFLHAALKF | seqid_70 |
| variant_60 | KKKLLLKIPPKFLHAALKF | seqid_71 |
| variant_61 | RKKLLLKIPPKFLHAALKF | seqid_72 |
| variant_62 | FKKLLLKIPPKFLHAALKF | seqid_73 |
| variant_63 | KWKLLLKIPPKFLHAALKF | seqid_74 |
| variant_64 | RWKLLLKIPPKFLHAALKF | seqid_75 |
| variant_65 | FWKLLLKIPPKFLHAALKF | seqid_76 |
| variant_66 | KKKKLLKIPPKFLHAALKF | seqid_77 |
| variant_67 | RKKKLLKIPPKFLHAALKF | seqid_78 |
| variant_68 | FKKKLLKIPPKFLHAALKF | seqid_79 |
| variant_69 | KWKKLLKIPPKFLHAALKF | seqid_80 |
| variant_70 | RWKKLLKIPPKFLHAALKF | seqid_81 |
| variant_71 | FWKKLLKIPPKFLHAALKF | seqid_82 |
| variant_72 | KKKLFKKIPPKFLHAALKF | seqid_83 |
| variant_73 | RKKLFKKIPPKFLHAALKF | seqid_84 |
| variant_74 | FKKLFKKIPPKFLHAALKF | seqid_85 |
| variant_75 | KWKLFKKIPPKFLHAALKF | seqid_86 |
| variant_76 | RWKLFKKIPPKFLHAALKF | seqid_87 |
| variant_77 | FWKLFKKIPPKFLHAALKF | seqid_88 |
| variant_78 | KKKKFKKIPPKFLHAALKF | seqid_89 |
| variant_79 | RKKKFKKIPPKFLHAALKF | seqid_90 |
| variant_80 | FKKKFKKIPPKFLHAALKF | seqid_91 |
| variant_81 | KWKKFKKIPPKFLHAALKF | seqid_92 |
| variant_82 | RWKKFKKIPPKFLHAALKF | seqid_93 |
| variant_83 | FWKKFKKIPPKFLHAALKF | seqid_94 |
| variant_84 | KKKLLKKIPPKFLHAALKF | seqid_95 |
| variant_85 | RKKLLKKIPPKFLHAALKF | seqid_96 |
| variant_86 | FKKLLKKIPPKFLHAALKF | seqid_97 |
| variant_87 | KWKLLKKIPPKFLHAALKF | seqid_98 |
| variant_88 | RWKLLKKIPPKFLHAALKF | seqid_99 |
| variant_89 | FWKLLKKIPPKFLHAALKF | seqid_100 |
| variant_90 | KKKKLKKIPPKFLHAALKF | seqid_101 |
| variant_91 | RKKKLKKIPPKFLHAALKF | seqid_102 |
| variant_92 | FKKKLKKIPPKFLHAALKF | seqid_103 |
| variant_93 | KWKKLKKIPPKFLHAALKF | seqid_104 |
| variant_94 | RWKKLKKIPPKFLHAALKF | seqid_105 |
| variant_95 | FWKKLKKIPPKFLHAALKF | seqid_106 |
| variant_144 | KKKLFLKIPKFLHAALKF | seqid_107 |
| variant_145 | RKKLFLKIPKFLHAALKF | seqid_108 |
| variant_146 | FKKLFLKIPKFLHAALKF | seqid_109 |
| variant_147 | KWKLFLKIPKFLHAALKF | seqid_110 |
| variant_148 | RWKLFLKIPKFLHAALKF | seqid_111 |
| variant_149 | FWKLFLKIPKFLHAALKF | seqid_112 |
| variant_150 | KKKKFLKIPKFLHAALKF | seqid_113 |
| variant_151 | RKKKFLKIPKFLHAALKF | seqid_114 |
| variant_152 | FKKKFLKIPKFLHAALKF | seqid_115 |
| variant_153 | KWKKFLKIPKFLHAALKF | seqid_116 |
| variant_154 | RWKKFLKIPKFLHAALKF | seqid_117 |
| variant_155 | FWKKFLKIPKFLHAALKF | seqid_118 |
| variant_156 | KKKLLLKIPKFLHAALKF | seqid_119 |
| variant_157 | RKKLLLKIPKFLHAALKF | seqid_120 |
| variant_158 | FKKLLLKIPKFLHAALKF | seqid_121 |
| variant_159 | KWKLLLKIPKFLHAALKF | seqid_122 |
| variant_160 | RWKLLLKIPKFLHAALKF | seqid_123 |
| variant_161 | FWKLLLKIPKFLHAALKF | seqid_124 |
| variant_162 | KKKKLLKIPKFLHAALKF | seqid_125 |
| variant_163 | RKKKLLKIPKFLHAALKF | seqid_126 |
| variant_164 | FKKKLLKIPKFLHAALKF | seqid_127 |
| variant_165 | KWKKLLKIPKFLHAALKF | seqid_128 |
| variant_166 | RWKKLLKIPKFLHAALKF | seqid_129 |
| variant_167 | FWKKLLKIPKFLHAALKF | seqid_130 |
| variant_168 | KKKLFKKIPKFLHAALKF | seqid_131 |
| variant_169 | RKKLFKKIPKFLHAALKF | seqid_132 |
| variant_170 | FKKLFKKIPKFLHAALKF | seqid_133 |
| variant_171 | KWKLFKKIPKFLHAALKF | seqid_134 |
| variant_172 | RWKLFKKIPKFLHAALKF | seqid_135 |
| variant_173 | FWKLFKKIPKFLHAALKF | seqid_136 |
| variant_174 | KKKKFKKIPKFLHAALKF | seqid_137 |
| variant_175 | RKKKFKKIPKFLHAALKF | seqid_138 |
| variant_176 | FKKKFKKIPKFLHAALKF | seqid_139 |
| variant_177 | KWKKFKKIPKFLHAALKF | seqid_140 |
| variant_178 | RWKKFKKIPKFLHAALKF | seqid_141 |
| variant_179 | FWKKFKKIPKFLHAALKF | seqid_142 |
| variant_180 | KKKLLKKIPKFLHAALKF | seqid_143 |
| variant_181 | RKKLLKKIPKFLHAALKF | seqid_144 |
| variant_182 | FKKLLKKIPKFLHAALKF | seqid_145 |
| variant_183 | KWKLLKKIPKFLHAALKF | seqid_146 |
| variant_184 | RWKLLKKIPKFLHAALKF | seqid_147 |
| variant_185 | FWKLLKKIPKFLHAALKF | seqid_148 |
| variant_186 | KKKKLKKIPKFLHAALKF | seqid_149 |
| variant_187 | RKKKLKKIPKFLHAALKF | seqid_150 |
| variant_188 | FKKKLKKIPKFLHAALKF | seqid_151 |
| variant_189 | KWKKLKKIPKFLHAALKF | seqid_152 |
| variant_190 | RWKKLKKIPKFLHAALKF | seqid_153 |
| variant_191 | FWKKLKKIPKFLHAALKF | seqid_154 |
| variant_192 | KKKLFLKIPPPKFKHAALKF | seqid_155 |
| variant_193 | RKKLFLKIPPPKFKHAALKF | seqid_156 |
| variant_194 | FKKLFLKIPPPKFKHAALKF | seqid_157 |
| variant_195 | KWKLFLKIPPPKFKHAALKF | seqid_158 |
| variant_196 | RWKLFLKIPPPKFKHAALKF | seqid_159 |
| variant_197 | FWKLFLKIPPPKFKHAALKF | seqid_160 |
| variant_198 | KKKKFLKIPPPKFKHAALKF | seqid_161 |
| variant_199 | RKKKFLKIPPPKFKHAALKF | seqid_162 |
| variant_200 | FKKKFLKIPPPKFKHAALKF | seqid_163 |
| variant_201 | KWKKFLKIPPPKFKHAALKF | seqid_164 |
| variant_202 | RWKKFLKIPPPKFKHAALKF | seqid_165 |
| variant_203 | FWKKFLKIPPPKFKHAALKF | seqid_166 |
| variant_204 | KKKLLLKIPPPKFKHAALKF | seqid_167 |
| variant_205 | RKKLLLKIPPPKFKHAALKF | seqid_168 |
| variant_206 | FKKLLLKIPPPKFKHAALKF | seqid_169 |
| variant_207 | KWKLLLKIPPPKFKHAALKF | seqid_170 |
| variant_208 | RWKLLLKIPPPKFKHAALKF | seqid_171 |
| variant_209 | FWKLLLKIPPPKFKHAALKF | seqid_172 |
| variant_210 | KKKKLLKIPPPKFKHAALKF | seqid_173 |
| variant_211 | RKKKLLKIPPPKFKHAALKF | seqid_174 |
| variant_212 | FKKKLLKIPPPKFKHAALKF | seqid_175 |
| variant_213 | KWKKLLKIPPPKFKHAALKF | seqid_176 |
| variant_214 | RWKKLLKIPPPKFKHAALKF | seqid_177 |
| variant_215 | FWKKLLKIPPPKFKHAALKF | seqid_178 |
| variant_216 | KKKLFKKIPPPKFKHAALKF | seqid_179 |
| variant_217 | RKKLFKKIPPPKFKHAALKF | seqid_180 |
| variant_218 | FKKLFKKIPPPKFKHAALKF | seqid_181 |
| variant_219 | KWKLFKKIPPPKFKHAALKF | seqid_182 |
| variant_220 | RWKLFKKIPPPKFKHAALKF | seqid_183 |
| variant_221 | FWKLFKKIPPPKFKHAALKF | seqid_184 |
| variant_222 | KKKKFKKIPPPKFKHAALKF | seqid_185 |
| variant_223 | RKKKFKKIPPPKFKHAALKF | seqid_186 |
| variant_224 | FKKKFKKIPPPKFKHAALKF | seqid_187 |
| variant_225 | KWKKFKKIPPPKFKHAALKF | seqid_188 |
| variant_226 | RWKKFKKIPPPKFKHAALKF | seqid_189 |
| variant_227 | FWKKFKKIPPPKFKHAALKF | seqid_190 |
| variant_228 | KKKLLKKIPPPKFKHAALKF | seqid_191 |
| variant_229 | RKKLLKKIPPPKFKHAALKF | seqid_192 |
| variant_230 | FKKLLKKIPPPKFKHAALKF | seqid_193 |
| variant_231 | KWKLLKKIPPPKFKHAALKF | seqid_194 |
| variant_232 | RWKLLKKIPPPKFKHAALKF | seqid_195 |
| variant_233 | FWKLLKKIPPPKFKHAALKF | seqid_196 |
| variant_234 | KKKKLKKIPPPKFKHAALKF | seqid_197 |
| variant_235 | RKKKLKKIPPPKFKHAALKF | seqid_198 |
| variant_236 | FKKKLKKIPPPKFKHAALKF | seqid_199 |
| variant_237 | KWKKLKKIPPPKFKHAALKF | seqid_200 |
| variant_238 | RWKKLKKIPPPKFKHAALKF | seqid_201 |
| variant_239 | FWKKLKKIPPPKFKHAALKF | seqid_202 |
| variant_240 | KKKLFLKIPPKFKHAALKF | seqid_203 |
| variant_241 | RKKLFLKIPPKFKHAALKF | seqid_204 |
| variant_242 | FKKLFLKIPPKFKHAALKF | seqid_205 |
| variant_243 | KWKLFLKIPPKFKHAALKF | seqid_206 |
| variant_244 | RWKLFLKIPPKFKHAALKF | seqid_207 |
| variant_245 | FWKLFLKIPPKFKHAALKF | seqid_208 |
| variant_246 | KKKKFLKIPPKFKHAALKF | seqid_209 |
| variant_247 | RKKKFLKIPPKFKHAALKF | seqid_210 |
| variant_248 | FKKKFLKIPPKFKHAALKF | seqid_211 |
| variant_249 | KWKKFLKIPPKFKHAALKF | seqid_212 |
| variant_250 | RWKKFLKIPPKFKHAALKF | seqid_213 |
| variant_251 | FWKKFLKIPPKFKHAALKF | seqid_214 |
| variant_252 | KKKLLLKIPPKFKHAALKF | seqid_215 |
| variant_253 | RKKLLLKIPPKFKHAALKF | seqid_216 |
| variant_254 | FKKLLLKIPPKFKHAALKF | seqid_217 |
| variant_255 | KWKLLLKIPPKFKHAALKF | seqid_218 |
| variant_256 | RWKLLLKIPPKFKHAALKF | seqid_219 |
| variant_257 | FWKLLLKIPPKFKHAALKF | seqid_220 |
| variant_258 | KKKKLLKIPPKFKHAALKF | seqid_221 |
| variant_259 | RKKKLLKIPPKFKHAALKF | seqid_222 |
| variant_260 | FKKKLLKIPPKFKHAALKF | seqid_223 |
| variant_261 | KWKKLLKIPPKFKHAALKF | seqid_224 |
| variant_262 | RWKKLLKIPPKFKHAALKF | seqid_225 |
| variant_263 | FWKKLLKIPPKFKHAALKF | seqid_226 |
| variant_264 | KKKLFKKIPPKFKHAALKF | seqid_227 |
| variant_265 | RKKLFKKIPPKFKHAALKF | seqid_228 |
| variant_266 | FKKLFKKIPPKFKHAALKF | seqid_229 |
| variant_267 | KWKLFKKIPPKFKHAALKF | seqid_230 |
| variant_268 | RWKLFKKIPPKFKHAALKF | seqid_231 |
| variant_269 | FWKLFKKIPPKFKHAALKF | seqid_232 |
| variant_270 | KKKKFKKIPPKFKHAALKF | seqid_233 |
| variant_271 | RKKKFKKIPPKFKHAALKF | seqid_234 |
| variant_272 | FKKKFKKIPPKFKHAALKF | seqid_235 |
| variant_273 | KWKKFKKIPPKFKHAALKF | seqid_236 |
| variant_274 | RWKKFKKIPPKFKHAALKF | seqid_237 |
| variant_275 | FWKKFKKIPPKFKHAALKF | seqid_238 |
| variant_276 | KKKLLKKIPPKFKHAALKF | seqid_239 |
| variant_277 | RKKLLKKIPPKFKHAALKF | seqid_240 |
| variant_278 | FKKLLKKIPPKFKHAALKF | seqid_241 |
| variant_279 | KWKLLKKIPPKFKHAALKF | seqid_242 |
| variant_280 | RWKLLKKIPPKFKHAALKF | seqid_243 |
| variant_281 | FWKLLKKIPPKFKHAALKF | seqid_244 |
| variant_282 | KKKKLKKIPPKFKHAALKF | seqid_245 |
| variant_283 | RKKKLKKIPPKFKHAALKF | seqid_246 |
| variant_284 | FKKKLKKIPPKFKHAALKF | seqid_247 |
| variant_285 | KWKKLKKIPPKFKHAALKF | seqid_248 |
| variant_286 | RWKKLKKIPPKFKHAALKF | seqid_249 |
| variant_287 | FWKKLKKIPPKFKHAALKF | seqid_250 |
| variant_336 | KKKLFLKIPKFKHAALKF | seqid_251 |
| variant_337 | RKKLFLKIPKFKHAALKF | seqid_252 |
| variant_338 | FKKLFLKIPKFKHAALKF | seqid_253 |
| variant_339 | KWKLFLKIPKFKHAALKF | seqid_254 |
| variant_340 | RWKLFLKIPKFKHAALKF | seqid_255 |
| variant_341 | FWKLFLKIPKFKHAALKF | seqid_256 |
| variant_342 | KKKKFLKIPKFKHAALKF | seqid_257 |
| variant_343 | RKKKFLKIPKFKHAALKF | seqid_258 |
| variant_344 | FKKKFLKIPKFKHAALKF | seqid_259 |
| variant_345 | KWKKFLKIPKFKHAALKF | seqid_260 |
| variant_346 | RWKKFLKIPKFKHAALKF | seqid_261 |
| variant_347 | FWKKFLKIPKFKHAALKF | seqid_262 |
| variant_348 | KKKLLLKIPKFKHAALKF | seqid_263 |
| variant_349 | RKKLLLKIPKFKHAALKF | seqid_264 |
| variant_350 | FKKLLLKIPKFKHAALKF | seqid_265 |
| variant_351 | KWKLLLKIPKFKHAALKF | seqid_266 |
| variant_352 | RWKLLLKIPKFKHAALKF | seqid_267 |
| variant_353 | FWKLLLKIPKFKHAALKF | seqid_268 |
| variant_354 | KKKKLLKIPKFKHAALKF | seqid_269 |
| variant_355 | RKKKLLKIPKFKHAALKF | seqid_270 |
| variant_356 | FKKKLLKIPKFKHAALKF | seqid_271 |
| variant_357 | KWKKLLKIPKFKHAALKF | seqid_272 |
| variant_358 | RWKKLLKIPKFKHAALKF | seqid_273 |
| variant_359 | FWKKLLKIPKFKHAALKF | seqid_274 |
| variant_360 | KKKLFKKIPKFKHAALKF | seqid_275 |
| variant_361 | RKKLFKKIPKFKHAALKF | seqid_276 |
| variant_362 | FKKLFKKIPKFKHAALKF | seqid_277 |
| variant_363 | KWKLFKKIPKFKHAALKF | seqid_278 |
| variant_364 | RWKLFKKIPKFKHAALKF | seqid_279 |
| variant_365 | FWKLFKKIPKFKHAALKF | seqid_280 |
| variant_366 | KKKKFKKIPKFKHAALKF | seqid_281 |
| variant_367 | RKKKFKKIPKFKHAALKF | seqid_282 |
| variant_368 | FKKKFKKIPKFKHAALKF | seqid_283 |
| variant_369 | KWKKFKKIPKFKHAALKF | seqid_284 |
| variant_370 | RWKKFKKIPKFKHAALKF | seqid_285 |
| variant_371 | FWKKFKKIPKFKHAALKF | seqid_286 |
| variant_372 | KKKLLKKIPKFKHAALKF | seqid_287 |
| variant_373 | RKKLLKKIPKFKHAALKF | seqid_288 |
| variant_374 | FKKLLKKIPKFKHAALKF | seqid_289 |
| variant_375 | KWKLLKKIPKFKHAALKF | seqid_290 |
| variant_376 | RWKLLKKIPKFKHAALKF | seqid_291 |
| variant_377 | FWKLLKKIPKFKHAALKF | seqid_292 |
| variant_378 | KKKKLKKIPKFKHAALKF | seqid_293 |
| variant_379 | RKKKLKKIPKFKHAALKF | seqid_294 |
| variant_380 | FKKKLKKIPKFKHAALKF | seqid_295 |
| variant_381 | KWKKLKKIPKFKHAALKF | seqid_296 |
| variant_382 | RWKKLKKIPKFKHAALKF | seqid_297 |
| variant_383 | FWKKLKKIPKFKHAALKF | seqid_298 |
| variant_384 | KKKLFLKIPPPKFLKAALKF | seqid_299 |
| variant_385 | RKKLFLKIPPPKFLKAALKF | seqid_300 |
| variant_386 | FKKLFLKIPPPKFLKAALKF | seqid_301 |
| variant_387 | KWKLFLKIPPPKFLKAALKF | seqid_302 |
| variant_388 | RWKLFLKIPPPKFLKAALKF | seqid_303 |
| variant_389 | FWKLFLKIPPPKFLKAALKF | seqid_304 |
| variant_390 | KKKKFLKIPPPKFLKAALKF | seqid_305 |
| variant_391 | RKKKFLKIPPPKFLKAALKF | seqid_306 |
| variant_392 | FKKKFLKIPPPKFLKAALKF | seqid_307 |
| variant_393 | KWKKFLKIPPPKFLKAALKF | seqid_308 |
| variant_394 | RWKKFLKIPPPKFLKAALKF | seqid_309 |
| variant_395 | FWKKFLKIPPPKFLKAALKF | seqid_310 |
| variant_396 | KKKLLLKIPPPKFLKAALKF | seqid_311 |
| variant_397 | RKKLLLKIPPPKFLKAALKF | seqid_312 |
| variant_398 | FKKLLLKIPPPKFLKAALKF | seqid_313 |
| variant_399 | KWKLLLKIPPPKFLKAALKF | seqid_314 |
| variant_400 | RWKLLLKIPPPKFLKAALKF | seqid_315 |
| variant_401 | FWKLLLKIPPPKFLKAALKF | seqid_316 |
| variant_402 | KKKKLLKIPPPKFLKAALKF | seqid_317 |
| variant_403 | RKKKLLKIPPPKFLKAALKF | seqid_318 |
| variant_404 | FKKKLLKIPPPKFLKAALKF | seqid_319 |
| variant_405 | KWKKLLKIPPPKFLKAALKF | seqid_320 |
| variant_406 | RWKKLLKIPPPKFLKAALKF | seqid_321 |
| variant_407 | FWKKLLKIPPPKFLKAALKF | seqid_322 |
| variant_408 | KKKLFKKIPPPKFLKAALKF | seqid_323 |
| variant_409 | RKKLFKKIPPPKFLKAALKF | seqid_324 |
| variant_410 | FKKLFKKIPPPKFLKAALKF | seqid_325 |
| variant_411 | KWKLFKKIPPPKFLKAALKF | seqid_326 |
| variant_412 | RWKLFKKIPPPKFLKAALKF | seqid_327 |
| variant_413 | FWKLFKKIPPPKFLKAALKF | seqid_328 |
| variant_414 | KKKKFKKIPPPKFLKAALKF | seqid_329 |
| variant_415 | RKKKFKKIPPPKFLKAALKF | seqid_330 |
| variant_416 | FKKKFKKIPPPKFLKAALKF | seqid_331 |
| variant_417 | KWKKFKKIPPPKFLKAALKF | seqid_332 |
| variant_418 | RWKKFKKIPPPKFLKAALKF | seqid_333 |
| variant_419 | FWKKFKKIPPPKFLKAALKF | seqid_334 |
| variant_420 | KKKLLKKIPPPKFLKAALKF | seqid_335 |
| variant_421 | RKKLLKKIPPPKFLKAALKF | seqid_336 |
| variant_422 | FKKLLKKIPPPKFLKAALKF | seqid_337 |
| variant_423 | KWKLLKKIPPPKFLKAALKF | seqid_338 |
| variant_424 | RWKLLKKIPPPKFLKAALKF | seqid_339 |
| variant_425 | FWKLLKKIPPPKFLKAALKF | seqid_340 |
| variant_426 | KKKKLKKIPPPKFLKAALKF | seqid_341 |
| variant_427 | RKKKLKKIPPPKFLKAALKF | seqid_342 |
| variant_428 | FKKKLKKIPPPKFLKAALKF | seqid_343 |
| variant_429 | KWKKLKKIPPPKFLKAALKF | seqid_344 |
| variant_430 | RWKKLKKIPPPKFLKAALKF | seqid_345 |
| variant_431 | FWKKLKKIPPPKFLKAALKF | seqid_346 |
| variant_432 | KKKLFLKIPPKFLKAALKF | seqid_347 |
| variant_433 | RKKLFLKIPPKFLKAALKF | seqid_348 |
| variant_434 | FKKLFLKIPPKFLKAALKF | seqid_349 |
| variant_435 | KWKLFLKIPPKFLKAALKF | seqid_350 |
| variant_436 | RWKLFLKIPPKFLKAALKF | seqid_351 |
| variant_437 | FWKLFLKIPPKFLKAALKF | seqid_352 |
| variant_438 | KKKKFLKIPPKFLKAALKF | seqid_353 |
| variant_439 | RKKKFLKIPPKFLKAALKF | seqid_354 |
| variant_440 | FKKKFLKIPPKFLKAALKF | seqid_355 |
| variant_441 | KWKKFLKIPPKFLKAALKF | seqid_356 |
| variant_442 | RWKKFLKIPPKFLKAALKF | seqid_357 |
| variant_443 | FWKKFLKIPPKFLKAALKF | seqid_358 |
| variant_444 | KKKLLLKIPPKFLKAALKF | seqid_359 |
| variant_445 | RKKLLLKIPPKFLKAALKF | seqid_360 |
| variant_446 | FKKLLLKIPPKFLKAALKF | seqid_361 |
| variant_447 | KWKLLLKIPPKFLKAALKF | seqid_362 |
| variant_448 | RWKLLLKIPPKFLKAALKF | seqid_363 |
| variant_449 | FWKLLLKIPPKFLKAALKF | seqid_364 |
| variant_450 | KKKKLLKIPPKFLKAALKF | seqid_365 |
| variant_451 | RKKKLLKIPPKFLKAALKF | seqid_366 |
| variant_452 | FKKKLLKIPPKFLKAALKF | seqid_367 |
| variant_453 | KWKKLLKIPPKFLKAALKF | seqid_368 |
| variant_454 | RWKKLLKIPPKFLKAALKF | seqid_369 |
| variant_455 | FWKKLLKIPPKFLKAALKF | seqid_370 |
| variant_456 | KKKLFKKIPPKFLKAALKF | seqid_371 |
| variant_457 | RKKLFKKIPPKFLKAALKF | seqid_372 |
| variant_458 | FKKLFKKIPPKFLKAALKF | seqid_373 |
| variant_459 | KWKLFKKIPPKFLKAALKF | seqid_374 |
| variant_460 | RWKLFKKIPPKFLKAALKF | seqid_375 |
| variant_461 | FWKLFKKIPPKFLKAALKF | seqid_376 |
| variant_462 | KKKKFKKIPPKFLKAALKF | seqid_377 |
| variant_463 | RKKKFKKIPPKFLKAALKF | seqid_378 |
| variant_464 | FKKKFKKIPPKFLKAALKF | seqid_379 |
| variant_465 | KWKKFKKIPPKFLKAALKF | seqid_380 |
| variant_466 | RWKKFKKIPPKFLKAALKF | seqid_381 |
| variant_467 | FWKKFKKIPPKFLKAALKF | seqid_382 |
| variant_468 | KKKLLKKIPPKFLKAALKF | seqid_383 |
| variant_469 | RKKLLKKIPPKFLKAALKF | seqid_384 |
| variant_470 | FKKLLKKIPPKFLKAALKF | seqid_385 |
| variant_471 | KWKLLKKIPPKFLKAALKF | seqid_386 |
| variant_472 | RWKLLKKIPPKFLKAALKF | seqid_387 |
| variant_473 | FWKLLKKIPPKFLKAALKF | seqid_388 |
| variant_474 | KKKKLKKIPPKFLKAALKF | seqid_389 |
| variant_475 | RKKKLKKIPPKFLKAALKF | seqid_390 |
| variant_476 | FKKKLKKIPPKFLKAALKF | seqid_391 |
| variant_477 | KWKKLKKIPPKFLKAALKF | seqid_392 |
| variant_478 | RWKKLKKIPPKFLKAALKF | seqid_393 |
| variant_479 | FWKKLKKIPPKFLKAALKF | seqid_394 |
| variant_528 | KKKLFLKIPKFLKAALKF | seqid_395 |
| variant_529 | RKKLFLKIPKFLKAALKF | seqid_396 |
| variant_530 | FKKLFLKIPKFLKAALKF | seqid_397 |
| variant_531 | KWKLFLKIPKFLKAALKF | seqid_398 |
| variant_532 | RWKLFLKIPKFLKAALKF | seqid_399 |
| variant_533 | FWKLFLKIPKFLKAALKF | seqid_400 |
| variant_534 | KKKKFLKIPKFLKAALKF | seqid_401 |
| variant_535 | RKKKFLKIPKFLKAALKF | seqid_402 |
| variant_536 | FKKKFLKIPKFLKAALKF | seqid_403 |
| variant_537 | KWKKFLKIPKFLKAALKF | seqid_404 |
| variant_538 | RWKKFLKIPKFLKAALKF | seqid_405 |
| variant_539 | FWKKFLKIPKFLKAALKF | seqid_406 |
| variant_540 | KKKLLLKIPKFLKAALKF | seqid_407 |
| variant_541 | RKKLLLKIPKFLKAALKF | seqid_408 |
| variant_542 | FKKLLLKIPKFLKAALKF | seqid_409 |
| variant_543 | KWKLLLKIPKFLKAALKF | seqid_410 |
| variant_544 | RWKLLLKIPKFLKAALKF | seqid_411 |
| variant_545 | FWKLLLKIPKFLKAALKF | seqid_412 |
| variant_546 | KKKKLLKIPKFLKAALKF | seqid_413 |
| variant_547 | RKKKLLKIPKFLKAALKF | seqid_414 |
| variant_548 | FKKKLLKIPKFLKAALKF | seqid_415 |
| variant_549 | KWKKLLKIPKFLKAALKF | seqid_416 |
| variant_550 | RWKKLLKIPKFLKAALKF | seqid_417 |
| variant_551 | FWKKLLKIPKFLKAALKF | seqid_418 |
| variant_552 | KKKLFKKIPKFLKAALKF | seqid_419 |
| variant_553 | RKKLFKKIPKFLKAALKF | seqid_420 |
| variant_554 | FKKLFKKIPKFLKAALKF | seqid_421 |
| variant_555 | KWKLFKKIPKFLKAALKF | seqid_422 |
| variant_556 | RWKLFKKIPKFLKAALKF | seqid_423 |
| variant_557 | FWKLFKKIPKFLKAALKF | seqid_424 |
| variant_558 | KKKKFKKIPKFLKAALKF | seqid_425 |
| variant_559 | RKKKFKKIPKFLKAALKF | seqid_426 |
| variant_560 | FKKKFKKIPKFLKAALKF | seqid_427 |
| variant_561 | KWKKFKKIPKFLKAALKF | seqid_428 |
| variant_562 | RWKKFKKIPKFLKAALKF | seqid_429 |
| variant_563 | FWKKFKKIPKFLKAALKF | seqid_430 |
| variant_564 | KKKLLKKIPKFLKAALKF | seqid_431 |
| variant_565 | RKKLLKKIPKFLKAALKF | seqid_432 |
| variant_566 | FKKLLKKIPKFLKAALKF | seqid_433 |
| variant_567 | KWKLLKKIPKFLKAALKF | seqid_434 |
| variant_568 | RWKLLKKIPKFLKAALKF | seqid_435 |
| variant_569 | FWKLLKKIPKFLKAALKF | seqid_436 |
| variant_570 | KKKKLKKIPKFLKAALKF | seqid_437 |
| variant_571 | RKKKLKKIPKFLKAALKF | seqid_438 |
| variant_572 | FKKKLKKIPKFLKAALKF | seqid_439 |
| variant_573 | KWKKLKKIPKFLKAALKF | seqid_440 |
| variant_574 | RWKKLKKIPKFLKAALKF | seqid_441 |
| variant_575 | FWKKLKKIPKFLKAALKF | seqid_442 |
| variant_576 | KKKLFLKIPPPKFKKAALKF | seqid_443 |
| variant_577 | RKKLFLKIPPPKFKKAALKF | seqid_444 |
| variant_578 | FKKLFLKIPPPKFKKAALKF | seqid_445 |
| variant_579 | KWKLFLKIPPPKFKKAALKF | seqid_446 |
| variant_580 | RWKLFLKIPPPKFKKAALKF | seqid_447 |
| variant_581 | FWKLFLKIPPPKFKKAALKF | seqid_448 |
| variant_582 | KKKKFLKIPPPKFKKAALKF | seqid_449 |
| variant_583 | RKKKFLKIPPPKFKKAALKF | seqid_450 |
| variant_584 | FKKKFLKIPPPKFKKAALKF | seqid_451 |
| variant_585 | KWKKFLKIPPPKFKKAALKF | seqid_452 |
| variant_586 | RWKKFLKIPPPKFKKAALKF | seqid_453 |
| variant_587 | FWKKFLKIPPPKFKKAALKF | seqid_454 |
| variant_588 | KKKLLLKIPPPKFKKAALKF | seqid_455 |
| variant_589 | RKKLLLKIPPPKFKKAALKF | seqid_456 |
| variant_590 | FKKLLLKIPPPKFKKAALKF | seqid_457 |
| variant_591 | KWKLLLKIPPPKFKKAALKF | seqid_458 |
| variant_592 | RWKLLLKIPPPKFKKAALKF | seqid_459 |
| variant_593 | FWKLLLKIPPPKFKKAALKF | seqid_460 |
| variant_594 | KKKKLLKIPPPKFKKAALKF | seqid_461 |
| variant_595 | RKKKLLKIPPPKFKKAALKF | seqid_462 |
| variant_596 | FKKKLLKIPPPKFKKAALKF | seqid_463 |
| variant_597 | KWKKLLKIPPPKFKKAALKF | seqid_464 |
| variant_598 | RWKKLLKIPPPKFKKAALKF | seqid_465 |
| variant_599 | FWKKLLKIPPPKFKKAALKF | seqid_466 |
| variant_600 | KKKLFKKIPPPKFKKAALKF | seqid_467 |
| variant_601 | RKKLFKKIPPPKFKKAALKF | seqid_468 |
| variant_602 | FKKLFKKIPPPKFKKAALKF | seqid_469 |
| variant_603 | KWKLFKKIPPPKFKKAALKF | seqid_470 |
| variant_604 | RWKLFKKIPPPKFKKAALKF | seqid_471 |
| variant_605 | FWKLFKKIPPPKFKKAALKF | seqid_472 |
| variant_606 | KKKKFKKIPPPKFKKAALKF | seqid_473 |
| variant_607 | RKKKFKKIPPPKFKKAALKF | seqid_474 |
| variant_608 | FKKKFKKIPPPKFKKAALKF | seqid_475 |
| variant_609 | KWKKFKKIPPPKFKKAALKF | seqid_476 |
| variant_610 | RWKKFKKIPPPKFKKAALKF | seqid_477 |
| variant_611 | FWKKFKKIPPPKFKKAALKF | seqid_478 |
| variant_612 | KKKLLKKIPPPKFKKAALKF | seqid_479 |
| variant_613 | RKKLLKKIPPPKFKKAALKF | seqid_480 |
| variant_614 | FKKLLKKIPPPKFKKAALKF | seqid_481 |
| variant_615 | KWKLLKKIPPPKFKKAALKF | seqid_482 |
| variant_616 | RWKLLKKIPPPKFKKAALKF | seqid_483 |
| variant_617 | FWKLLKKIPPPKFKKAALKF | seqid_484 |
| variant_618 | KKKKLKKIPPPKFKKAALKF | seqid_485 |
| variant_619 | RKKKLKKIPPPKFKKAALKF | seqid_486 |
| variant_620 | FKKKLKKIPPPKFKKAALKF | seqid_487 |
| variant_621 | KWKKLKKIPPPKFKKAALKF | seqid_488 |
| variant_622 | RWKKLKKIPPPKFKKAALKF | seqid_489 |
| variant_623 | FWKKLKKIPPPKFKKAALKF | seqid_490 |
| variant_624 | KKKLFLKIPPKFKKAALKF | seqid_491 |
| variant_625 | RKKLFLKIPPKFKKAALKF | seqid_492 |
| variant_626 | FKKLFLKIPPKFKKAALKF | seqid_493 |
| variant_627 | KWKLFLKIPPKFKKAALKF | seqid_494 |
| variant_628 | RWKLFLKIPPKFKKAALKF | seqid_495 |
| variant_629 | FWKLFLKIPPKFKKAALKF | seqid_496 |
| variant_630 | KKKKFLKIPPKFKKAALKF | seqid_497 |
| variant_631 | RKKKFLKIPPKFKKAALKF | seqid_498 |
| variant_632 | FKKKFLKIPPKFKKAALKF | seqid_499 |
| variant_633 | KWKKFLKIPPKFKKAALKF | seqid_500 |
| variant_634 | RWKKFLKIPPKFKKAALKF | seqid_501 |
| variant_635 | FWKKFLKIPPKFKKAALKF | seqid_502 |
| variant_636 | KKKLLLKIPPKFKKAALKF | seqid_503 |
| variant_637 | RKKLLLKIPPKFKKAALKF | seqid_504 |
| variant_638 | FKKLLLKIPPKFKKAALKF | seqid_505 |
| variant_639 | KWKLLLKIPPKFKKAALKF | seqid_506 |
| variant_640 | RWKLLLKIPPKFKKAALKF | seqid_507 |
| variant_641 | FWKLLLKIPPKFKKAALKF | seqid_508 |
| variant_642 | KKKKLLKIPPKFKKAALKF | seqid_509 |
| variant_643 | RKKKLLKIPPKFKKAALKF | seqid_510 |
| variant_644 | FKKKLLKIPPKFKKAALKF | seqid_511 |
| variant_645 | KWKKLLKIPPKFKKAALKF | seqid_512 |
| variant_646 | RWKKLLKIPPKFKKAALKF | seqid_513 |
| variant_647 | FWKKLLKIPPKFKKAALKF | seqid_514 |
| variant_648 | KKKLFKKIPPKFKKAALKF | seqid_515 |
| variant_649 | RKKLFKKIPPKFKKAALKF | seqid_516 |
| variant_650 | FKKLFKKIPPKFKKAALKF | seqid_517 |
| variant_651 | KWKLFKKIPPKFKKAALKF | seqid_518 |
| variant_652 | RWKLFKKIPPKFKKAALKF | seqid_519 |
| variant_653 | FWKLFKKIPPKFKKAALKF | seqid_520 |
| variant_654 | KKKKFKKIPPKFKKAALKF | seqid_521 |
| variant_655 | RKKKFKKIPPKFKKAALKF | seqid_522 |
| variant_656 | FKKKFKKIPPKFKKAALKF | seqid_523 |
| variant_657 | KWKKFKKIPPKFKKAALKF | seqid_524 |
| variant_658 | RWKKFKKIPPKFKKAALKF | seqid_525 |
| variant_659 | FWKKFKKIPPKFKKAALKF | seqid_526 |
| variant_660 | KKKLLKKIPPKFKKAALKF | seqid_527 |
| variant_661 | RKKLLKKIPPKFKKAALKF | seqid_528 |
| variant_662 | FKKLLKKIPPKFKKAALKF | seqid_529 |
| variant_663 | KWKLLKKIPPKFKKAALKF | seqid_530 |
| variant_664 | RWKLLKKIPPKFKKAALKF | seqid_531 |
| variant_665 | FWKLLKKIPPKFKKAALKF | seqid_532 |
| variant_666 | KKKKLKKIPPKFKKAALKF | seqid_533 |
| variant_667 | RKKKLKKIPPKFKKAALKF | seqid_534 |
| variant_668 | FKKKLKKIPPKFKKAALKF | seqid_535 |
| variant_669 | KWKKLKKIPPKFKKAALKF | seqid_536 |
| variant_670 | RWKKLKKIPPKFKKAALKF | seqid_537 |
| variant_671 | FWKKLKKIPPKFKKAALKF | seqid_538 |
| variant_720 | KKKLFLKIPKFKKAALKF | seqid_539 |
| variant_721 | RKKLFLKIPKFKKAALKF | seqid_540 |
| variant_722 | FKKLFLKIPKFKKAALKF | seqid_541 |
| variant_723 | KWKLFLKIPKFKKAALKF | seqid_542 |
| variant_724 | RWKLFLKIPKFKKAALKF | seqid_543 |
| variant_725 | FWKLFLKIPKFKKAALKF | seqid_544 |
| variant_726 | KKKKFLKIPKFKKAALKF | seqid_545 |
| variant_727 | RKKKFLKIPKFKKAALKF | seqid_546 |
| variant_728 | FKKKFLKIPKFKKAALKF | seqid_547 |
| variant_729 | KWKKFLKIPKFKKAALKF | seqid_548 |
| variant_730 | RWKKFLKIPKFKKAALKF | seqid_549 |
| variant_731 | FWKKFLKIPKFKKAALKF | seqid_550 |
| variant_732 | KKKLLLKIPKFKKAALKF | seqid_551 |
| variant_733 | RKKLLLKIPKFKKAALKF | seqid_552 |
| variant_734 | FKKLLLKIPKFKKAALKF | seqid_553 |
| variant_735 | KWKLLLKIPKFKKAALKF | seqid_554 |
| variant_736 | RWKLLLKIPKFKKAALKF | seqid_555 |
| variant_737 | FWKLLLKIPKFKKAALKF | seqid_556 |
| variant_738 | KKKKLLKIPKFKKAALKF | seqid_557 |
| variant_739 | RKKKLLKIPKFKKAALKF | seqid_558 |
| variant_740 | FKKKLLKIPKFKKAALKF | seqid_559 |
| variant_741 | KWKKLLKIPKFKKAALKF | seqid_560 |
| variant_742 | RWKKLLKIPKFKKAALKF | seqid_561 |
| variant_743 | FWKKLLKIPKFKKAALKF | seqid_562 |
| variant_744 | KKKLFKKIPKFKKAALKF | seqid_563 |
| variant_745 | RKKLFKKIPKFKKAALKF | seqid_564 |
| variant_746 | FKKLFKKIPKFKKAALKF | seqid_565 |
| variant_747 | KWKLFKKIPKFKKAALKF | seqid_566 |
| variant_748 | RWKLFKKIPKFKKAALKF | seqid_567 |
| variant_749 | FWKLFKKIPKFKKAALKF | seqid_568 |
| variant_750 | KKKKFKKIPKFKKAALKF | seqid_569 |
| variant_751 | RKKKFKKIPKFKKAALKF | seqid_570 |
| variant_752 | FKKKFKKIPKFKKAALKF | seqid_571 |
| variant_753 | KWKKFKKIPKFKKAALKF | seqid_572 |
| variant_754 | RWKKFKKIPKFKKAALKF | seqid_573 |
| variant_755 | FWKKFKKIPKFKKAALKF | seqid_574 |
| variant_756 | KKKLLKKIPKFKKAALKF | seqid_575 |
| variant_757 | RKKLLKKIPKFKKAALKF | seqid_576 |
| variant_758 | FKKLLKKIPKFKKAALKF | seqid_577 |
| variant_759 | KWKLLKKIPKFKKAALKF | seqid_578 |
| variant_760 | RWKLLKKIPKFKKAALKF | seqid_579 |
| variant_761 | FWKLLKKIPKFKKAALKF | seqid_580 |
| variant_762 | KKKKLKKIPKFKKAALKF | seqid_581 |
| variant_763 | RKKKLKKIPKFKKAALKF | seqid_582 |
| variant_764 | FKKKLKKIPKFKKAALKF | seqid_583 |
| variant_765 | KWKKLKKIPKFKKAALKF | seqid_584 |
| variant_766 | RWKKLKKIPKFKKAALKF | seqid_585 |
| variant_767 | FWKKLKKIPKFKKAALKF | seqid_586 |
| variant_768 | KKKLFLKIPPPKFLHLALKF | seqid_587 |
| variant_769 | RKKLFLKIPPPKFLHLALKF | seqid_588 |
| variant_770 | FKKLFLKIPPPKFLHLALKF | seqid_589 |
| variant_771 | KWKLFLKIPPPKFLHLALKF | seqid_590 |
| variant_772 | RWKLFLKIPPPKFLHLALKF | seqid_591 |
| variant_773 | FWKLFLKIPPPKFLHLALKF | seqid_592 |
| variant_774 | KKKKFLKIPPPKFLHLALKF | seqid_593 |
| variant_775 | RKKKFLKIPPPKFLHLALKF | seqid_594 |
| variant_776 | FKKKFLKIPPPKFLHLALKF | seqid_595 |
| variant_777 | KWKKFLKIPPPKFLHLALKF | seqid_596 |
| variant_778 | RWKKFLKIPPPKFLHLALKF | seqid_597 |
| variant_779 | FWKKFLKIPPPKFLHLALKF | seqid_598 |
| variant_780 | KKKLLLKIPPPKFLHLALKF | seqid_599 |
| variant_781 | RKKLLLKIPPPKFLHLALKF | seqid_600 |
| variant_782 | FKKLLLKIPPPKFLHLALKF | seqid_601 |
| variant_783 | KWKLLLKIPPPKFLHLALKF | seqid_602 |
| variant_784 | RWKLLLKIPPPKFLHLALKF | seqid_603 |
| variant_785 | FWKLLLKIPPPKFLHLALKF | seqid_604 |
| variant_786 | KKKKLLKIPPPKFLHLALKF | seqid_605 |
| variant_787 | RKKKLLKIPPPKFLHLALKF | seqid_606 |
| variant_788 | FKKKLLKIPPPKFLHLALKF | seqid_607 |
| variant_789 | KWKKLLKIPPPKFLHLALKF | seqid_608 |
| variant_790 | RWKKLLKIPPPKFLHLALKF | seqid_609 |
| variant_791 | FWKKLLKIPPPKFLHLALKF | seqid_610 |
| variant_792 | KKKLFKKIPPPKFLHLALKF | seqid_611 |
| variant_793 | RKKLFKKIPPPKFLHLALKF | seqid_612 |
| variant_794 | FKKLFKKIPPPKFLHLALKF | seqid_613 |
| variant_795 | KWKLFKKIPPPKFLHLALKF | seqid_614 |
| variant_796 | RWKLFKKIPPPKFLHLALKF | seqid_615 |
| variant_797 | FWKLFKKIPPPKFLHLALKF | seqid_616 |
| variant_798 | KKKKFKKIPPPKFLHLALKF | seqid_617 |
| variant_799 | RKKKFKKIPPPKFLHLALKF | seqid_618 |
| variant_800 | FKKKFKKIPPPKFLHLALKF | seqid_619 |
| variant_801 | KWKKFKKIPPPKFLHLALKF | seqid_620 |
| variant_802 | RWKKFKKIPPPKFLHLALKF | seqid_621 |
| variant_803 | FWKKFKKIPPPKFLHLALKF | seqid_622 |
| variant_804 | KKKLLKKIPPPKFLHLALKF | seqid_623 |
| variant_805 | RKKLLKKIPPPKFLHLALKF | seqid_624 |
| variant_806 | FKKLLKKIPPPKFLHLALKF | seqid_625 |
| variant_807 | KWKLLKKIPPPKFLHLALKF | seqid_626 |
| variant_808 | RWKLLKKIPPPKFLHLALKF | seqid_627 |
| variant_809 | FWKLLKKIPPPKFLHLALKF | seqid_628 |
| variant_810 | KKKKLKKIPPPKFLHLALKF | seqid_629 |
| variant_811 | RKKKLKKIPPPKFLHLALKF | seqid_630 |
| variant_812 | FKKKLKKIPPPKFLHLALKF | seqid_631 |
| variant_813 | KWKKLKKIPPPKFLHLALKF | seqid_632 |
| variant_814 | RWKKLKKIPPPKFLHLALKF | seqid_633 |
| variant_815 | FWKKLKKIPPPKFLHLALKF | seqid_634 |
| variant_816 | KKKLFLKIPPKFLHLALKF | seqid_635 |
| variant_817 | RKKLFLKIPPKFLHLALKF | seqid_636 |
| variant_818 | FKKLFLKIPPKFLHLALKF | seqid_637 |
| variant_819 | KWKLFLKIPPKFLHLALKF | seqid_638 |
| variant_820 | RWKLFLKIPPKFLHLALKF | seqid_639 |
| variant_821 | FWKLFLKIPPKFLHLALKF | seqid_640 |
| variant_822 | KKKKFLKIPPKFLHLALKF | seqid_641 |
| variant_823 | RKKKFLKIPPKFLHLALKF | seqid_642 |
| variant_824 | FKKKFLKIPPKFLHLALKF | seqid_643 |
| variant_825 | KWKKFLKIPPKFLHLALKF | seqid_644 |
| variant_826 | RWKKFLKIPPKFLHLALKF | seqid_645 |
| variant_827 | FWKKFLKIPPKFLHLALKF | seqid_646 |
| variant_828 | KKKLLLKIPPKFLHLALKF | seqid_647 |
| variant_829 | RKKLLLKIPPKFLHLALKF | seqid_648 |
| variant_830 | FKKLLLKIPPKFLHLALKF | seqid_649 |
| variant_831 | KWKLLLKIPPKFLHLALKF | seqid_650 |
| variant_832 | RWKLLLKIPPKFLHLALKF | seqid_651 |
| variant_833 | FWKLLLKIPPKFLHLALKF | seqid_652 |
| variant_834 | KKKKLLKIPPKFLHLALKF | seqid_653 |
| variant_835 | RKKKLLKIPPKFLHLALKF | seqid_654 |
| variant_836 | FKKKLLKIPPKFLHLALKF | seqid_655 |
| variant_837 | KWKKLLKIPPKFLHLALKF | seqid_656 |
| variant_838 | RWKKLLKIPPKFLHLALKF | seqid_657 |
| variant_839 | FWKKLLKIPPKFLHLALKF | seqid_658 |
| variant_840 | KKKLFKKIPPKFLHLALKF | seqid_659 |
| variant_841 | RKKLFKKIPPKFLHLALKF | seqid_660 |
| variant_842 | FKKLFKKIPPKFLHLALKF | seqid_661 |
| variant_843 | KWKLFKKIPPKFLHLALKF | seqid_662 |
| variant_844 | RWKLFKKIPPKFLHLALKF | seqid_663 |
| variant_845 | FWKLFKKIPPKFLHLALKF | seqid_664 |
| variant_846 | KKKKFKKIPPKFLHLALKF | seqid_665 |
| variant_847 | RKKKFKKIPPKFLHLALKF | seqid_666 |
| variant_848 | FKKKFKKIPPKFLHLALKF | seqid_667 |
| variant_849 | KWKKFKKIPPKFLHLALKF | seqid_668 |
| variant_850 | RWKKFKKIPPKFLHLALKF | seqid_669 |
| variant_851 | FWKKFKKIPPKFLHLALKF | seqid_670 |
| variant_852 | KKKLLKKIPPKFLHLALKF | seqid_671 |
| variant_853 | RKKLLKKIPPKFLHLALKF | seqid_672 |
| variant_854 | FKKLLKKIPPKFLHLALKF | seqid_673 |
| variant_855 | KWKLLKKIPPKFLHLALKF | seqid_674 |
| variant_856 | RWKLLKKIPPKFLHLALKF | seqid_675 |
| variant_857 | FWKLLKKIPPKFLHLALKF | seqid_676 |
| variant_858 | KKKKLKKIPPKFLHLALKF | seqid_677 |
| variant_859 | RKKKLKKIPPKFLHLALKF | seqid_678 |
| variant_860 | FKKKLKKIPPKFLHLALKF | seqid_679 |
| variant_861 | KWKKLKKIPPKFLHLALKF | seqid_680 |
| variant_862 | RWKKLKKIPPKFLHLALKF | seqid_681 |
| variant_863 | FWKKLKKIPPKFLHLALKF | seqid_682 |
| variant_912 | KKKLFLKIPKFLHLALKF | seqid_683 |
| variant_913 | RKKLFLKIPKFLHLALKF | seqid_684 |
| variant_914 | FKKLFLKIPKFLHLALKF | seqid_685 |
| variant_915 | KWKLFLKIPKFLHLALKF | seqid_686 |
| variant_916 | RWKLFLKIPKFLHLALKF | seqid_687 |
| variant_917 | FWKLFLKIPKFLHLALKF | seqid_688 |
| variant_918 | KKKKFLKIPKFLHLALKF | seqid_689 |
| variant_919 | RKKKFLKIPKFLHLALKF | seqid_690 |
| variant_920 | FKKKFLKIPKFLHLALKF | seqid_691 |
| variant_921 | KWKKFLKIPKFLHLALKF | seqid_692 |
| variant_922 | RWKKFLKIPKFLHLALKF | seqid_693 |
| variant_923 | FWKKFLKIPKFLHLALKF | seqid_694 |
| variant_924 | KKKLLLKIPKFLHLALKF | seqid_695 |
| variant_925 | RKKLLLKIPKFLHLALKF | seqid_696 |
| variant_926 | FKKLLLKIPKFLHLALKF | seqid_697 |
| variant_927 | KWKLLLKIPKFLHLALKF | seqid_698 |
| variant_928 | RWKLLLKIPKFLHLALKF | seqid_699 |
| variant_929 | FWKLLLKIPKFLHLALKF | seqid_700 |
| variant_930 | KKKKLLKIPKFLHLALKF | seqid_701 |
| variant_931 | RKKKLLKIPKFLHLALKF | seqid_702 |
| variant_932 | FKKKLLKIPKFLHLALKF | seqid_703 |
| variant_933 | KWKKLLKIPKFLHLALKF | seqid_704 |
| variant_934 | RWKKLLKIPKFLHLALKF | seqid_705 |
| variant_935 | FWKKLLKIPKFLHLALKF | seqid_706 |
| variant_936 | KKKLFKKIPKFLHLALKF | seqid_707 |
| variant_937 | RKKLFKKIPKFLHLALKF | seqid_708 |
| variant_938 | FKKLFKKIPKFLHLALKF | seqid_709 |
| variant_939 | KWKLFKKIPKFLHLALKF | seqid_710 |
| variant_940 | RWKLFKKIPKFLHLALKF | seqid_711 |
| variant_941 | FWKLFKKIPKFLHLALKF | seqid_712 |
| variant_942 | KKKKFKKIPKFLHLALKF | seqid_713 |
| variant_943 | RKKKFKKIPKFLHLALKF | seqid_714 |
| variant_944 | FKKKFKKIPKFLHLALKF | seqid_715 |
| variant_945 | KWKKFKKIPKFLHLALKF | seqid_716 |
| variant_946 | RWKKFKKIPKFLHLALKF | seqid_717 |
| variant_947 | FWKKFKKIPKFLHLALKF | seqid_718 |
| variant_948 | KKKLLKKIPKFLHLALKF | seqid_719 |
| variant_949 | RKKLLKKIPKFLHLALKF | seqid_720 |
| variant_950 | FKKLLKKIPKFLHLALKF | seqid_721 |
| variant_951 | KWKLLKKIPKFLHLALKF | seqid_722 |
| variant_952 | RWKLLKKIPKFLHLALKF | seqid_723 |
| variant_953 | FWKLLKKIPKFLHLALKF | seqid_724 |
| variant_954 | KKKKLKKIPKFLHLALKF | seqid_725 |
| variant_955 | RKKKLKKIPKFLHLALKF | seqid_726 |
| variant_956 | FKKKLKKIPKFLHLALKF | seqid_727 |
| variant_957 | KWKKLKKIPKFLHLALKF | seqid_728 |
| variant_958 | RWKKLKKIPKFLHLALKF | seqid_729 |
| variant_959 | FWKKLKKIPKFLHLALKF | seqid_730 |
| variant_960 | KKKLFLKIPPPKFKHLALKF | seqid_731 |
| variant_961 | RKKLFLKIPPPKFKHLALKF | seqid_732 |
| variant_962 | FKKLFLKIPPPKFKHLALKF | seqid_733 |
| variant_963 | KWKLFLKIPPPKFKHLALKF | seqid_734 |
| variant_964 | RWKLFLKIPPPKFKHLALKF | seqid_735 |
| variant_965 | FWKLFLKIPPPKFKHLALKF | seqid_736 |
| variant_966 | KKKKFLKIPPPKFKHLALKF | seqid_737 |
| variant_967 | RKKKFLKIPPPKFKHLALKF | seqid_738 |
| variant_968 | FKKKFLKIPPPKFKHLALKF | seqid_739 |
| variant_969 | KWKKFLKIPPPKFKHLALKF | seqid_740 |
| variant_970 | RWKKFLKIPPPKFKHLALKF | seqid_741 |
| variant_971 | FWKKFLKIPPPKFKHLALKF | seqid_742 |
| variant_972 | KKKLLLKIPPPKFKHLALKF | seqid_743 |
| variant_973 | RKKLLLKIPPPKFKHLALKF | seqid_744 |
| variant_974 | FKKLLLKIPPPKFKHLALKF | seqid_745 |
| variant_975 | KWKLLLKIPPPKFKHLALKF | seqid_746 |
| variant_976 | RWKLLLKIPPPKFKHLALKF | seqid_747 |
| variant_977 | FWKLLLKIPPPKFKHLALKF | seqid_748 |
| variant_978 | KKKKLLKIPPPKFKHLALKF | seqid_749 |
| variant_979 | RKKKLLKIPPPKFKHLALKF | seqid_750 |
| variant_980 | FKKKLLKIPPPKFKHLALKF | seqid_751 |
| variant_981 | KWKKLLKIPPPKFKHLALKF | seqid_752 |
| variant_982 | RWKKLLKIPPPKFKHLALKF | seqid_753 |
| variant_983 | FWKKLLKIPPPKFKHLALKF | seqid_754 |
| variant_984 | KKKLFKKIPPPKFKHLALKF | seqid_755 |
| variant_985 | RKKLFKKIPPPKFKHLALKF | seqid_756 |
| variant_986 | FKKLFKKIPPPKFKHLALKF | seqid_757 |
| variant_987 | KWKLFKKIPPPKFKHLALKF | seqid_758 |
| variant_988 | RWKLFKKIPPPKFKHLALKF | seqid_759 |
| variant_989 | FWKLFKKIPPPKFKHLALKF | seqid_760 |
| variant_990 | KKKKFKKIPPPKFKHLALKF | seqid_761 |
| variant_991 | RKKKFKKIPPPKFKHLALKF | seqid_762 |
| variant_992 | FKKKFKKIPPPKFKHLALKF | seqid_763 |
| variant_993 | KWKKFKKIPPPKFKHLALKF | seqid_764 |
| variant_994 | RWKKFKKIPPPKFKHLALKF | seqid_765 |
| variant_995 | FWKKFKKIPPPKFKHLALKF | seqid_766 |
| variant_996 | KKKLLKKIPPPKFKHLALKF | seqid_767 |
| variant_997 | RKKLLKKIPPPKFKHLALKF | seqid_768 |
| variant_998 | FKKLLKKIPPPKFKHLALKF | seqid_769 |
| variant_999 | KWKLLKKIPPPKFKHLALKF | seqid_770 |
| variant_1000 | RWKLLKKIPPPKFKHLALKF | seqid_771 |
| variant_1001 | FWKLLKKIPPPKFKHLALKF | seqid_772 |
| variant_1002 | KKKKLKKIPPPKFKHLALKF | seqid_773 |
| variant_1003 | RKKKLKKIPPPKFKHLALKF | seqid_774 |
| variant_1004 | FKKKLKKIPPPKFKHLALKF | seqid_775 |
| variant_1005 | KWKKLKKIPPPKFKHLALKF | seqid_776 |
| variant_1006 | RWKKLKKIPPPKFKHLALKF | seqid_777 |
| variant_1007 | FWKKLKKIPPPKFKHLALKF | seqid_778 |
| variant_1008 | KKKLFLKIPPKFKHLALKF | seqid_779 |
| variant_1009 | RKKLFLKIPPKFKHLALKF | seqid_780 |
| variant_1010 | FKKLFLKIPPKFKHLALKF | seqid_781 |
| variant_1011 | KWKLFLKIPPKFKHLALKF | seqid_782 |
| variant_1012 | RWKLFLKIPPKFKHLALKF | seqid_783 |
| variant_1013 | FWKLFLKIPPKFKHLALKF | seqid_784 |
| variant_1014 | KKKKFLKIPPKFKHLALKF | seqid_785 |
| variant_1015 | RKKKFLKIPPKFKHLALKF | seqid_786 |
| variant_1016 | FKKKFLKIPPKFKHLALKF | seqid_787 |
| variant_1017 | KWKKFLKIPPKFKHLALKF | seqid_788 |
| variant_1018 | RWKKFLKIPPKFKHLALKF | seqid_789 |
| variant_1019 | FWKKFLKIPPKFKHLALKF | seqid_790 |
| variant_1020 | KKKLLLKIPPKFKHLALKF | seqid_791 |
| variant_1021 | RKKLLLKIPPKFKHLALKF | seqid_792 |
| variant_1022 | FKKLLLKIPPKFKHLALKF | seqid_793 |
| variant_1023 | KWKLLLKIPPKFKHLALKF | seqid_794 |
| variant_1024 | RWKLLLKIPPKFKHLALKF | seqid_795 |
| variant_1025 | FWKLLLKIPPKFKHLALKF | seqid_796 |
| variant_1026 | KKKKLLKIPPKFKHLALKF | seqid_797 |
| variant_1027 | RKKKLLKIPPKFKHLALKF | seqid_798 |
| variant_1028 | FKKKLLKIPPKFKHLALKF | seqid_799 |
| variant_1029 | KWKKLLKIPPKFKHLALKF | seqid_800 |
| variant_1030 | RWKKLLKIPPKFKHLALKF | seqid_801 |
| variant_1031 | FWKKLLKIPPKFKHLALKF | seqid_802 |
| variant_1032 | KKKLFKKIPPKFKHLALKF | seqid_803 |
| variant_1033 | RKKLFKKIPPKFKHLALKF | seqid_804 |
| variant_1034 | FKKLFKKIPPKFKHLALKF | seqid_805 |
| variant_1035 | KWKLFKKIPPKFKHLALKF | seqid_806 |
| variant_1036 | RWKLFKKIPPKFKHLALKF | seqid_807 |
| variant_1037 | FWKLFKKIPPKFKHLALKF | seqid_808 |
| variant_1038 | KKKKFKKIPPKFKHLALKF | seqid_809 |
| variant_1039 | RKKKFKKIPPKFKHLALKF | seqid_810 |
| variant_1040 | FKKKFKKIPPKFKHLALKF | seqid_811 |
| variant_1041 | KWKKFKKIPPKFKHLALKF | seqid_812 |
| variant_1042 | RWKKFKKIPPKFKHLALKF | seqid_813 |
| variant_1043 | FWKKFKKIPPKFKHLALKF | seqid_814 |
| variant_1044 | KKKLLKKIPPKFKHLALKF | seqid_815 |
| variant_1045 | RKKLLKKIPPKFKHLALKF | seqid_816 |
| variant_1046 | FKKLLKKIPPKFKHLALKF | seqid_817 |
| variant_1047 | KWKLLKKIPPKFKHLALKF | seqid_818 |
| variant_1048 | RWKLLKKIPPKFKHLALKF | seqid_819 |
| variant_1049 | FWKLLKKIPPKFKHLALKF | seqid_820 |
| variant_1050 | KKKKLKKIPPKFKHLALKF | seqid_821 |
| variant_1051 | RKKKLKKIPPKFKHLALKF | seqid_822 |
| variant_1052 | FKKKLKKIPPKFKHLALKF | seqid_823 |
| variant_1053 | KWKKLKKIPPKFKHLALKF | seqid_824 |
| variant_1054 | RWKKLKKIPPKFKHLALKF | seqid_825 |
| variant_1055 | FWKKLKKIPPKFKHLALKF | seqid_826 |
| variant_1104 | KKKLFLKIPKFKHLALKF | seqid_827 |
| variant_1105 | RKKLFLKIPKFKHLALKF | seqid_828 |
| variant_1106 | FKKLFLKIPKFKHLALKF | seqid_829 |
| variant_1107 | KWKLFLKIPKFKHLALKF | seqid_830 |
| variant_1108 | RWKLFLKIPKFKHLALKF | seqid_831 |
| variant_1109 | FWKLFLKIPKFKHLALKF | seqid_832 |
| variant_1110 | KKKKFLKIPKFKHLALKF | seqid_833 |
| variant_1111 | RKKKFLKIPKFKHLALKF | seqid_834 |
| variant_1112 | FKKKFLKIPKFKHLALKF | seqid_835 |
| variant_1113 | KWKKFLKIPKFKHLALKF | seqid_836 |
| variant_1114 | RWKKFLKIPKFKHLALKF | seqid_837 |
| variant_1115 | FWKKFLKIPKFKHLALKF | seqid_838 |
| variant_1116 | KKKLLLKIPKFKHLALKF | seqid_839 |
| variant_1117 | RKKLLLKIPKFKHLALKF | seqid_840 |
| variant_1118 | FKKLLLKIPKFKHLALKF | seqid_841 |
| variant_1119 | KWKLLLKIPKFKHLALKF | seqid_842 |
| variant_1120 | RWKLLLKIPKFKHLALKF | seqid_843 |
| variant_1121 | FWKLLLKIPKFKHLALKF | seqid_844 |
| variant_1122 | KKKKLLKIPKFKHLALKF | seqid_845 |
| variant_1123 | RKKKLLKIPKFKHLALKF | seqid_846 |
| variant_1124 | FKKKLLKIPKFKHLALKF | seqid_847 |
| variant_1125 | KWKKLLKIPKFKHLALKF | seqid_848 |
| variant_1126 | RWKKLLKIPKFKHLALKF | seqid_849 |
| variant_1127 | FWKKLLKIPKFKHLALKF | seqid_850 |
| variant_1128 | KKKLFKKIPKFKHLALKF | seqid_851 |
| variant_1129 | RKKLFKKIPKFKHLALKF | seqid_852 |
| variant_1130 | FKKLFKKIPKFKHLALKF | seqid_853 |
| variant_1131 | KWKLFKKIPKFKHLALKF | seqid_854 |
| variant_1132 | RWKLFKKIPKFKHLALKF | seqid_855 |
| variant_1133 | FWKLFKKIPKFKHLALKF | seqid_856 |
| variant_1134 | KKKKFKKIPKFKHLALKF | seqid_857 |
| variant_1135 | RKKKFKKIPKFKHLALKF | seqid_858 |
| variant_1136 | FKKKFKKIPKFKHLALKF | seqid_859 |
| variant_1137 | KWKKFKKIPKFKHLALKF | seqid_860 |
| variant_1138 | RWKKFKKIPKFKHLALKF | seqid_861 |
| variant_1139 | FWKKFKKIPKFKHLALKF | seqid_862 |
| variant_1140 | KKKLLKKIPKFKHLALKF | seqid_863 |
| variant_1141 | RKKLLKKIPKFKHLALKF | seqid_864 |
| variant_1142 | FKKLLKKIPKFKHLALKF | seqid_865 |
| variant_1143 | KWKLLKKIPKFKHLALKF | seqid_866 |
| variant_1144 | RWKLLKKIPKFKHLALKF | seqid_867 |
| variant_1145 | FWKLLKKIPKFKHLALKF | seqid_868 |
| variant_1146 | KKKKLKKIPKFKHLALKF | seqid_869 |
| variant_1147 | RKKKLKKIPKFKHLALKF | seqid_870 |
| variant_1148 | FKKKLKKIPKFKHLALKF | seqid_871 |
| variant_1149 | KWKKLKKIPKFKHLALKF | seqid_872 |
| variant_1150 | RWKKLKKIPKFKHLALKF | seqid_873 |
| variant_1151 | FWKKLKKIPKFKHLALKF | seqid_874 |
| variant_1152 | KKKLFLKIPPPKFLKLALKF | seqid_875 |
| variant_1153 | RKKLFLKIPPPKFLKLALKF | seqid_876 |
| variant_1154 | FKKLFLKIPPPKFLKLALKF | seqid_877 |
| variant_1155 | KWKLFLKIPPPKFLKLALKF | seqid_878 |
| variant_1156 | RWKLFLKIPPPKFLKLALKF | seqid_879 |
| variant_1157 | FWKLFLKIPPPKFLKLALKF | seqid_880 |
| variant_1158 | KKKKFLKIPPPKFLKLALKF | seqid_881 |
| variant_1159 | RKKKFLKIPPPKFLKLALKF | seqid_882 |
| variant_1160 | FKKKFLKIPPPKFLKLALKF | seqid_883 |
| variant_1161 | KWKKFLKIPPPKFLKLALKF | seqid_884 |
| variant_1162 | RWKKFLKIPPPKFLKLALKF | seqid_885 |
| variant_1163 | FWKKFLKIPPPKFLKLALKF | seqid_886 |
| variant_1164 | KKKLLLKIPPPKFLKLALKF | seqid_887 |
| variant_1165 | RKKLLLKIPPPKFLKLALKF | seqid_888 |
| variant_1166 | FKKLLLKIPPPKFLKLALKF | seqid_889 |
| variant_1167 | KWKLLLKIPPPKFLKLALKF | seqid_890 |
| variant_1168 | RWKLLLKIPPPKFLKLALKF | seqid_891 |
| variant_1169 | FWKLLLKIPPPKFLKLALKF | seqid_892 |
| variant_1170 | KKKKLLKIPPPKFLKLALKF | seqid_893 |
| variant_1171 | RKKKLLKIPPPKFLKLALKF | seqid_894 |
| variant_1172 | FKKKLLKIPPPKFLKLALKF | seqid_895 |
| variant_1173 | KWKKLLKIPPPKFLKLALKF | seqid_896 |
| variant_1174 | RWKKLLKIPPPKFLKLALKF | seqid_897 |
| variant_1175 | FWKKLLKIPPPKFLKLALKF | seqid_898 |
| variant_1176 | KKKLFKKIPPPKFLKLALKF | seqid_899 |
| variant_1177 | RKKLFKKIPPPKFLKLALKF | seqid_900 |
| variant_1178 | FKKLFKKIPPPKFLKLALKF | seqid_901 |
| variant_1179 | KWKLFKKIPPPKFLKLALKF | seqid_902 |
| variant_1180 | RWKLFKKIPPPKFLKLALKF | seqid_903 |
| variant_1181 | FWKLFKKIPPPKFLKLALKF | seqid_904 |
| variant_1182 | KKKKFKKIPPPKFLKLALKF | seqid_905 |
| variant_1183 | RKKKFKKIPPPKFLKLALKF | seqid_906 |
| variant_1184 | FKKKFKKIPPPKFLKLALKF | seqid_907 |
| variant_1185 | KWKKFKKIPPPKFLKLALKF | seqid_908 |
| variant_1186 | RWKKFKKIPPPKFLKLALKF | seqid_909 |
| variant_1187 | FWKKFKKIPPPKFLKLALKF | seqid_910 |
| variant_1188 | KKKLLKKIPPPKFLKLALKF | seqid_911 |
| variant_1189 | RKKLLKKIPPPKFLKLALKF | seqid_912 |
| variant_1190 | FKKLLKKIPPPKFLKLALKF | seqid_913 |
| variant_1191 | KWKLLKKIPPPKFLKLALKF | seqid_914 |
| variant_1192 | RWKLLKKIPPPKFLKLALKF | seqid_915 |
| variant_1193 | FWKLLKKIPPPKFLKLALKF | seqid_916 |
| variant_1194 | KKKKLKKIPPPKFLKLALKF | seqid_917 |
| variant_1195 | RKKKLKKIPPPKFLKLALKF | seqid_918 |
| variant_1196 | FKKKLKKIPPPKFLKLALKF | seqid_919 |
| variant_1197 | KWKKLKKIPPPKFLKLALKF | seqid_920 |
| variant_1198 | RWKKLKKIPPPKFLKLALKF | seqid_921 |
| variant_1199 | FWKKLKKIPPPKFLKLALKF | seqid_922 |
| variant_1200 | KKKLFLKIPPKFLKLALKF | seqid_923 |
| variant_1201 | RKKLFLKIPPKFLKLALKF | seqid_924 |
| variant_1202 | FKKLFLKIPPKFLKLALKF | seqid_925 |
| variant_1203 | KWKLFLKIPPKFLKLALKF | seqid_926 |
| variant_1204 | RWKLFLKIPPKFLKLALKF | seqid_927 |
| variant_1205 | FWKLFLKIPPKFLKLALKF | seqid_928 |
| variant_1206 | KKKKFLKIPPKFLKLALKF | seqid_929 |
| variant_1207 | RKKKFLKIPPKFLKLALKF | seqid_930 |
| variant_1208 | FKKKFLKIPPKFLKLALKF | seqid_931 |
| variant_1209 | KWKKFLKIPPKFLKLALKF | seqid_932 |
| variant_1210 | RWKKFLKIPPKFLKLALKF | seqid_933 |
| variant_1211 | FWKKFLKIPPKFLKLALKF | seqid_934 |
| variant_1212 | KKKLLLKIPPKFLKLALKF | seqid_935 |
| variant_1213 | RKKLLLKIPPKFLKLALKF | seqid_936 |
| variant_1214 | FKKLLLKIPPKFLKLALKF | seqid_937 |
| variant_1215 | KWKLLLKIPPKFLKLALKF | seqid_938 |
| variant_1216 | RWKLLLKIPPKFLKLALKF | seqid_939 |
| variant_1217 | FWKLLLKIPPKFLKLALKF | seqid_940 |
| variant_1218 | KKKKLLKIPPKFLKLALKF | seqid_941 |
| variant_1219 | RKKKLLKIPPKFLKLALKF | seqid_942 |
| variant_1220 | FKKKLLKIPPKFLKLALKF | seqid_943 |
| variant_1221 | KWKKLLKIPPKFLKLALKF | seqid_944 |
| variant_1222 | RWKKLLKIPPKFLKLALKF | seqid_945 |
| variant_1223 | FWKKLLKIPPKFLKLALKF | seqid_946 |
| variant_1224 | KKKLFKKIPPKFLKLALKF | seqid_947 |
| variant_1225 | RKKLFKKIPPKFLKLALKF | seqid_948 |
| variant_1226 | FKKLFKKIPPKFLKLALKF | seqid_949 |
| variant_1227 | KWKLFKKIPPKFLKLALKF | seqid_950 |
| variant_1228 | RWKLFKKIPPKFLKLALKF | seqid_951 |
| variant_1229 | FWKLFKKIPPKFLKLALKF | seqid_952 |
| variant_1230 | KKKKFKKIPPKFLKLALKF | seqid_953 |
| variant_1231 | RKKKFKKIPPKFLKLALKF | seqid_954 |
| variant_1232 | FKKKFKKIPPKFLKLALKF | seqid_955 |
| variant_1233 | KWKKFKKIPPKFLKLALKF | seqid_956 |
| variant_1234 | RWKKFKKIPPKFLKLALKF | seqid_957 |
| variant_1235 | FWKKFKKIPPKFLKLALKF | seqid_958 |
| variant_1236 | KKKLLKKIPPKFLKLALKF | seqid_959 |
| variant_1237 | RKKLLKKIPPKFLKLALKF | seqid_960 |
| variant_1238 | FKKLLKKIPPKFLKLALKF | seqid_961 |
| variant_1239 | KWKLLKKIPPKFLKLALKF | seqid_962 |
| variant_1240 | RWKLLKKIPPKFLKLALKF | seqid_963 |
| variant_1241 | FWKLLKKIPPKFLKLALKF | seqid_964 |
| variant_1242 | KKKKLKKIPPKFLKLALKF | seqid_965 |
| variant_1243 | RKKKLKKIPPKFLKLALKF | seqid_966 |
| variant_1244 | FKKKLKKIPPKFLKLALKF | seqid_967 |
| variant_1245 | KWKKLKKIPPKFLKLALKF | seqid_968 |
| variant_1246 | RWKKLKKIPPKFLKLALKF | seqid_969 |
| variant_1247 | FWKKLKKIPPKFLKLALKF | seqid_970 |
| variant_1296 | KKKLFLKIPKFLKLALKF | seqid_971 |
| variant_1297 | RKKLFLKIPKFLKLALKF | seqid_972 |
| variant_1298 | FKKLFLKIPKFLKLALKF | seqid_973 |
| variant_1299 | KWKLFLKIPKFLKLALKF | seqid_974 |
| variant_1300 | RWKLFLKIPKFLKLALKF | seqid_975 |
| variant_1301 | FWKLFLKIPKFLKLALKF | seqid_976 |
| variant_1302 | KKKKFLKIPKFLKLALKF | seqid_977 |
| variant_1303 | RKKKFLKIPKFLKLALKF | seqid_978 |
| variant_1304 | FKKKFLKIPKFLKLALKF | seqid_979 |
| variant_1305 | KWKKFLKIPKFLKLALKF | seqid_980 |
| variant_1306 | RWKKFLKIPKFLKLALKF | seqid_981 |
| variant_1307 | FWKKFLKIPKFLKLALKF | seqid_982 |
| variant_1308 | KKKLLLKIPKFLKLALKF | seqid_983 |
| variant_1309 | RKKLLLKIPKFLKLALKF | seqid_984 |
| variant_1310 | FKKLLLKIPKFLKLALKF | seqid_985 |
| variant_1311 | KWKLLLKIPKFLKLALKF | seqid_986 |
| variant_1312 | RWKLLLKIPKFLKLALKF | seqid_987 |
| variant_1313 | FWKLLLKIPKFLKLALKF | seqid_988 |
| variant_1314 | KKKKLLKIPKFLKLALKF | seqid_989 |
| variant_1315 | RKKKLLKIPKFLKLALKF | seqid_990 |
| variant_1316 | FKKKLLKIPKFLKLALKF | seqid_991 |
| variant_1317 | KWKKLLKIPKFLKLALKF | seqid_992 |
| variant_1318 | RWKKLLKIPKFLKLALKF | seqid_993 |
| variant_1319 | FWKKLLKIPKFLKLALKF | seqid_994 |
| variant_1320 | KKKLFKKIPKFLKLALKF | seqid_995 |
| variant_1321 | RKKLFKKIPKFLKLALKF | seqid_996 |
| variant_1322 | FKKLFKKIPKFLKLALKF | seqid_997 |
| variant_1323 | KWKLFKKIPKFLKLALKF | seqid_998 |
| variant_1324 | RWKLFKKIPKFLKLALKF | seqid_999 |
| variant_1325 | FWKLFKKIPKFLKLALKF | seqid_1000 |
| variant_1326 | KKKKFKKIPKFLKLALKF | seqid_1001 |
| variant_1327 | RKKKFKKIPKFLKLALKF | seqid_1002 |
| variant_1328 | FKKKFKKIPKFLKLALKF | seqid_1003 |
| variant_1329 | KWKKFKKIPKFLKLALKF | seqid_1004 |
| variant_1330 | RWKKFKKIPKFLKLALKF | seqid_1005 |
| variant_1331 | FWKKFKKIPKFLKLALKF | seqid_1006 |
| variant_1332 | KKKLLKKIPKFLKLALKF | seqid_1007 |
| variant_1333 | RKKLLKKIPKFLKLALKF | seqid_1008 |
| variant_1334 | FKKLLKKIPKFLKLALKF | seqid_1009 |
| variant_1335 | KWKLLKKIPKFLKLALKF | seqid_1010 |
| variant_1336 | RWKLLKKIPKFLKLALKF | seqid_1011 |
| variant_1337 | FWKLLKKIPKFLKLALKF | seqid_1012 |
| variant_1338 | KKKKLKKIPKFLKLALKF | seqid_1013 |
| variant_1339 | RKKKLKKIPKFLKLALKF | seqid_1014 |
| variant_1340 | FKKKLKKIPKFLKLALKF | seqid_1015 |
| variant_1341 | KWKKLKKIPKFLKLALKF | seqid_1016 |
| variant_1342 | RWKKLKKIPKFLKLALKF | seqid_1017 |
| variant_1343 | FWKKLKKIPKFLKLALKF | seqid_1018 |
| variant_1344 | KKKLFLKIPPPKFKKLALKF | seqid_1019 |
| variant_1345 | RKKLFLKIPPPKFKKLALKF | seqid_1020 |
| variant_1346 | FKKLFLKIPPPKFKKLALKF | seqid_1021 |
| variant_1347 | KWKLFLKIPPPKFKKLALKF | seqid_1022 |
| variant_1348 | RWKLFLKIPPPKFKKLALKF | seqid_1023 |
| variant_1349 | FWKLFLKIPPPKFKKLALKF | seqid_1024 |
| variant_1350 | KKKKFLKIPPPKFKKLALKF | seqid_1025 |
| variant_1351 | RKKKFLKIPPPKFKKLALKF | seqid_1026 |
| variant_1352 | FKKKFLKIPPPKFKKLALKF | seqid_1027 |
| variant_1353 | KWKKFLKIPPPKFKKLALKF | seqid_1028 |
| variant_1354 | RWKKFLKIPPPKFKKLALKF | seqid_1029 |
| variant_1355 | FWKKFLKIPPPKFKKLALKF | seqid_1030 |
| variant_1356 | KKKLLLKIPPPKFKKLALKF | seqid_1031 |
| variant_1357 | RKKLLLKIPPPKFKKLALKF | seqid_1032 |
| variant_1358 | FKKLLLKIPPPKFKKLALKF | seqid_1033 |
| variant_1359 | KWKLLLKIPPPKFKKLALKF | seqid_1034 |
| variant_1360 | RWKLLLKIPPPKFKKLALKF | seqid_1035 |
| variant_1361 | FWKLLLKIPPPKFKKLALKF | seqid_1036 |
| variant_1362 | KKKKLLKIPPPKFKKLALKF | seqid_1037 |
| variant_1363 | RKKKLLKIPPPKFKKLALKF | seqid_1038 |
| variant_1364 | FKKKLLKIPPPKFKKLALKF | seqid_1039 |
| variant_1365 | KWKKLLKIPPPKFKKLALKF | seqid_1040 |
| variant_1366 | RWKKLLKIPPPKFKKLALKF | seqid_1041 |
| variant_1367 | FWKKLLKIPPPKFKKLALKF | seqid_1042 |
| variant_1368 | KKKLFKKIPPPKFKKLALKF | seqid_1043 |
| variant_1369 | RKKLFKKIPPPKFKKLALKF | seqid_1044 |
| variant_1370 | FKKLFKKIPPPKFKKLALKF | seqid_1045 |
| variant_1371 | KWKLFKKIPPPKFKKLALKF | seqid_1046 |
| variant_1372 | RWKLFKKIPPPKFKKLALKF | seqid_1047 |
| variant_1373 | FWKLFKKIPPPKFKKLALKF | seqid_1048 |
| variant_1374 | KKKKFKKIPPPKFKKLALKF | seqid_1049 |
| variant_1375 | RKKKFKKIPPPKFKKLALKF | seqid_1050 |
| variant_1376 | FKKKFKKIPPPKFKKLALKF | seqid_1051 |
| variant_1377 | KWKKFKKIPPPKFKKLALKF | seqid_1052 |
| variant_1378 | RWKKFKKIPPPKFKKLALKF | seqid_1053 |
| variant_1379 | FWKKFKKIPPPKFKKLALKF | seqid_1054 |
| variant_1380 | KKKLLKKIPPPKFKKLALKF | seqid_1055 |
| variant_1381 | RKKLLKKIPPPKFKKLALKF | seqid_1056 |
| variant_1382 | FKKLLKKIPPPKFKKLALKF | seqid_1057 |
| variant_1383 | KWKLLKKIPPPKFKKLALKF | seqid_1058 |
| variant_1384 | RWKLLKKIPPPKFKKLALKF | seqid_1059 |
| variant_1385 | FWKLLKKIPPPKFKKLALKF | seqid_1060 |
| variant_1386 | KKKKLKKIPPPKFKKLALKF | seqid_1061 |
| variant_1387 | RKKKLKKIPPPKFKKLALKF | seqid_1062 |
| variant_1388 | FKKKLKKIPPPKFKKLALKF | seqid_1063 |
| variant_1389 | KWKKLKKIPPPKFKKLALKF | seqid_1064 |
| variant_1390 | RWKKLKKIPPPKFKKLALKF | seqid_1065 |
| variant_1391 | FWKKLKKIPPPKFKKLALKF | seqid_1066 |
| variant_1392 | KKKLFLKIPPKFKKLALKF | seqid_1067 |
| variant_1393 | RKKLFLKIPPKFKKLALKF | seqid_1068 |
| variant_1394 | FKKLFLKIPPKFKKLALKF | seqid_1069 |
| variant_1395 | KWKLFLKIPPKFKKLALKF | seqid_1070 |
| variant_1396 | RWKLFLKIPPKFKKLALKF | seqid_1071 |
| variant_1397 | FWKLFLKIPPKFKKLALKF | seqid_1072 |
| variant_1398 | KKKKFLKIPPKFKKLALKF | seqid_1073 |
| variant_1399 | RKKKFLKIPPKFKKLALKF | seqid_1074 |
| variant_1400 | FKKKFLKIPPKFKKLALKF | seqid_1075 |
| variant_1401 | KWKKFLKIPPKFKKLALKF | seqid_1076 |
| variant_1402 | RWKKFLKIPPKFKKLALKF | seqid_1077 |
| variant_1403 | FWKKFLKIPPKFKKLALKF | seqid_1078 |
| variant_1404 | KKKLLLKIPPKFKKLALKF | seqid_1079 |
| variant_1405 | RKKLLLKIPPKFKKLALKF | seqid_1080 |
| variant_1406 | FKKLLLKIPPKFKKLALKF | seqid_1081 |
| variant_1407 | KWKLLLKIPPKFKKLALKF | seqid_1082 |
| variant_1408 | RWKLLLKIPPKFKKLALKF | seqid_1083 |
| variant_1409 | FWKLLLKIPPKFKKLALKF | seqid_1084 |
| variant_1410 | KKKKLLKIPPKFKKLALKF | seqid_1085 |
| variant_1411 | RKKKLLKIPPKFKKLALKF | seqid_1086 |
| variant_1412 | FKKKLLKIPPKFKKLALKF | seqid_1087 |
| variant_1413 | KWKKLLKIPPKFKKLALKF | seqid_1088 |
| variant_1414 | RWKKLLKIPPKFKKLALKF | seqid_1089 |
| variant_1415 | FWKKLLKIPPKFKKLALKF | seqid_1090 |
| variant_1416 | KKKLFKKIPPKFKKLALKF | seqid_1091 |
| variant_1417 | RKKLFKKIPPKFKKLALKF | seqid_1092 |
| variant_1418 | FKKLFKKIPPKFKKLALKF | seqid_1093 |
| variant_1419 | KWKLFKKIPPKFKKLALKF | seqid_1094 |
| variant_1420 | RWKLFKKIPPKFKKLALKF | seqid_1095 |
| variant_1421 | FWKLFKKIPPKFKKLALKF | seqid_1096 |
| variant_1422 | KKKKFKKIPPKFKKLALKF | seqid_1097 |
| variant_1423 | RKKKFKKIPPKFKKLALKF | seqid_1098 |
| variant_1424 | FKKKFKKIPPKFKKLALKF | seqid_1099 |
| variant_1425 | KWKKFKKIPPKFKKLALKF | seqid_1100 |
| variant_1426 | RWKKFKKIPPKFKKLALKF | seqid_1101 |
| variant_1427 | FWKKFKKIPPKFKKLALKF | seqid_1102 |
| variant_1428 | KKKLLKKIPPKFKKLALKF | seqid_1103 |
| variant_1429 | RKKLLKKIPPKFKKLALKF | seqid_1104 |
| variant_1430 | FKKLLKKIPPKFKKLALKF | seqid_1105 |
| variant_1431 | KWKLLKKIPPKFKKLALKF | seqid_1106 |
| variant_1432 | RWKLLKKIPPKFKKLALKF | seqid_1107 |
| variant_1433 | FWKLLKKIPPKFKKLALKF | seqid_1108 |
| variant_1434 | KKKKLKKIPPKFKKLALKF | seqid_1109 |
| variant_1435 | RKKKLKKIPPKFKKLALKF | seqid_1110 |
| variant_1436 | FKKKLKKIPPKFKKLALKF | seqid_1111 |
| variant_1437 | KWKKLKKIPPKFKKLALKF | seqid_1112 |
| variant_1438 | RWKKLKKIPPKFKKLALKF | seqid_1113 |
| variant_1439 | FWKKLKKIPPKFKKLALKF | seqid_1114 |
| variant_1488 | KKKLFLKIPKFKKLALKF | seqid_1115 |
| variant_1489 | RKKLFLKIPKFKKLALKF | seqid_1116 |
| variant_1490 | FKKLFLKIPKFKKLALKF | seqid_1117 |
| variant_1491 | KWKLFLKIPKFKKLALKF | seqid_1118 |
| variant_1492 | RWKLFLKIPKFKKLALKF | seqid_1119 |
| variant_1493 | FWKLFLKIPKFKKLALKF | seqid_1120 |
| variant_1494 | KKKKFLKIPKFKKLALKF | seqid_1121 |
| variant_1495 | RKKKFLKIPKFKKLALKF | seqid_1122 |
| variant_1496 | FKKKFLKIPKFKKLALKF | seqid_1123 |
| variant_1497 | KWKKFLKIPKFKKLALKF | seqid_1124 |
| variant_1498 | RWKKFLKIPKFKKLALKF | seqid_1125 |
| variant_1499 | FWKKFLKIPKFKKLALKF | seqid_1126 |
| variant_1500 | KKKLLLKIPKFKKLALKF | seqid_1127 |
| variant_1501 | RKKLLLKIPKFKKLALKF | seqid_1128 |
| variant_1502 | FKKLLLKIPKFKKLALKF | seqid_1129 |
| variant_1503 | KWKLLLKIPKFKKLALKF | seqid_1130 |
| variant_1504 | RWKLLLKIPKFKKLALKF | seqid_1131 |
| variant_1505 | FWKLLLKIPKFKKLALKF | seqid_1132 |
| variant_1506 | KKKKLLKIPKFKKLALKF | seqid_1133 |
| variant_1507 | RKKKLLKIPKFKKLALKF | seqid_1134 |
| variant_1508 | FKKKLLKIPKFKKLALKF | seqid_1135 |
| variant_1509 | KWKKLLKIPKFKKLALKF | seqid_1136 |
| variant_1510 | RWKKLLKIPKFKKLALKF | seqid_1137 |
| variant_1511 | FWKKLLKIPKFKKLALKF | seqid_1138 |
| variant_1512 | KKKLFKKIPKFKKLALKF | seqid_1139 |
| variant_1513 | RKKLFKKIPKFKKLALKF | seqid_1140 |
| variant_1514 | FKKLFKKIPKFKKLALKF | seqid_1141 |
| variant_1515 | KWKLFKKIPKFKKLALKF | seqid_1142 |
| variant_1516 | RWKLFKKIPKFKKLALKF | seqid_1143 |
| variant_1517 | FWKLFKKIPKFKKLALKF | seqid_1144 |
| variant_1518 | KKKKFKKIPKFKKLALKF | seqid_1145 |
| variant_1519 | RKKKFKKIPKFKKLALKF | seqid_1146 |
| variant_1520 | FKKKFKKIPKFKKLALKF | seqid_1147 |
| variant_1521 | KWKKFKKIPKFKKLALKF | seqid_1148 |
| variant_1522 | RWKKFKKIPKFKKLALKF | seqid_1149 |
| variant_1523 | FWKKFKKIPKFKKLALKF | seqid_1150 |
| variant_1524 | KKKLLKKIPKFKKLALKF | seqid_1151 |
| variant_1525 | RKKLLKKIPKFKKLALKF | seqid_1152 |
| variant_1526 | FKKLLKKIPKFKKLALKF | seqid_1153 |
| variant_1527 | KWKLLKKIPKFKKLALKF | seqid_6 |
| variant_1528 | RWKLLKKIPKFKKLALKF | seqid_1154 |
| variant_1529 | FWKLLKKIPKFKKLALKF | seqid_1155 |
| variant_1530 | KKKKLKKIPKFKKLALKF | seqid_1156 |
| variant_1531 | RKKKLKKIPKFKKLALKF | seqid_1157 |
| variant_1532 | FKKKLKKIPKFKKLALKF | seqid_1158 |
| variant_1533 | KWKKLKKIPKFKKLALKF | seqid_1159 |
| variant_1534 | RWKKLKKIPKFKKLALKF | seqid_1160 |
| variant_1535 | FWKKLKKIPKFKKLALKF | seqid_1161 |
| variant_1536 | KKKLFLKIPPPKFLHVALKF | seqid_1162 |
| variant_1537 | RKKLFLKIPPPKFLHVALKF | seqid_1163 |
| variant_1538 | FKKLFLKIPPPKFLHVALKF | seqid_1164 |
| variant_1539 | KWKLFLKIPPPKFLHVALKF | seqid_1165 |
| variant_1540 | RWKLFLKIPPPKFLHVALKF | seqid_1166 |
| variant_1541 | FWKLFLKIPPPKFLHVALKF | seqid_1167 |
| variant_1542 | KKKKFLKIPPPKFLHVALKF | seqid_1168 |
| variant_1543 | RKKKFLKIPPPKFLHVALKF | seqid_1169 |
| variant_1544 | FKKKFLKIPPPKFLHVALKF | seqid_1170 |
| variant_1545 | KWKKFLKIPPPKFLHVALKF | seqid_1171 |
| variant_1546 | RWKKFLKIPPPKFLHVALKF | seqid_1172 |
| variant_1547 | FWKKFLKIPPPKFLHVALKF | seqid_1173 |
| variant_1548 | KKKLLLKIPPPKFLHVALKF | seqid_1174 |
| variant_1549 | RKKLLLKIPPPKFLHVALKF | seqid_1175 |
| variant_1550 | FKKLLLKIPPPKFLHVALKF | seqid_1176 |
| variant_1551 | KWKLLLKIPPPKFLHVALKF | seqid_1177 |
| variant_1552 | RWKLLLKIPPPKFLHVALKF | seqid_1178 |
| variant_1553 | FWKLLLKIPPPKFLHVALKF | seqid_1179 |
| variant_1554 | KKKKLLKIPPPKFLHVALKF | seqid_1180 |
| variant_1555 | RKKKLLKIPPPKFLHVALKF | seqid_1181 |
| variant_1556 | FKKKLLKIPPPKFLHVALKF | seqid_1182 |
| variant_1557 | KWKKLLKIPPPKFLHVALKF | seqid_1183 |
| variant_1558 | RWKKLLKIPPPKFLHVALKF | seqid_1184 |
| variant_1559 | FWKKLLKIPPPKFLHVALKF | seqid_1185 |
| variant_1560 | KKKLFKKIPPPKFLHVALKF | seqid_1186 |
| variant_1561 | RKKLFKKIPPPKFLHVALKF | seqid_1187 |
| variant_1562 | FKKLFKKIPPPKFLHVALKF | seqid_1188 |
| variant_1563 | KWKLFKKIPPPKFLHVALKF | seqid_1189 |
| variant_1564 | RWKLFKKIPPPKFLHVALKF | seqid_1190 |
| variant_1565 | FWKLFKKIPPPKFLHVALKF | seqid_1191 |
| variant_1566 | KKKKFKKIPPPKFLHVALKF | seqid_1192 |
| variant_1567 | RKKKFKKIPPPKFLHVALKF | seqid_1193 |
| variant_1568 | FKKKFKKIPPPKFLHVALKF | seqid_1194 |
| variant_1569 | KWKKFKKIPPPKFLHVALKF | seqid_1195 |
| variant_1570 | RWKKFKKIPPPKFLHVALKF | seqid_1196 |
| variant_1571 | FWKKFKKIPPPKFLHVALKF | seqid_1197 |
| variant_1572 | KKKLLKKIPPPKFLHVALKF | seqid_1198 |
| variant_1573 | RKKLLKKIPPPKFLHVALKF | seqid_1199 |
| variant_1574 | FKKLLKKIPPPKFLHVALKF | seqid_1200 |
| variant_1575 | KWKLLKKIPPPKFLHVALKF | seqid_1201 |
| variant_1576 | RWKLLKKIPPPKFLHVALKF | seqid_1202 |
| variant_1577 | FWKLLKKIPPPKFLHVALKF | seqid_1203 |
| variant_1578 | KKKKLKKIPPPKFLHVALKF | seqid_1204 |
| variant_1579 | RKKKLKKIPPPKFLHVALKF | seqid_1205 |
| variant_1580 | FKKKLKKIPPPKFLHVALKF | seqid_1206 |
| variant_1581 | KWKKLKKIPPPKFLHVALKF | seqid_1207 |
| variant_1582 | RWKKLKKIPPPKFLHVALKF | seqid_1208 |
| variant_1583 | FWKKLKKIPPPKFLHVALKF | seqid_1209 |
| variant_1584 | KKKLFLKIPPKFLHVALKF | seqid_1210 |
| variant_1585 | RKKLFLKIPPKFLHVALKF | seqid_1211 |
| variant_1586 | FKKLFLKIPPKFLHVALKF | seqid_1212 |
| variant_1587 | KWKLFLKIPPKFLHVALKF | seqid_1213 |
| variant_1588 | RWKLFLKIPPKFLHVALKF | seqid_1214 |
| variant_1589 | FWKLFLKIPPKFLHVALKF | seqid_1215 |
| variant_1590 | KKKKFLKIPPKFLHVALKF | seqid_1216 |
| variant_1591 | RKKKFLKIPPKFLHVALKF | seqid_1217 |
| variant_1592 | FKKKFLKIPPKFLHVALKF | seqid_1218 |
| variant_1593 | KWKKFLKIPPKFLHVALKF | seqid_1219 |
| variant_1594 | RWKKFLKIPPKFLHVALKF | seqid_1220 |
| variant_1595 | FWKKFLKIPPKFLHVALKF | seqid_1221 |
| variant_1596 | KKKLLLKIPPKFLHVALKF | seqid_1222 |
| variant_1597 | RKKLLLKIPPKFLHVALKF | seqid_1223 |
| variant_1598 | FKKLLLKIPPKFLHVALKF | seqid_1224 |
| variant_1599 | KWKLLLKIPPKFLHVALKF | seqid_1225 |
| variant_1600 | RWKLLLKIPPKFLHVALKF | seqid_1226 |
| variant_1601 | FWKLLLKIPPKFLHVALKF | seqid_1227 |
| variant_1602 | KKKKLLKIPPKFLHVALKF | seqid_1228 |
| variant_1603 | RKKKLLKIPPKFLHVALKF | seqid_1229 |
| variant_1604 | FKKKLLKIPPKFLHVALKF | seqid_1230 |
| variant_1605 | KWKKLLKIPPKFLHVALKF | seqid_1231 |
| variant_1606 | RWKKLLKIPPKFLHVALKF | seqid_1232 |
| variant_1607 | FWKKLLKIPPKFLHVALKF | seqid_1233 |
| variant_1608 | KKKLFKKIPPKFLHVALKF | seqid_1234 |
| variant_1609 | RKKLFKKIPPKFLHVALKF | seqid_1235 |
| variant_1610 | FKKLFKKIPPKFLHVALKF | seqid_1236 |
| variant_1611 | KWKLFKKIPPKFLHVALKF | seqid_1237 |
| variant_1612 | RWKLFKKIPPKFLHVALKF | seqid_1238 |
| variant_1613 | FWKLFKKIPPKFLHVALKF | seqid_1239 |
| variant_1614 | KKKKFKKIPPKFLHVALKF | seqid_1240 |
| variant_1615 | RKKKFKKIPPKFLHVALKF | seqid_1241 |
| variant_1616 | FKKKFKKIPPKFLHVALKF | seqid_1242 |
| variant_1617 | KWKKFKKIPPKFLHVALKF | seqid_1243 |
| variant_1618 | RWKKFKKIPPKFLHVALKF | seqid_1244 |
| variant_1619 | FWKKFKKIPPKFLHVALKF | seqid_1245 |
| variant_1620 | KKKLLKKIPPKFLHVALKF | seqid_1246 |
| variant_1621 | RKKLLKKIPPKFLHVALKF | seqid_1247 |
| variant_1622 | FKKLLKKIPPKFLHVALKF | seqid_1248 |
| variant_1623 | KWKLLKKIPPKFLHVALKF | seqid_1249 |
| variant_1624 | RWKLLKKIPPKFLHVALKF | seqid_1250 |
| variant_1625 | FWKLLKKIPPKFLHVALKF | seqid_1251 |
| variant_1626 | KKKKLKKIPPKFLHVALKF | seqid_1252 |
| variant_1627 | RKKKLKKIPPKFLHVALKF | seqid_1253 |
| variant_1628 | FKKKLKKIPPKFLHVALKF | seqid_1254 |
| variant_1629 | KWKKLKKIPPKFLHVALKF | seqid_1255 |
| variant_1630 | RWKKLKKIPPKFLHVALKF | seqid_1256 |
| variant_1631 | FWKKLKKIPPKFLHVALKF | seqid_1257 |
| variant_1680 | KKKLFLKIPKFLHVALKF | seqid_1258 |
| variant_1681 | RKKLFLKIPKFLHVALKF | seqid_1259 |
| variant_1682 | FKKLFLKIPKFLHVALKF | seqid_1260 |
| variant_1683 | KWKLFLKIPKFLHVALKF | seqid_1261 |
| variant_1684 | RWKLFLKIPKFLHVALKF | seqid_1262 |
| variant_1685 | FWKLFLKIPKFLHVALKF | seqid_1263 |
| variant_1686 | KKKKFLKIPKFLHVALKF | seqid_1264 |
| variant_1687 | RKKKFLKIPKFLHVALKF | seqid_1265 |
| variant_1688 | FKKKFLKIPKFLHVALKF | seqid_1266 |
| variant_1689 | KWKKFLKIPKFLHVALKF | seqid_1267 |
| variant_1690 | RWKKFLKIPKFLHVALKF | seqid_1268 |
| variant_1691 | FWKKFLKIPKFLHVALKF | seqid_1269 |
| variant_1692 | KKKLLLKIPKFLHVALKF | seqid_1270 |
| variant_1693 | RKKLLLKIPKFLHVALKF | seqid_1271 |
| variant_1694 | FKKLLLKIPKFLHVALKF | seqid_1272 |
| variant_1695 | KWKLLLKIPKFLHVALKF | seqid_1273 |
| variant_1696 | RWKLLLKIPKFLHVALKF | seqid_1274 |
| variant_1697 | FWKLLLKIPKFLHVALKF | seqid_1275 |
| variant_1698 | KKKKLLKIPKFLHVALKF | seqid_1276 |
| variant_1699 | RKKKLLKIPKFLHVALKF | seqid_1277 |
| variant_1700 | FKKKLLKIPKFLHVALKF | seqid_1278 |
| variant_1701 | KWKKLLKIPKFLHVALKF | seqid_1279 |
| variant_1702 | RWKKLLKIPKFLHVALKF | seqid_1280 |
| variant_1703 | FWKKLLKIPKFLHVALKF | seqid_1281 |
| variant_1704 | KKKLFKKIPKFLHVALKF | seqid_1282 |
| variant_1705 | RKKLFKKIPKFLHVALKF | seqid_1283 |
| variant_1706 | FKKLFKKIPKFLHVALKF | seqid_1284 |
| variant_1707 | KWKLFKKIPKFLHVALKF | seqid_1285 |
| variant_1708 | RWKLFKKIPKFLHVALKF | seqid_1286 |
| variant_1709 | FWKLFKKIPKFLHVALKF | seqid_1287 |
| variant_1710 | KKKKFKKIPKFLHVALKF | seqid_1288 |
| variant_1711 | RKKKFKKIPKFLHVALKF | seqid_1289 |
| variant_1712 | FKKKFKKIPKFLHVALKF | seqid_1290 |
| variant_1713 | KWKKFKKIPKFLHVALKF | seqid_1291 |
| variant_1714 | RWKKFKKIPKFLHVALKF | seqid_1292 |
| variant_1715 | FWKKFKKIPKFLHVALKF | seqid_1293 |
| variant_1716 | KKKLLKKIPKFLHVALKF | seqid_1294 |
| variant_1717 | RKKLLKKIPKFLHVALKF | seqid_1295 |
| variant_1718 | FKKLLKKIPKFLHVALKF | seqid_1296 |
| variant_1719 | KWKLLKKIPKFLHVALKF | seqid_1297 |
| variant_1720 | RWKLLKKIPKFLHVALKF | seqid_1298 |
| variant_1721 | FWKLLKKIPKFLHVALKF | seqid_1299 |
| variant_1722 | KKKKLKKIPKFLHVALKF | seqid_1300 |
| variant_1723 | RKKKLKKIPKFLHVALKF | seqid_1301 |
| variant_1724 | FKKKLKKIPKFLHVALKF | seqid_1302 |
| variant_1725 | KWKKLKKIPKFLHVALKF | seqid_1303 |
| variant_1726 | RWKKLKKIPKFLHVALKF | seqid_1304 |
| variant_1727 | FWKKLKKIPKFLHVALKF | seqid_1305 |
| variant_1728 | KKKLFLKIPPPKFKHVALKF | seqid_1306 |
| variant_1729 | RKKLFLKIPPPKFKHVALKF | seqid_1307 |
| variant_1730 | FKKLFLKIPPPKFKHVALKF | seqid_1308 |
| variant_1731 | KWKLFLKIPPPKFKHVALKF | seqid_1309 |
| variant_1732 | RWKLFLKIPPPKFKHVALKF | seqid_1310 |
| variant_1733 | FWKLFLKIPPPKFKHVALKF | seqid_1311 |
| variant_1734 | KKKKFLKIPPPKFKHVALKF | seqid_1312 |
| variant_1735 | RKKKFLKIPPPKFKHVALKF | seqid_1313 |
| variant_1736 | FKKKFLKIPPPKFKHVALKF | seqid_1314 |
| variant_1737 | KWKKFLKIPPPKFKHVALKF | seqid_1315 |
| variant_1738 | RWKKFLKIPPPKFKHVALKF | seqid_1316 |
| variant_1739 | FWKKFLKIPPPKFKHVALKF | seqid_1317 |
| variant_1740 | KKKLLLKIPPPKFKHVALKF | seqid_1318 |
| variant_1741 | RKKLLLKIPPPKFKHVALKF | seqid_1319 |
| variant_1742 | FKKLLLKIPPPKFKHVALKF | seqid_1320 |
| variant_1743 | KWKLLLKIPPPKFKHVALKF | seqid_1321 |
| variant_1744 | RWKLLLKIPPPKFKHVALKF | seqid_1322 |
| variant_1745 | FWKLLLKIPPPKFKHVALKF | seqid_1323 |
| variant_1746 | KKKKLLKIPPPKFKHVALKF | seqid_1324 |
| variant_1747 | RKKKLLKIPPPKFKHVALKF | seqid_1325 |
| variant_1748 | FKKKLLKIPPPKFKHVALKF | seqid_1326 |
| variant_1749 | KWKKLLKIPPPKFKHVALKF | seqid_1327 |
| variant_1750 | RWKKLLKIPPPKFKHVALKF | seqid_1328 |
| variant_1751 | FWKKLLKIPPPKFKHVALKF | seqid_1329 |
| variant_1752 | KKKLFKKIPPPKFKHVALKF | seqid_1330 |
| variant_1753 | RKKLFKKIPPPKFKHVALKF | seqid_1331 |
| variant_1754 | FKKLFKKIPPPKFKHVALKF | seqid_1332 |
| variant_1755 | KWKLFKKIPPPKFKHVALKF | seqid_1333 |
| variant_1756 | RWKLFKKIPPPKFKHVALKF | seqid_1334 |
| variant_1757 | FWKLFKKIPPPKFKHVALKF | seqid_1335 |
| variant_1758 | KKKKFKKIPPPKFKHVALKF | seqid_1336 |
| variant_1759 | RKKKFKKIPPPKFKHVALKF | seqid_1337 |
| variant_1760 | FKKKFKKIPPPKFKHVALKF | seqid_1338 |
| variant_1761 | KWKKFKKIPPPKFKHVALKF | seqid_1339 |
| variant_1762 | RWKKFKKIPPPKFKHVALKF | seqid_1340 |
| variant_1763 | FWKKFKKIPPPKFKHVALKF | seqid_1341 |
| variant_1764 | KKKLLKKIPPPKFKHVALKF | seqid_1342 |
| variant_1765 | RKKLLKKIPPPKFKHVALKF | seqid_1343 |
| variant_1766 | FKKLLKKIPPPKFKHVALKF | seqid_1344 |
| variant_1767 | KWKLLKKIPPPKFKHVALKF | seqid_1345 |
| variant_1768 | RWKLLKKIPPPKFKHVALKF | seqid_1346 |
| variant_1769 | FWKLLKKIPPPKFKHVALKF | seqid_1347 |
| variant_1770 | KKKKLKKIPPPKFKHVALKF | seqid_1348 |
| variant_1771 | RKKKLKKIPPPKFKHVALKF | seqid_1349 |
| variant_1772 | FKKKLKKIPPPKFKHVALKF | seqid_1350 |
| variant_1773 | KWKKLKKIPPPKFKHVALKF | seqid_1351 |
| variant_1774 | RWKKLKKIPPPKFKHVALKF | seqid_1352 |
| variant_1775 | FWKKLKKIPPPKFKHVALKF | seqid_1353 |
| variant_1776 | KKKLFLKIPPKFKHVALKF | seqid_1354 |
| variant_1777 | RKKLFLKIPPKFKHVALKF | seqid_1355 |
| variant_1778 | FKKLFLKIPPKFKHVALKF | seqid_1356 |
| variant_1779 | KWKLFLKIPPKFKHVALKF | seqid_1357 |
| variant_1780 | RWKLFLKIPPKFKHVALKF | seqid_1358 |
| variant_1781 | FWKLFLKIPPKFKHVALKF | seqid_1359 |
| variant_1782 | KKKKFLKIPPKFKHVALKF | seqid_1360 |
| variant_1783 | RKKKFLKIPPKFKHVALKF | seqid_1361 |
| variant_1784 | FKKKFLKIPPKFKHVALKF | seqid_1362 |
| variant_1785 | KWKKFLKIPPKFKHVALKF | seqid_1363 |
| variant_1786 | RWKKFLKIPPKFKHVALKF | seqid_1364 |
| variant_1787 | FWKKFLKIPPKFKHVALKF | seqid_1365 |
| variant_1788 | KKKLLLKIPPKFKHVALKF | seqid_1366 |
| variant_1789 | RKKLLLKIPPKFKHVALKF | seqid_1367 |
| variant_1790 | FKKLLLKIPPKFKHVALKF | seqid_1368 |
| variant_1791 | KWKLLLKIPPKFKHVALKF | seqid_1369 |
| variant_1792 | RWKLLLKIPPKFKHVALKF | seqid_1370 |
| variant_1793 | FWKLLLKIPPKFKHVALKF | seqid_1371 |
| variant_1794 | KKKKLLKIPPKFKHVALKF | seqid_1372 |
| variant_1795 | RKKKLLKIPPKFKHVALKF | seqid_1373 |
| variant_1796 | FKKKLLKIPPKFKHVALKF | seqid_1374 |
| variant_1797 | KWKKLLKIPPKFKHVALKF | seqid_1375 |
| variant_1798 | RWKKLLKIPPKFKHVALKF | seqid_1376 |
| variant_1799 | FWKKLLKIPPKFKHVALKF | seqid_1377 |
| variant_1800 | KKKLFKKIPPKFKHVALKF | seqid_1378 |
| variant_1801 | RKKLFKKIPPKFKHVALKF | seqid_1379 |
| variant_1802 | FKKLFKKIPPKFKHVALKF | seqid_1380 |
| variant_1803 | KWKLFKKIPPKFKHVALKF | seqid_1381 |
| variant_1804 | RWKLFKKIPPKFKHVALKF | seqid_1382 |
| variant_1805 | FWKLFKKIPPKFKHVALKF | seqid_1383 |
| variant_1806 | KKKKFKKIPPKFKHVALKF | seqid_1384 |
| variant_1807 | RKKKFKKIPPKFKHVALKF | seqid_1385 |
| variant_1808 | FKKKFKKIPPKFKHVALKF | seqid_1386 |
| variant_1809 | KWKKFKKIPPKFKHVALKF | seqid_1387 |
| variant_1810 | RWKKFKKIPPKFKHVALKF | seqid_1388 |
| variant_1811 | FWKKFKKIPPKFKHVALKF | seqid_1389 |
| variant_1812 | KKKLLKKIPPKFKHVALKF | seqid_1390 |
| variant_1813 | RKKLLKKIPPKFKHVALKF | seqid_1391 |
| variant_1814 | FKKLLKKIPPKFKHVALKF | seqid_1392 |
| variant_1815 | KWKLLKKIPPKFKHVALKF | seqid_1393 |
| variant_1816 | RWKLLKKIPPKFKHVALKF | seqid_1394 |
| variant_1817 | FWKLLKKIPPKFKHVALKF | seqid_1395 |
| variant_1818 | KKKKLKKIPPKFKHVALKF | seqid_1396 |
| variant_1819 | RKKKLKKIPPKFKHVALKF | seqid_1397 |
| variant_1820 | FKKKLKKIPPKFKHVALKF | seqid_1398 |
| variant_1821 | KWKKLKKIPPKFKHVALKF | seqid_1399 |
| variant_1822 | RWKKLKKIPPKFKHVALKF | seqid_1400 |
| variant_1823 | FWKKLKKIPPKFKHVALKF | seqid_1401 |
| variant_1872 | KKKLFLKIPKFKHVALKF | seqid_1402 |
| variant_1873 | RKKLFLKIPKFKHVALKF | seqid_1403 |
| variant_1874 | FKKLFLKIPKFKHVALKF | seqid_1404 |
| variant_1875 | KWKLFLKIPKFKHVALKF | seqid_1405 |
| variant_1876 | RWKLFLKIPKFKHVALKF | seqid_1406 |
| variant_1877 | FWKLFLKIPKFKHVALKF | seqid_1407 |
| variant_1878 | KKKKFLKIPKFKHVALKF | seqid_1408 |
| variant_1879 | RKKKFLKIPKFKHVALKF | seqid_1409 |
| variant_1880 | FKKKFLKIPKFKHVALKF | seqid_1410 |
| variant_1881 | KWKKFLKIPKFKHVALKF | seqid_1411 |
| variant_1882 | RWKKFLKIPKFKHVALKF | seqid_1412 |
| variant_1883 | FWKKFLKIPKFKHVALKF | seqid_1413 |
| variant_1884 | KKKLLLKIPKFKHVALKF | seqid_1414 |
| variant_1885 | RKKLLLKIPKFKHVALKF | seqid_1415 |
| variant_1886 | FKKLLLKIPKFKHVALKF | seqid_1416 |
| variant_1887 | KWKLLLKIPKFKHVALKF | seqid_1417 |
| variant_1888 | RWKLLLKIPKFKHVALKF | seqid_1418 |
| variant_1889 | FWKLLLKIPKFKHVALKF | seqid_1419 |
| variant_1890 | KKKKLLKIPKFKHVALKF | seqid_1420 |
| variant_1891 | RKKKLLKIPKFKHVALKF | seqid_1421 |
| variant_1892 | FKKKLLKIPKFKHVALKF | seqid_1422 |
| variant_1893 | KWKKLLKIPKFKHVALKF | seqid_1423 |
| variant_1894 | RWKKLLKIPKFKHVALKF | seqid_1424 |
| variant_1895 | FWKKLLKIPKFKHVALKF | seqid_1425 |
| variant_1896 | KKKLFKKIPKFKHVALKF | seqid_1426 |
| variant_1897 | RKKLFKKIPKFKHVALKF | seqid_1427 |
| variant_1898 | FKKLFKKIPKFKHVALKF | seqid_1428 |
| variant_1899 | KWKLFKKIPKFKHVALKF | seqid_1429 |
| variant_1900 | RWKLFKKIPKFKHVALKF | seqid_1430 |
| variant_1901 | FWKLFKKIPKFKHVALKF | seqid_1431 |
| variant_1902 | KKKKFKKIPKFKHVALKF | seqid_1432 |
| variant_1903 | RKKKFKKIPKFKHVALKF | seqid_1433 |
| variant_1904 | FKKKFKKIPKFKHVALKF | seqid_1434 |
| variant_1905 | KWKKFKKIPKFKHVALKF | seqid_1435 |
| variant_1906 | RWKKFKKIPKFKHVALKF | seqid_1436 |
| variant_1907 | FWKKFKKIPKFKHVALKF | seqid_1437 |
| variant_1908 | KKKLLKKIPKFKHVALKF | seqid_1438 |
| variant_1909 | RKKLLKKIPKFKHVALKF | seqid_1439 |
| variant_1910 | FKKLLKKIPKFKHVALKF | seqid_1440 |
| variant_1911 | KWKLLKKIPKFKHVALKF | seqid_1441 |
| variant_1912 | RWKLLKKIPKFKHVALKF | seqid_1442 |
| variant_1913 | FWKLLKKIPKFKHVALKF | seqid_1443 |
| variant_1914 | KKKKLKKIPKFKHVALKF | seqid_1444 |
| variant_1915 | RKKKLKKIPKFKHVALKF | seqid_1445 |
| variant_1916 | FKKKLKKIPKFKHVALKF | seqid_1446 |
| variant_1917 | KWKKLKKIPKFKHVALKF | seqid_1447 |
| variant_1918 | RWKKLKKIPKFKHVALKF | seqid_1448 |
| variant_1919 | FWKKLKKIPKFKHVALKF | seqid_1449 |
| variant_1920 | KKKLFLKIPPPKFLKVALKF | seqid_1450 |
| variant_1921 | RKKLFLKIPPPKFLKVALKF | seqid_1451 |
| variant_1922 | FKKLFLKIPPPKFLKVALKF | seqid_1452 |
| variant_1923 | KWKLFLKIPPPKFLKVALKF | seqid_1453 |
| variant_1924 | RWKLFLKIPPPKFLKVALKF | seqid_1454 |
| variant_1925 | FWKLFLKIPPPKFLKVALKF | seqid_1455 |
| variant_1926 | KKKKFLKIPPPKFLKVALKF | seqid_1456 |
| variant_1927 | RKKKFLKIPPPKFLKVALKF | seqid_1457 |
| variant_1928 | FKKKFLKIPPPKFLKVALKF | seqid_1458 |
| variant_1929 | KWKKFLKIPPPKFLKVALKF | seqid_1459 |
| variant_1930 | RWKKFLKIPPPKFLKVALKF | seqid_1460 |
| variant_1931 | FWKKFLKIPPPKFLKVALKF | seqid_1461 |
| variant_1932 | KKKLLLKIPPPKFLKVALKF | seqid_1462 |
| variant_1933 | RKKLLLKIPPPKFLKVALKF | seqid_1463 |
| variant_1934 | FKKLLLKIPPPKFLKVALKF | seqid_1464 |
| variant_1935 | KWKLLLKIPPPKFLKVALKF | seqid_1465 |
| variant_1936 | RWKLLLKIPPPKFLKVALKF | seqid_1466 |
| variant_1937 | FWKLLLKIPPPKFLKVALKF | seqid_1467 |
| variant_1938 | KKKKLLKIPPPKFLKVALKF | seqid_1468 |
| variant_1939 | RKKKLLKIPPPKFLKVALKF | seqid_1469 |
| variant_1940 | FKKKLLKIPPPKFLKVALKF | seqid_1470 |
| variant_1941 | KWKKLLKIPPPKFLKVALKF | seqid_1471 |
| variant_1942 | RWKKLLKIPPPKFLKVALKF | seqid_1472 |
| variant_1943 | FWKKLLKIPPPKFLKVALKF | seqid_1473 |
| variant_1944 | KKKLFKKIPPPKFLKVALKF | seqid_1474 |
| variant_1945 | RKKLFKKIPPPKFLKVALKF | seqid_1475 |
| variant_1946 | FKKLFKKIPPPKFLKVALKF | seqid_1476 |
| variant_1947 | KWKLFKKIPPPKFLKVALKF | seqid_1477 |
| variant_1948 | RWKLFKKIPPPKFLKVALKF | seqid_1478 |
| variant_1949 | FWKLFKKIPPPKFLKVALKF | seqid_1479 |
| variant_1950 | KKKKFKKIPPPKFLKVALKF | seqid_1480 |
| variant_1951 | RKKKFKKIPPPKFLKVALKF | seqid_1481 |
| variant_1952 | FKKKFKKIPPPKFLKVALKF | seqid_1482 |
| variant_1953 | KWKKFKKIPPPKFLKVALKF | seqid_1483 |
| variant_1954 | RWKKFKKIPPPKFLKVALKF | seqid_1484 |
| variant_1955 | FWKKFKKIPPPKFLKVALKF | seqid_1485 |
| variant_1956 | KKKLLKKIPPPKFLKVALKF | seqid_1486 |
| variant_1957 | RKKLLKKIPPPKFLKVALKF | seqid_1487 |
| variant_1958 | FKKLLKKIPPPKFLKVALKF | seqid_1488 |
| variant_1959 | KWKLLKKIPPPKFLKVALKF | seqid_1489 |
| variant_1960 | RWKLLKKIPPPKFLKVALKF | seqid_1490 |
| variant_1961 | FWKLLKKIPPPKFLKVALKF | seqid_1491 |
| variant_1962 | KKKKLKKIPPPKFLKVALKF | seqid_1492 |
| variant_1963 | RKKKLKKIPPPKFLKVALKF | seqid_1493 |
| variant_1964 | FKKKLKKIPPPKFLKVALKF | seqid_1494 |
| variant_1965 | KWKKLKKIPPPKFLKVALKF | seqid_1495 |
| variant_1966 | RWKKLKKIPPPKFLKVALKF | seqid_1496 |
| variant_1967 | FWKKLKKIPPPKFLKVALKF | seqid_1497 |
| variant_1968 | KKKLFLKIPPKFLKVALKF | seqid_1498 |
| variant_1969 | RKKLFLKIPPKFLKVALKF | seqid_1499 |
| variant_1970 | FKKLFLKIPPKFLKVALKF | seqid_1500 |
| variant_1971 | KWKLFLKIPPKFLKVALKF | seqid_1501 |
| variant_1972 | RWKLFLKIPPKFLKVALKF | seqid_1502 |
| variant_1973 | FWKLFLKIPPKFLKVALKF | seqid_1503 |
| variant_1974 | KKKKFLKIPPKFLKVALKF | seqid_1504 |
| variant_1975 | RKKKFLKIPPKFLKVALKF | seqid_1505 |
| variant_1976 | FKKKFLKIPPKFLKVALKF | seqid_1506 |
| variant_1977 | KWKKFLKIPPKFLKVALKF | seqid_1507 |
| variant_1978 | RWKKFLKIPPKFLKVALKF | seqid_1508 |
| variant_1979 | FWKKFLKIPPKFLKVALKF | seqid_1509 |
| variant_1980 | KKKLLLKIPPKFLKVALKF | seqid_1510 |
| variant_1981 | RKKLLLKIPPKFLKVALKF | seqid_1511 |
| variant_1982 | FKKLLLKIPPKFLKVALKF | seqid_1512 |
| variant_1983 | KWKLLLKIPPKFLKVALKF | seqid_1513 |
| variant_1984 | RWKLLLKIPPKFLKVALKF | seqid_1514 |
| variant_1985 | FWKLLLKIPPKFLKVALKF | seqid_1515 |
| variant_1986 | KKKKLLKIPPKFLKVALKF | seqid_1516 |
| variant_1987 | RKKKLLKIPPKFLKVALKF | seqid_1517 |
| variant_1988 | FKKKLLKIPPKFLKVALKF | seqid_1518 |
| variant_1989 | KWKKLLKIPPKFLKVALKF | seqid_1519 |
| variant_1990 | RWKKLLKIPPKFLKVALKF | seqid_1520 |
| variant_1991 | FWKKLLKIPPKFLKVALKF | seqid_1521 |
| variant_1992 | KKKLFKKIPPKFLKVALKF | seqid_1522 |
| variant_1993 | RKKLFKKIPPKFLKVALKF | seqid_1523 |
| variant_1994 | FKKLFKKIPPKFLKVALKF | seqid_1524 |
| variant_1995 | KWKLFKKIPPKFLKVALKF | seqid_1525 |
| variant_1996 | RWKLFKKIPPKFLKVALKF | seqid_1526 |
| variant_1997 | FWKLFKKIPPKFLKVALKF | seqid_1527 |
| variant_1998 | KKKKFKKIPPKFLKVALKF | seqid_1528 |
| variant_1999 | RKKKFKKIPPKFLKVALKF | seqid_1529 |
| variant_2000 | FKKKFKKIPPKFLKVALKF | seqid_1530 |
| variant_2001 | KWKKFKKIPPKFLKVALKF | seqid_1531 |
| variant_2002 | RWKKFKKIPPKFLKVALKF | seqid_1532 |
| variant_2003 | FWKKFKKIPPKFLKVALKF | seqid_1533 |
| variant_2004 | KKKLLKKIPPKFLKVALKF | seqid_1534 |
| variant_2005 | RKKLLKKIPPKFLKVALKF | seqid_1535 |
| variant_2006 | FKKLLKKIPPKFLKVALKF | seqid_1536 |
| variant_2007 | KWKLLKKIPPKFLKVALKF | seqid_1537 |
| variant_2008 | RWKLLKKIPPKFLKVALKF | seqid_1538 |
| variant_2009 | FWKLLKKIPPKFLKVALKF | seqid_1539 |
| variant_2010 | KKKKLKKIPPKFLKVALKF | seqid_1540 |
| variant_2011 | RKKKLKKIPPKFLKVALKF | seqid_1541 |
| variant_2012 | FKKKLKKIPPKFLKVALKF | seqid_1542 |
| variant_2013 | KWKKLKKIPPKFLKVALKF | seqid_1543 |
| variant_2014 | RWKKLKKIPPKFLKVALKF | seqid_1544 |
| variant_2015 | FWKKLKKIPPKFLKVALKF | seqid_1545 |
| variant_2064 | KKKLFLKIPKFLKVALKF | seqid_1546 |
| variant_2065 | RKKLFLKIPKFLKVALKF | seqid_1547 |
| variant_2066 | FKKLFLKIPKFLKVALKF | seqid_1548 |
| variant_2067 | KWKLFLKIPKFLKVALKF | seqid_1549 |
| variant_2068 | RWKLFLKIPKFLKVALKF | seqid_1550 |
| variant_2069 | FWKLFLKIPKFLKVALKF | seqid_1551 |
| variant_2070 | KKKKFLKIPKFLKVALKF | seqid_1552 |
| variant_2071 | RKKKFLKIPKFLKVALKF | seqid_1553 |
| variant_2072 | FKKKFLKIPKFLKVALKF | seqid_1554 |
| variant_2073 | KWKKFLKIPKFLKVALKF | seqid_1555 |
| variant_2074 | RWKKFLKIPKFLKVALKF | seqid_1556 |
| variant_2075 | FWKKFLKIPKFLKVALKF | seqid_1557 |
| variant_2076 | KKKLLLKIPKFLKVALKF | seqid_1558 |
| variant_2077 | RKKLLLKIPKFLKVALKF | seqid_1559 |
| variant_2078 | FKKLLLKIPKFLKVALKF | seqid_1560 |
| variant_2079 | KWKLLLKIPKFLKVALKF | seqid_1561 |
| variant_2080 | RWKLLLKIPKFLKVALKF | seqid_1562 |
| variant_2081 | FWKLLLKIPKFLKVALKF | seqid_1563 |
| variant_2082 | KKKKLLKIPKFLKVALKF | seqid_1564 |
| variant_2083 | RKKKLLKIPKFLKVALKF | seqid_1565 |
| variant_2084 | FKKKLLKIPKFLKVALKF | seqid_1566 |
| variant_2085 | KWKKLLKIPKFLKVALKF | seqid_1567 |
| variant_2086 | RWKKLLKIPKFLKVALKF | seqid_1568 |
| variant_2087 | FWKKLLKIPKFLKVALKF | seqid_1569 |
| variant_2088 | KKKLFKKIPKFLKVALKF | seqid_1570 |
| variant_2089 | RKKLFKKIPKFLKVALKF | seqid_1571 |
| variant_2090 | FKKLFKKIPKFLKVALKF | seqid_1572 |
| variant_2091 | KWKLFKKIPKFLKVALKF | seqid_1573 |
| variant_2092 | RWKLFKKIPKFLKVALKF | seqid_1574 |
| variant_2093 | FWKLFKKIPKFLKVALKF | seqid_1575 |
| variant_2094 | KKKKFKKIPKFLKVALKF | seqid_1576 |
| variant_2095 | RKKKFKKIPKFLKVALKF | seqid_1577 |
| variant_2096 | FKKKFKKIPKFLKVALKF | seqid_1578 |
| variant_2097 | KWKKFKKIPKFLKVALKF | seqid_1579 |
| variant_2098 | RWKKFKKIPKFLKVALKF | seqid_1580 |
| variant_2099 | FWKKFKKIPKFLKVALKF | seqid_1581 |
| variant_2100 | KKKLLKKIPKFLKVALKF | seqid_1582 |
| variant_2101 | RKKLLKKIPKFLKVALKF | seqid_1583 |
| variant_2102 | FKKLLKKIPKFLKVALKF | seqid_1584 |
| variant_2103 | KWKLLKKIPKFLKVALKF | seqid_1585 |
| variant_2104 | RWKLLKKIPKFLKVALKF | seqid_1586 |
| variant_2105 | FWKLLKKIPKFLKVALKF | seqid_1587 |
| variant_2106 | KKKKLKKIPKFLKVALKF | seqid_1588 |
| variant_2107 | RKKKLKKIPKFLKVALKF | seqid_1589 |
| variant_2108 | FKKKLKKIPKFLKVALKF | seqid_1590 |
| variant_2109 | KWKKLKKIPKFLKVALKF | seqid_1591 |
| variant_2110 | RWKKLKKIPKFLKVALKF | seqid_1592 |
| variant_2111 | FWKKLKKIPKFLKVALKF | seqid_1593 |
| variant_2112 | KKKLFLKIPPPKFKKVALKF | seqid_1594 |
| variant_2113 | RKKLFLKIPPPKFKKVALKF | seqid_1595 |
| variant_2114 | FKKLFLKIPPPKFKKVALKF | seqid_1596 |
| variant_2115 | KWKLFLKIPPPKFKKVALKF | seqid_1597 |
| variant_2116 | RWKLFLKIPPPKFKKVALKF | seqid_1598 |
| variant_2117 | FWKLFLKIPPPKFKKVALKF | seqid_1599 |
| variant_2118 | KKKKFLKIPPPKFKKVALKF | seqid_1600 |
| variant_2119 | RKKKFLKIPPPKFKKVALKF | seqid_1601 |
| variant_2120 | FKKKFLKIPPPKFKKVALKF | seqid_1602 |
| variant_2121 | KWKKFLKIPPPKFKKVALKF | seqid_1603 |
| variant_2122 | RWKKFLKIPPPKFKKVALKF | seqid_1604 |
| variant_2123 | FWKKFLKIPPPKFKKVALKF | seqid_1605 |
| variant_2124 | KKKLLLKIPPPKFKKVALKF | seqid_1606 |
| variant_2125 | RKKLLLKIPPPKFKKVALKF | seqid_1607 |
| variant_2126 | FKKLLLKIPPPKFKKVALKF | seqid_1608 |
| variant_2127 | KWKLLLKIPPPKFKKVALKF | seqid_1609 |
| variant_2128 | RWKLLLKIPPPKFKKVALKF | seqid_1610 |
| variant_2129 | FWKLLLKIPPPKFKKVALKF | seqid_1611 |
| variant_2130 | KKKKLLKIPPPKFKKVALKF | seqid_1612 |
| variant_2131 | RKKKLLKIPPPKFKKVALKF | seqid_1613 |
| variant_2132 | FKKKLLKIPPPKFKKVALKF | seqid_1614 |
| variant_2133 | KWKKLLKIPPPKFKKVALKF | seqid_1615 |
| variant_2134 | RWKKLLKIPPPKFKKVALKF | seqid_1616 |
| variant_2135 | FWKKLLKIPPPKFKKVALKF | seqid_1617 |
| variant_2136 | KKKLFKKIPPPKFKKVALKF | seqid_1618 |
| variant_2137 | RKKLFKKIPPPKFKKVALKF | seqid_1619 |
| variant_2138 | FKKLFKKIPPPKFKKVALKF | seqid_1620 |
| variant_2139 | KWKLFKKIPPPKFKKVALKF | seqid_1621 |
| variant_2140 | RWKLFKKIPPPKFKKVALKF | seqid_1622 |
| variant_2141 | FWKLFKKIPPPKFKKVALKF | seqid_1623 |
| variant_2142 | KKKKFKKIPPPKFKKVALKF | seqid_1624 |
| variant_2143 | RKKKFKKIPPPKFKKVALKF | seqid_1625 |
| variant_2144 | FKKKFKKIPPPKFKKVALKF | seqid_1626 |
| variant_2145 | KWKKFKKIPPPKFKKVALKF | seqid_1627 |
| variant_2146 | RWKKFKKIPPPKFKKVALKF | seqid_1628 |
| variant_2147 | FWKKFKKIPPPKFKKVALKF | seqid_1629 |
| variant_2148 | KKKLLKKIPPPKFKKVALKF | seqid_1630 |
| variant_2149 | RKKLLKKIPPPKFKKVALKF | seqid_1631 |
| variant_2150 | FKKLLKKIPPPKFKKVALKF | seqid_1632 |
| variant_2151 | KWKLLKKIPPPKFKKVALKF | seqid_1633 |
| variant_2152 | RWKLLKKIPPPKFKKVALKF | seqid_1634 |
| variant_2153 | FWKLLKKIPPPKFKKVALKF | seqid_1635 |
| variant_2154 | KKKKLKKIPPPKFKKVALKF | seqid_1636 |
| variant_2155 | RKKKLKKIPPPKFKKVALKF | seqid_1637 |
| variant_2156 | FKKKLKKIPPPKFKKVALKF | seqid_1638 |
| variant_2157 | KWKKLKKIPPPKFKKVALKF | seqid_1639 |
| variant_2158 | RWKKLKKIPPPKFKKVALKF | seqid_1640 |
| variant_2159 | FWKKLKKIPPPKFKKVALKF | seqid_1641 |
| variant_2160 | KKKLFLKIPPKFKKVALKF | seqid_1642 |
| variant_2161 | RKKLFLKIPPKFKKVALKF | seqid_1643 |
| variant_2162 | FKKLFLKIPPKFKKVALKF | seqid_1644 |
| variant_2163 | KWKLFLKIPPKFKKVALKF | seqid_1645 |
| variant_2164 | RWKLFLKIPPKFKKVALKF | seqid_1646 |
| variant_2165 | FWKLFLKIPPKFKKVALKF | seqid_1647 |
| variant_2166 | KKKKFLKIPPKFKKVALKF | seqid_1648 |
| variant_2167 | RKKKFLKIPPKFKKVALKF | seqid_1649 |
| variant_2168 | FKKKFLKIPPKFKKVALKF | seqid_1650 |
| variant_2169 | KWKKFLKIPPKFKKVALKF | seqid_1651 |
| variant_2170 | RWKKFLKIPPKFKKVALKF | seqid_1652 |
| variant_2171 | FWKKFLKIPPKFKKVALKF | seqid_1653 |
| variant_2172 | KKKLLLKIPPKFKKVALKF | seqid_1654 |
| variant_2173 | RKKLLLKIPPKFKKVALKF | seqid_1655 |
| variant_2174 | FKKLLLKIPPKFKKVALKF | seqid_1656 |
| variant_2175 | KWKLLLKIPPKFKKVALKF | seqid_1657 |
| variant_2176 | RWKLLLKIPPKFKKVALKF | seqid_1658 |
| variant_2177 | FWKLLLKIPPKFKKVALKF | seqid_1659 |
| variant_2178 | KKKKLLKIPPKFKKVALKF | seqid_1660 |
| variant_2179 | RKKKLLKIPPKFKKVALKF | seqid_1661 |
| variant_2180 | FKKKLLKIPPKFKKVALKF | seqid_1662 |
| variant_2181 | KWKKLLKIPPKFKKVALKF | seqid_1663 |
| variant_2182 | RWKKLLKIPPKFKKVALKF | seqid_1664 |
| variant_2183 | FWKKLLKIPPKFKKVALKF | seqid_1665 |
| variant_2184 | KKKLFKKIPPKFKKVALKF | seqid_1666 |
| variant_2185 | RKKLFKKIPPKFKKVALKF | seqid_1667 |
| variant_2186 | FKKLFKKIPPKFKKVALKF | seqid_1668 |
| variant_2187 | KWKLFKKIPPKFKKVALKF | seqid_1669 |
| variant_2188 | RWKLFKKIPPKFKKVALKF | seqid_1670 |
| variant_2189 | FWKLFKKIPPKFKKVALKF | seqid_1671 |
| variant_2190 | KKKKFKKIPPKFKKVALKF | seqid_1672 |
| variant_2191 | RKKKFKKIPPKFKKVALKF | seqid_1673 |
| variant_2192 | FKKKFKKIPPKFKKVALKF | seqid_1674 |
| variant_2193 | KWKKFKKIPPKFKKVALKF | seqid_1675 |
| variant_2194 | RWKKFKKIPPKFKKVALKF | seqid_1676 |
| variant_2195 | FWKKFKKIPPKFKKVALKF | seqid_1677 |
| variant_2196 | KKKLLKKIPPKFKKVALKF | seqid_1678 |
| variant_2197 | RKKLLKKIPPKFKKVALKF | seqid_1679 |
| variant_2198 | FKKLLKKIPPKFKKVALKF | seqid_1680 |
| variant_2199 | KWKLLKKIPPKFKKVALKF | seqid_1681 |
| variant_2200 | RWKLLKKIPPKFKKVALKF | seqid_1682 |
| variant_2201 | FWKLLKKIPPKFKKVALKF | seqid_1683 |
| variant_2202 | KKKKLKKIPPKFKKVALKF | seqid_1684 |
| variant_2203 | RKKKLKKIPPKFKKVALKF | seqid_1685 |
| variant_2204 | FKKKLKKIPPKFKKVALKF | seqid_1686 |
| variant_2205 | KWKKLKKIPPKFKKVALKF | seqid_1687 |
| variant_2206 | RWKKLKKIPPKFKKVALKF | seqid_1688 |
| variant_2207 | FWKKLKKIPPKFKKVALKF | seqid_1689 |
| variant_2256 | KKKLFLKIPKFKKVALKF | seqid_1690 |
| variant_2257 | RKKLFLKIPKFKKVALKF | seqid_1691 |
| variant_2258 | FKKLFLKIPKFKKVALKF | seqid_1692 |
| variant_2259 | KWKLFLKIPKFKKVALKF | seqid_1693 |
| variant_2260 | RWKLFLKIPKFKKVALKF | seqid_1694 |
| variant_2261 | FWKLFLKIPKFKKVALKF | seqid_1695 |
| variant_2262 | KKKKFLKIPKFKKVALKF | seqid_1696 |
| variant_2263 | RKKKFLKIPKFKKVALKF | seqid_1697 |
| variant_2264 | FKKKFLKIPKFKKVALKF | seqid_1698 |
| variant_2265 | KWKKFLKIPKFKKVALKF | seqid_1699 |
| variant_2266 | RWKKFLKIPKFKKVALKF | seqid_1700 |
| variant_2267 | FWKKFLKIPKFKKVALKF | seqid_1701 |
| variant_2268 | KKKLLLKIPKFKKVALKF | seqid_1702 |
| variant_2269 | RKKLLLKIPKFKKVALKF | seqid_1703 |
| variant_2270 | FKKLLLKIPKFKKVALKF | seqid_1704 |
| variant_2271 | KWKLLLKIPKFKKVALKF | seqid_1705 |
| variant_2272 | RWKLLLKIPKFKKVALKF | seqid_1706 |
| variant_2273 | FWKLLLKIPKFKKVALKF | seqid_1707 |
| variant_2274 | KKKKLLKIPKFKKVALKF | seqid_1708 |
| variant_2275 | RKKKLLKIPKFKKVALKF | seqid_1709 |
| variant_2276 | FKKKLLKIPKFKKVALKF | seqid_1710 |
| variant_2277 | KWKKLLKIPKFKKVALKF | seqid_1711 |
| variant_2278 | RWKKLLKIPKFKKVALKF | seqid_1712 |
| variant_2279 | FWKKLLKIPKFKKVALKF | seqid_1713 |
| variant_2280 | KKKLFKKIPKFKKVALKF | seqid_1714 |
| variant_2281 | RKKLFKKIPKFKKVALKF | seqid_1715 |
| variant_2282 | FKKLFKKIPKFKKVALKF | seqid_1716 |
| variant_2283 | KWKLFKKIPKFKKVALKF | seqid_1717 |
| variant_2284 | RWKLFKKIPKFKKVALKF | seqid_1718 |
| variant_2285 | FWKLFKKIPKFKKVALKF | seqid_1719 |
| variant_2286 | KKKKFKKIPKFKKVALKF | seqid_1720 |
| variant_2287 | RKKKFKKIPKFKKVALKF | seqid_1721 |
| variant_2288 | FKKKFKKIPKFKKVALKF | seqid_1722 |
| variant_2289 | KWKKFKKIPKFKKVALKF | seqid_1723 |
| variant_2290 | RWKKFKKIPKFKKVALKF | seqid_1724 |
| variant_2291 | FWKKFKKIPKFKKVALKF | seqid_1725 |
| variant_2292 | KKKLLKKIPKFKKVALKF | seqid_1726 |
| variant_2293 | RKKLLKKIPKFKKVALKF | seqid_1727 |
| variant_2294 | FKKLLKKIPKFKKVALKF | seqid_1728 |
| variant_2295 | KWKLLKKIPKFKKVALKF | seqid_1729 |
| variant_2296 | RWKLLKKIPKFKKVALKF | seqid_1730 |
| variant_2297 | FWKLLKKIPKFKKVALKF | seqid_1731 |
| variant_2298 | KKKKLKKIPKFKKVALKF | seqid_1732 |
| variant_2299 | RKKKLKKIPKFKKVALKF | seqid_1733 |
| variant_2300 | FKKKLKKIPKFKKVALKF | seqid_1734 |
| variant_2301 | KWKKLKKIPKFKKVALKF | seqid_1735 |
| variant_2302 | RWKKLKKIPKFKKVALKF | seqid_1736 |
| variant_2303 | FWKKLKKIPKFKKVALKF | seqid_1737 |
| variant_2304 | KKKLFLKIPPPKFLHSALKF | seqid_1738 |
| variant_2305 | RKKLFLKIPPPKFLHSALKF | seqid_1739 |
| variant_2306 | FKKLFLKIPPPKFLHSALKF | seqid_1740 |
| variant_2307 | KWKLFLKIPPPKFLHSALKF | seqid_1741 |
| variant_2308 | RWKLFLKIPPPKFLHSALKF | seqid_1742 |
| variant_2309 | FWKLFLKIPPPKFLHSALKF | seqid_1743 |
| variant_2310 | KKKKFLKIPPPKFLHSALKF | seqid_1744 |
| variant_2311 | RKKKFLKIPPPKFLHSALKF | seqid_1745 |
| variant_2312 | FKKKFLKIPPPKFLHSALKF | seqid_1746 |
| variant_2313 | KWKKFLKIPPPKFLHSALKF | seqid_1747 |
| variant_2314 | RWKKFLKIPPPKFLHSALKF | seqid_1748 |
| variant_2315 | FWKKFLKIPPPKFLHSALKF | seqid_1749 |
| variant_2316 | KKKLLLKIPPPKFLHSALKF | seqid_1750 |
| variant_2317 | RKKLLLKIPPPKFLHSALKF | seqid_1751 |
| variant_2318 | FKKLLLKIPPPKFLHSALKF | seqid_1752 |
| variant_2319 | KWKLLLKIPPPKFLHSALKF | seqid_1753 |
| variant_2320 | RWKLLLKIPPPKFLHSALKF | seqid_1754 |
| variant_2321 | FWKLLLKIPPPKFLHSALKF | seqid_1755 |
| variant_2322 | KKKKLLKIPPPKFLHSALKF | seqid_1756 |
| variant_2323 | RKKKLLKIPPPKFLHSALKF | seqid_1757 |
| variant_2324 | FKKKLLKIPPPKFLHSALKF | seqid_1758 |
| variant_2325 | KWKKLLKIPPPKFLHSALKF | seqid_1759 |
| variant_2326 | RWKKLLKIPPPKFLHSALKF | seqid_1760 |
| variant_2327 | FWKKLLKIPPPKFLHSALKF | seqid_1761 |
| variant_2328 | KKKLFKKIPPPKFLHSALKF | seqid_1762 |
| variant_2329 | RKKLFKKIPPPKFLHSALKF | seqid_1763 |
| variant_2330 | FKKLFKKIPPPKFLHSALKF | seqid_1764 |
| variant_2331 | KWKLFKKIPPPKFLHSALKF | seqid_1765 |
| variant_2332 | RWKLFKKIPPPKFLHSALKF | seqid_1766 |
| variant_2333 | FWKLFKKIPPPKFLHSALKF | seqid_1767 |
| variant_2334 | KKKKFKKIPPPKFLHSALKF | seqid_1768 |
| variant_2335 | RKKKFKKIPPPKFLHSALKF | seqid_1769 |
| variant_2336 | FKKKFKKIPPPKFLHSALKF | seqid_1770 |
| variant_2337 | KWKKFKKIPPPKFLHSALKF | seqid_1771 |
| variant_2338 | RWKKFKKIPPPKFLHSALKF | seqid_1772 |
| variant_2339 | FWKKFKKIPPPKFLHSALKF | seqid_1773 |
| variant_2340 | KKKLLKKIPPPKFLHSALKF | seqid_1774 |
| variant_2341 | RKKLLKKIPPPKFLHSALKF | seqid_1775 |
| variant_2342 | FKKLLKKIPPPKFLHSALKF | seqid_1776 |
| variant_2343 | KWKLLKKIPPPKFLHSALKF | seqid_1777 |
| variant_2344 | RWKLLKKIPPPKFLHSALKF | seqid_1778 |
| variant_2345 | FWKLLKKIPPPKFLHSALKF | seqid_1779 |
| variant_2346 | KKKKLKKIPPPKFLHSALKF | seqid_1780 |
| variant_2347 | RKKKLKKIPPPKFLHSALKF | seqid_1781 |
| variant_2348 | FKKKLKKIPPPKFLHSALKF | seqid_1782 |
| variant_2349 | KWKKLKKIPPPKFLHSALKF | seqid_1783 |
| variant_2350 | RWKKLKKIPPPKFLHSALKF | seqid_1784 |
| variant_2351 | FWKKLKKIPPPKFLHSALKF | seqid_1785 |
| variant_2352 | KKKLFLKIPPKFLHSALKF | seqid_1786 |
| variant_2353 | RKKLFLKIPPKFLHSALKF | seqid_1787 |
| variant_2354 | FKKLFLKIPPKFLHSALKF | seqid_1788 |
| variant_2355 | KWKLFLKIPPKFLHSALKF | seqid_1789 |
| variant_2356 | RWKLFLKIPPKFLHSALKF | seqid_1790 |
| variant_2357 | FWKLFLKIPPKFLHSALKF | seqid_1791 |
| variant_2358 | KKKKFLKIPPKFLHSALKF | seqid_1792 |
| variant_2359 | RKKKFLKIPPKFLHSALKF | seqid_1793 |
| variant_2360 | FKKKFLKIPPKFLHSALKF | seqid_1794 |
| variant_2361 | KWKKFLKIPPKFLHSALKF | seqid_1795 |
| variant_2362 | RWKKFLKIPPKFLHSALKF | seqid_1796 |
| variant_2363 | FWKKFLKIPPKFLHSALKF | seqid_1797 |
| variant_2364 | KKKLLLKIPPKFLHSALKF | seqid_1798 |
| variant_2365 | RKKLLLKIPPKFLHSALKF | seqid_1799 |
| variant_2366 | FKKLLLKIPPKFLHSALKF | seqid_1800 |
| variant_2367 | KWKLLLKIPPKFLHSALKF | seqid_1801 |
| variant_2368 | RWKLLLKIPPKFLHSALKF | seqid_1802 |
| variant_2369 | FWKLLLKIPPKFLHSALKF | seqid_1803 |
| variant_2370 | KKKKLLKIPPKFLHSALKF | seqid_1804 |
| variant_2371 | RKKKLLKIPPKFLHSALKF | seqid_1805 |
| variant_2372 | FKKKLLKIPPKFLHSALKF | seqid_1806 |
| variant_2373 | KWKKLLKIPPKFLHSALKF | seqid_1807 |
| variant_2374 | RWKKLLKIPPKFLHSALKF | seqid_1808 |
| variant_2375 | FWKKLLKIPPKFLHSALKF | seqid_1809 |
| variant_2376 | KKKLFKKIPPKFLHSALKF | seqid_1810 |
| variant_2377 | RKKLFKKIPPKFLHSALKF | seqid_1811 |
| variant_2378 | FKKLFKKIPPKFLHSALKF | seqid_1812 |
| variant_2379 | KWKLFKKIPPKFLHSALKF | seqid_1813 |
| variant_2380 | RWKLFKKIPPKFLHSALKF | seqid_1814 |
| variant_2381 | FWKLFKKIPPKFLHSALKF | seqid_1815 |
| variant_2382 | KKKKFKKIPPKFLHSALKF | seqid_1816 |
| variant_2383 | RKKKFKKIPPKFLHSALKF | seqid_1817 |
| variant_2384 | FKKKFKKIPPKFLHSALKF | seqid_1818 |
| variant_2385 | KWKKFKKIPPKFLHSALKF | seqid_1819 |
| variant_2386 | RWKKFKKIPPKFLHSALKF | seqid_1820 |
| variant_2387 | FWKKFKKIPPKFLHSALKF | seqid_1821 |
| variant_2388 | KKKLLKKIPPKFLHSALKF | seqid_1822 |
| variant_2389 | RKKLLKKIPPKFLHSALKF | seqid_1823 |
| variant_2390 | FKKLLKKIPPKFLHSALKF | seqid_1824 |
| variant_2391 | KWKLLKKIPPKFLHSALKF | seqid_1825 |
| variant_2392 | RWKLLKKIPPKFLHSALKF | seqid_1826 |
| variant_2393 | FWKLLKKIPPKFLHSALKF | seqid_1827 |
| variant_2394 | KKKKLKKIPPKFLHSALKF | seqid_1828 |
| variant_2395 | RKKKLKKIPPKFLHSALKF | seqid_1829 |
| variant_2396 | FKKKLKKIPPKFLHSALKF | seqid_1830 |
| variant_2397 | KWKKLKKIPPKFLHSALKF | seqid_1831 |
| variant_2398 | RWKKLKKIPPKFLHSALKF | seqid_1832 |
| variant_2399 | FWKKLKKIPPKFLHSALKF | seqid_1833 |
| variant_2448 | KKKLFLKIPKFLHSALKF | seqid_1834 |
| variant_2449 | RKKLFLKIPKFLHSALKF | seqid_1835 |
| variant_2450 | FKKLFLKIPKFLHSALKF | seqid_1836 |
| variant_2451 | KWKLFLKIPKFLHSALKF | seqid_1837 |
| variant_2452 | RWKLFLKIPKFLHSALKF | seqid_1838 |
| variant_2453 | FWKLFLKIPKFLHSALKF | seqid_1839 |
| variant_2454 | KKKKFLKIPKFLHSALKF | seqid_1840 |
| variant_2455 | RKKKFLKIPKFLHSALKF | seqid_1841 |
| variant_2456 | FKKKFLKIPKFLHSALKF | seqid_1842 |
| variant_2457 | KWKKFLKIPKFLHSALKF | seqid_1843 |
| variant_2458 | RWKKFLKIPKFLHSALKF | seqid_1844 |
| variant_2459 | FWKKFLKIPKFLHSALKF | seqid_1845 |
| variant_2460 | KKKLLLKIPKFLHSALKF | seqid_1846 |
| variant_2461 | RKKLLLKIPKFLHSALKF | seqid_1847 |
| variant_2462 | FKKLLLKIPKFLHSALKF | seqid_1848 |
| variant_2463 | KWKLLLKIPKFLHSALKF | seqid_1849 |
| variant_2464 | RWKLLLKIPKFLHSALKF | seqid_1850 |
| variant_2465 | FWKLLLKIPKFLHSALKF | seqid_1851 |
| variant_2466 | KKKKLLKIPKFLHSALKF | seqid_1852 |
| variant_2467 | RKKKLLKIPKFLHSALKF | seqid_1853 |
| variant_2468 | FKKKLLKIPKFLHSALKF | seqid_1854 |
| variant_2469 | KWKKLLKIPKFLHSALKF | seqid_1855 |
| variant_2470 | RWKKLLKIPKFLHSALKF | seqid_1856 |
| variant_2471 | FWKKLLKIPKFLHSALKF | seqid_1857 |
| variant_2472 | KKKLFKKIPKFLHSALKF | seqid_1858 |
| variant_2473 | RKKLFKKIPKFLHSALKF | seqid_1859 |
| variant_2474 | FKKLFKKIPKFLHSALKF | seqid_1860 |
| variant_2475 | KWKLFKKIPKFLHSALKF | seqid_1861 |
| variant_2476 | RWKLFKKIPKFLHSALKF | seqid_1862 |
| variant_2477 | FWKLFKKIPKFLHSALKF | seqid_1863 |
| variant_2478 | KKKKFKKIPKFLHSALKF | seqid_1864 |
| variant_2479 | RKKKFKKIPKFLHSALKF | seqid_1865 |
| variant_2480 | FKKKFKKIPKFLHSALKF | seqid_1866 |
| variant_2481 | KWKKFKKIPKFLHSALKF | seqid_1867 |
| variant_2482 | RWKKFKKIPKFLHSALKF | seqid_1868 |
| variant_2483 | FWKKFKKIPKFLHSALKF | seqid_1869 |
| variant_2484 | KKKLLKKIPKFLHSALKF | seqid_1870 |
| variant_2485 | RKKLLKKIPKFLHSALKF | seqid_1871 |
| variant_2486 | FKKLLKKIPKFLHSALKF | seqid_1872 |
| variant_2487 | KWKLLKKIPKFLHSALKF | seqid_1873 |
| variant_2488 | RWKLLKKIPKFLHSALKF | seqid_1874 |
| variant_2489 | FWKLLKKIPKFLHSALKF | seqid_1875 |
| variant_2490 | KKKKLKKIPKFLHSALKF | seqid_1876 |
| variant_2491 | RKKKLKKIPKFLHSALKF | seqid_1877 |
| variant_2492 | FKKKLKKIPKFLHSALKF | seqid_1878 |
| variant_2493 | KWKKLKKIPKFLHSALKF | seqid_1879 |
| variant_2494 | RWKKLKKIPKFLHSALKF | seqid_1880 |
| variant_2495 | FWKKLKKIPKFLHSALKF | seqid_1881 |
| variant_2496 | KKKLFLKIPPPKFKHSALKF | seqid_1882 |
| variant_2497 | RKKLFLKIPPPKFKHSALKF | seqid_1883 |
| variant_2498 | FKKLFLKIPPPKFKHSALKF | seqid_1884 |
| variant_2499 | KWKLFLKIPPPKFKHSALKF | seqid_1885 |
| variant_2500 | RWKLFLKIPPPKFKHSALKF | seqid_1886 |
| variant_2501 | FWKLFLKIPPPKFKHSALKF | seqid_1887 |
| variant_2502 | KKKKFLKIPPPKFKHSALKF | seqid_1888 |
| variant_2503 | RKKKFLKIPPPKFKHSALKF | seqid_1889 |
| variant_2504 | FKKKFLKIPPPKFKHSALKF | seqid_1890 |
| variant_2505 | KWKKFLKIPPPKFKHSALKF | seqid_1891 |
| variant_2506 | RWKKFLKIPPPKFKHSALKF | seqid_1892 |
| variant_2507 | FWKKFLKIPPPKFKHSALKF | seqid_1893 |
| variant_2508 | KKKLLLKIPPPKFKHSALKF | seqid_1894 |
| variant_2509 | RKKLLLKIPPPKFKHSALKF | seqid_1895 |
| variant_2510 | FKKLLLKIPPPKFKHSALKF | seqid_1896 |
| variant_2511 | KWKLLLKIPPPKFKHSALKF | seqid_1897 |
| variant_2512 | RWKLLLKIPPPKFKHSALKF | seqid_1898 |
| variant_2513 | FWKLLLKIPPPKFKHSALKF | seqid_1899 |
| variant_2514 | KKKKLLKIPPPKFKHSALKF | seqid_1900 |
| variant_2515 | RKKKLLKIPPPKFKHSALKF | seqid_1901 |
| variant_2516 | FKKKLLKIPPPKFKHSALKF | seqid_1902 |
| variant_2517 | KWKKLLKIPPPKFKHSALKF | seqid_1903 |
| variant_2518 | RWKKLLKIPPPKFKHSALKF | seqid_1904 |
| variant_2519 | FWKKLLKIPPPKFKHSALKF | seqid_1905 |
| variant_2520 | KKKLFKKIPPPKFKHSALKF | seqid_1906 |
| variant_2521 | RKKLFKKIPPPKFKHSALKF | seqid_1907 |
| variant_2522 | FKKLFKKIPPPKFKHSALKF | seqid_1908 |
| variant_2523 | KWKLFKKIPPPKFKHSALKF | seqid_1909 |
| variant_2524 | RWKLFKKIPPPKFKHSALKF | seqid_1910 |
| variant_2525 | FWKLFKKIPPPKFKHSALKF | seqid_1911 |
| variant_2526 | KKKKFKKIPPPKFKHSALKF | seqid_1912 |
| variant_2527 | RKKKFKKIPPPKFKHSALKF | seqid_1913 |
| variant_2528 | FKKKFKKIPPPKFKHSALKF | seqid_1914 |
| variant_2529 | KWKKFKKIPPPKFKHSALKF | seqid_1915 |
| variant_2530 | RWKKFKKIPPPKFKHSALKF | seqid_1916 |
| variant_2531 | FWKKFKKIPPPKFKHSALKF | seqid_1917 |
| variant_2532 | KKKLLKKIPPPKFKHSALKF | seqid_1918 |
| variant_2533 | RKKLLKKIPPPKFKHSALKF | seqid_1919 |
| variant_2534 | FKKLLKKIPPPKFKHSALKF | seqid_1920 |
| variant_2535 | KWKLLKKIPPPKFKHSALKF | seqid_1921 |
| variant_2536 | RWKLLKKIPPPKFKHSALKF | seqid_1922 |
| variant_2537 | FWKLLKKIPPPKFKHSALKF | seqid_1923 |
| variant_2538 | KKKKLKKIPPPKFKHSALKF | seqid_1924 |
| variant_2539 | RKKKLKKIPPPKFKHSALKF | seqid_1925 |
| variant_2540 | FKKKLKKIPPPKFKHSALKF | seqid_1926 |
| variant_2541 | KWKKLKKIPPPKFKHSALKF | seqid_1927 |
| variant_2542 | RWKKLKKIPPPKFKHSALKF | seqid_1928 |
| variant_2543 | FWKKLKKIPPPKFKHSALKF | seqid_1929 |
| variant_2544 | KKKLFLKIPPKFKHSALKF | seqid_1930 |
| variant_2545 | RKKLFLKIPPKFKHSALKF | seqid_1931 |
| variant_2546 | FKKLFLKIPPKFKHSALKF | seqid_1932 |
| variant_2547 | KWKLFLKIPPKFKHSALKF | seqid_1933 |
| variant_2548 | RWKLFLKIPPKFKHSALKF | seqid_1934 |
| variant_2549 | FWKLFLKIPPKFKHSALKF | seqid_1935 |
| variant_2550 | KKKKFLKIPPKFKHSALKF | seqid_1936 |
| variant_2551 | RKKKFLKIPPKFKHSALKF | seqid_1937 |
| variant_2552 | FKKKFLKIPPKFKHSALKF | seqid_1938 |
| variant_2553 | KWKKFLKIPPKFKHSALKF | seqid_1939 |
| variant_2554 | RWKKFLKIPPKFKHSALKF | seqid_1940 |
| variant_2555 | FWKKFLKIPPKFKHSALKF | seqid_1941 |
| variant_2556 | KKKLLLKIPPKFKHSALKF | seqid_1942 |
| variant_2557 | RKKLLLKIPPKFKHSALKF | seqid_1943 |
| variant_2558 | FKKLLLKIPPKFKHSALKF | seqid_1944 |
| variant_2559 | KWKLLLKIPPKFKHSALKF | seqid_1945 |
| variant_2560 | RWKLLLKIPPKFKHSALKF | seqid_1946 |
| variant_2561 | FWKLLLKIPPKFKHSALKF | seqid_1947 |
| variant_2562 | KKKKLLKIPPKFKHSALKF | seqid_1948 |
| variant_2563 | RKKKLLKIPPKFKHSALKF | seqid_1949 |
| variant_2564 | FKKKLLKIPPKFKHSALKF | seqid_1950 |
| variant_2565 | KWKKLLKIPPKFKHSALKF | seqid_1951 |
| variant_2566 | RWKKLLKIPPKFKHSALKF | seqid_1952 |
| variant_2567 | FWKKLLKIPPKFKHSALKF | seqid_1953 |
| variant_2568 | KKKLFKKIPPKFKHSALKF | seqid_1954 |
| variant_2569 | RKKLFKKIPPKFKHSALKF | seqid_1955 |
| variant_2570 | FKKLFKKIPPKFKHSALKF | seqid_1956 |
| variant_2571 | KWKLFKKIPPKFKHSALKF | seqid_1957 |
| variant_2572 | RWKLFKKIPPKFKHSALKF | seqid_1958 |
| variant_2573 | FWKLFKKIPPKFKHSALKF | seqid_1959 |
| variant_2574 | KKKKFKKIPPKFKHSALKF | seqid_1960 |
| variant_2575 | RKKKFKKIPPKFKHSALKF | seqid_1961 |
| variant_2576 | FKKKFKKIPPKFKHSALKF | seqid_1962 |
| variant_2577 | KWKKFKKIPPKFKHSALKF | seqid_1963 |
| variant_2578 | RWKKFKKIPPKFKHSALKF | seqid_1964 |
| variant_2579 | FWKKFKKIPPKFKHSALKF | seqid_1965 |
| variant_2580 | KKKLLKKIPPKFKHSALKF | seqid_1966 |
| variant_2581 | RKKLLKKIPPKFKHSALKF | seqid_1967 |
| variant_2582 | FKKLLKKIPPKFKHSALKF | seqid_1968 |
| variant_2583 | KWKLLKKIPPKFKHSALKF | seqid_1969 |
| variant_2584 | RWKLLKKIPPKFKHSALKF | seqid_1970 |
| variant_2585 | FWKLLKKIPPKFKHSALKF | seqid_1971 |
| variant_2586 | KKKKLKKIPPKFKHSALKF | seqid_1972 |
| variant_2587 | RKKKLKKIPPKFKHSALKF | seqid_1973 |
| variant_2588 | FKKKLKKIPPKFKHSALKF | seqid_1974 |
| variant_2589 | KWKKLKKIPPKFKHSALKF | seqid_1975 |
| variant_2590 | RWKKLKKIPPKFKHSALKF | seqid_1976 |
| variant_2591 | FWKKLKKIPPKFKHSALKF | seqid_1977 |
| variant_2640 | KKKLFLKIPKFKHSALKF | seqid_1978 |
| variant_2641 | RKKLFLKIPKFKHSALKF | seqid_1979 |
| variant_2642 | FKKLFLKIPKFKHSALKF | seqid_1980 |
| variant_2643 | KWKLFLKIPKFKHSALKF | seqid_1981 |
| variant_2644 | RWKLFLKIPKFKHSALKF | seqid_1982 |
| variant_2645 | FWKLFLKIPKFKHSALKF | seqid_1983 |
| variant_2646 | KKKKFLKIPKFKHSALKF | seqid_1984 |
| variant_2647 | RKKKFLKIPKFKHSALKF | seqid_1985 |
| variant_2648 | FKKKFLKIPKFKHSALKF | seqid_1986 |
| variant_2649 | KWKKFLKIPKFKHSALKF | seqid_1987 |
| variant_2650 | RWKKFLKIPKFKHSALKF | seqid_1988 |
| variant_2651 | FWKKFLKIPKFKHSALKF | seqid_1989 |
| variant_2652 | KKKLLLKIPKFKHSALKF | seqid_1990 |
| variant_2653 | RKKLLLKIPKFKHSALKF | seqid_1991 |
| variant_2654 | FKKLLLKIPKFKHSALKF | seqid_1992 |
| variant_2655 | KWKLLLKIPKFKHSALKF | seqid_1993 |
| variant_2656 | RWKLLLKIPKFKHSALKF | seqid_1994 |
| variant_2657 | FWKLLLKIPKFKHSALKF | seqid_1995 |
| variant_2658 | KKKKLLKIPKFKHSALKF | seqid_1996 |
| variant_2659 | RKKKLLKIPKFKHSALKF | seqid_1997 |
| variant_2660 | FKKKLLKIPKFKHSALKF | seqid_1998 |
| variant_2661 | KWKKLLKIPKFKHSALKF | seqid_1999 |
| variant_2662 | RWKKLLKIPKFKHSALKF | seqid_2000 |
| variant_2663 | FWKKLLKIPKFKHSALKF | seqid_2001 |
| variant_2664 | KKKLFKKIPKFKHSALKF | seqid_2002 |
| variant_2665 | RKKLFKKIPKFKHSALKF | seqid_2003 |
| variant_2666 | FKKLFKKIPKFKHSALKF | seqid_2004 |
| variant_2667 | KWKLFKKIPKFKHSALKF | seqid_2005 |
| variant_2668 | RWKLFKKIPKFKHSALKF | seqid_2006 |
| variant_2669 | FWKLFKKIPKFKHSALKF | seqid_2007 |
| variant_2670 | KKKKFKKIPKFKHSALKF | seqid_2008 |
| variant_2671 | RKKKFKKIPKFKHSALKF | seqid_2009 |
| variant_2672 | FKKKFKKIPKFKHSALKF | seqid_2010 |
| variant_2673 | KWKKFKKIPKFKHSALKF | seqid_2011 |
| variant_2674 | RWKKFKKIPKFKHSALKF | seqid_2012 |
| variant_2675 | FWKKFKKIPKFKHSALKF | seqid_2013 |
| variant_2676 | KKKLLKKIPKFKHSALKF | seqid_2014 |
| variant_2677 | RKKLLKKIPKFKHSALKF | seqid_2015 |
| variant_2678 | FKKLLKKIPKFKHSALKF | seqid_2016 |
| variant_2679 | KWKLLKKIPKFKHSALKF | seqid_2017 |
| variant_2680 | RWKLLKKIPKFKHSALKF | seqid_2018 |
| variant_2681 | FWKLLKKIPKFKHSALKF | seqid_2019 |
| variant_2682 | KKKKLKKIPKFKHSALKF | seqid_2020 |
| variant_2683 | RKKKLKKIPKFKHSALKF | seqid_2021 |
| variant_2684 | FKKKLKKIPKFKHSALKF | seqid_2022 |
| variant_2685 | KWKKLKKIPKFKHSALKF | seqid_2023 |
| variant_2686 | RWKKLKKIPKFKHSALKF | seqid_2024 |
| variant_2687 | FWKKLKKIPKFKHSALKF | seqid_2025 |
| variant_2688 | KKKLFLKIPPPKFLKSALKF | seqid_2026 |
| variant_2689 | RKKLFLKIPPPKFLKSALKF | seqid_2027 |
| variant_2690 | FKKLFLKIPPPKFLKSALKF | seqid_2028 |
| variant_2691 | KWKLFLKIPPPKFLKSALKF | seqid_2029 |
| variant_2692 | RWKLFLKIPPPKFLKSALKF | seqid_2030 |
| variant_2693 | FWKLFLKIPPPKFLKSALKF | seqid_2031 |
| variant_2694 | KKKKFLKIPPPKFLKSALKF | seqid_2032 |
| variant_2695 | RKKKFLKIPPPKFLKSALKF | seqid_2033 |
| variant_2696 | FKKKFLKIPPPKFLKSALKF | seqid_2034 |
| variant_2697 | KWKKFLKIPPPKFLKSALKF | seqid_2035 |
| variant_2698 | RWKKFLKIPPPKFLKSALKF | seqid_2036 |
| variant_2699 | FWKKFLKIPPPKFLKSALKF | seqid_2037 |
| variant_2700 | KKKLLLKIPPPKFLKSALKF | seqid_2038 |
| variant_2701 | RKKLLLKIPPPKFLKSALKF | seqid_2039 |
| variant_2702 | FKKLLLKIPPPKFLKSALKF | seqid_2040 |
| variant_2703 | KWKLLLKIPPPKFLKSALKF | seqid_2041 |
| variant_2704 | RWKLLLKIPPPKFLKSALKF | seqid_2042 |
| variant_2705 | FWKLLLKIPPPKFLKSALKF | seqid_2043 |
| variant_2706 | KKKKLLKIPPPKFLKSALKF | seqid_2044 |
| variant_2707 | RKKKLLKIPPPKFLKSALKF | seqid_2045 |
| variant_2708 | FKKKLLKIPPPKFLKSALKF | seqid_2046 |
| variant_2709 | KWKKLLKIPPPKFLKSALKF | seqid_2047 |
| variant_2710 | RWKKLLKIPPPKFLKSALKF | seqid_2048 |
| variant_2711 | FWKKLLKIPPPKFLKSALKF | seqid_2049 |
| variant_2712 | KKKLFKKIPPPKFLKSALKF | seqid_2050 |
| variant_2713 | RKKLFKKIPPPKFLKSALKF | seqid_2051 |
| variant_2714 | FKKLFKKIPPPKFLKSALKF | seqid_2052 |
| variant_2715 | KWKLFKKIPPPKFLKSALKF | seqid_2053 |
| variant_2716 | RWKLFKKIPPPKFLKSALKF | seqid_2054 |
| variant_2717 | FWKLFKKIPPPKFLKSALKF | seqid_2055 |
| variant_2718 | KKKKFKKIPPPKFLKSALKF | seqid_2056 |
| variant_2719 | RKKKFKKIPPPKFLKSALKF | seqid_2057 |
| variant_2720 | FKKKFKKIPPPKFLKSALKF | seqid_2058 |
| variant_2721 | KWKKFKKIPPPKFLKSALKF | seqid_2059 |
| variant_2722 | RWKKFKKIPPPKFLKSALKF | seqid_2060 |
| variant_2723 | FWKKFKKIPPPKFLKSALKF | seqid_2061 |
| variant_2724 | KKKLLKKIPPPKFLKSALKF | seqid_2062 |
| variant_2725 | RKKLLKKIPPPKFLKSALKF | seqid_2063 |
| variant_2726 | FKKLLKKIPPPKFLKSALKF | seqid_2064 |
| variant_2727 | KWKLLKKIPPPKFLKSALKF | seqid_2065 |
| variant_2728 | RWKLLKKIPPPKFLKSALKF | seqid_2066 |
| variant_2729 | FWKLLKKIPPPKFLKSALKF | seqid_2067 |
| variant_2730 | KKKKLKKIPPPKFLKSALKF | seqid_2068 |
| variant_2731 | RKKKLKKIPPPKFLKSALKF | seqid_2069 |
| variant_2732 | FKKKLKKIPPPKFLKSALKF | seqid_2070 |
| variant_2733 | KWKKLKKIPPPKFLKSALKF | seqid_2071 |
| variant_2734 | RWKKLKKIPPPKFLKSALKF | seqid_2072 |
| variant_2735 | FWKKLKKIPPPKFLKSALKF | seqid_2073 |
| variant_2736 | KKKLFLKIPPKFLKSALKF | seqid_2074 |
| variant_2737 | RKKLFLKIPPKFLKSALKF | seqid_2075 |
| variant_2738 | FKKLFLKIPPKFLKSALKF | seqid_2076 |
| variant_2739 | KWKLFLKIPPKFLKSALKF | seqid_2077 |
| variant_2740 | RWKLFLKIPPKFLKSALKF | seqid_2078 |
| variant_2741 | FWKLFLKIPPKFLKSALKF | seqid_2079 |
| variant_2742 | KKKKFLKIPPKFLKSALKF | seqid_2080 |
| variant_2743 | RKKKFLKIPPKFLKSALKF | seqid_2081 |
| variant_2744 | FKKKFLKIPPKFLKSALKF | seqid_2082 |
| variant_2745 | KWKKFLKIPPKFLKSALKF | seqid_2083 |
| variant_2746 | RWKKFLKIPPKFLKSALKF | seqid_2084 |
| variant_2747 | FWKKFLKIPPKFLKSALKF | seqid_2085 |
| variant_2748 | KKKLLLKIPPKFLKSALKF | seqid_2086 |
| variant_2749 | RKKLLLKIPPKFLKSALKF | seqid_2087 |
| variant_2750 | FKKLLLKIPPKFLKSALKF | seqid_2088 |
| variant_2751 | KWKLLLKIPPKFLKSALKF | seqid_2089 |
| variant_2752 | RWKLLLKIPPKFLKSALKF | seqid_2090 |
| variant_2753 | FWKLLLKIPPKFLKSALKF | seqid_2091 |
| variant_2754 | KKKKLLKIPPKFLKSALKF | seqid_2092 |
| variant_2755 | RKKKLLKIPPKFLKSALKF | seqid_2093 |
| variant_2756 | FKKKLLKIPPKFLKSALKF | seqid_2094 |
| variant_2757 | KWKKLLKIPPKFLKSALKF | seqid_2095 |
| variant_2758 | RWKKLLKIPPKFLKSALKF | seqid_2096 |
| variant_2759 | FWKKLLKIPPKFLKSALKF | seqid_2097 |
| variant_2760 | KKKLFKKIPPKFLKSALKF | seqid_2098 |
| variant_2761 | RKKLFKKIPPKFLKSALKF | seqid_2099 |
| variant_2762 | FKKLFKKIPPKFLKSALKF | seqid_2100 |
| variant_2763 | KWKLFKKIPPKFLKSALKF | seqid_2101 |
| variant_2764 | RWKLFKKIPPKFLKSALKF | seqid_2102 |
| variant_2765 | FWKLFKKIPPKFLKSALKF | seqid_2103 |
| variant_2766 | KKKKFKKIPPKFLKSALKF | seqid_2104 |
| variant_2767 | RKKKFKKIPPKFLKSALKF | seqid_2105 |
| variant_2768 | FKKKFKKIPPKFLKSALKF | seqid_2106 |
| variant_2769 | KWKKFKKIPPKFLKSALKF | seqid_2107 |
| variant_2770 | RWKKFKKIPPKFLKSALKF | seqid_2108 |
| variant_2771 | FWKKFKKIPPKFLKSALKF | seqid_2109 |
| variant_2772 | KKKLLKKIPPKFLKSALKF | seqid_2110 |
| variant_2773 | RKKLLKKIPPKFLKSALKF | seqid_2111 |
| variant_2774 | FKKLLKKIPPKFLKSALKF | seqid_2112 |
| variant_2775 | KWKLLKKIPPKFLKSALKF | seqid_2113 |
| variant_2776 | RWKLLKKIPPKFLKSALKF | seqid_2114 |
| variant_2777 | FWKLLKKIPPKFLKSALKF | seqid_2115 |
| variant_2778 | KKKKLKKIPPKFLKSALKF | seqid_2116 |
| variant_2779 | RKKKLKKIPPKFLKSALKF | seqid_2117 |
| variant_2780 | FKKKLKKIPPKFLKSALKF | seqid_2118 |
| variant_2781 | KWKKLKKIPPKFLKSALKF | seqid_2119 |
| variant_2782 | RWKKLKKIPPKFLKSALKF | seqid_2120 |
| variant_2783 | FWKKLKKIPPKFLKSALKF | seqid_2121 |
| variant_2832 | KKKLFLKIPKFLKSALKF | seqid_2122 |
| variant_2833 | RKKLFLKIPKFLKSALKF | seqid_2123 |
| variant_2834 | FKKLFLKIPKFLKSALKF | seqid_2124 |
| variant_2835 | KWKLFLKIPKFLKSALKF | seqid_2125 |
| variant_2836 | RWKLFLKIPKFLKSALKF | seqid_2126 |
| variant_2837 | FWKLFLKIPKFLKSALKF | seqid_2127 |
| variant_2838 | KKKKFLKIPKFLKSALKF | seqid_2128 |
| variant_2839 | RKKKFLKIPKFLKSALKF | seqid_2129 |
| variant_2840 | FKKKFLKIPKFLKSALKF | seqid_2130 |
| variant_2841 | KWKKFLKIPKFLKSALKF | seqid_2131 |
| variant_2842 | RWKKFLKIPKFLKSALKF | seqid_2132 |
| variant_2843 | FWKKFLKIPKFLKSALKF | seqid_2133 |
| variant_2844 | KKKLLLKIPKFLKSALKF | seqid_2134 |
| variant_2845 | RKKLLLKIPKFLKSALKF | seqid_2135 |
| variant_2846 | FKKLLLKIPKFLKSALKF | seqid_2136 |
| variant_2847 | KWKLLLKIPKFLKSALKF | seqid_2137 |
| variant_2848 | RWKLLLKIPKFLKSALKF | seqid_2138 |
| variant_2849 | FWKLLLKIPKFLKSALKF | seqid_2139 |
| variant_2850 | KKKKLLKIPKFLKSALKF | seqid_2140 |
| variant_2851 | RKKKLLKIPKFLKSALKF | seqid_2141 |
| variant_2852 | FKKKLLKIPKFLKSALKF | seqid_2142 |
| variant_2853 | KWKKLLKIPKFLKSALKF | seqid_2143 |
| variant_2854 | RWKKLLKIPKFLKSALKF | seqid_2144 |
| variant_2855 | FWKKLLKIPKFLKSALKF | seqid_2145 |
| variant_2856 | KKKLFKKIPKFLKSALKF | seqid_2146 |
| variant_2857 | RKKLFKKIPKFLKSALKF | seqid_2147 |
| variant_2858 | FKKLFKKIPKFLKSALKF | seqid_2148 |
| variant_2859 | KWKLFKKIPKFLKSALKF | seqid_2149 |
| variant_2860 | RWKLFKKIPKFLKSALKF | seqid_2150 |
| variant_2861 | FWKLFKKIPKFLKSALKF | seqid_2151 |
| variant_2862 | KKKKFKKIPKFLKSALKF | seqid_2152 |
| variant_2863 | RKKKFKKIPKFLKSALKF | seqid_2153 |
| variant_2864 | FKKKFKKIPKFLKSALKF | seqid_2154 |
| variant_2865 | KWKKFKKIPKFLKSALKF | seqid_2155 |
| variant_2866 | RWKKFKKIPKFLKSALKF | seqid_2156 |
| variant_2867 | FWKKFKKIPKFLKSALKF | seqid_2157 |
| variant_2868 | KKKLLKKIPKFLKSALKF | seqid_2158 |
| variant_2869 | RKKLLKKIPKFLKSALKF | seqid_2159 |
| variant_2870 | FKKLLKKIPKFLKSALKF | seqid_2160 |
| variant_2871 | KWKLLKKIPKFLKSALKF | seqid_2161 |
| variant_2872 | RWKLLKKIPKFLKSALKF | seqid_2162 |
| variant_2873 | FWKLLKKIPKFLKSALKF | seqid_2163 |
| variant_2874 | KKKKLKKIPKFLKSALKF | seqid_2164 |
| variant_2875 | RKKKLKKIPKFLKSALKF | seqid_2165 |
| variant_2876 | FKKKLKKIPKFLKSALKF | seqid_2166 |
| variant_2877 | KWKKLKKIPKFLKSALKF | seqid_2167 |
| variant_2878 | RWKKLKKIPKFLKSALKF | seqid_2168 |
| variant_2879 | FWKKLKKIPKFLKSALKF | seqid_2169 |
| variant_2880 | KKKLFLKIPPPKFKKSALKF | seqid_2170 |
| variant_2881 | RKKLFLKIPPPKFKKSALKF | seqid_2171 |
| variant_2882 | FKKLFLKIPPPKFKKSALKF | seqid_2172 |
| variant_2883 | KWKLFLKIPPPKFKKSALKF | seqid_2173 |
| variant_2884 | RWKLFLKIPPPKFKKSALKF | seqid_2174 |
| variant_2885 | FWKLFLKIPPPKFKKSALKF | seqid_2175 |
| variant_2886 | KKKKFLKIPPPKFKKSALKF | seqid_2176 |
| variant_2887 | RKKKFLKIPPPKFKKSALKF | seqid_2177 |
| variant_2888 | FKKKFLKIPPPKFKKSALKF | seqid_2178 |
| variant_2889 | KWKKFLKIPPPKFKKSALKF | seqid_2179 |
| variant_2890 | RWKKFLKIPPPKFKKSALKF | seqid_2180 |
| variant_2891 | FWKKFLKIPPPKFKKSALKF | seqid_2181 |
| variant_2892 | KKKLLLKIPPPKFKKSALKF | seqid_2182 |
| variant_2893 | RKKLLLKIPPPKFKKSALKF | seqid_2183 |
| variant_2894 | FKKLLLKIPPPKFKKSALKF | seqid_2184 |
| variant_2895 | KWKLLLKIPPPKFKKSALKF | seqid_2185 |
| variant_2896 | RWKLLLKIPPPKFKKSALKF | seqid_2186 |
| variant_2897 | FWKLLLKIPPPKFKKSALKF | seqid_2187 |
| variant_2898 | KKKKLLKIPPPKFKKSALKF | seqid_2188 |
| variant_2899 | RKKKLLKIPPPKFKKSALKF | seqid_2189 |
| variant_2900 | FKKKLLKIPPPKFKKSALKF | seqid_2190 |
| variant_2901 | KWKKLLKIPPPKFKKSALKF | seqid_2191 |
| variant_2902 | RWKKLLKIPPPKFKKSALKF | seqid_2192 |
| variant_2903 | FWKKLLKIPPPKFKKSALKF | seqid_2193 |
| variant_2904 | KKKLFKKIPPPKFKKSALKF | seqid_2194 |
| variant_2905 | RKKLFKKIPPPKFKKSALKF | seqid_2195 |
| variant_2906 | FKKLFKKIPPPKFKKSALKF | seqid_2196 |
| variant_2907 | KWKLFKKIPPPKFKKSALKF | seqid_2197 |
| variant_2908 | RWKLFKKIPPPKFKKSALKF | seqid_2198 |
| variant_2909 | FWKLFKKIPPPKFKKSALKF | seqid_2199 |
| variant_2910 | KKKKFKKIPPPKFKKSALKF | seqid_2200 |
| variant_2911 | RKKKFKKIPPPKFKKSALKF | seqid_2201 |
| variant_2912 | FKKKFKKIPPPKFKKSALKF | seqid_2202 |
| variant_2913 | KWKKFKKIPPPKFKKSALKF | seqid_2203 |
| variant_2914 | RWKKFKKIPPPKFKKSALKF | seqid_2204 |
| variant_2915 | FWKKFKKIPPPKFKKSALKF | seqid_2205 |
| variant_2916 | KKKLLKKIPPPKFKKSALKF | seqid_2206 |
| variant_2917 | RKKLLKKIPPPKFKKSALKF | seqid_2207 |
| variant_2918 | FKKLLKKIPPPKFKKSALKF | seqid_2208 |
| variant_2919 | KWKLLKKIPPPKFKKSALKF | seqid_2209 |
| variant_2920 | RWKLLKKIPPPKFKKSALKF | seqid_2210 |
| variant_2921 | FWKLLKKIPPPKFKKSALKF | seqid_2211 |
| variant_2922 | KKKKLKKIPPPKFKKSALKF | seqid_2212 |
| variant_2923 | RKKKLKKIPPPKFKKSALKF | seqid_2213 |
| variant_2924 | FKKKLKKIPPPKFKKSALKF | seqid_2214 |
| variant_2925 | KWKKLKKIPPPKFKKSALKF | seqid_2215 |
| variant_2926 | RWKKLKKIPPPKFKKSALKF | seqid_2216 |
| variant_2927 | FWKKLKKIPPPKFKKSALKF | seqid_2217 |
| variant_2928 | KKKLFLKIPPKFKKSALKF | seqid_2218 |
| variant_2929 | RKKLFLKIPPKFKKSALKF | seqid_2219 |
| variant_2930 | FKKLFLKIPPKFKKSALKF | seqid_2220 |
| variant_2931 | KWKLFLKIPPKFKKSALKF | seqid_2221 |
| variant_2932 | RWKLFLKIPPKFKKSALKF | seqid_2222 |
| variant_2933 | FWKLFLKIPPKFKKSALKF | seqid_2223 |
| variant_2934 | KKKKFLKIPPKFKKSALKF | seqid_2224 |
| variant_2935 | RKKKFLKIPPKFKKSALKF | seqid_2225 |
| variant_2936 | FKKKFLKIPPKFKKSALKF | seqid_2226 |
| variant_2937 | KWKKFLKIPPKFKKSALKF | seqid_2227 |
| variant_2938 | RWKKFLKIPPKFKKSALKF | seqid_2228 |
| variant_2939 | FWKKFLKIPPKFKKSALKF | seqid_2229 |
| variant_2940 | KKKLLLKIPPKFKKSALKF | seqid_2230 |
| variant_2941 | RKKLLLKIPPKFKKSALKF | seqid_2231 |
| variant_2942 | FKKLLLKIPPKFKKSALKF | seqid_2232 |
| variant_2943 | KWKLLLKIPPKFKKSALKF | seqid_2233 |
| variant_2944 | RWKLLLKIPPKFKKSALKF | seqid_2234 |
| variant_2945 | FWKLLLKIPPKFKKSALKF | seqid_2235 |
| variant_2946 | KKKKLLKIPPKFKKSALKF | seqid_2236 |
| variant_2947 | RKKKLLKIPPKFKKSALKF | seqid_2237 |
| variant_2948 | FKKKLLKIPPKFKKSALKF | seqid_2238 |
| variant_2949 | KWKKLLKIPPKFKKSALKF | seqid_2239 |
| variant_2950 | RWKKLLKIPPKFKKSALKF | seqid_2240 |
| variant_2951 | FWKKLLKIPPKFKKSALKF | seqid_2241 |
| variant_2952 | KKKLFKKIPPKFKKSALKF | seqid_2242 |
| variant_2953 | RKKLFKKIPPKFKKSALKF | seqid_2243 |
| variant_2954 | FKKLFKKIPPKFKKSALKF | seqid_2244 |
| variant_2955 | KWKLFKKIPPKFKKSALKF | seqid_2245 |
| variant_2956 | RWKLFKKIPPKFKKSALKF | seqid_2246 |
| variant_2957 | FWKLFKKIPPKFKKSALKF | seqid_2247 |
| variant_2958 | KKKKFKKIPPKFKKSALKF | seqid_2248 |
| variant_2959 | RKKKFKKIPPKFKKSALKF | seqid_2249 |
| variant_2960 | FKKKFKKIPPKFKKSALKF | seqid_2250 |
| variant_2961 | KWKKFKKIPPKFKKSALKF | seqid_2251 |
| variant_2962 | RWKKFKKIPPKFKKSALKF | seqid_2252 |
| variant_2963 | FWKKFKKIPPKFKKSALKF | seqid_2253 |
| variant_2964 | KKKLLKKIPPKFKKSALKF | seqid_2254 |
| variant_2965 | RKKLLKKIPPKFKKSALKF | seqid_2255 |
| variant_2966 | FKKLLKKIPPKFKKSALKF | seqid_2256 |
| variant_2967 | KWKLLKKIPPKFKKSALKF | seqid_2257 |
| variant_2968 | RWKLLKKIPPKFKKSALKF | seqid_2258 |
| variant_2969 | FWKLLKKIPPKFKKSALKF | seqid_2259 |
| variant_2970 | KKKKLKKIPPKFKKSALKF | seqid_2260 |
| variant_2971 | RKKKLKKIPPKFKKSALKF | seqid_2261 |
| variant_2972 | FKKKLKKIPPKFKKSALKF | seqid_2262 |
| variant_2973 | KWKKLKKIPPKFKKSALKF | seqid_2263 |
| variant_2974 | RWKKLKKIPPKFKKSALKF | seqid_2264 |
| variant_2975 | FWKKLKKIPPKFKKSALKF | seqid_2265 |
| variant_3024 | KKKLFLKIPKFKKSALKF | seqid_2266 |
| variant_3025 | RKKLFLKIPKFKKSALKF | seqid_2267 |
| variant_3026 | FKKLFLKIPKFKKSALKF | seqid_2268 |
| variant_3027 | KWKLFLKIPKFKKSALKF | seqid_2269 |
| variant_3028 | RWKLFLKIPKFKKSALKF | seqid_2270 |
| variant_3029 | FWKLFLKIPKFKKSALKF | seqid_2271 |
| variant_3030 | KKKKFLKIPKFKKSALKF | seqid_2272 |
| variant_3031 | RKKKFLKIPKFKKSALKF | seqid_2273 |
| variant_3032 | FKKKFLKIPKFKKSALKF | seqid_2274 |
| variant_3033 | KWKKFLKIPKFKKSALKF | seqid_2275 |
| variant_3034 | RWKKFLKIPKFKKSALKF | seqid_2276 |
| variant_3035 | FWKKFLKIPKFKKSALKF | seqid_2277 |
| variant_3036 | KKKLLLKIPKFKKSALKF | seqid_2278 |
| variant_3037 | RKKLLLKIPKFKKSALKF | seqid_2279 |
| variant_3038 | FKKLLLKIPKFKKSALKF | seqid_2280 |
| variant_3039 | KWKLLLKIPKFKKSALKF | seqid_2281 |
| variant_3040 | RWKLLLKIPKFKKSALKF | seqid_2282 |
| variant_3041 | FWKLLLKIPKFKKSALKF | seqid_2283 |
| variant_3042 | KKKKLLKIPKFKKSALKF | seqid_2284 |
| variant_3043 | RKKKLLKIPKFKKSALKF | seqid_2285 |
| variant_3044 | FKKKLLKIPKFKKSALKF | seqid_2286 |
| variant_3045 | KWKKLLKIPKFKKSALKF | seqid_2287 |
| variant_3046 | RWKKLLKIPKFKKSALKF | seqid_2288 |
| variant_3047 | FWKKLLKIPKFKKSALKF | seqid_2289 |
| variant_3048 | KKKLFKKIPKFKKSALKF | seqid_2290 |
| variant_3049 | RKKLFKKIPKFKKSALKF | seqid_2291 |
| variant_3050 | FKKLFKKIPKFKKSALKF | seqid_2292 |
| variant_3051 | KWKLFKKIPKFKKSALKF | seqid_2293 |
| variant_3052 | RWKLFKKIPKFKKSALKF | seqid_2294 |
| variant_3053 | FWKLFKKIPKFKKSALKF | seqid_2295 |
| variant_3054 | KKKKFKKIPKFKKSALKF | seqid_2296 |
| variant_3055 | RKKKFKKIPKFKKSALKF | seqid_2297 |
| variant_3056 | FKKKFKKIPKFKKSALKF | seqid_2298 |
| variant_3057 | KWKKFKKIPKFKKSALKF | seqid_2299 |
| variant_3058 | RWKKFKKIPKFKKSALKF | seqid_2300 |
| variant_3059 | FWKKFKKIPKFKKSALKF | seqid_2301 |
| variant_3060 | KKKLLKKIPKFKKSALKF | seqid_2302 |
| variant_3061 | RKKLLKKIPKFKKSALKF | seqid_2303 |
| variant_3062 | FKKLLKKIPKFKKSALKF | seqid_2304 |
| variant_3063 | KWKLLKKIPKFKKSALKF | seqid_2305 |
| variant_3064 | RWKLLKKIPKFKKSALKF | seqid_2306 |
| variant_3065 | FWKLLKKIPKFKKSALKF | seqid_2307 |
| variant_3066 | KKKKLKKIPKFKKSALKF | seqid_2308 |
| variant_3067 | RKKKLKKIPKFKKSALKF | seqid_2309 |
| variant_3068 | FKKKLKKIPKFKKSALKF | seqid_2310 |
| variant_3069 | KWKKLKKIPKFKKSALKF | seqid_2311 |
| variant_3070 | RWKKLKKIPKFKKSALKF | seqid_2312 |
| variant_3071 | FWKKLKKIPKFKKSALKF | seqid_2313 |
| variant_3072 | KKKLFLKIPPPKFLHAAKKF | seqid_2314 |
| variant_3073 | RKKLFLKIPPPKFLHAAKKF | seqid_2315 |
| variant_3074 | FKKLFLKIPPPKFLHAAKKF | seqid_2316 |
| variant_3075 | KWKLFLKIPPPKFLHAAKKF | seqid_2317 |
| variant_3076 | RWKLFLKIPPPKFLHAAKKF | seqid_2318 |
| variant_3077 | FWKLFLKIPPPKFLHAAKKF | seqid_2319 |
| variant_3078 | KKKKFLKIPPPKFLHAAKKF | seqid_2320 |
| variant_3079 | RKKKFLKIPPPKFLHAAKKF | seqid_2321 |
| variant_3080 | FKKKFLKIPPPKFLHAAKKF | seqid_2322 |
| variant_3081 | KWKKFLKIPPPKFLHAAKKF | seqid_2323 |
| variant_3082 | RWKKFLKIPPPKFLHAAKKF | seqid_2324 |
| variant_3083 | FWKKFLKIPPPKFLHAAKKF | seqid_2325 |
| variant_3084 | KKKLLLKIPPPKFLHAAKKF | seqid_2326 |
| variant_3085 | RKKLLLKIPPPKFLHAAKKF | seqid_2327 |
| variant_3086 | FKKLLLKIPPPKFLHAAKKF | seqid_2328 |
| variant_3087 | KWKLLLKIPPPKFLHAAKKF | seqid_2329 |
| variant_3088 | RWKLLLKIPPPKFLHAAKKF | seqid_2330 |
| variant_3089 | FWKLLLKIPPPKFLHAAKKF | seqid_2331 |
| variant_3090 | KKKKLLKIPPPKFLHAAKKF | seqid_2332 |
| variant_3091 | RKKKLLKIPPPKFLHAAKKF | seqid_2333 |
| variant_3092 | FKKKLLKIPPPKFLHAAKKF | seqid_2334 |
| variant_3093 | KWKKLLKIPPPKFLHAAKKF | seqid_2335 |
| variant_3094 | RWKKLLKIPPPKFLHAAKKF | seqid_2336 |
| variant_3095 | FWKKLLKIPPPKFLHAAKKF | seqid_2337 |
| variant_3096 | KKKLFKKIPPPKFLHAAKKF | seqid_2338 |
| variant_3097 | RKKLFKKIPPPKFLHAAKKF | seqid_2339 |
| variant_3098 | FKKLFKKIPPPKFLHAAKKF | seqid_2340 |
| variant_3099 | KWKLFKKIPPPKFLHAAKKF | seqid_2341 |
| variant_3100 | RWKLFKKIPPPKFLHAAKKF | seqid_2342 |
| variant_3101 | FWKLFKKIPPPKFLHAAKKF | seqid_2343 |
| variant_3102 | KKKKFKKIPPPKFLHAAKKF | seqid_2344 |
| variant_3103 | RKKKFKKIPPPKFLHAAKKF | seqid_2345 |
| variant_3104 | FKKKFKKIPPPKFLHAAKKF | seqid_2346 |
| variant_3105 | KWKKFKKIPPPKFLHAAKKF | seqid_2347 |
| variant_3106 | RWKKFKKIPPPKFLHAAKKF | seqid_2348 |
| variant_3107 | FWKKFKKIPPPKFLHAAKKF | seqid_2349 |
| variant_3108 | KKKLLKKIPPPKFLHAAKKF | seqid_2350 |
| variant_3109 | RKKLLKKIPPPKFLHAAKKF | seqid_2351 |
| variant_3110 | FKKLLKKIPPPKFLHAAKKF | seqid_2352 |
| variant_3111 | KWKLLKKIPPPKFLHAAKKF | seqid_2353 |
| variant_3112 | RWKLLKKIPPPKFLHAAKKF | seqid_2354 |
| variant_3113 | FWKLLKKIPPPKFLHAAKKF | seqid_2355 |
| variant_3114 | KKKKLKKIPPPKFLHAAKKF | seqid_2356 |
| variant_3115 | RKKKLKKIPPPKFLHAAKKF | seqid_2357 |
| variant_3116 | FKKKLKKIPPPKFLHAAKKF | seqid_2358 |
| variant_3117 | KWKKLKKIPPPKFLHAAKKF | seqid_2359 |
| variant_3118 | RWKKLKKIPPPKFLHAAKKF | seqid_2360 |
| variant_3119 | FWKKLKKIPPPKFLHAAKKF | seqid_2361 |
| variant_3120 | KKKLFLKIPPKFLHAAKKF | seqid_2362 |
| variant_3121 | RKKLFLKIPPKFLHAAKKF | seqid_2363 |
| variant_3122 | FKKLFLKIPPKFLHAAKKF | seqid_2364 |
| variant_3123 | KWKLFLKIPPKFLHAAKKF | seqid_2365 |
| variant_3124 | RWKLFLKIPPKFLHAAKKF | seqid_2366 |
| variant_3125 | FWKLFLKIPPKFLHAAKKF | seqid_2367 |
| variant_3126 | KKKKFLKIPPKFLHAAKKF | seqid_2368 |
| variant_3127 | RKKKFLKIPPKFLHAAKKF | seqid_2369 |
| variant_3128 | FKKKFLKIPPKFLHAAKKF | seqid_2370 |
| variant_3129 | KWKKFLKIPPKFLHAAKKF | seqid_2371 |
| variant_3130 | RWKKFLKIPPKFLHAAKKF | seqid_2372 |
| variant_3131 | FWKKFLKIPPKFLHAAKKF | seqid_2373 |
| variant_3132 | KKKLLLKIPPKFLHAAKKF | seqid_2374 |
| variant_3133 | RKKLLLKIPPKFLHAAKKF | seqid_2375 |
| variant_3134 | FKKLLLKIPPKFLHAAKKF | seqid_2376 |
| variant_3135 | KWKLLLKIPPKFLHAAKKF | seqid_2377 |
| variant_3136 | RWKLLLKIPPKFLHAAKKF | seqid_2378 |
| variant_3137 | FWKLLLKIPPKFLHAAKKF | seqid_2379 |
| variant_3138 | KKKKLLKIPPKFLHAAKKF | seqid_2380 |
| variant_3139 | RKKKLLKIPPKFLHAAKKF | seqid_2381 |
| variant_3140 | FKKKLLKIPPKFLHAAKKF | seqid_2382 |
| variant_3141 | KWKKLLKIPPKFLHAAKKF | seqid_2383 |
| variant_3142 | RWKKLLKIPPKFLHAAKKF | seqid_2384 |
| variant_3143 | FWKKLLKIPPKFLHAAKKF | seqid_2385 |
| variant_3144 | KKKLFKKIPPKFLHAAKKF | seqid_2386 |
| variant_3145 | RKKLFKKIPPKFLHAAKKF | seqid_2387 |
| variant_3146 | FKKLFKKIPPKFLHAAKKF | seqid_2388 |
| variant_3147 | KWKLFKKIPPKFLHAAKKF | seqid_2389 |
| variant_3148 | RWKLFKKIPPKFLHAAKKF | seqid_2390 |
| variant_3149 | FWKLFKKIPPKFLHAAKKF | seqid_2391 |
| variant_3150 | KKKKFKKIPPKFLHAAKKF | seqid_2392 |
| variant_3151 | RKKKFKKIPPKFLHAAKKF | seqid_2393 |
| variant_3152 | FKKKFKKIPPKFLHAAKKF | seqid_2394 |
| variant_3153 | KWKKFKKIPPKFLHAAKKF | seqid_2395 |
| variant_3154 | RWKKFKKIPPKFLHAAKKF | seqid_2396 |
| variant_3155 | FWKKFKKIPPKFLHAAKKF | seqid_2397 |
| variant_3156 | KKKLLKKIPPKFLHAAKKF | seqid_2398 |
| variant_3157 | RKKLLKKIPPKFLHAAKKF | seqid_2399 |
| variant_3158 | FKKLLKKIPPKFLHAAKKF | seqid_2400 |
| variant_3159 | KWKLLKKIPPKFLHAAKKF | seqid_2401 |
| variant_3160 | RWKLLKKIPPKFLHAAKKF | seqid_2402 |
| variant_3161 | FWKLLKKIPPKFLHAAKKF | seqid_2403 |
| variant_3162 | KKKKLKKIPPKFLHAAKKF | seqid_2404 |
| variant_3163 | RKKKLKKIPPKFLHAAKKF | seqid_2405 |
| variant_3164 | FKKKLKKIPPKFLHAAKKF | seqid_2406 |
| variant_3165 | KWKKLKKIPPKFLHAAKKF | seqid_2407 |
| variant_3166 | RWKKLKKIPPKFLHAAKKF | seqid_2408 |
| variant_3167 | FWKKLKKIPPKFLHAAKKF | seqid_2409 |
| variant_3216 | KKKLFLKIPKFLHAAKKF | seqid_2410 |
| variant_3217 | RKKLFLKIPKFLHAAKKF | seqid_2411 |
| variant_3218 | FKKLFLKIPKFLHAAKKF | seqid_2412 |
| variant_3219 | KWKLFLKIPKFLHAAKKF | seqid_2413 |
| variant_3220 | RWKLFLKIPKFLHAAKKF | seqid_2414 |
| variant_3221 | FWKLFLKIPKFLHAAKKF | seqid_2415 |
| variant_3222 | KKKKFLKIPKFLHAAKKF | seqid_2416 |
| variant_3223 | RKKKFLKIPKFLHAAKKF | seqid_2417 |
| variant_3224 | FKKKFLKIPKFLHAAKKF | seqid_2418 |
| variant_3225 | KWKKFLKIPKFLHAAKKF | seqid_8 |
| variant_3226 | RWKKFLKIPKFLHAAKKF | seqid_2419 |
| variant_3227 | FWKKFLKIPKFLHAAKKF | seqid_2420 |
| variant_3228 | KKKLLLKIPKFLHAAKKF | seqid_2421 |
| variant_3229 | RKKLLLKIPKFLHAAKKF | seqid_2422 |
| variant_3230 | FKKLLLKIPKFLHAAKKF | seqid_2423 |
| variant_3231 | KWKLLLKIPKFLHAAKKF | seqid_2424 |
| variant_3232 | RWKLLLKIPKFLHAAKKF | seqid_2425 |
| variant_3233 | FWKLLLKIPKFLHAAKKF | seqid_2426 |
| variant_3234 | KKKKLLKIPKFLHAAKKF | seqid_2427 |
| variant_3235 | RKKKLLKIPKFLHAAKKF | seqid_2428 |
| variant_3236 | FKKKLLKIPKFLHAAKKF | seqid_2429 |
| variant_3237 | KWKKLLKIPKFLHAAKKF | seqid_9 |
| variant_3238 | RWKKLLKIPKFLHAAKKF | seqid_2430 |
| variant_3239 | FWKKLLKIPKFLHAAKKF | seqid_2431 |
| variant_3240 | KKKLFKKIPKFLHAAKKF | seqid_2432 |
| variant_3241 | RKKLFKKIPKFLHAAKKF | seqid_2433 |
| variant_3242 | FKKLFKKIPKFLHAAKKF | seqid_5 |
| variant_3243 | KWKLFKKIPKFLHAAKKF | seqid_7 |
| variant_3244 | RWKLFKKIPKFLHAAKKF | seqid_2434 |
| variant_3245 | FWKLFKKIPKFLHAAKKF | seqid_2435 |
| variant_3246 | KKKKFKKIPKFLHAAKKF | seqid_2436 |
| variant_3247 | RKKKFKKIPKFLHAAKKF | seqid_2437 |
| variant_3248 | FKKKFKKIPKFLHAAKKF | seqid_2438 |
| variant_3249 | KWKKFKKIPKFLHAAKKF | seqid_2439 |
| variant_3250 | RWKKFKKIPKFLHAAKKF | seqid_2440 |
| variant_3251 | FWKKFKKIPKFLHAAKKF | seqid_2441 |
| variant_3252 | KKKLLKKIPKFLHAAKKF | seqid_2442 |
| variant_3253 | RKKLLKKIPKFLHAAKKF | seqid_2443 |
| variant_3254 | FKKLLKKIPKFLHAAKKF | seqid_2444 |
| variant_3255 | KWKLLKKIPKFLHAAKKF | seqid_2445 |
| variant_3256 | RWKLLKKIPKFLHAAKKF | seqid_2446 |
| variant_3257 | FWKLLKKIPKFLHAAKKF | seqid_2447 |
| variant_3258 | KKKKLKKIPKFLHAAKKF | seqid_2448 |
| variant_3259 | RKKKLKKIPKFLHAAKKF | seqid_2449 |
| variant_3260 | FKKKLKKIPKFLHAAKKF | seqid_2450 |
| variant_3261 | KWKKLKKIPKFLHAAKKF | seqid_2451 |
| variant_3262 | RWKKLKKIPKFLHAAKKF | seqid_2452 |
| variant_3263 | FWKKLKKIPKFLHAAKKF | seqid_2453 |
| variant_3264 | KKKLFLKIPPPKFKHAAKKF | seqid_2454 |
| variant_3265 | RKKLFLKIPPPKFKHAAKKF | seqid_2455 |
| variant_3266 | FKKLFLKIPPPKFKHAAKKF | seqid_2456 |
| variant_3267 | KWKLFLKIPPPKFKHAAKKF | seqid_2457 |
| variant_3268 | RWKLFLKIPPPKFKHAAKKF | seqid_2458 |
| variant_3269 | FWKLFLKIPPPKFKHAAKKF | seqid_2459 |
| variant_3270 | KKKKFLKIPPPKFKHAAKKF | seqid_2460 |
| variant_3271 | RKKKFLKIPPPKFKHAAKKF | seqid_2461 |
| variant_3272 | FKKKFLKIPPPKFKHAAKKF | seqid_2462 |
| variant_3273 | KWKKFLKIPPPKFKHAAKKF | seqid_2463 |
| variant_3274 | RWKKFLKIPPPKFKHAAKKF | seqid_2464 |
| variant_3275 | FWKKFLKIPPPKFKHAAKKF | seqid_2465 |
| variant_3276 | KKKLLLKIPPPKFKHAAKKF | seqid_2466 |
| variant_3277 | RKKLLLKIPPPKFKHAAKKF | seqid_2467 |
| variant_3278 | FKKLLLKIPPPKFKHAAKKF | seqid_2468 |
| variant_3279 | KWKLLLKIPPPKFKHAAKKF | seqid_2469 |
| variant_3280 | RWKLLLKIPPPKFKHAAKKF | seqid_2470 |
| variant_3281 | FWKLLLKIPPPKFKHAAKKF | seqid_2471 |
| variant_3282 | KKKKLLKIPPPKFKHAAKKF | seqid_2472 |
| variant_3283 | RKKKLLKIPPPKFKHAAKKF | seqid_2473 |
| variant_3284 | FKKKLLKIPPPKFKHAAKKF | seqid_2474 |
| variant_3285 | KWKKLLKIPPPKFKHAAKKF | seqid_2475 |
| variant_3286 | RWKKLLKIPPPKFKHAAKKF | seqid_2476 |
| variant_3287 | FWKKLLKIPPPKFKHAAKKF | seqid_2477 |
| variant_3288 | KKKLFKKIPPPKFKHAAKKF | seqid_2478 |
| variant_3289 | RKKLFKKIPPPKFKHAAKKF | seqid_2479 |
| variant_3290 | FKKLFKKIPPPKFKHAAKKF | seqid_2480 |
| variant_3291 | KWKLFKKIPPPKFKHAAKKF | seqid_2481 |
| variant_3292 | RWKLFKKIPPPKFKHAAKKF | seqid_2482 |
| variant_3293 | FWKLFKKIPPPKFKHAAKKF | seqid_2483 |
| variant_3294 | KKKKFKKIPPPKFKHAAKKF | seqid_2484 |
| variant_3295 | RKKKFKKIPPPKFKHAAKKF | seqid_2485 |
| variant_3296 | FKKKFKKIPPPKFKHAAKKF | seqid_2486 |
| variant_3297 | KWKKFKKIPPPKFKHAAKKF | seqid_2487 |
| variant_3298 | RWKKFKKIPPPKFKHAAKKF | seqid_2488 |
| variant_3299 | FWKKFKKIPPPKFKHAAKKF | seqid_2489 |
| variant_3300 | KKKLLKKIPPPKFKHAAKKF | seqid_2490 |
| variant_3301 | RKKLLKKIPPPKFKHAAKKF | seqid_2491 |
| variant_3302 | FKKLLKKIPPPKFKHAAKKF | seqid_2492 |
| variant_3303 | KWKLLKKIPPPKFKHAAKKF | seqid_2493 |
| variant_3304 | RWKLLKKIPPPKFKHAAKKF | seqid_2494 |
| variant_3305 | FWKLLKKIPPPKFKHAAKKF | seqid_2495 |
| variant_3306 | KKKKLKKIPPPKFKHAAKKF | seqid_2496 |
| variant_3307 | RKKKLKKIPPPKFKHAAKKF | seqid_2497 |
| variant_3308 | FKKKLKKIPPPKFKHAAKKF | seqid_2498 |
| variant_3309 | KWKKLKKIPPPKFKHAAKKF | seqid_2499 |
| variant_3310 | RWKKLKKIPPPKFKHAAKKF | seqid_2500 |
| variant_3311 | FWKKLKKIPPPKFKHAAKKF | seqid_2501 |
| variant_3312 | KKKLFLKIPPKFKHAAKKF | seqid_2502 |
| variant_3313 | RKKLFLKIPPKFKHAAKKF | seqid_2503 |
| variant_3314 | FKKLFLKIPPKFKHAAKKF | seqid_2504 |
| variant_3315 | KWKLFLKIPPKFKHAAKKF | seqid_2505 |
| variant_3316 | RWKLFLKIPPKFKHAAKKF | seqid_2506 |
| variant_3317 | FWKLFLKIPPKFKHAAKKF | seqid_2507 |
| variant_3318 | KKKKFLKIPPKFKHAAKKF | seqid_2508 |
| variant_3319 | RKKKFLKIPPKFKHAAKKF | seqid_2509 |
| variant_3320 | FKKKFLKIPPKFKHAAKKF | seqid_2510 |
| variant_3321 | KWKKFLKIPPKFKHAAKKF | seqid_2511 |
| variant_3322 | RWKKFLKIPPKFKHAAKKF | seqid_2512 |
| variant_3323 | FWKKFLKIPPKFKHAAKKF | seqid_2513 |
| variant_3324 | KKKLLLKIPPKFKHAAKKF | seqid_2514 |
| variant_3325 | RKKLLLKIPPKFKHAAKKF | seqid_2515 |
| variant_3326 | FKKLLLKIPPKFKHAAKKF | seqid_2516 |
| variant_3327 | KWKLLLKIPPKFKHAAKKF | seqid_2517 |
| variant_3328 | RWKLLLKIPPKFKHAAKKF | seqid_2518 |
| variant_3329 | FWKLLLKIPPKFKHAAKKF | seqid_2519 |
| variant_3330 | KKKKLLKIPPKFKHAAKKF | seqid_2520 |
| variant_3331 | RKKKLLKIPPKFKHAAKKF | seqid_2521 |
| variant_3332 | FKKKLLKIPPKFKHAAKKF | seqid_2522 |
| variant_3333 | KWKKLLKIPPKFKHAAKKF | seqid_2523 |
| variant_3334 | RWKKLLKIPPKFKHAAKKF | seqid_2524 |
| variant_3335 | FWKKLLKIPPKFKHAAKKF | seqid_2525 |
| variant_3336 | KKKLFKKIPPKFKHAAKKF | seqid_2526 |
| variant_3337 | RKKLFKKIPPKFKHAAKKF | seqid_2527 |
| variant_3338 | FKKLFKKIPPKFKHAAKKF | seqid_2528 |
| variant_3339 | KWKLFKKIPPKFKHAAKKF | seqid_2529 |
| variant_3340 | RWKLFKKIPPKFKHAAKKF | seqid_2530 |
| variant_3341 | FWKLFKKIPPKFKHAAKKF | seqid_2531 |
| variant_3342 | KKKKFKKIPPKFKHAAKKF | seqid_2532 |
| variant_3343 | RKKKFKKIPPKFKHAAKKF | seqid_2533 |
| variant_3344 | FKKKFKKIPPKFKHAAKKF | seqid_2534 |
| variant_3345 | KWKKFKKIPPKFKHAAKKF | seqid_2535 |
| variant_3346 | RWKKFKKIPPKFKHAAKKF | seqid_2536 |
| variant_3347 | FWKKFKKIPPKFKHAAKKF | seqid_2537 |
| variant_3348 | KKKLLKKIPPKFKHAAKKF | seqid_2538 |
| variant_3349 | RKKLLKKIPPKFKHAAKKF | seqid_2539 |
| variant_3350 | FKKLLKKIPPKFKHAAKKF | seqid_2540 |
| variant_3351 | KWKLLKKIPPKFKHAAKKF | seqid_2541 |
| variant_3352 | RWKLLKKIPPKFKHAAKKF | seqid_2542 |
| variant_3353 | FWKLLKKIPPKFKHAAKKF | seqid_2543 |
| variant_3354 | KKKKLKKIPPKFKHAAKKF | seqid_2544 |
| variant_3355 | RKKKLKKIPPKFKHAAKKF | seqid_2545 |
| variant_3356 | FKKKLKKIPPKFKHAAKKF | seqid_2546 |
| variant_3357 | KWKKLKKIPPKFKHAAKKF | seqid_2547 |
| variant_3358 | RWKKLKKIPPKFKHAAKKF | seqid_2548 |
| variant_3359 | FWKKLKKIPPKFKHAAKKF | seqid_2549 |
| variant_3408 | KKKLFLKIPKFKHAAKKF | seqid_2550 |
| variant_3409 | RKKLFLKIPKFKHAAKKF | seqid_2551 |
| variant_3410 | FKKLFLKIPKFKHAAKKF | seqid_2552 |
| variant_3411 | KWKLFLKIPKFKHAAKKF | seqid_2553 |
| variant_3412 | RWKLFLKIPKFKHAAKKF | seqid_2554 |
| variant_3413 | FWKLFLKIPKFKHAAKKF | seqid_2555 |
| variant_3414 | KKKKFLKIPKFKHAAKKF | seqid_2556 |
| variant_3415 | RKKKFLKIPKFKHAAKKF | seqid_2557 |
| variant_3416 | FKKKFLKIPKFKHAAKKF | seqid_2558 |
| variant_3417 | KWKKFLKIPKFKHAAKKF | seqid_2559 |
| variant_3418 | RWKKFLKIPKFKHAAKKF | seqid_2560 |
| variant_3419 | FWKKFLKIPKFKHAAKKF | seqid_2561 |
| variant_3420 | KKKLLLKIPKFKHAAKKF | seqid_2562 |
| variant_3421 | RKKLLLKIPKFKHAAKKF | seqid_2563 |
| variant_3422 | FKKLLLKIPKFKHAAKKF | seqid_2564 |
| variant_3423 | KWKLLLKIPKFKHAAKKF | seqid_2565 |
| variant_3424 | RWKLLLKIPKFKHAAKKF | seqid_2566 |
| variant_3425 | FWKLLLKIPKFKHAAKKF | seqid_2567 |
| variant_3426 | KKKKLLKIPKFKHAAKKF | seqid_2568 |
| variant_3427 | RKKKLLKIPKFKHAAKKF | seqid_2569 |
| variant_3428 | FKKKLLKIPKFKHAAKKF | seqid_2570 |
| variant_3429 | KWKKLLKIPKFKHAAKKF | seqid_2571 |
| variant_3430 | RWKKLLKIPKFKHAAKKF | seqid_2572 |
| variant_3431 | FWKKLLKIPKFKHAAKKF | seqid_2573 |
| variant_3432 | KKKLFKKIPKFKHAAKKF | seqid_2574 |
| variant_3433 | RKKLFKKIPKFKHAAKKF | seqid_2575 |
| variant_3434 | FKKLFKKIPKFKHAAKKF | seqid_2576 |
| variant_3435 | KWKLFKKIPKFKHAAKKF | seqid_2577 |
| variant_3436 | RWKLFKKIPKFKHAAKKF | seqid_2578 |
| variant_3437 | FWKLFKKIPKFKHAAKKF | seqid_2579 |
| variant_3438 | KKKKFKKIPKFKHAAKKF | seqid_2580 |
| variant_3439 | RKKKFKKIPKFKHAAKKF | seqid_2581 |
| variant_3440 | FKKKFKKIPKFKHAAKKF | seqid_2582 |
| variant_3441 | KWKKFKKIPKFKHAAKKF | seqid_2583 |
| variant_3442 | RWKKFKKIPKFKHAAKKF | seqid_2584 |
| variant_3443 | FWKKFKKIPKFKHAAKKF | seqid_2585 |
| variant_3444 | KKKLLKKIPKFKHAAKKF | seqid_2586 |
| variant_3445 | RKKLLKKIPKFKHAAKKF | seqid_2587 |
| variant_3446 | FKKLLKKIPKFKHAAKKF | seqid_2588 |
| variant_3447 | KWKLLKKIPKFKHAAKKF | seqid_2589 |
| variant_3448 | RWKLLKKIPKFKHAAKKF | seqid_2590 |
| variant_3449 | FWKLLKKIPKFKHAAKKF | seqid_2591 |
| variant_3450 | KKKKLKKIPKFKHAAKKF | seqid_2592 |
| variant_3451 | RKKKLKKIPKFKHAAKKF | seqid_2593 |
| variant_3452 | FKKKLKKIPKFKHAAKKF | seqid_2594 |
| variant_3453 | KWKKLKKIPKFKHAAKKF | seqid_2595 |
| variant_3454 | RWKKLKKIPKFKHAAKKF | seqid_2596 |
| variant_3455 | FWKKLKKIPKFKHAAKKF | seqid_2597 |
| variant_3456 | KKKLFLKIPPPKFLKAAKKF | seqid_2598 |
| variant_3457 | RKKLFLKIPPPKFLKAAKKF | seqid_2599 |
| variant_3458 | FKKLFLKIPPPKFLKAAKKF | seqid_2600 |
| variant_3459 | KWKLFLKIPPPKFLKAAKKF | seqid_2601 |
| variant_3460 | RWKLFLKIPPPKFLKAAKKF | seqid_2602 |
| variant_3461 | FWKLFLKIPPPKFLKAAKKF | seqid_2603 |
| variant_3462 | KKKKFLKIPPPKFLKAAKKF | seqid_2604 |
| variant_3463 | RKKKFLKIPPPKFLKAAKKF | seqid_2605 |
| variant_3464 | FKKKFLKIPPPKFLKAAKKF | seqid_2606 |
| variant_3465 | KWKKFLKIPPPKFLKAAKKF | seqid_2607 |
| variant_3466 | RWKKFLKIPPPKFLKAAKKF | seqid_2608 |
| variant_3467 | FWKKFLKIPPPKFLKAAKKF | seqid_2609 |
| variant_3468 | KKKLLLKIPPPKFLKAAKKF | seqid_2610 |
| variant_3469 | RKKLLLKIPPPKFLKAAKKF | seqid_2611 |
| variant_3470 | FKKLLLKIPPPKFLKAAKKF | seqid_2612 |
| variant_3471 | KWKLLLKIPPPKFLKAAKKF | seqid_2613 |
| variant_3472 | RWKLLLKIPPPKFLKAAKKF | seqid_2614 |
| variant_3473 | FWKLLLKIPPPKFLKAAKKF | seqid_2615 |
| variant_3474 | KKKKLLKIPPPKFLKAAKKF | seqid_2616 |
| variant_3475 | RKKKLLKIPPPKFLKAAKKF | seqid_2617 |
| variant_3476 | FKKKLLKIPPPKFLKAAKKF | seqid_2618 |
| variant_3477 | KWKKLLKIPPPKFLKAAKKF | seqid_2619 |
| variant_3478 | RWKKLLKIPPPKFLKAAKKF | seqid_2620 |
| variant_3479 | FWKKLLKIPPPKFLKAAKKF | seqid_2621 |
| variant_3480 | KKKLFKKIPPPKFLKAAKKF | seqid_2622 |
| variant_3481 | RKKLFKKIPPPKFLKAAKKF | seqid_2623 |
| variant_3482 | FKKLFKKIPPPKFLKAAKKF | seqid_2624 |
| variant_3483 | KWKLFKKIPPPKFLKAAKKF | seqid_2625 |
| variant_3484 | RWKLFKKIPPPKFLKAAKKF | seqid_2626 |
| variant_3485 | FWKLFKKIPPPKFLKAAKKF | seqid_2627 |
| variant_3486 | KKKKFKKIPPPKFLKAAKKF | seqid_2628 |
| variant_3487 | RKKKFKKIPPPKFLKAAKKF | seqid_2629 |
| variant_3488 | FKKKFKKIPPPKFLKAAKKF | seqid_2630 |
| variant_3489 | KWKKFKKIPPPKFLKAAKKF | seqid_2631 |
| variant_3490 | RWKKFKKIPPPKFLKAAKKF | seqid_2632 |
| variant_3491 | FWKKFKKIPPPKFLKAAKKF | seqid_2633 |
| variant_3492 | KKKLLKKIPPPKFLKAAKKF | seqid_2634 |
| variant_3493 | RKKLLKKIPPPKFLKAAKKF | seqid_2635 |
| variant_3494 | FKKLLKKIPPPKFLKAAKKF | seqid_2636 |
| variant_3495 | KWKLLKKIPPPKFLKAAKKF | seqid_2637 |
| variant_3496 | RWKLLKKIPPPKFLKAAKKF | seqid_2638 |
| variant_3497 | FWKLLKKIPPPKFLKAAKKF | seqid_2639 |
| variant_3498 | KKKKLKKIPPPKFLKAAKKF | seqid_2640 |
| variant_3499 | RKKKLKKIPPPKFLKAAKKF | seqid_2641 |
| variant_3500 | FKKKLKKIPPPKFLKAAKKF | seqid_2642 |
| variant_3501 | KWKKLKKIPPPKFLKAAKKF | seqid_2643 |
| variant_3502 | RWKKLKKIPPPKFLKAAKKF | seqid_2644 |
| variant_3503 | FWKKLKKIPPPKFLKAAKKF | seqid_2645 |
| variant_3504 | KKKLFLKIPPKFLKAAKKF | seqid_2646 |
| variant_3505 | RKKLFLKIPPKFLKAAKKF | seqid_2647 |
| variant_3506 | FKKLFLKIPPKFLKAAKKF | seqid_2648 |
| variant_3507 | KWKLFLKIPPKFLKAAKKF | seqid_2649 |
| variant_3508 | RWKLFLKIPPKFLKAAKKF | seqid_2650 |
| variant_3509 | FWKLFLKIPPKFLKAAKKF | seqid_2651 |
| variant_3510 | KKKKFLKIPPKFLKAAKKF | seqid_2652 |
| variant_3511 | RKKKFLKIPPKFLKAAKKF | seqid_2653 |
| variant_3512 | FKKKFLKIPPKFLKAAKKF | seqid_2654 |
| variant_3513 | KWKKFLKIPPKFLKAAKKF | seqid_2655 |
| variant_3514 | RWKKFLKIPPKFLKAAKKF | seqid_2656 |
| variant_3515 | FWKKFLKIPPKFLKAAKKF | seqid_2657 |
| variant_3516 | KKKLLLKIPPKFLKAAKKF | seqid_2658 |
| variant_3517 | RKKLLLKIPPKFLKAAKKF | seqid_2659 |
| variant_3518 | FKKLLLKIPPKFLKAAKKF | seqid_2660 |
| variant_3519 | KWKLLLKIPPKFLKAAKKF | seqid_2661 |
| variant_3520 | RWKLLLKIPPKFLKAAKKF | seqid_2662 |
| variant_3521 | FWKLLLKIPPKFLKAAKKF | seqid_2663 |
| variant_3522 | KKKKLLKIPPKFLKAAKKF | seqid_2664 |
| variant_3523 | RKKKLLKIPPKFLKAAKKF | seqid_2665 |
| variant_3524 | FKKKLLKIPPKFLKAAKKF | seqid_2666 |
| variant_3525 | KWKKLLKIPPKFLKAAKKF | seqid_2667 |
| variant_3526 | RWKKLLKIPPKFLKAAKKF | seqid_2668 |
| variant_3527 | FWKKLLKIPPKFLKAAKKF | seqid_2669 |
| variant_3528 | KKKLFKKIPPKFLKAAKKF | seqid_2670 |
| variant_3529 | RKKLFKKIPPKFLKAAKKF | seqid_2671 |
| variant_3530 | FKKLFKKIPPKFLKAAKKF | seqid_2672 |
| variant_3531 | KWKLFKKIPPKFLKAAKKF | seqid_2673 |
| variant_3532 | RWKLFKKIPPKFLKAAKKF | seqid_2674 |
| variant_3533 | FWKLFKKIPPKFLKAAKKF | seqid_2675 |
| variant_3534 | KKKKFKKIPPKFLKAAKKF | seqid_2676 |
| variant_3535 | RKKKFKKIPPKFLKAAKKF | seqid_2677 |
| variant_3536 | FKKKFKKIPPKFLKAAKKF | seqid_2678 |
| variant_3537 | KWKKFKKIPPKFLKAAKKF | seqid_2679 |
| variant_3538 | RWKKFKKIPPKFLKAAKKF | seqid_2680 |
| variant_3539 | FWKKFKKIPPKFLKAAKKF | seqid_2681 |
| variant_3540 | KKKLLKKIPPKFLKAAKKF | seqid_2682 |
| variant_3541 | RKKLLKKIPPKFLKAAKKF | seqid_2683 |
| variant_3542 | FKKLLKKIPPKFLKAAKKF | seqid_2684 |
| variant_3543 | KWKLLKKIPPKFLKAAKKF | seqid_2685 |
| variant_3544 | RWKLLKKIPPKFLKAAKKF | seqid_2686 |
| variant_3545 | FWKLLKKIPPKFLKAAKKF | seqid_2687 |
| variant_3546 | KKKKLKKIPPKFLKAAKKF | seqid_2688 |
| variant_3547 | RKKKLKKIPPKFLKAAKKF | seqid_2689 |
| variant_3548 | FKKKLKKIPPKFLKAAKKF | seqid_2690 |
| variant_3549 | KWKKLKKIPPKFLKAAKKF | seqid_2691 |
| variant_3550 | RWKKLKKIPPKFLKAAKKF | seqid_2692 |
| variant_3551 | FWKKLKKIPPKFLKAAKKF | seqid_2693 |
| variant_3600 | KKKLFLKIPKFLKAAKKF | seqid_2694 |
| variant_3601 | RKKLFLKIPKFLKAAKKF | seqid_2695 |
| variant_3602 | FKKLFLKIPKFLKAAKKF | seqid_2696 |
| variant_3603 | KWKLFLKIPKFLKAAKKF | seqid_2697 |
| variant_3604 | RWKLFLKIPKFLKAAKKF | seqid_2698 |
| variant_3605 | FWKLFLKIPKFLKAAKKF | seqid_2699 |
| variant_3606 | KKKKFLKIPKFLKAAKKF | seqid_2700 |
| variant_3607 | RKKKFLKIPKFLKAAKKF | seqid_2701 |
| variant_3608 | FKKKFLKIPKFLKAAKKF | seqid_2702 |
| variant_3609 | KWKKFLKIPKFLKAAKKF | seqid_2703 |
| variant_3610 | RWKKFLKIPKFLKAAKKF | seqid_2704 |
| variant_3611 | FWKKFLKIPKFLKAAKKF | seqid_2705 |
| variant_3612 | KKKLLLKIPKFLKAAKKF | seqid_2706 |
| variant_3613 | RKKLLLKIPKFLKAAKKF | seqid_2707 |
| variant_3614 | FKKLLLKIPKFLKAAKKF | seqid_2708 |
| variant_3615 | KWKLLLKIPKFLKAAKKF | seqid_2709 |
| variant_3616 | RWKLLLKIPKFLKAAKKF | seqid_2710 |
| variant_3617 | FWKLLLKIPKFLKAAKKF | seqid_2711 |
| variant_3618 | KKKKLLKIPKFLKAAKKF | seqid_2712 |
| variant_3619 | RKKKLLKIPKFLKAAKKF | seqid_2713 |
| variant_3620 | FKKKLLKIPKFLKAAKKF | seqid_2714 |
| variant_3621 | KWKKLLKIPKFLKAAKKF | seqid_2715 |
| variant_3622 | RWKKLLKIPKFLKAAKKF | seqid_2716 |
| variant_3623 | FWKKLLKIPKFLKAAKKF | seqid_2717 |
| variant_3624 | KKKLFKKIPKFLKAAKKF | seqid_2718 |
| variant_3625 | RKKLFKKIPKFLKAAKKF | seqid_2719 |
| variant_3626 | FKKLFKKIPKFLKAAKKF | seqid_2720 |
| variant_3627 | KWKLFKKIPKFLKAAKKF | seqid_2721 |
| variant_3628 | RWKLFKKIPKFLKAAKKF | seqid_2722 |
| variant_3629 | FWKLFKKIPKFLKAAKKF | seqid_2723 |
| variant_3630 | KKKKFKKIPKFLKAAKKF | seqid_2724 |
| variant_3631 | RKKKFKKIPKFLKAAKKF | seqid_2725 |
| variant_3632 | FKKKFKKIPKFLKAAKKF | seqid_2726 |
| variant_3633 | KWKKFKKIPKFLKAAKKF | seqid_2727 |
| variant_3634 | RWKKFKKIPKFLKAAKKF | seqid_2728 |
| variant_3635 | FWKKFKKIPKFLKAAKKF | seqid_2729 |
| variant_3636 | KKKLLKKIPKFLKAAKKF | seqid_2730 |
| variant_3637 | RKKLLKKIPKFLKAAKKF | seqid_2731 |
| variant_3638 | FKKLLKKIPKFLKAAKKF | seqid_2732 |
| variant_3639 | KWKLLKKIPKFLKAAKKF | seqid_2733 |
| variant_3640 | RWKLLKKIPKFLKAAKKF | seqid_2734 |
| variant_3641 | FWKLLKKIPKFLKAAKKF | seqid_2735 |
| variant_3642 | KKKKLKKIPKFLKAAKKF | seqid_2736 |
| variant_3643 | RKKKLKKIPKFLKAAKKF | seqid_2737 |
| variant_3644 | FKKKLKKIPKFLKAAKKF | seqid_2738 |
| variant_3645 | KWKKLKKIPKFLKAAKKF | seqid_2739 |
| variant_3646 | RWKKLKKIPKFLKAAKKF | seqid_2740 |
| variant_3647 | FWKKLKKIPKFLKAAKKF | seqid_2741 |
| variant_3648 | KKKLFLKIPPPKFKKAAKKF | seqid_2742 |
| variant_3649 | RKKLFLKIPPPKFKKAAKKF | seqid_2743 |
| variant_3650 | FKKLFLKIPPPKFKKAAKKF | seqid_2744 |
| variant_3651 | KWKLFLKIPPPKFKKAAKKF | seqid_2745 |
| variant_3652 | RWKLFLKIPPPKFKKAAKKF | seqid_2746 |
| variant_3653 | FWKLFLKIPPPKFKKAAKKF | seqid_2747 |
| variant_3654 | KKKKFLKIPPPKFKKAAKKF | seqid_2748 |
| variant_3655 | RKKKFLKIPPPKFKKAAKKF | seqid_2749 |
| variant_3656 | FKKKFLKIPPPKFKKAAKKF | seqid_2750 |
| variant_3657 | KWKKFLKIPPPKFKKAAKKF | seqid_2751 |
| variant_3658 | RWKKFLKIPPPKFKKAAKKF | seqid_2752 |
| variant_3659 | FWKKFLKIPPPKFKKAAKKF | seqid_2753 |
| variant_3660 | KKKLLLKIPPPKFKKAAKKF | seqid_2754 |
| variant_3661 | RKKLLLKIPPPKFKKAAKKF | seqid_2755 |
| variant_3662 | FKKLLLKIPPPKFKKAAKKF | seqid_2756 |
| variant_3663 | KWKLLLKIPPPKFKKAAKKF | seqid_2757 |
| variant_3664 | RWKLLLKIPPPKFKKAAKKF | seqid_2758 |
| variant_3665 | FWKLLLKIPPPKFKKAAKKF | seqid_2759 |
| variant_3666 | KKKKLLKIPPPKFKKAAKKF | seqid_2760 |
| variant_3667 | RKKKLLKIPPPKFKKAAKKF | seqid_2761 |
| variant_3668 | FKKKLLKIPPPKFKKAAKKF | seqid_2762 |
| variant_3669 | KWKKLLKIPPPKFKKAAKKF | seqid_2763 |
| variant_3670 | RWKKLLKIPPPKFKKAAKKF | seqid_2764 |
| variant_3671 | FWKKLLKIPPPKFKKAAKKF | seqid_2765 |
| variant_3672 | KKKLFKKIPPPKFKKAAKKF | seqid_2766 |
| variant_3673 | RKKLFKKIPPPKFKKAAKKF | seqid_2767 |
| variant_3674 | FKKLFKKIPPPKFKKAAKKF | seqid_2768 |
| variant_3675 | KWKLFKKIPPPKFKKAAKKF | seqid_2769 |
| variant_3676 | RWKLFKKIPPPKFKKAAKKF | seqid_2770 |
| variant_3677 | FWKLFKKIPPPKFKKAAKKF | seqid_2771 |
| variant_3678 | KKKKFKKIPPPKFKKAAKKF | seqid_2772 |
| variant_3679 | RKKKFKKIPPPKFKKAAKKF | seqid_2773 |
| variant_3680 | FKKKFKKIPPPKFKKAAKKF | seqid_2774 |
| variant_3681 | KWKKFKKIPPPKFKKAAKKF | seqid_2775 |
| variant_3682 | RWKKFKKIPPPKFKKAAKKF | seqid_2776 |
| variant_3683 | FWKKFKKIPPPKFKKAAKKF | seqid_2777 |
| variant_3684 | KKKLLKKIPPPKFKKAAKKF | seqid_2778 |
| variant_3685 | RKKLLKKIPPPKFKKAAKKF | seqid_2779 |
| variant_3686 | FKKLLKKIPPPKFKKAAKKF | seqid_2780 |
| variant_3687 | KWKLLKKIPPPKFKKAAKKF | seqid_2781 |
| variant_3688 | RWKLLKKIPPPKFKKAAKKF | seqid_2782 |
| variant_3689 | FWKLLKKIPPPKFKKAAKKF | seqid_2783 |
| variant_3690 | KKKKLKKIPPPKFKKAAKKF | seqid_2784 |
| variant_3691 | RKKKLKKIPPPKFKKAAKKF | seqid_2785 |
| variant_3692 | FKKKLKKIPPPKFKKAAKKF | seqid_2786 |
| variant_3693 | KWKKLKKIPPPKFKKAAKKF | seqid_2787 |
| variant_3694 | RWKKLKKIPPPKFKKAAKKF | seqid_2788 |
| variant_3695 | FWKKLKKIPPPKFKKAAKKF | seqid_2789 |
| variant_3696 | KKKLFLKIPPKFKKAAKKF | seqid_2790 |
| variant_3697 | RKKLFLKIPPKFKKAAKKF | seqid_2791 |
| variant_3698 | FKKLFLKIPPKFKKAAKKF | seqid_2792 |
| variant_3699 | KWKLFLKIPPKFKKAAKKF | seqid_2793 |
| variant_3700 | RWKLFLKIPPKFKKAAKKF | seqid_2794 |
| variant_3701 | FWKLFLKIPPKFKKAAKKF | seqid_2795 |
| variant_3702 | KKKKFLKIPPKFKKAAKKF | seqid_2796 |
| variant_3703 | RKKKFLKIPPKFKKAAKKF | seqid_2797 |
| variant_3704 | FKKKFLKIPPKFKKAAKKF | seqid_2798 |
| variant_3705 | KWKKFLKIPPKFKKAAKKF | seqid_2799 |
| variant_3706 | RWKKFLKIPPKFKKAAKKF | seqid_2800 |
| variant_3707 | FWKKFLKIPPKFKKAAKKF | seqid_2801 |
| variant_3708 | KKKLLLKIPPKFKKAAKKF | seqid_2802 |
| variant_3709 | RKKLLLKIPPKFKKAAKKF | seqid_2803 |
| variant_3710 | FKKLLLKIPPKFKKAAKKF | seqid_2804 |
| variant_3711 | KWKLLLKIPPKFKKAAKKF | seqid_2805 |
| variant_3712 | RWKLLLKIPPKFKKAAKKF | seqid_2806 |
| variant_3713 | FWKLLLKIPPKFKKAAKKF | seqid_2807 |
| variant_3714 | KKKKLLKIPPKFKKAAKKF | seqid_2808 |
| variant_3715 | RKKKLLKIPPKFKKAAKKF | seqid_2809 |
| variant_3716 | FKKKLLKIPPKFKKAAKKF | seqid_2810 |
| variant_3717 | KWKKLLKIPPKFKKAAKKF | seqid_2811 |
| variant_3718 | RWKKLLKIPPKFKKAAKKF | seqid_2812 |
| variant_3719 | FWKKLLKIPPKFKKAAKKF | seqid_2813 |
| variant_3720 | KKKLFKKIPPKFKKAAKKF | seqid_2814 |
| variant_3721 | RKKLFKKIPPKFKKAAKKF | seqid_2815 |
| variant_3722 | FKKLFKKIPPKFKKAAKKF | seqid_2816 |
| variant_3723 | KWKLFKKIPPKFKKAAKKF | seqid_2817 |
| variant_3724 | RWKLFKKIPPKFKKAAKKF | seqid_2818 |
| variant_3725 | FWKLFKKIPPKFKKAAKKF | seqid_2819 |
| variant_3726 | KKKKFKKIPPKFKKAAKKF | seqid_2820 |
| variant_3727 | RKKKFKKIPPKFKKAAKKF | seqid_2821 |
| variant_3728 | FKKKFKKIPPKFKKAAKKF | seqid_2822 |
| variant_3729 | KWKKFKKIPPKFKKAAKKF | seqid_2823 |
| variant_3730 | RWKKFKKIPPKFKKAAKKF | seqid_2824 |
| variant_3731 | FWKKFKKIPPKFKKAAKKF | seqid_2825 |
| variant_3732 | KKKLLKKIPPKFKKAAKKF | seqid_2826 |
| variant_3733 | RKKLLKKIPPKFKKAAKKF | seqid_2827 |
| variant_3734 | FKKLLKKIPPKFKKAAKKF | seqid_2828 |
| variant_3735 | KWKLLKKIPPKFKKAAKKF | seqid_2829 |
| variant_3736 | RWKLLKKIPPKFKKAAKKF | seqid_2830 |
| variant_3737 | FWKLLKKIPPKFKKAAKKF | seqid_2831 |
| variant_3738 | KKKKLKKIPPKFKKAAKKF | seqid_2832 |
| variant_3739 | RKKKLKKIPPKFKKAAKKF | seqid_2833 |
| variant_3740 | FKKKLKKIPPKFKKAAKKF | seqid_2834 |
| variant_3741 | KWKKLKKIPPKFKKAAKKF | seqid_2835 |
| variant_3742 | RWKKLKKIPPKFKKAAKKF | seqid_2836 |
| variant_3743 | FWKKLKKIPPKFKKAAKKF | seqid_2837 |
| variant_3792 | KKKLFLKIPKFKKAAKKF | seqid_2838 |
| variant_3793 | RKKLFLKIPKFKKAAKKF | seqid_2839 |
| variant_3794 | FKKLFLKIPKFKKAAKKF | seqid_2840 |
| variant_3795 | KWKLFLKIPKFKKAAKKF | seqid_2841 |
| variant_3796 | RWKLFLKIPKFKKAAKKF | seqid_2842 |
| variant_3797 | FWKLFLKIPKFKKAAKKF | seqid_2843 |
| variant_3798 | KKKKFLKIPKFKKAAKKF | seqid_2844 |
| variant_3799 | RKKKFLKIPKFKKAAKKF | seqid_2845 |
| variant_3800 | FKKKFLKIPKFKKAAKKF | seqid_2846 |
| variant_3801 | KWKKFLKIPKFKKAAKKF | seqid_2847 |
| variant_3802 | RWKKFLKIPKFKKAAKKF | seqid_2848 |
| variant_3803 | FWKKFLKIPKFKKAAKKF | seqid_2849 |
| variant_3804 | KKKLLLKIPKFKKAAKKF | seqid_2850 |
| variant_3805 | RKKLLLKIPKFKKAAKKF | seqid_2851 |
| variant_3806 | FKKLLLKIPKFKKAAKKF | seqid_2852 |
| variant_3807 | KWKLLLKIPKFKKAAKKF | seqid_2853 |
| variant_3808 | RWKLLLKIPKFKKAAKKF | seqid_2854 |
| variant_3809 | FWKLLLKIPKFKKAAKKF | seqid_2855 |
| variant_3810 | KKKKLLKIPKFKKAAKKF | seqid_2856 |
| variant_3811 | RKKKLLKIPKFKKAAKKF | seqid_2857 |
| variant_3812 | FKKKLLKIPKFKKAAKKF | seqid_2858 |
| variant_3813 | KWKKLLKIPKFKKAAKKF | seqid_2859 |
| variant_3814 | RWKKLLKIPKFKKAAKKF | seqid_2860 |
| variant_3815 | FWKKLLKIPKFKKAAKKF | seqid_2861 |
| variant_3816 | KKKLFKKIPKFKKAAKKF | seqid_2862 |
| variant_3817 | RKKLFKKIPKFKKAAKKF | seqid_2863 |
| variant_3818 | FKKLFKKIPKFKKAAKKF | seqid_2864 |
| variant_3819 | KWKLFKKIPKFKKAAKKF | seqid_2865 |
| variant_3820 | RWKLFKKIPKFKKAAKKF | seqid_2866 |
| variant_3821 | FWKLFKKIPKFKKAAKKF | seqid_2867 |
| variant_3822 | KKKKFKKIPKFKKAAKKF | seqid_2868 |
| variant_3823 | RKKKFKKIPKFKKAAKKF | seqid_2869 |
| variant_3824 | FKKKFKKIPKFKKAAKKF | seqid_2870 |
| variant_3825 | KWKKFKKIPKFKKAAKKF | seqid_2871 |
| variant_3826 | RWKKFKKIPKFKKAAKKF | seqid_2872 |
| variant_3827 | FWKKFKKIPKFKKAAKKF | seqid_2873 |
| variant_3828 | KKKLLKKIPKFKKAAKKF | seqid_2874 |
| variant_3829 | RKKLLKKIPKFKKAAKKF | seqid_2875 |
| variant_3830 | FKKLLKKIPKFKKAAKKF | seqid_2876 |
| variant_3831 | KWKLLKKIPKFKKAAKKF | seqid_2877 |
| variant_3832 | RWKLLKKIPKFKKAAKKF | seqid_2878 |
| variant_3833 | FWKLLKKIPKFKKAAKKF | seqid_2879 |
| variant_3834 | KKKKLKKIPKFKKAAKKF | seqid_2880 |
| variant_3835 | RKKKLKKIPKFKKAAKKF | seqid_2881 |
| variant_3836 | FKKKLKKIPKFKKAAKKF | seqid_2882 |
| variant_3837 | KWKKLKKIPKFKKAAKKF | seqid_2883 |
| variant_3838 | RWKKLKKIPKFKKAAKKF | seqid_2884 |
| variant_3839 | FWKKLKKIPKFKKAAKKF | seqid_2885 |
| variant_3840 | KKKLFLKIPPPKFLHLAKKF | seqid_2886 |
| variant_3841 | RKKLFLKIPPPKFLHLAKKF | seqid_2887 |
| variant_3842 | FKKLFLKIPPPKFLHLAKKF | seqid_2888 |
| variant_3843 | KWKLFLKIPPPKFLHLAKKF | seqid_2889 |
| variant_3844 | RWKLFLKIPPPKFLHLAKKF | seqid_2890 |
| variant_3845 | FWKLFLKIPPPKFLHLAKKF | seqid_2891 |
| variant_3846 | KKKKFLKIPPPKFLHLAKKF | seqid_2892 |
| variant_3847 | RKKKFLKIPPPKFLHLAKKF | seqid_2893 |
| variant_3848 | FKKKFLKIPPPKFLHLAKKF | seqid_2894 |
| variant_3849 | KWKKFLKIPPPKFLHLAKKF | seqid_2895 |
| variant_3850 | RWKKFLKIPPPKFLHLAKKF | seqid_2896 |
| variant_3851 | FWKKFLKIPPPKFLHLAKKF | seqid_2897 |
| variant_3852 | KKKLLLKIPPPKFLHLAKKF | seqid_2898 |
| variant_3853 | RKKLLLKIPPPKFLHLAKKF | seqid_2899 |
| variant_3854 | FKKLLLKIPPPKFLHLAKKF | seqid_2900 |
| variant_3855 | KWKLLLKIPPPKFLHLAKKF | seqid_2901 |
| variant_3856 | RWKLLLKIPPPKFLHLAKKF | seqid_2902 |
| variant_3857 | FWKLLLKIPPPKFLHLAKKF | seqid_2903 |
| variant_3858 | KKKKLLKIPPPKFLHLAKKF | seqid_2904 |
| variant_3859 | RKKKLLKIPPPKFLHLAKKF | seqid_2905 |
| variant_3860 | FKKKLLKIPPPKFLHLAKKF | seqid_2906 |
| variant_3861 | KWKKLLKIPPPKFLHLAKKF | seqid_2907 |
| variant_3862 | RWKKLLKIPPPKFLHLAKKF | seqid_2908 |
| variant_3863 | FWKKLLKIPPPKFLHLAKKF | seqid_2909 |
| variant_3864 | KKKLFKKIPPPKFLHLAKKF | seqid_2910 |
| variant_3865 | RKKLFKKIPPPKFLHLAKKF | seqid_2911 |
| variant_3866 | FKKLFKKIPPPKFLHLAKKF | seqid_2912 |
| variant_3867 | KWKLFKKIPPPKFLHLAKKF | seqid_2913 |
| variant_3868 | RWKLFKKIPPPKFLHLAKKF | seqid_2914 |
| variant_3869 | FWKLFKKIPPPKFLHLAKKF | seqid_2915 |
| variant_3870 | KKKKFKKIPPPKFLHLAKKF | seqid_2916 |
| variant_3871 | RKKKFKKIPPPKFLHLAKKF | seqid_2917 |
| variant_3872 | FKKKFKKIPPPKFLHLAKKF | seqid_2918 |
| variant_3873 | KWKKFKKIPPPKFLHLAKKF | seqid_2919 |
| variant_3874 | RWKKFKKIPPPKFLHLAKKF | seqid_2920 |
| variant_3875 | FWKKFKKIPPPKFLHLAKKF | seqid_2921 |
| variant_3876 | KKKLLKKIPPPKFLHLAKKF | seqid_2922 |
| variant_3877 | RKKLLKKIPPPKFLHLAKKF | seqid_2923 |
| variant_3878 | FKKLLKKIPPPKFLHLAKKF | seqid_2924 |
| variant_3879 | KWKLLKKIPPPKFLHLAKKF | seqid_2925 |
| variant_3880 | RWKLLKKIPPPKFLHLAKKF | seqid_2926 |
| variant_3881 | FWKLLKKIPPPKFLHLAKKF | seqid_2927 |
| variant_3882 | KKKKLKKIPPPKFLHLAKKF | seqid_2928 |
| variant_3883 | RKKKLKKIPPPKFLHLAKKF | seqid_2929 |
| variant_3884 | FKKKLKKIPPPKFLHLAKKF | seqid_2930 |
| variant_3885 | KWKKLKKIPPPKFLHLAKKF | seqid_2931 |
| variant_3886 | RWKKLKKIPPPKFLHLAKKF | seqid_2932 |
| variant_3887 | FWKKLKKIPPPKFLHLAKKF | seqid_2933 |
| variant_3888 | KKKLFLKIPPKFLHLAKKF | seqid_2934 |
| variant_3889 | RKKLFLKIPPKFLHLAKKF | seqid_2935 |
| variant_3890 | FKKLFLKIPPKFLHLAKKF | seqid_2936 |
| variant_3891 | KWKLFLKIPPKFLHLAKKF | seqid_2937 |
| variant_3892 | RWKLFLKIPPKFLHLAKKF | seqid_2938 |
| variant_3893 | FWKLFLKIPPKFLHLAKKF | seqid_2939 |
| variant_3894 | KKKKFLKIPPKFLHLAKKF | seqid_2940 |
| variant_3895 | RKKKFLKIPPKFLHLAKKF | seqid_2941 |
| variant_3896 | FKKKFLKIPPKFLHLAKKF | seqid_2942 |
| variant_3897 | KWKKFLKIPPKFLHLAKKF | seqid_2943 |
| variant_3898 | RWKKFLKIPPKFLHLAKKF | seqid_2944 |
| variant_3899 | FWKKFLKIPPKFLHLAKKF | seqid_2945 |
| variant_3900 | KKKLLLKIPPKFLHLAKKF | seqid_2946 |
| variant_3901 | RKKLLLKIPPKFLHLAKKF | seqid_2947 |
| variant_3902 | FKKLLLKIPPKFLHLAKKF | seqid_2948 |
| variant_3903 | KWKLLLKIPPKFLHLAKKF | seqid_2949 |
| variant_3904 | RWKLLLKIPPKFLHLAKKF | seqid_2950 |
| variant_3905 | FWKLLLKIPPKFLHLAKKF | seqid_2951 |
| variant_3906 | KKKKLLKIPPKFLHLAKKF | seqid_2952 |
| variant_3907 | RKKKLLKIPPKFLHLAKKF | seqid_2953 |
| variant_3908 | FKKKLLKIPPKFLHLAKKF | seqid_2954 |
| variant_3909 | KWKKLLKIPPKFLHLAKKF | seqid_2955 |
| variant_3910 | RWKKLLKIPPKFLHLAKKF | seqid_2956 |
| variant_3911 | FWKKLLKIPPKFLHLAKKF | seqid_2957 |
| variant_3912 | KKKLFKKIPPKFLHLAKKF | seqid_2958 |
| variant_3913 | RKKLFKKIPPKFLHLAKKF | seqid_2959 |
| variant_3914 | FKKLFKKIPPKFLHLAKKF | seqid_2960 |
| variant_3915 | KWKLFKKIPPKFLHLAKKF | seqid_2961 |
| variant_3916 | RWKLFKKIPPKFLHLAKKF | seqid_2962 |
| variant_3917 | FWKLFKKIPPKFLHLAKKF | seqid_2963 |
| variant_3918 | KKKKFKKIPPKFLHLAKKF | seqid_2964 |
| variant_3919 | RKKKFKKIPPKFLHLAKKF | seqid_2965 |
| variant_3920 | FKKKFKKIPPKFLHLAKKF | seqid_2966 |
| variant_3921 | KWKKFKKIPPKFLHLAKKF | seqid_2967 |
| variant_3922 | RWKKFKKIPPKFLHLAKKF | seqid_2968 |
| variant_3923 | FWKKFKKIPPKFLHLAKKF | seqid_2969 |
| variant_3924 | KKKLLKKIPPKFLHLAKKF | seqid_2970 |
| variant_3925 | RKKLLKKIPPKFLHLAKKF | seqid_2971 |
| variant_3926 | FKKLLKKIPPKFLHLAKKF | seqid_2972 |
| variant_3927 | KWKLLKKIPPKFLHLAKKF | seqid_2973 |
| variant_3928 | RWKLLKKIPPKFLHLAKKF | seqid_2974 |
| variant_3929 | FWKLLKKIPPKFLHLAKKF | seqid_2975 |
| variant_3930 | KKKKLKKIPPKFLHLAKKF | seqid_2976 |
| variant_3931 | RKKKLKKIPPKFLHLAKKF | seqid_2977 |
| variant_3932 | FKKKLKKIPPKFLHLAKKF | seqid_2978 |
| variant_3933 | KWKKLKKIPPKFLHLAKKF | seqid_2979 |
| variant_3934 | RWKKLKKIPPKFLHLAKKF | seqid_2980 |
| variant_3935 | FWKKLKKIPPKFLHLAKKF | seqid_2981 |
| variant_3984 | KKKLFLKIPKFLHLAKKF | seqid_2982 |
| variant_3985 | RKKLFLKIPKFLHLAKKF | seqid_2983 |
| variant_3986 | FKKLFLKIPKFLHLAKKF | seqid_2984 |
| variant_3987 | KWKLFLKIPKFLHLAKKF | seqid_2985 |
| variant_3988 | RWKLFLKIPKFLHLAKKF | seqid_2986 |
| variant_3989 | FWKLFLKIPKFLHLAKKF | seqid_2987 |
| variant_3990 | KKKKFLKIPKFLHLAKKF | seqid_2988 |
| variant_3991 | RKKKFLKIPKFLHLAKKF | seqid_2989 |
| variant_3992 | FKKKFLKIPKFLHLAKKF | seqid_2990 |
| variant_3993 | KWKKFLKIPKFLHLAKKF | seqid_2991 |
| variant_3994 | RWKKFLKIPKFLHLAKKF | seqid_2992 |
| variant_3995 | FWKKFLKIPKFLHLAKKF | seqid_2993 |
| variant_3996 | KKKLLLKIPKFLHLAKKF | seqid_2994 |
| variant_3997 | RKKLLLKIPKFLHLAKKF | seqid_2995 |
| variant_3998 | FKKLLLKIPKFLHLAKKF | seqid_2996 |
| variant_3999 | KWKLLLKIPKFLHLAKKF | seqid_2997 |
| variant_4000 | RWKLLLKIPKFLHLAKKF | seqid_2998 |
| variant_4001 | FWKLLLKIPKFLHLAKKF | seqid_2999 |
| variant_4002 | KKKKLLKIPKFLHLAKKF | seqid_3000 |
| variant_4003 | RKKKLLKIPKFLHLAKKF | seqid_3001 |
| variant_4004 | FKKKLLKIPKFLHLAKKF | seqid_3002 |
| variant_4005 | KWKKLLKIPKFLHLAKKF | seqid_3003 |
| variant_4006 | RWKKLLKIPKFLHLAKKF | seqid_3004 |
| variant_4007 | FWKKLLKIPKFLHLAKKF | seqid_3005 |
| variant_4008 | KKKLFKKIPKFLHLAKKF | seqid_3006 |
| variant_4009 | RKKLFKKIPKFLHLAKKF | seqid_3007 |
| variant_4010 | FKKLFKKIPKFLHLAKKF | seqid_3008 |
| variant_4011 | KWKLFKKIPKFLHLAKKF | seqid_3 |
| variant_4012 | RWKLFKKIPKFLHLAKKF | seqid_4 |
| variant_4013 | FWKLFKKIPKFLHLAKKF | seqid_3009 |
| variant_4014 | KKKKFKKIPKFLHLAKKF | seqid_3010 |
| variant_4015 | RKKKFKKIPKFLHLAKKF | seqid_3011 |
| variant_4016 | FKKKFKKIPKFLHLAKKF | seqid_3012 |
| variant_4017 | KWKKFKKIPKFLHLAKKF | seqid_3013 |
| variant_4018 | RWKKFKKIPKFLHLAKKF | seqid_3014 |
| variant_4019 | FWKKFKKIPKFLHLAKKF | seqid_3015 |
| variant_4020 | KKKLLKKIPKFLHLAKKF | seqid_3016 |
| variant_4021 | RKKLLKKIPKFLHLAKKF | seqid_3017 |
| variant_4022 | FKKLLKKIPKFLHLAKKF | seqid_3018 |
| variant_4023 | KWKLLKKIPKFLHLAKKF | seqid_3019 |
| variant_4024 | RWKLLKKIPKFLHLAKKF | seqid_3020 |
| variant_4025 | FWKLLKKIPKFLHLAKKF | seqid_3021 |
| variant_4026 | KKKKLKKIPKFLHLAKKF | seqid_3022 |
| variant_4027 | RKKKLKKIPKFLHLAKKF | seqid_3023 |
| variant_4028 | FKKKLKKIPKFLHLAKKF | seqid_3024 |
| variant_4029 | KWKKLKKIPKFLHLAKKF | seqid_3025 |
| variant_4030 | RWKKLKKIPKFLHLAKKF | seqid_3026 |
| variant_4031 | FWKKLKKIPKFLHLAKKF | seqid_3027 |
| variant_4032 | KKKLFLKIPPPKFKHLAKKF | seqid_3028 |
| variant_4033 | RKKLFLKIPPPKFKHLAKKF | seqid_3029 |
| variant_4034 | FKKLFLKIPPPKFKHLAKKF | seqid_3030 |
| variant_4035 | KWKLFLKIPPPKFKHLAKKF | seqid_3031 |
| variant_4036 | RWKLFLKIPPPKFKHLAKKF | seqid_3032 |
| variant_4037 | FWKLFLKIPPPKFKHLAKKF | seqid_3033 |
| variant_4038 | KKKKFLKIPPPKFKHLAKKF | seqid_3034 |
| variant_4039 | RKKKFLKIPPPKFKHLAKKF | seqid_3035 |
| variant_4040 | FKKKFLKIPPPKFKHLAKKF | seqid_3036 |
| variant_4041 | KWKKFLKIPPPKFKHLAKKF | seqid_3037 |
| variant_4042 | RWKKFLKIPPPKFKHLAKKF | seqid_3038 |
| variant_4043 | FWKKFLKIPPPKFKHLAKKF | seqid_3039 |
| variant_4044 | KKKLLLKIPPPKFKHLAKKF | seqid_3040 |
| variant_4045 | RKKLLLKIPPPKFKHLAKKF | seqid_3041 |
| variant_4046 | FKKLLLKIPPPKFKHLAKKF | seqid_3042 |
| variant_4047 | KWKLLLKIPPPKFKHLAKKF | seqid_3043 |
| variant_4048 | RWKLLLKIPPPKFKHLAKKF | seqid_3044 |
| variant_4049 | FWKLLLKIPPPKFKHLAKKF | seqid_3045 |
| variant_4050 | KKKKLLKIPPPKFKHLAKKF | seqid_3046 |
| variant_4051 | RKKKLLKIPPPKFKHLAKKF | seqid_3047 |
| variant_4052 | FKKKLLKIPPPKFKHLAKKF | seqid_3048 |
| variant_4053 | KWKKLLKIPPPKFKHLAKKF | seqid_3049 |
| variant_4054 | RWKKLLKIPPPKFKHLAKKF | seqid_3050 |
| variant_4055 | FWKKLLKIPPPKFKHLAKKF | seqid_3051 |
| variant_4056 | KKKLFKKIPPPKFKHLAKKF | seqid_3052 |
| variant_4057 | RKKLFKKIPPPKFKHLAKKF | seqid_3053 |
| variant_4058 | FKKLFKKIPPPKFKHLAKKF | seqid_3054 |
| variant_4059 | KWKLFKKIPPPKFKHLAKKF | seqid_3055 |
| variant_4060 | RWKLFKKIPPPKFKHLAKKF | seqid_3056 |
| variant_4061 | FWKLFKKIPPPKFKHLAKKF | seqid_3057 |
| variant_4062 | KKKKFKKIPPPKFKHLAKKF | seqid_3058 |
| variant_4063 | RKKKFKKIPPPKFKHLAKKF | seqid_3059 |
| variant_4064 | FKKKFKKIPPPKFKHLAKKF | seqid_3060 |
| variant_4065 | KWKKFKKIPPPKFKHLAKKF | seqid_3061 |
| variant_4066 | RWKKFKKIPPPKFKHLAKKF | seqid_3062 |
| variant_4067 | FWKKFKKIPPPKFKHLAKKF | seqid_3063 |
| variant_4068 | KKKLLKKIPPPKFKHLAKKF | seqid_3064 |
| variant_4069 | RKKLLKKIPPPKFKHLAKKF | seqid_3065 |
| variant_4070 | FKKLLKKIPPPKFKHLAKKF | seqid_3066 |
| variant_4071 | KWKLLKKIPPPKFKHLAKKF | seqid_3067 |
| variant_4072 | RWKLLKKIPPPKFKHLAKKF | seqid_3068 |
| variant_4073 | FWKLLKKIPPPKFKHLAKKF | seqid_3069 |
| variant_4074 | KKKKLKKIPPPKFKHLAKKF | seqid_3070 |
| variant_4075 | RKKKLKKIPPPKFKHLAKKF | seqid_3071 |
| variant_4076 | FKKKLKKIPPPKFKHLAKKF | seqid_3072 |
| variant_4077 | KWKKLKKIPPPKFKHLAKKF | seqid_3073 |
| variant_4078 | RWKKLKKIPPPKFKHLAKKF | seqid_3074 |
| variant_4079 | FWKKLKKIPPPKFKHLAKKF | seqid_3075 |
| variant_4080 | KKKLFLKIPPKFKHLAKKF | seqid_3076 |
| variant_4081 | RKKLFLKIPPKFKHLAKKF | seqid_3077 |
| variant_4082 | FKKLFLKIPPKFKHLAKKF | seqid_3078 |
| variant_4083 | KWKLFLKIPPKFKHLAKKF | seqid_3079 |
| variant_4084 | RWKLFLKIPPKFKHLAKKF | seqid_3080 |
| variant_4085 | FWKLFLKIPPKFKHLAKKF | seqid_3081 |
| variant_4086 | KKKKFLKIPPKFKHLAKKF | seqid_3082 |
| variant_4087 | RKKKFLKIPPKFKHLAKKF | seqid_3083 |
| variant_4088 | FKKKFLKIPPKFKHLAKKF | seqid_3084 |
| variant_4089 | KWKKFLKIPPKFKHLAKKF | seqid_3085 |
| variant_4090 | RWKKFLKIPPKFKHLAKKF | seqid_3086 |
| variant_4091 | FWKKFLKIPPKFKHLAKKF | seqid_3087 |
| variant_4092 | KKKLLLKIPPKFKHLAKKF | seqid_3088 |
| variant_4093 | RKKLLLKIPPKFKHLAKKF | seqid_3089 |
| variant_4094 | FKKLLLKIPPKFKHLAKKF | seqid_3090 |
| variant_4095 | KWKLLLKIPPKFKHLAKKF | seqid_3091 |
| variant_4096 | RWKLLLKIPPKFKHLAKKF | seqid_3092 |
| variant_4097 | FWKLLLKIPPKFKHLAKKF | seqid_3093 |
| variant_4098 | KKKKLLKIPPKFKHLAKKF | seqid_3094 |
| variant_4099 | RKKKLLKIPPKFKHLAKKF | seqid_3095 |
| variant_4100 | FKKKLLKIPPKFKHLAKKF | seqid_3096 |
| variant_4101 | KWKKLLKIPPKFKHLAKKF | seqid_3097 |
| variant_4102 | RWKKLLKIPPKFKHLAKKF | seqid_3098 |
| variant_4103 | FWKKLLKIPPKFKHLAKKF | seqid_3099 |
| variant_4104 | KKKLFKKIPPKFKHLAKKF | seqid_3100 |
| variant_4105 | RKKLFKKIPPKFKHLAKKF | seqid_3101 |
| variant_4106 | FKKLFKKIPPKFKHLAKKF | seqid_3102 |
| variant_4107 | KWKLFKKIPPKFKHLAKKF | seqid_3103 |
| variant_4108 | RWKLFKKIPPKFKHLAKKF | seqid_3104 |
| variant_4109 | FWKLFKKIPPKFKHLAKKF | seqid_3105 |
| variant_4110 | KKKKFKKIPPKFKHLAKKF | seqid_3106 |
| variant_4111 | RKKKFKKIPPKFKHLAKKF | seqid_3107 |
| variant_4112 | FKKKFKKIPPKFKHLAKKF | seqid_3108 |
| variant_4113 | KWKKFKKIPPKFKHLAKKF | seqid_3109 |
| variant_4114 | RWKKFKKIPPKFKHLAKKF | seqid_3110 |
| variant_4115 | FWKKFKKIPPKFKHLAKKF | seqid_3111 |
| variant_4116 | KKKLLKKIPPKFKHLAKKF | seqid_3112 |
| variant_4117 | RKKLLKKIPPKFKHLAKKF | seqid_3113 |
| variant_4118 | FKKLLKKIPPKFKHLAKKF | seqid_3114 |
| variant_4119 | KWKLLKKIPPKFKHLAKKF | seqid_3115 |
| variant_4120 | RWKLLKKIPPKFKHLAKKF | seqid_3116 |
| variant_4121 | FWKLLKKIPPKFKHLAKKF | seqid_3117 |
| variant_4122 | KKKKLKKIPPKFKHLAKKF | seqid_3118 |
| variant_4123 | RKKKLKKIPPKFKHLAKKF | seqid_3119 |
| variant_4124 | FKKKLKKIPPKFKHLAKKF | seqid_3120 |
| variant_4125 | KWKKLKKIPPKFKHLAKKF | seqid_3121 |
| variant_4126 | RWKKLKKIPPKFKHLAKKF | seqid_3122 |
| variant_4127 | FWKKLKKIPPKFKHLAKKF | seqid_3123 |
| variant_4176 | KKKLFLKIPKFKHLAKKF | seqid_3124 |
| variant_4177 | RKKLFLKIPKFKHLAKKF | seqid_3125 |
| variant_4178 | FKKLFLKIPKFKHLAKKF | seqid_3126 |
| variant_4179 | KWKLFLKIPKFKHLAKKF | seqid_3127 |
| variant_4180 | RWKLFLKIPKFKHLAKKF | seqid_3128 |
| variant_4181 | FWKLFLKIPKFKHLAKKF | seqid_3129 |
| variant_4182 | KKKKFLKIPKFKHLAKKF | seqid_3130 |
| variant_4183 | RKKKFLKIPKFKHLAKKF | seqid_3131 |
| variant_4184 | FKKKFLKIPKFKHLAKKF | seqid_3132 |
| variant_4185 | KWKKFLKIPKFKHLAKKF | seqid_3133 |
| variant_4186 | RWKKFLKIPKFKHLAKKF | seqid_3134 |
| variant_4187 | FWKKFLKIPKFKHLAKKF | seqid_3135 |
| variant_4188 | KKKLLLKIPKFKHLAKKF | seqid_3136 |
| variant_4189 | RKKLLLKIPKFKHLAKKF | seqid_3137 |
| variant_4190 | FKKLLLKIPKFKHLAKKF | seqid_3138 |
| variant_4191 | KWKLLLKIPKFKHLAKKF | seqid_3139 |
| variant_4192 | RWKLLLKIPKFKHLAKKF | seqid_3140 |
| variant_4193 | FWKLLLKIPKFKHLAKKF | seqid_3141 |
| variant_4194 | KKKKLLKIPKFKHLAKKF | seqid_3142 |
| variant_4195 | RKKKLLKIPKFKHLAKKF | seqid_3143 |
| variant_4196 | FKKKLLKIPKFKHLAKKF | seqid_3144 |
| variant_4197 | KWKKLLKIPKFKHLAKKF | seqid_3145 |
| variant_4198 | RWKKLLKIPKFKHLAKKF | seqid_3146 |
| variant_4199 | FWKKLLKIPKFKHLAKKF | seqid_3147 |
| variant_4200 | KKKLFKKIPKFKHLAKKF | seqid_3148 |
| variant_4201 | RKKLFKKIPKFKHLAKKF | seqid_3149 |
| variant_4202 | FKKLFKKIPKFKHLAKKF | seqid_3150 |
| variant_4203 | KWKLFKKIPKFKHLAKKF | seqid_3151 |
| variant_4204 | RWKLFKKIPKFKHLAKKF | seqid_3152 |
| variant_4205 | FWKLFKKIPKFKHLAKKF | seqid_3153 |
| variant_4206 | KKKKFKKIPKFKHLAKKF | seqid_3154 |
| variant_4207 | RKKKFKKIPKFKHLAKKF | seqid_3155 |
| variant_4208 | FKKKFKKIPKFKHLAKKF | seqid_3156 |
| variant_4209 | KWKKFKKIPKFKHLAKKF | seqid_3157 |
| variant_4210 | RWKKFKKIPKFKHLAKKF | seqid_3158 |
| variant_4211 | FWKKFKKIPKFKHLAKKF | seqid_3159 |
| variant_4212 | KKKLLKKIPKFKHLAKKF | seqid_3160 |
| variant_4213 | RKKLLKKIPKFKHLAKKF | seqid_3161 |
| variant_4214 | FKKLLKKIPKFKHLAKKF | seqid_3162 |
| variant_4215 | KWKLLKKIPKFKHLAKKF | seqid_3163 |
| variant_4216 | RWKLLKKIPKFKHLAKKF | seqid_3164 |
| variant_4217 | FWKLLKKIPKFKHLAKKF | seqid_3165 |
| variant_4218 | KKKKLKKIPKFKHLAKKF | seqid_3166 |
| variant_4219 | RKKKLKKIPKFKHLAKKF | seqid_3167 |
| variant_4220 | FKKKLKKIPKFKHLAKKF | seqid_3168 |
| variant_4221 | KWKKLKKIPKFKHLAKKF | seqid_3169 |
| variant_4222 | RWKKLKKIPKFKHLAKKF | seqid_3170 |
| variant_4223 | FWKKLKKIPKFKHLAKKF | seqid_3171 |
| variant_4224 | KKKLFLKIPPPKFLKLAKKF | seqid_3172 |
| variant_4225 | RKKLFLKIPPPKFLKLAKKF | seqid_3173 |
| variant_4226 | FKKLFLKIPPPKFLKLAKKF | seqid_3174 |
| variant_4227 | KWKLFLKIPPPKFLKLAKKF | seqid_3175 |
| variant_4228 | RWKLFLKIPPPKFLKLAKKF | seqid_3176 |
| variant_4229 | FWKLFLKIPPPKFLKLAKKF | seqid_3177 |
| variant_4230 | KKKKFLKIPPPKFLKLAKKF | seqid_3178 |
| variant_4231 | RKKKFLKIPPPKFLKLAKKF | seqid_3179 |
| variant_4232 | FKKKFLKIPPPKFLKLAKKF | seqid_3180 |
| variant_4233 | KWKKFLKIPPPKFLKLAKKF | seqid_3181 |
| variant_4234 | RWKKFLKIPPPKFLKLAKKF | seqid_3182 |
| variant_4235 | FWKKFLKIPPPKFLKLAKKF | seqid_3183 |
| variant_4236 | KKKLLLKIPPPKFLKLAKKF | seqid_3184 |
| variant_4237 | RKKLLLKIPPPKFLKLAKKF | seqid_3185 |
| variant_4238 | FKKLLLKIPPPKFLKLAKKF | seqid_3186 |
| variant_4239 | KWKLLLKIPPPKFLKLAKKF | seqid_3187 |
| variant_4240 | RWKLLLKIPPPKFLKLAKKF | seqid_3188 |
| variant_4241 | FWKLLLKIPPPKFLKLAKKF | seqid_3189 |
| variant_4242 | KKKKLLKIPPPKFLKLAKKF | seqid_3190 |
| variant_4243 | RKKKLLKIPPPKFLKLAKKF | seqid_3191 |
| variant_4244 | FKKKLLKIPPPKFLKLAKKF | seqid_3192 |
| variant_4245 | KWKKLLKIPPPKFLKLAKKF | seqid_3193 |
| variant_4246 | RWKKLLKIPPPKFLKLAKKF | seqid_3194 |
| variant_4247 | FWKKLLKIPPPKFLKLAKKF | seqid_3195 |
| variant_4248 | KKKLFKKIPPPKFLKLAKKF | seqid_3196 |
| variant_4249 | RKKLFKKIPPPKFLKLAKKF | seqid_3197 |
| variant_4250 | FKKLFKKIPPPKFLKLAKKF | seqid_3198 |
| variant_4251 | KWKLFKKIPPPKFLKLAKKF | seqid_3199 |
| variant_4252 | RWKLFKKIPPPKFLKLAKKF | seqid_3200 |
| variant_4253 | FWKLFKKIPPPKFLKLAKKF | seqid_3201 |
| variant_4254 | KKKKFKKIPPPKFLKLAKKF | seqid_3202 |
| variant_4255 | RKKKFKKIPPPKFLKLAKKF | seqid_3203 |
| variant_4256 | FKKKFKKIPPPKFLKLAKKF | seqid_3204 |
| variant_4257 | KWKKFKKIPPPKFLKLAKKF | seqid_3205 |
| variant_4258 | RWKKFKKIPPPKFLKLAKKF | seqid_3206 |
| variant_4259 | FWKKFKKIPPPKFLKLAKKF | seqid_3207 |
| variant_4260 | KKKLLKKIPPPKFLKLAKKF | seqid_3208 |
| variant_4261 | RKKLLKKIPPPKFLKLAKKF | seqid_3209 |
| variant_4262 | FKKLLKKIPPPKFLKLAKKF | seqid_3210 |
| variant_4263 | KWKLLKKIPPPKFLKLAKKF | seqid_3211 |
| variant_4264 | RWKLLKKIPPPKFLKLAKKF | seqid_3212 |
| variant_4265 | FWKLLKKIPPPKFLKLAKKF | seqid_3213 |
| variant_4266 | KKKKLKKIPPPKFLKLAKKF | seqid_3214 |
| variant_4267 | RKKKLKKIPPPKFLKLAKKF | seqid_3215 |
| variant_4268 | FKKKLKKIPPPKFLKLAKKF | seqid_3216 |
| variant_4269 | KWKKLKKIPPPKFLKLAKKF | seqid_3217 |
| variant_4270 | RWKKLKKIPPPKFLKLAKKF | seqid_3218 |
| variant_4271 | FWKKLKKIPPPKFLKLAKKF | seqid_3219 |
| variant_4272 | KKKLFLKIPPKFLKLAKKF | seqid_3220 |
| variant_4273 | RKKLFLKIPPKFLKLAKKF | seqid_3221 |
| variant_4274 | FKKLFLKIPPKFLKLAKKF | seqid_3222 |
| variant_4275 | KWKLFLKIPPKFLKLAKKF | seqid_3223 |
| variant_4276 | RWKLFLKIPPKFLKLAKKF | seqid_3224 |
| variant_4277 | FWKLFLKIPPKFLKLAKKF | seqid_3225 |
| variant_4278 | KKKKFLKIPPKFLKLAKKF | seqid_3226 |
| variant_4279 | RKKKFLKIPPKFLKLAKKF | seqid_3227 |
| variant_4280 | FKKKFLKIPPKFLKLAKKF | seqid_3228 |
| variant_4281 | KWKKFLKIPPKFLKLAKKF | seqid_3229 |
| variant_4282 | RWKKFLKIPPKFLKLAKKF | seqid_3230 |
| variant_4283 | FWKKFLKIPPKFLKLAKKF | seqid_3231 |
| variant_4284 | KKKLLLKIPPKFLKLAKKF | seqid_3232 |
| variant_4285 | RKKLLLKIPPKFLKLAKKF | seqid_3233 |
| variant_4286 | FKKLLLKIPPKFLKLAKKF | seqid_3234 |
| variant_4287 | KWKLLLKIPPKFLKLAKKF | seqid_3235 |
| variant_4288 | RWKLLLKIPPKFLKLAKKF | seqid_3236 |
| variant_4289 | FWKLLLKIPPKFLKLAKKF | seqid_3237 |
| variant_4290 | KKKKLLKIPPKFLKLAKKF | seqid_3238 |
| variant_4291 | RKKKLLKIPPKFLKLAKKF | seqid_3239 |
| variant_4292 | FKKKLLKIPPKFLKLAKKF | seqid_3240 |
| variant_4293 | KWKKLLKIPPKFLKLAKKF | seqid_3241 |
| variant_4294 | RWKKLLKIPPKFLKLAKKF | seqid_3242 |
| variant_4295 | FWKKLLKIPPKFLKLAKKF | seqid_3243 |
| variant_4296 | KKKLFKKIPPKFLKLAKKF | seqid_3244 |
| variant_4297 | RKKLFKKIPPKFLKLAKKF | seqid_3245 |
| variant_4298 | FKKLFKKIPPKFLKLAKKF | seqid_3246 |
| variant_4299 | KWKLFKKIPPKFLKLAKKF | seqid_3247 |
| variant_4300 | RWKLFKKIPPKFLKLAKKF | seqid_3248 |
| variant_4301 | FWKLFKKIPPKFLKLAKKF | seqid_3249 |
| variant_4302 | KKKKFKKIPPKFLKLAKKF | seqid_3250 |
| variant_4303 | RKKKFKKIPPKFLKLAKKF | seqid_3251 |
| variant_4304 | FKKKFKKIPPKFLKLAKKF | seqid_3252 |
| variant_4305 | KWKKFKKIPPKFLKLAKKF | seqid_3253 |
| variant_4306 | RWKKFKKIPPKFLKLAKKF | seqid_3254 |
| variant_4307 | FWKKFKKIPPKFLKLAKKF | seqid_3255 |
| variant_4308 | KKKLLKKIPPKFLKLAKKF | seqid_3256 |
| variant_4309 | RKKLLKKIPPKFLKLAKKF | seqid_3257 |
| variant_4310 | FKKLLKKIPPKFLKLAKKF | seqid_3258 |
| variant_4311 | KWKLLKKIPPKFLKLAKKF | seqid_3259 |
| variant_4312 | RWKLLKKIPPKFLKLAKKF | seqid_3260 |
| variant_4313 | FWKLLKKIPPKFLKLAKKF | seqid_3261 |
| variant_4314 | KKKKLKKIPPKFLKLAKKF | seqid_3262 |
| variant_4315 | RKKKLKKIPPKFLKLAKKF | seqid_3263 |
| variant_4316 | FKKKLKKIPPKFLKLAKKF | seqid_3264 |
| variant_4317 | KWKKLKKIPPKFLKLAKKF | seqid_3265 |
| variant_4318 | RWKKLKKIPPKFLKLAKKF | seqid_3266 |
| variant_4319 | FWKKLKKIPPKFLKLAKKF | seqid_3267 |
| variant_4368 | KKKLFLKIPKFLKLAKKF | seqid_3268 |
| variant_4369 | RKKLFLKIPKFLKLAKKF | seqid_3269 |
| variant_4370 | FKKLFLKIPKFLKLAKKF | seqid_3270 |
| variant_4371 | KWKLFLKIPKFLKLAKKF | seqid_3271 |
| variant_4372 | RWKLFLKIPKFLKLAKKF | seqid_3272 |
| variant_4373 | FWKLFLKIPKFLKLAKKF | seqid_3273 |
| variant_4374 | KKKKFLKIPKFLKLAKKF | seqid_3274 |
| variant_4375 | RKKKFLKIPKFLKLAKKF | seqid_3275 |
| variant_4376 | FKKKFLKIPKFLKLAKKF | seqid_3276 |
| variant_4377 | KWKKFLKIPKFLKLAKKF | seqid_3277 |
| variant_4378 | RWKKFLKIPKFLKLAKKF | seqid_3278 |
| variant_4379 | FWKKFLKIPKFLKLAKKF | seqid_3279 |
| variant_4380 | KKKLLLKIPKFLKLAKKF | seqid_3280 |
| variant_4381 | RKKLLLKIPKFLKLAKKF | seqid_3281 |
| variant_4382 | FKKLLLKIPKFLKLAKKF | seqid_3282 |
| variant_4383 | KWKLLLKIPKFLKLAKKF | seqid_3283 |
| variant_4384 | RWKLLLKIPKFLKLAKKF | seqid_3284 |
| variant_4385 | FWKLLLKIPKFLKLAKKF | seqid_3285 |
| variant_4386 | KKKKLLKIPKFLKLAKKF | seqid_3286 |
| variant_4387 | RKKKLLKIPKFLKLAKKF | seqid_3287 |
| variant_4388 | FKKKLLKIPKFLKLAKKF | seqid_3288 |
| variant_4389 | KWKKLLKIPKFLKLAKKF | seqid_3289 |
| variant_4390 | RWKKLLKIPKFLKLAKKF | seqid_3290 |
| variant_4391 | FWKKLLKIPKFLKLAKKF | seqid_3291 |
| variant_4392 | KKKLFKKIPKFLKLAKKF | seqid_3292 |
| variant_4393 | RKKLFKKIPKFLKLAKKF | seqid_3293 |
| variant_4394 | FKKLFKKIPKFLKLAKKF | seqid_3294 |
| variant_4395 | KWKLFKKIPKFLKLAKKF | seqid_3295 |
| variant_4396 | RWKLFKKIPKFLKLAKKF | seqid_3296 |
| variant_4397 | FWKLFKKIPKFLKLAKKF | seqid_3297 |
| variant_4398 | KKKKFKKIPKFLKLAKKF | seqid_3298 |
| variant_4399 | RKKKFKKIPKFLKLAKKF | seqid_3299 |
| variant_4400 | FKKKFKKIPKFLKLAKKF | seqid_3300 |
| variant_4401 | KWKKFKKIPKFLKLAKKF | seqid_3301 |
| variant_4402 | RWKKFKKIPKFLKLAKKF | seqid_3302 |
| variant_4403 | FWKKFKKIPKFLKLAKKF | seqid_3303 |
| variant_4404 | KKKLLKKIPKFLKLAKKF | seqid_3304 |
| variant_4405 | RKKLLKKIPKFLKLAKKF | seqid_3305 |
| variant_4406 | FKKLLKKIPKFLKLAKKF | seqid_3306 |
| variant_4407 | KWKLLKKIPKFLKLAKKF | seqid_3307 |
| variant_4408 | RWKLLKKIPKFLKLAKKF | seqid_3308 |
| variant_4409 | FWKLLKKIPKFLKLAKKF | seqid_3309 |
| variant_4410 | KKKKLKKIPKFLKLAKKF | seqid_3310 |
| variant_4411 | RKKKLKKIPKFLKLAKKF | seqid_3311 |
| variant_4412 | FKKKLKKIPKFLKLAKKF | seqid_3312 |
| variant_4413 | KWKKLKKIPKFLKLAKKF | seqid_3313 |
| variant_4414 | RWKKLKKIPKFLKLAKKF | seqid_3314 |
| variant_4415 | FWKKLKKIPKFLKLAKKF | seqid_3315 |
| variant_4416 | KKKLFLKIPPPKFKKLAKKF | seqid_3316 |
| variant_4417 | RKKLFLKIPPPKFKKLAKKF | seqid_3317 |
| variant_4418 | FKKLFLKIPPPKFKKLAKKF | seqid_3318 |
| variant_4419 | KWKLFLKIPPPKFKKLAKKF | seqid_3319 |
| variant_4420 | RWKLFLKIPPPKFKKLAKKF | seqid_3320 |
| variant_4421 | FWKLFLKIPPPKFKKLAKKF | seqid_3321 |
| variant_4422 | KKKKFLKIPPPKFKKLAKKF | seqid_3322 |
| variant_4423 | RKKKFLKIPPPKFKKLAKKF | seqid_3323 |
| variant_4424 | FKKKFLKIPPPKFKKLAKKF | seqid_3324 |
| variant_4425 | KWKKFLKIPPPKFKKLAKKF | seqid_3325 |
| variant_4426 | RWKKFLKIPPPKFKKLAKKF | seqid_3326 |
| variant_4427 | FWKKFLKIPPPKFKKLAKKF | seqid_3327 |
| variant_4428 | KKKLLLKIPPPKFKKLAKKF | seqid_3328 |
| variant_4429 | RKKLLLKIPPPKFKKLAKKF | seqid_3329 |
| variant_4430 | FKKLLLKIPPPKFKKLAKKF | seqid_3330 |
| variant_4431 | KWKLLLKIPPPKFKKLAKKF | seqid_3331 |
| variant_4432 | RWKLLLKIPPPKFKKLAKKF | seqid_3332 |
| variant_4433 | FWKLLLKIPPPKFKKLAKKF | seqid_3333 |
| variant_4434 | KKKKLLKIPPPKFKKLAKKF | seqid_3334 |
| variant_4435 | RKKKLLKIPPPKFKKLAKKF | seqid_3335 |
| variant_4436 | FKKKLLKIPPPKFKKLAKKF | seqid_3336 |
| variant_4437 | KWKKLLKIPPPKFKKLAKKF | seqid_3337 |
| variant_4438 | RWKKLLKIPPPKFKKLAKKF | seqid_3338 |
| variant_4439 | FWKKLLKIPPPKFKKLAKKF | seqid_3339 |
| variant_4440 | KKKLFKKIPPPKFKKLAKKF | seqid_3340 |
| variant_4441 | RKKLFKKIPPPKFKKLAKKF | seqid_3341 |
| variant_4442 | FKKLFKKIPPPKFKKLAKKF | seqid_3342 |
| variant_4443 | KWKLFKKIPPPKFKKLAKKF | seqid_3343 |
| variant_4444 | RWKLFKKIPPPKFKKLAKKF | seqid_3344 |
| variant_4445 | FWKLFKKIPPPKFKKLAKKF | seqid_3345 |
| variant_4446 | KKKKFKKIPPPKFKKLAKKF | seqid_3346 |
| variant_4447 | RKKKFKKIPPPKFKKLAKKF | seqid_3347 |
| variant_4448 | FKKKFKKIPPPKFKKLAKKF | seqid_3348 |
| variant_4449 | KWKKFKKIPPPKFKKLAKKF | seqid_3349 |
| variant_4450 | RWKKFKKIPPPKFKKLAKKF | seqid_3350 |
| variant_4451 | FWKKFKKIPPPKFKKLAKKF | seqid_3351 |
| variant_4452 | KKKLLKKIPPPKFKKLAKKF | seqid_3352 |
| variant_4453 | RKKLLKKIPPPKFKKLAKKF | seqid_3353 |
| variant_4454 | FKKLLKKIPPPKFKKLAKKF | seqid_3354 |
| variant_4455 | KWKLLKKIPPPKFKKLAKKF | seqid_3355 |
| variant_4456 | RWKLLKKIPPPKFKKLAKKF | seqid_3356 |
| variant_4457 | FWKLLKKIPPPKFKKLAKKF | seqid_3357 |
| variant_4458 | KKKKLKKIPPPKFKKLAKKF | seqid_3358 |
| variant_4459 | RKKKLKKIPPPKFKKLAKKF | seqid_3359 |
| variant_4460 | FKKKLKKIPPPKFKKLAKKF | seqid_3360 |
| variant_4461 | KWKKLKKIPPPKFKKLAKKF | seqid_3361 |
| variant_4462 | RWKKLKKIPPPKFKKLAKKF | seqid_3362 |
| variant_4463 | FWKKLKKIPPPKFKKLAKKF | seqid_3363 |
| variant_4464 | KKKLFLKIPPKFKKLAKKF | seqid_3364 |
| variant_4465 | RKKLFLKIPPKFKKLAKKF | seqid_3365 |
| variant_4466 | FKKLFLKIPPKFKKLAKKF | seqid_3366 |
| variant_4467 | KWKLFLKIPPKFKKLAKKF | seqid_3367 |
| variant_4468 | RWKLFLKIPPKFKKLAKKF | seqid_3368 |
| variant_4469 | FWKLFLKIPPKFKKLAKKF | seqid_3369 |
| variant_4470 | KKKKFLKIPPKFKKLAKKF | seqid_3370 |
| variant_4471 | RKKKFLKIPPKFKKLAKKF | seqid_3371 |
| variant_4472 | FKKKFLKIPPKFKKLAKKF | seqid_3372 |
| variant_4473 | KWKKFLKIPPKFKKLAKKF | seqid_3373 |
| variant_4474 | RWKKFLKIPPKFKKLAKKF | seqid_3374 |
| variant_4475 | FWKKFLKIPPKFKKLAKKF | seqid_3375 |
| variant_4476 | KKKLLLKIPPKFKKLAKKF | seqid_3376 |
| variant_4477 | RKKLLLKIPPKFKKLAKKF | seqid_3377 |
| variant_4478 | FKKLLLKIPPKFKKLAKKF | seqid_3378 |
| variant_4479 | KWKLLLKIPPKFKKLAKKF | seqid_3379 |
| variant_4480 | RWKLLLKIPPKFKKLAKKF | seqid_3380 |
| variant_4481 | FWKLLLKIPPKFKKLAKKF | seqid_3381 |
| variant_4482 | KKKKLLKIPPKFKKLAKKF | seqid_3382 |
| variant_4483 | RKKKLLKIPPKFKKLAKKF | seqid_3383 |
| variant_4484 | FKKKLLKIPPKFKKLAKKF | seqid_3384 |
| variant_4485 | KWKKLLKIPPKFKKLAKKF | seqid_3385 |
| variant_4486 | RWKKLLKIPPKFKKLAKKF | seqid_3386 |
| variant_4487 | FWKKLLKIPPKFKKLAKKF | seqid_3387 |
| variant_4488 | KKKLFKKIPPKFKKLAKKF | seqid_3388 |
| variant_4489 | RKKLFKKIPPKFKKLAKKF | seqid_3389 |
| variant_4490 | FKKLFKKIPPKFKKLAKKF | seqid_3390 |
| variant_4491 | KWKLFKKIPPKFKKLAKKF | seqid_3391 |
| variant_4492 | RWKLFKKIPPKFKKLAKKF | seqid_3392 |
| variant_4493 | FWKLFKKIPPKFKKLAKKF | seqid_3393 |
| variant_4494 | KKKKFKKIPPKFKKLAKKF | seqid_3394 |
| variant_4495 | RKKKFKKIPPKFKKLAKKF | seqid_3395 |
| variant_4496 | FKKKFKKIPPKFKKLAKKF | seqid_3396 |
| variant_4497 | KWKKFKKIPPKFKKLAKKF | seqid_3397 |
| variant_4498 | RWKKFKKIPPKFKKLAKKF | seqid_3398 |
| variant_4499 | FWKKFKKIPPKFKKLAKKF | seqid_3399 |
| variant_4500 | KKKLLKKIPPKFKKLAKKF | seqid_3400 |
| variant_4501 | RKKLLKKIPPKFKKLAKKF | seqid_3401 |
| variant_4502 | FKKLLKKIPPKFKKLAKKF | seqid_3402 |
| variant_4503 | KWKLLKKIPPKFKKLAKKF | seqid_3403 |
| variant_4504 | RWKLLKKIPPKFKKLAKKF | seqid_3404 |
| variant_4505 | FWKLLKKIPPKFKKLAKKF | seqid_3405 |
| variant_4506 | KKKKLKKIPPKFKKLAKKF | seqid_3406 |
| variant_4507 | RKKKLKKIPPKFKKLAKKF | seqid_3407 |
| variant_4508 | FKKKLKKIPPKFKKLAKKF | seqid_3408 |
| variant_4509 | KWKKLKKIPPKFKKLAKKF | seqid_3409 |
| variant_4510 | RWKKLKKIPPKFKKLAKKF | seqid_3410 |
| variant_4511 | FWKKLKKIPPKFKKLAKKF | seqid_3411 |
| variant_4560 | KKKLFLKIPKFKKLAKKF | seqid_3412 |
| variant_4561 | RKKLFLKIPKFKKLAKKF | seqid_3413 |
| variant_4562 | FKKLFLKIPKFKKLAKKF | seqid_3414 |
| variant_4563 | KWKLFLKIPKFKKLAKKF | seqid_3415 |
| variant_4564 | RWKLFLKIPKFKKLAKKF | seqid_3416 |
| variant_4565 | FWKLFLKIPKFKKLAKKF | seqid_3417 |
| variant_4566 | KKKKFLKIPKFKKLAKKF | seqid_3418 |
| variant_4567 | RKKKFLKIPKFKKLAKKF | seqid_3419 |
| variant_4568 | FKKKFLKIPKFKKLAKKF | seqid_3420 |
| variant_4569 | KWKKFLKIPKFKKLAKKF | seqid_3421 |
| variant_4570 | RWKKFLKIPKFKKLAKKF | seqid_3422 |
| variant_4571 | FWKKFLKIPKFKKLAKKF | seqid_3423 |
| variant_4572 | KKKLLLKIPKFKKLAKKF | seqid_3424 |
| variant_4573 | RKKLLLKIPKFKKLAKKF | seqid_3425 |
| variant_4574 | FKKLLLKIPKFKKLAKKF | seqid_3426 |
| variant_4575 | KWKLLLKIPKFKKLAKKF | seqid_3427 |
| variant_4576 | RWKLLLKIPKFKKLAKKF | seqid_3428 |
| variant_4577 | FWKLLLKIPKFKKLAKKF | seqid_3429 |
| variant_4578 | KKKKLLKIPKFKKLAKKF | seqid_3430 |
| variant_4579 | RKKKLLKIPKFKKLAKKF | seqid_3431 |
| variant_4580 | FKKKLLKIPKFKKLAKKF | seqid_3432 |
| variant_4581 | KWKKLLKIPKFKKLAKKF | seqid_3433 |
| variant_4582 | RWKKLLKIPKFKKLAKKF | seqid_3434 |
| variant_4583 | FWKKLLKIPKFKKLAKKF | seqid_3435 |
| variant_4584 | KKKLFKKIPKFKKLAKKF | seqid_3436 |
| variant_4585 | RKKLFKKIPKFKKLAKKF | seqid_3437 |
| variant_4586 | FKKLFKKIPKFKKLAKKF | seqid_3438 |
| variant_4587 | KWKLFKKIPKFKKLAKKF | seqid_3439 |
| variant_4588 | RWKLFKKIPKFKKLAKKF | seqid_3440 |
| variant_4589 | FWKLFKKIPKFKKLAKKF | seqid_3441 |
| variant_4590 | KKKKFKKIPKFKKLAKKF | seqid_3442 |
| variant_4591 | RKKKFKKIPKFKKLAKKF | seqid_3443 |
| variant_4592 | FKKKFKKIPKFKKLAKKF | seqid_3444 |
| variant_4593 | KWKKFKKIPKFKKLAKKF | seqid_3445 |
| variant_4594 | RWKKFKKIPKFKKLAKKF | seqid_3446 |
| variant_4595 | FWKKFKKIPKFKKLAKKF | seqid_3447 |
| variant_4596 | KKKLLKKIPKFKKLAKKF | seqid_3448 |
| variant_4597 | RKKLLKKIPKFKKLAKKF | seqid_3449 |
| variant_4598 | FKKLLKKIPKFKKLAKKF | seqid_3450 |
| variant_4599 | KWKLLKKIPKFKKLAKKF | seqid_3451 |
| variant_4600 | RWKLLKKIPKFKKLAKKF | seqid_3452 |
| variant_4601 | FWKLLKKIPKFKKLAKKF | seqid_3453 |
| variant_4602 | KKKKLKKIPKFKKLAKKF | seqid_3454 |
| variant_4603 | RKKKLKKIPKFKKLAKKF | seqid_3455 |
| variant_4604 | FKKKLKKIPKFKKLAKKF | seqid_3456 |
| variant_4605 | KWKKLKKIPKFKKLAKKF | seqid_3457 |
| variant_4606 | RWKKLKKIPKFKKLAKKF | seqid_3458 |
| variant_4607 | FWKKLKKIPKFKKLAKKF | seqid_3459 |
| variant_4608 | KKKLFLKIPPPKFLHVAKKF | seqid_3460 |
| variant_4609 | RKKLFLKIPPPKFLHVAKKF | seqid_3461 |
| variant_4610 | FKKLFLKIPPPKFLHVAKKF | seqid_3462 |
| variant_4611 | KWKLFLKIPPPKFLHVAKKF | seqid_3463 |
| variant_4612 | RWKLFLKIPPPKFLHVAKKF | seqid_3464 |
| variant_4613 | FWKLFLKIPPPKFLHVAKKF | seqid_3465 |
| variant_4614 | KKKKFLKIPPPKFLHVAKKF | seqid_3466 |
| variant_4615 | RKKKFLKIPPPKFLHVAKKF | seqid_3467 |
| variant_4616 | FKKKFLKIPPPKFLHVAKKF | seqid_3468 |
| variant_4617 | KWKKFLKIPPPKFLHVAKKF | seqid_3469 |
| variant_4618 | RWKKFLKIPPPKFLHVAKKF | seqid_3470 |
| variant_4619 | FWKKFLKIPPPKFLHVAKKF | seqid_3471 |
| variant_4620 | KKKLLLKIPPPKFLHVAKKF | seqid_3472 |
| variant_4621 | RKKLLLKIPPPKFLHVAKKF | seqid_3473 |
| variant_4622 | FKKLLLKIPPPKFLHVAKKF | seqid_3474 |
| variant_4623 | KWKLLLKIPPPKFLHVAKKF | seqid_3475 |
| variant_4624 | RWKLLLKIPPPKFLHVAKKF | seqid_3476 |
| variant_4625 | FWKLLLKIPPPKFLHVAKKF | seqid_3477 |
| variant_4626 | KKKKLLKIPPPKFLHVAKKF | seqid_3478 |
| variant_4627 | RKKKLLKIPPPKFLHVAKKF | seqid_3479 |
| variant_4628 | FKKKLLKIPPPKFLHVAKKF | seqid_3480 |
| variant_4629 | KWKKLLKIPPPKFLHVAKKF | seqid_3481 |
| variant_4630 | RWKKLLKIPPPKFLHVAKKF | seqid_3482 |
| variant_4631 | FWKKLLKIPPPKFLHVAKKF | seqid_3483 |
| variant_4632 | KKKLFKKIPPPKFLHVAKKF | seqid_3484 |
| variant_4633 | RKKLFKKIPPPKFLHVAKKF | seqid_3485 |
| variant_4634 | FKKLFKKIPPPKFLHVAKKF | seqid_3486 |
| variant_4635 | KWKLFKKIPPPKFLHVAKKF | seqid_3487 |
| variant_4636 | RWKLFKKIPPPKFLHVAKKF | seqid_3488 |
| variant_4637 | FWKLFKKIPPPKFLHVAKKF | seqid_3489 |
| variant_4638 | KKKKFKKIPPPKFLHVAKKF | seqid_3490 |
| variant_4639 | RKKKFKKIPPPKFLHVAKKF | seqid_3491 |
| variant_4640 | FKKKFKKIPPPKFLHVAKKF | seqid_3492 |
| variant_4641 | KWKKFKKIPPPKFLHVAKKF | seqid_3493 |
| variant_4642 | RWKKFKKIPPPKFLHVAKKF | seqid_3494 |
| variant_4643 | FWKKFKKIPPPKFLHVAKKF | seqid_3495 |
| variant_4644 | KKKLLKKIPPPKFLHVAKKF | seqid_3496 |
| variant_4645 | RKKLLKKIPPPKFLHVAKKF | seqid_3497 |
| variant_4646 | FKKLLKKIPPPKFLHVAKKF | seqid_3498 |
| variant_4647 | KWKLLKKIPPPKFLHVAKKF | seqid_3499 |
| variant_4648 | RWKLLKKIPPPKFLHVAKKF | seqid_3500 |
| variant_4649 | FWKLLKKIPPPKFLHVAKKF | seqid_3501 |
| variant_4650 | KKKKLKKIPPPKFLHVAKKF | seqid_3502 |
| variant_4651 | RKKKLKKIPPPKFLHVAKKF | seqid_3503 |
| variant_4652 | FKKKLKKIPPPKFLHVAKKF | seqid_3504 |
| variant_4653 | KWKKLKKIPPPKFLHVAKKF | seqid_3505 |
| variant_4654 | RWKKLKKIPPPKFLHVAKKF | seqid_3506 |
| variant_4655 | FWKKLKKIPPPKFLHVAKKF | seqid_3507 |
| variant_4656 | KKKLFLKIPPKFLHVAKKF | seqid_3508 |
| variant_4657 | RKKLFLKIPPKFLHVAKKF | seqid_3509 |
| variant_4658 | FKKLFLKIPPKFLHVAKKF | seqid_3510 |
| variant_4659 | KWKLFLKIPPKFLHVAKKF | seqid_3511 |
| variant_4660 | RWKLFLKIPPKFLHVAKKF | seqid_3512 |
| variant_4661 | FWKLFLKIPPKFLHVAKKF | seqid_3513 |
| variant_4662 | KKKKFLKIPPKFLHVAKKF | seqid_3514 |
| variant_4663 | RKKKFLKIPPKFLHVAKKF | seqid_3515 |
| variant_4664 | FKKKFLKIPPKFLHVAKKF | seqid_3516 |
| variant_4665 | KWKKFLKIPPKFLHVAKKF | seqid_3517 |
| variant_4666 | RWKKFLKIPPKFLHVAKKF | seqid_3518 |
| variant_4667 | FWKKFLKIPPKFLHVAKKF | seqid_3519 |
| variant_4668 | KKKLLLKIPPKFLHVAKKF | seqid_3520 |
| variant_4669 | RKKLLLKIPPKFLHVAKKF | seqid_3521 |
| variant_4670 | FKKLLLKIPPKFLHVAKKF | seqid_3522 |
| variant_4671 | KWKLLLKIPPKFLHVAKKF | seqid_3523 |
| variant_4672 | RWKLLLKIPPKFLHVAKKF | seqid_3524 |
| variant_4673 | FWKLLLKIPPKFLHVAKKF | seqid_3525 |
| variant_4674 | KKKKLLKIPPKFLHVAKKF | seqid_3526 |
| variant_4675 | RKKKLLKIPPKFLHVAKKF | seqid_3527 |
| variant_4676 | FKKKLLKIPPKFLHVAKKF | seqid_3528 |
| variant_4677 | KWKKLLKIPPKFLHVAKKF | seqid_3529 |
| variant_4678 | RWKKLLKIPPKFLHVAKKF | seqid_3530 |
| variant_4679 | FWKKLLKIPPKFLHVAKKF | seqid_3531 |
| variant_4680 | KKKLFKKIPPKFLHVAKKF | seqid_3532 |
| variant_4681 | RKKLFKKIPPKFLHVAKKF | seqid_3533 |
| variant_4682 | FKKLFKKIPPKFLHVAKKF | seqid_3534 |
| variant_4683 | KWKLFKKIPPKFLHVAKKF | seqid_3535 |
| variant_4684 | RWKLFKKIPPKFLHVAKKF | seqid_3536 |
| variant_4685 | FWKLFKKIPPKFLHVAKKF | seqid_3537 |
| variant_4686 | KKKKFKKIPPKFLHVAKKF | seqid_3538 |
| variant_4687 | RKKKFKKIPPKFLHVAKKF | seqid_3539 |
| variant_4688 | FKKKFKKIPPKFLHVAKKF | seqid_3540 |
| variant_4689 | KWKKFKKIPPKFLHVAKKF | seqid_3541 |
| variant_4690 | RWKKFKKIPPKFLHVAKKF | seqid_3542 |
| variant_4691 | FWKKFKKIPPKFLHVAKKF | seqid_3543 |
| variant_4692 | KKKLLKKIPPKFLHVAKKF | seqid_3544 |
| variant_4693 | RKKLLKKIPPKFLHVAKKF | seqid_3545 |
| variant_4694 | FKKLLKKIPPKFLHVAKKF | seqid_3546 |
| variant_4695 | KWKLLKKIPPKFLHVAKKF | seqid_3547 |
| variant_4696 | RWKLLKKIPPKFLHVAKKF | seqid_3548 |
| variant_4697 | FWKLLKKIPPKFLHVAKKF | seqid_3549 |
| variant_4698 | KKKKLKKIPPKFLHVAKKF | seqid_3550 |
| variant_4699 | RKKKLKKIPPKFLHVAKKF | seqid_3551 |
| variant_4700 | FKKKLKKIPPKFLHVAKKF | seqid_3552 |
| variant_4701 | KWKKLKKIPPKFLHVAKKF | seqid_3553 |
| variant_4702 | RWKKLKKIPPKFLHVAKKF | seqid_3554 |
| variant_4703 | FWKKLKKIPPKFLHVAKKF | seqid_3555 |
| variant_4752 | KKKLFLKIPKFLHVAKKF | seqid_3556 |
| variant_4753 | RKKLFLKIPKFLHVAKKF | seqid_3557 |
| variant_4754 | FKKLFLKIPKFLHVAKKF | seqid_3558 |
| variant_4755 | KWKLFLKIPKFLHVAKKF | seqid_3559 |
| variant_4756 | RWKLFLKIPKFLHVAKKF | seqid_3560 |
| variant_4757 | FWKLFLKIPKFLHVAKKF | seqid_3561 |
| variant_4758 | KKKKFLKIPKFLHVAKKF | seqid_3562 |
| variant_4759 | RKKKFLKIPKFLHVAKKF | seqid_3563 |
| variant_4760 | FKKKFLKIPKFLHVAKKF | seqid_3564 |
| variant_4761 | KWKKFLKIPKFLHVAKKF | seqid_3565 |
| variant_4762 | RWKKFLKIPKFLHVAKKF | seqid_3566 |
| variant_4763 | FWKKFLKIPKFLHVAKKF | seqid_3567 |
| variant_4764 | KKKLLLKIPKFLHVAKKF | seqid_3568 |
| variant_4765 | RKKLLLKIPKFLHVAKKF | seqid_3569 |
| variant_4766 | FKKLLLKIPKFLHVAKKF | seqid_3570 |
| variant_4767 | KWKLLLKIPKFLHVAKKF | seqid_3571 |
| variant_4768 | RWKLLLKIPKFLHVAKKF | seqid_3572 |
| variant_4769 | FWKLLLKIPKFLHVAKKF | seqid_3573 |
| variant_4770 | KKKKLLKIPKFLHVAKKF | seqid_3574 |
| variant_4771 | RKKKLLKIPKFLHVAKKF | seqid_3575 |
| variant_4772 | FKKKLLKIPKFLHVAKKF | seqid_3576 |
| variant_4773 | KWKKLLKIPKFLHVAKKF | seqid_3577 |
| variant_4774 | RWKKLLKIPKFLHVAKKF | seqid_3578 |
| variant_4775 | FWKKLLKIPKFLHVAKKF | seqid_3579 |
| variant_4776 | KKKLFKKIPKFLHVAKKF | seqid_3580 |
| variant_4777 | RKKLFKKIPKFLHVAKKF | seqid_3581 |
| variant_4778 | FKKLFKKIPKFLHVAKKF | seqid_3582 |
| variant_4779 | KWKLFKKIPKFLHVAKKF | seqid_3583 |
| variant_4780 | RWKLFKKIPKFLHVAKKF | seqid_3584 |
| variant_4781 | FWKLFKKIPKFLHVAKKF | seqid_3585 |
| variant_4782 | KKKKFKKIPKFLHVAKKF | seqid_3586 |
| variant_4783 | RKKKFKKIPKFLHVAKKF | seqid_3587 |
| variant_4784 | FKKKFKKIPKFLHVAKKF | seqid_3588 |
| variant_4785 | KWKKFKKIPKFLHVAKKF | seqid_3589 |
| variant_4786 | RWKKFKKIPKFLHVAKKF | seqid_3590 |
| variant_4787 | FWKKFKKIPKFLHVAKKF | seqid_3591 |
| variant_4788 | KKKLLKKIPKFLHVAKKF | seqid_3592 |
| variant_4789 | RKKLLKKIPKFLHVAKKF | seqid_3593 |
| variant_4790 | FKKLLKKIPKFLHVAKKF | seqid_3594 |
| variant_4791 | KWKLLKKIPKFLHVAKKF | seqid_3595 |
| variant_4792 | RWKLLKKIPKFLHVAKKF | seqid_3596 |
| variant_4793 | FWKLLKKIPKFLHVAKKF | seqid_3597 |
| variant_4794 | KKKKLKKIPKFLHVAKKF | seqid_3598 |
| variant_4795 | RKKKLKKIPKFLHVAKKF | seqid_3599 |
| variant_4796 | FKKKLKKIPKFLHVAKKF | seqid_3600 |
| variant_4797 | KWKKLKKIPKFLHVAKKF | seqid_3601 |
| variant_4798 | RWKKLKKIPKFLHVAKKF | seqid_3602 |
| variant_4799 | FWKKLKKIPKFLHVAKKF | seqid_3603 |
| variant_4800 | KKKLFLKIPPPKFKHVAKKF | seqid_3604 |
| variant_4801 | RKKLFLKIPPPKFKHVAKKF | seqid_3605 |
| variant_4802 | FKKLFLKIPPPKFKHVAKKF | seqid_3606 |
| variant_4803 | KWKLFLKIPPPKFKHVAKKF | seqid_3607 |
| variant_4804 | RWKLFLKIPPPKFKHVAKKF | seqid_3608 |
| variant_4805 | FWKLFLKIPPPKFKHVAKKF | seqid_3609 |
| variant_4806 | KKKKFLKIPPPKFKHVAKKF | seqid_3610 |
| variant_4807 | RKKKFLKIPPPKFKHVAKKF | seqid_3611 |
| variant_4808 | FKKKFLKIPPPKFKHVAKKF | seqid_3612 |
| variant_4809 | KWKKFLKIPPPKFKHVAKKF | seqid_3613 |
| variant_4810 | RWKKFLKIPPPKFKHVAKKF | seqid_3614 |
| variant_4811 | FWKKFLKIPPPKFKHVAKKF | seqid_3615 |
| variant_4812 | KKKLLLKIPPPKFKHVAKKF | seqid_3616 |
| variant_4813 | RKKLLLKIPPPKFKHVAKKF | seqid_3617 |
| variant_4814 | FKKLLLKIPPPKFKHVAKKF | seqid_3618 |
| variant_4815 | KWKLLLKIPPPKFKHVAKKF | seqid_3619 |
| variant_4816 | RWKLLLKIPPPKFKHVAKKF | seqid_3620 |
| variant_4817 | FWKLLLKIPPPKFKHVAKKF | seqid_3621 |
| variant_4818 | KKKKLLKIPPPKFKHVAKKF | seqid_3622 |
| variant_4819 | RKKKLLKIPPPKFKHVAKKF | seqid_3623 |
| variant_4820 | FKKKLLKIPPPKFKHVAKKF | seqid_3624 |
| variant_4821 | KWKKLLKIPPPKFKHVAKKF | seqid_3625 |
| variant_4822 | RWKKLLKIPPPKFKHVAKKF | seqid_3626 |
| variant_4823 | FWKKLLKIPPPKFKHVAKKF | seqid_3627 |
| variant_4824 | KKKLFKKIPPPKFKHVAKKF | seqid_3628 |
| variant_4825 | RKKLFKKIPPPKFKHVAKKF | seqid_3629 |
| variant_4826 | FKKLFKKIPPPKFKHVAKKF | seqid_3630 |
| variant_4827 | KWKLFKKIPPPKFKHVAKKF | seqid_3631 |
| variant_4828 | RWKLFKKIPPPKFKHVAKKF | seqid_3632 |
| variant_4829 | FWKLFKKIPPPKFKHVAKKF | seqid_3633 |
| variant_4830 | KKKKFKKIPPPKFKHVAKKF | seqid_3634 |
| variant_4831 | RKKKFKKIPPPKFKHVAKKF | seqid_3635 |
| variant_4832 | FKKKFKKIPPPKFKHVAKKF | seqid_3636 |
| variant_4833 | KWKKFKKIPPPKFKHVAKKF | seqid_3637 |
| variant_4834 | RWKKFKKIPPPKFKHVAKKF | seqid_3638 |
| variant_4835 | FWKKFKKIPPPKFKHVAKKF | seqid_3639 |
| variant_4836 | KKKLLKKIPPPKFKHVAKKF | seqid_3640 |
| variant_4837 | RKKLLKKIPPPKFKHVAKKF | seqid_3641 |
| variant_4838 | FKKLLKKIPPPKFKHVAKKF | seqid_3642 |
| variant_4839 | KWKLLKKIPPPKFKHVAKKF | seqid_3643 |
| variant_4840 | RWKLLKKIPPPKFKHVAKKF | seqid_3644 |
| variant_4841 | FWKLLKKIPPPKFKHVAKKF | seqid_3645 |
| variant_4842 | KKKKLKKIPPPKFKHVAKKF | seqid_3646 |
| variant_4843 | RKKKLKKIPPPKFKHVAKKF | seqid_3647 |
| variant_4844 | FKKKLKKIPPPKFKHVAKKF | seqid_3648 |
| variant_4845 | KWKKLKKIPPPKFKHVAKKF | seqid_3649 |
| variant_4846 | RWKKLKKIPPPKFKHVAKKF | seqid_3650 |
| variant_4847 | FWKKLKKIPPPKFKHVAKKF | seqid_3651 |
| variant_4848 | KKKLFLKIPPKFKHVAKKF | seqid_3652 |
| variant_4849 | RKKLFLKIPPKFKHVAKKF | seqid_3653 |
| variant_4850 | FKKLFLKIPPKFKHVAKKF | seqid_3654 |
| variant_4851 | KWKLFLKIPPKFKHVAKKF | seqid_3655 |
| variant_4852 | RWKLFLKIPPKFKHVAKKF | seqid_3656 |
| variant_4853 | FWKLFLKIPPKFKHVAKKF | seqid_3657 |
| variant_4854 | KKKKFLKIPPKFKHVAKKF | seqid_3658 |
| variant_4855 | RKKKFLKIPPKFKHVAKKF | seqid_3659 |
| variant_4856 | FKKKFLKIPPKFKHVAKKF | seqid_3660 |
| variant_4857 | KWKKFLKIPPKFKHVAKKF | seqid_3661 |
| variant_4858 | RWKKFLKIPPKFKHVAKKF | seqid_3662 |
| variant_4859 | FWKKFLKIPPKFKHVAKKF | seqid_3663 |
| variant_4860 | KKKLLLKIPPKFKHVAKKF | seqid_3664 |
| variant_4861 | RKKLLLKIPPKFKHVAKKF | seqid_3665 |
| variant_4862 | FKKLLLKIPPKFKHVAKKF | seqid_3666 |
| variant_4863 | KWKLLLKIPPKFKHVAKKF | seqid_3667 |
| variant_4864 | RWKLLLKIPPKFKHVAKKF | seqid_3668 |
| variant_4865 | FWKLLLKIPPKFKHVAKKF | seqid_3669 |
| variant_4866 | KKKKLLKIPPKFKHVAKKF | seqid_3670 |
| variant_4867 | RKKKLLKIPPKFKHVAKKF | seqid_3671 |
| variant_4868 | FKKKLLKIPPKFKHVAKKF | seqid_3672 |
| variant_4869 | KWKKLLKIPPKFKHVAKKF | seqid_3673 |
| variant_4870 | RWKKLLKIPPKFKHVAKKF | seqid_3674 |
| variant_4871 | FWKKLLKIPPKFKHVAKKF | seqid_3675 |
| variant_4872 | KKKLFKKIPPKFKHVAKKF | seqid_3676 |
| variant_4873 | RKKLFKKIPPKFKHVAKKF | seqid_3677 |
| variant_4874 | FKKLFKKIPPKFKHVAKKF | seqid_3678 |
| variant_4875 | KWKLFKKIPPKFKHVAKKF | seqid_3679 |
| variant_4876 | RWKLFKKIPPKFKHVAKKF | seqid_3680 |
| variant_4877 | FWKLFKKIPPKFKHVAKKF | seqid_3681 |
| variant_4878 | KKKKFKKIPPKFKHVAKKF | seqid_3682 |
| variant_4879 | RKKKFKKIPPKFKHVAKKF | seqid_3683 |
| variant_4880 | FKKKFKKIPPKFKHVAKKF | seqid_3684 |
| variant_4881 | KWKKFKKIPPKFKHVAKKF | seqid_3685 |
| variant_4882 | RWKKFKKIPPKFKHVAKKF | seqid_3686 |
| variant_4883 | FWKKFKKIPPKFKHVAKKF | seqid_3687 |
| variant_4884 | KKKLLKKIPPKFKHVAKKF | seqid_3688 |
| variant_4885 | RKKLLKKIPPKFKHVAKKF | seqid_3689 |
| variant_4886 | FKKLLKKIPPKFKHVAKKF | seqid_3690 |
| variant_4887 | KWKLLKKIPPKFKHVAKKF | seqid_3691 |
| variant_4888 | RWKLLKKIPPKFKHVAKKF | seqid_3692 |
| variant_4889 | FWKLLKKIPPKFKHVAKKF | seqid_3693 |
| variant_4890 | KKKKLKKIPPKFKHVAKKF | seqid_3694 |
| variant_4891 | RKKKLKKIPPKFKHVAKKF | seqid_3695 |
| variant_4892 | FKKKLKKIPPKFKHVAKKF | seqid_3696 |
| variant_4893 | KWKKLKKIPPKFKHVAKKF | seqid_3697 |
| variant_4894 | RWKKLKKIPPKFKHVAKKF | seqid_3698 |
| variant_4895 | FWKKLKKIPPKFKHVAKKF | seqid_3699 |
| variant_4944 | KKKLFLKIPKFKHVAKKF | seqid_3700 |
| variant_4945 | RKKLFLKIPKFKHVAKKF | seqid_3701 |
| variant_4946 | FKKLFLKIPKFKHVAKKF | seqid_3702 |
| variant_4947 | KWKLFLKIPKFKHVAKKF | seqid_3703 |
| variant_4948 | RWKLFLKIPKFKHVAKKF | seqid_3704 |
| variant_4949 | FWKLFLKIPKFKHVAKKF | seqid_3705 |
| variant_4950 | KKKKFLKIPKFKHVAKKF | seqid_3706 |
| variant_4951 | RKKKFLKIPKFKHVAKKF | seqid_3707 |
| variant_4952 | FKKKFLKIPKFKHVAKKF | seqid_3708 |
| variant_4953 | KWKKFLKIPKFKHVAKKF | seqid_3709 |
| variant_4954 | RWKKFLKIPKFKHVAKKF | seqid_3710 |
| variant_4955 | FWKKFLKIPKFKHVAKKF | seqid_3711 |
| variant_4956 | KKKLLLKIPKFKHVAKKF | seqid_3712 |
| variant_4957 | RKKLLLKIPKFKHVAKKF | seqid_3713 |
| variant_4958 | FKKLLLKIPKFKHVAKKF | seqid_3714 |
| variant_4959 | KWKLLLKIPKFKHVAKKF | seqid_3715 |
| variant_4960 | RWKLLLKIPKFKHVAKKF | seqid_3716 |
| variant_4961 | FWKLLLKIPKFKHVAKKF | seqid_3717 |
| variant_4962 | KKKKLLKIPKFKHVAKKF | seqid_3718 |
| variant_4963 | RKKKLLKIPKFKHVAKKF | seqid_3719 |
| variant_4964 | FKKKLLKIPKFKHVAKKF | seqid_3720 |
| variant_4965 | KWKKLLKIPKFKHVAKKF | seqid_3721 |
| variant_4966 | RWKKLLKIPKFKHVAKKF | seqid_3722 |
| variant_4967 | FWKKLLKIPKFKHVAKKF | seqid_3723 |
| variant_4968 | KKKLFKKIPKFKHVAKKF | seqid_3724 |
| variant_4969 | RKKLFKKIPKFKHVAKKF | seqid_3725 |
| variant_4970 | FKKLFKKIPKFKHVAKKF | seqid_3726 |
| variant_4971 | KWKLFKKIPKFKHVAKKF | seqid_3727 |
| variant_4972 | RWKLFKKIPKFKHVAKKF | seqid_3728 |
| variant_4973 | FWKLFKKIPKFKHVAKKF | seqid_3729 |
| variant_4974 | KKKKFKKIPKFKHVAKKF | seqid_3730 |
| variant_4975 | RKKKFKKIPKFKHVAKKF | seqid_3731 |
| variant_4976 | FKKKFKKIPKFKHVAKKF | seqid_3732 |
| variant_4977 | KWKKFKKIPKFKHVAKKF | seqid_3733 |
| variant_4978 | RWKKFKKIPKFKHVAKKF | seqid_3734 |
| variant_4979 | FWKKFKKIPKFKHVAKKF | seqid_3735 |
| variant_4980 | KKKLLKKIPKFKHVAKKF | seqid_3736 |
| variant_4981 | RKKLLKKIPKFKHVAKKF | seqid_3737 |
| variant_4982 | FKKLLKKIPKFKHVAKKF | seqid_3738 |
| variant_4983 | KWKLLKKIPKFKHVAKKF | seqid_3739 |
| variant_4984 | RWKLLKKIPKFKHVAKKF | seqid_3740 |
| variant_4985 | FWKLLKKIPKFKHVAKKF | seqid_3741 |
| variant_4986 | KKKKLKKIPKFKHVAKKF | seqid_3742 |
| variant_4987 | RKKKLKKIPKFKHVAKKF | seqid_3743 |
| variant_4988 | FKKKLKKIPKFKHVAKKF | seqid_3744 |
| variant_4989 | KWKKLKKIPKFKHVAKKF | seqid_3745 |
| variant_4990 | RWKKLKKIPKFKHVAKKF | seqid_3746 |
| variant_4991 | FWKKLKKIPKFKHVAKKF | seqid_3747 |
| variant_4992 | KKKLFLKIPPPKFLKVAKKF | seqid_3748 |
| variant_4993 | RKKLFLKIPPPKFLKVAKKF | seqid_3749 |
| variant_4994 | FKKLFLKIPPPKFLKVAKKF | seqid_3750 |
| variant_4995 | KWKLFLKIPPPKFLKVAKKF | seqid_3751 |
| variant_4996 | RWKLFLKIPPPKFLKVAKKF | seqid_3752 |
| variant_4997 | FWKLFLKIPPPKFLKVAKKF | seqid_3753 |
| variant_4998 | KKKKFLKIPPPKFLKVAKKF | seqid_3754 |
| variant_4999 | RKKKFLKIPPPKFLKVAKKF | seqid_3755 |
| variant_5000 | FKKKFLKIPPPKFLKVAKKF | seqid_3756 |
| variant_5001 | KWKKFLKIPPPKFLKVAKKF | seqid_3757 |
| variant_5002 | RWKKFLKIPPPKFLKVAKKF | seqid_3758 |
| variant_5003 | FWKKFLKIPPPKFLKVAKKF | seqid_3759 |
| variant_5004 | KKKLLLKIPPPKFLKVAKKF | seqid_3760 |
| variant_5005 | RKKLLLKIPPPKFLKVAKKF | seqid_3761 |
| variant_5006 | FKKLLLKIPPPKFLKVAKKF | seqid_3762 |
| variant_5007 | KWKLLLKIPPPKFLKVAKKF | seqid_3763 |
| variant_5008 | RWKLLLKIPPPKFLKVAKKF | seqid_3764 |
| variant_5009 | FWKLLLKIPPPKFLKVAKKF | seqid_3765 |
| variant_5010 | KKKKLLKIPPPKFLKVAKKF | seqid_3766 |
| variant_5011 | RKKKLLKIPPPKFLKVAKKF | seqid_3767 |
| variant_5012 | FKKKLLKIPPPKFLKVAKKF | seqid_3768 |
| variant_5013 | KWKKLLKIPPPKFLKVAKKF | seqid_3769 |
| variant_5014 | RWKKLLKIPPPKFLKVAKKF | seqid_3770 |
| variant_5015 | FWKKLLKIPPPKFLKVAKKF | seqid_3771 |
| variant_5016 | KKKLFKKIPPPKFLKVAKKF | seqid_3772 |
| variant_5017 | RKKLFKKIPPPKFLKVAKKF | seqid_3773 |
| variant_5018 | FKKLFKKIPPPKFLKVAKKF | seqid_3774 |
| variant_5019 | KWKLFKKIPPPKFLKVAKKF | seqid_3775 |
| variant_5020 | RWKLFKKIPPPKFLKVAKKF | seqid_3776 |
| variant_5021 | FWKLFKKIPPPKFLKVAKKF | seqid_3777 |
| variant_5022 | KKKKFKKIPPPKFLKVAKKF | seqid_3778 |
| variant_5023 | RKKKFKKIPPPKFLKVAKKF | seqid_3779 |
| variant_5024 | FKKKFKKIPPPKFLKVAKKF | seqid_3780 |
| variant_5025 | KWKKFKKIPPPKFLKVAKKF | seqid_3781 |
| variant_5026 | RWKKFKKIPPPKFLKVAKKF | seqid_3782 |
| variant_5027 | FWKKFKKIPPPKFLKVAKKF | seqid_3783 |
| variant_5028 | KKKLLKKIPPPKFLKVAKKF | seqid_3784 |
| variant_5029 | RKKLLKKIPPPKFLKVAKKF | seqid_3785 |
| variant_5030 | FKKLLKKIPPPKFLKVAKKF | seqid_3786 |
| variant_5031 | KWKLLKKIPPPKFLKVAKKF | seqid_3787 |
| variant_5032 | RWKLLKKIPPPKFLKVAKKF | seqid_3788 |
| variant_5033 | FWKLLKKIPPPKFLKVAKKF | seqid_3789 |
| variant_5034 | KKKKLKKIPPPKFLKVAKKF | seqid_3790 |
| variant_5035 | RKKKLKKIPPPKFLKVAKKF | seqid_3791 |
| variant_5036 | FKKKLKKIPPPKFLKVAKKF | seqid_3792 |
| variant_5037 | KWKKLKKIPPPKFLKVAKKF | seqid_3793 |
| variant_5038 | RWKKLKKIPPPKFLKVAKKF | seqid_3794 |
| variant_5039 | FWKKLKKIPPPKFLKVAKKF | seqid_3795 |
| variant_5040 | KKKLFLKIPPKFLKVAKKF | seqid_3796 |
| variant_5041 | RKKLFLKIPPKFLKVAKKF | seqid_3797 |
| variant_5042 | FKKLFLKIPPKFLKVAKKF | seqid_3798 |
| variant_5043 | KWKLFLKIPPKFLKVAKKF | seqid_3799 |
| variant_5044 | RWKLFLKIPPKFLKVAKKF | seqid_3800 |
| variant_5045 | FWKLFLKIPPKFLKVAKKF | seqid_3801 |
| variant_5046 | KKKKFLKIPPKFLKVAKKF | seqid_3802 |
| variant_5047 | RKKKFLKIPPKFLKVAKKF | seqid_3803 |
| variant_5048 | FKKKFLKIPPKFLKVAKKF | seqid_3804 |
| variant_5049 | KWKKFLKIPPKFLKVAKKF | seqid_3805 |
| variant_5050 | RWKKFLKIPPKFLKVAKKF | seqid_3806 |
| variant_5051 | FWKKFLKIPPKFLKVAKKF | seqid_3807 |
| variant_5052 | KKKLLLKIPPKFLKVAKKF | seqid_3808 |
| variant_5053 | RKKLLLKIPPKFLKVAKKF | seqid_3809 |
| variant_5054 | FKKLLLKIPPKFLKVAKKF | seqid_3810 |
| variant_5055 | KWKLLLKIPPKFLKVAKKF | seqid_3811 |
| variant_5056 | RWKLLLKIPPKFLKVAKKF | seqid_3812 |
| variant_5057 | FWKLLLKIPPKFLKVAKKF | seqid_3813 |
| variant_5058 | KKKKLLKIPPKFLKVAKKF | seqid_3814 |
| variant_5059 | RKKKLLKIPPKFLKVAKKF | seqid_3815 |
| variant_5060 | FKKKLLKIPPKFLKVAKKF | seqid_3816 |
| variant_5061 | KWKKLLKIPPKFLKVAKKF | seqid_3817 |
| variant_5062 | RWKKLLKIPPKFLKVAKKF | seqid_3818 |
| variant_5063 | FWKKLLKIPPKFLKVAKKF | seqid_3819 |
| variant_5064 | KKKLFKKIPPKFLKVAKKF | seqid_3820 |
| variant_5065 | RKKLFKKIPPKFLKVAKKF | seqid_3821 |
| variant_5066 | FKKLFKKIPPKFLKVAKKF | seqid_3822 |
| variant_5067 | KWKLFKKIPPKFLKVAKKF | seqid_3823 |
| variant_5068 | RWKLFKKIPPKFLKVAKKF | seqid_3824 |
| variant_5069 | FWKLFKKIPPKFLKVAKKF | seqid_3825 |
| variant_5070 | KKKKFKKIPPKFLKVAKKF | seqid_3826 |
| variant_5071 | RKKKFKKIPPKFLKVAKKF | seqid_3827 |
| variant_5072 | FKKKFKKIPPKFLKVAKKF | seqid_3828 |
| variant_5073 | KWKKFKKIPPKFLKVAKKF | seqid_3829 |
| variant_5074 | RWKKFKKIPPKFLKVAKKF | seqid_3830 |
| variant_5075 | FWKKFKKIPPKFLKVAKKF | seqid_3831 |
| variant_5076 | KKKLLKKIPPKFLKVAKKF | seqid_3832 |
| variant_5077 | RKKLLKKIPPKFLKVAKKF | seqid_3833 |
| variant_5078 | FKKLLKKIPPKFLKVAKKF | seqid_3834 |
| variant_5079 | KWKLLKKIPPKFLKVAKKF | seqid_3835 |
| variant_5080 | RWKLLKKIPPKFLKVAKKF | seqid_3836 |
| variant_5081 | FWKLLKKIPPKFLKVAKKF | seqid_3837 |
| variant_5082 | KKKKLKKIPPKFLKVAKKF | seqid_3838 |
| variant_5083 | RKKKLKKIPPKFLKVAKKF | seqid_3839 |
| variant_5084 | FKKKLKKIPPKFLKVAKKF | seqid_3840 |
| variant_5085 | KWKKLKKIPPKFLKVAKKF | seqid_3841 |
| variant_5086 | RWKKLKKIPPKFLKVAKKF | seqid_3842 |
| variant_5087 | FWKKLKKIPPKFLKVAKKF | seqid_3843 |
| variant_5136 | KKKLFLKIPKFLKVAKKF | seqid_3844 |
| variant_5137 | RKKLFLKIPKFLKVAKKF | seqid_3845 |
| variant_5138 | FKKLFLKIPKFLKVAKKF | seqid_3846 |
| variant_5139 | KWKLFLKIPKFLKVAKKF | seqid_3847 |
| variant_5140 | RWKLFLKIPKFLKVAKKF | seqid_3848 |
| variant_5141 | FWKLFLKIPKFLKVAKKF | seqid_3849 |
| variant_5142 | KKKKFLKIPKFLKVAKKF | seqid_3850 |
| variant_5143 | RKKKFLKIPKFLKVAKKF | seqid_3851 |
| variant_5144 | FKKKFLKIPKFLKVAKKF | seqid_3852 |
| variant_5145 | KWKKFLKIPKFLKVAKKF | seqid_3853 |
| variant_5146 | RWKKFLKIPKFLKVAKKF | seqid_3854 |
| variant_5147 | FWKKFLKIPKFLKVAKKF | seqid_3855 |
| variant_5148 | KKKLLLKIPKFLKVAKKF | seqid_3856 |
| variant_5149 | RKKLLLKIPKFLKVAKKF | seqid_3857 |
| variant_5150 | FKKLLLKIPKFLKVAKKF | seqid_3858 |
| variant_5151 | KWKLLLKIPKFLKVAKKF | seqid_3859 |
| variant_5152 | RWKLLLKIPKFLKVAKKF | seqid_3860 |
| variant_5153 | FWKLLLKIPKFLKVAKKF | seqid_3861 |
| variant_5154 | KKKKLLKIPKFLKVAKKF | seqid_3862 |
| variant_5155 | RKKKLLKIPKFLKVAKKF | seqid_3863 |
| variant_5156 | FKKKLLKIPKFLKVAKKF | seqid_3864 |
| variant_5157 | KWKKLLKIPKFLKVAKKF | seqid_3865 |
| variant_5158 | RWKKLLKIPKFLKVAKKF | seqid_3866 |
| variant_5159 | FWKKLLKIPKFLKVAKKF | seqid_3867 |
| variant_5160 | KKKLFKKIPKFLKVAKKF | seqid_3868 |
| variant_5161 | RKKLFKKIPKFLKVAKKF | seqid_3869 |
| variant_5162 | FKKLFKKIPKFLKVAKKF | seqid_3870 |
| variant_5163 | KWKLFKKIPKFLKVAKKF | seqid_3871 |
| variant_5164 | RWKLFKKIPKFLKVAKKF | seqid_3872 |
| variant_5165 | FWKLFKKIPKFLKVAKKF | seqid_3873 |
| variant_5166 | KKKKFKKIPKFLKVAKKF | seqid_3874 |
| variant_5167 | RKKKFKKIPKFLKVAKKF | seqid_3875 |
| variant_5168 | FKKKFKKIPKFLKVAKKF | seqid_3876 |
| variant_5169 | KWKKFKKIPKFLKVAKKF | seqid_3877 |
| variant_5170 | RWKKFKKIPKFLKVAKKF | seqid_3878 |
| variant_5171 | FWKKFKKIPKFLKVAKKF | seqid_3879 |
| variant_5172 | KKKLLKKIPKFLKVAKKF | seqid_3880 |
| variant_5173 | RKKLLKKIPKFLKVAKKF | seqid_3881 |
| variant_5174 | FKKLLKKIPKFLKVAKKF | seqid_3882 |
| variant_5175 | KWKLLKKIPKFLKVAKKF | seqid_3883 |
| variant_5176 | RWKLLKKIPKFLKVAKKF | seqid_3884 |
| variant_5177 | FWKLLKKIPKFLKVAKKF | seqid_3885 |
| variant_5178 | KKKKLKKIPKFLKVAKKF | seqid_3886 |
| variant_5179 | RKKKLKKIPKFLKVAKKF | seqid_3887 |
| variant_5180 | FKKKLKKIPKFLKVAKKF | seqid_3888 |
| variant_5181 | KWKKLKKIPKFLKVAKKF | seqid_3889 |
| variant_5182 | RWKKLKKIPKFLKVAKKF | seqid_3890 |
| variant_5183 | FWKKLKKIPKFLKVAKKF | seqid_3891 |
| variant_5184 | KKKLFLKIPPPKFKKVAKKF | seqid_3892 |
| variant_5185 | RKKLFLKIPPPKFKKVAKKF | seqid_3893 |
| variant_5186 | FKKLFLKIPPPKFKKVAKKF | seqid_3894 |
| variant_5187 | KWKLFLKIPPPKFKKVAKKF | seqid_3895 |
| variant_5188 | RWKLFLKIPPPKFKKVAKKF | seqid_3896 |
| variant_5189 | FWKLFLKIPPPKFKKVAKKF | seqid_3897 |
| variant_5190 | KKKKFLKIPPPKFKKVAKKF | seqid_3898 |
| variant_5191 | RKKKFLKIPPPKFKKVAKKF | seqid_3899 |
| variant_5192 | FKKKFLKIPPPKFKKVAKKF | seqid_3900 |
| variant_5193 | KWKKFLKIPPPKFKKVAKKF | seqid_3901 |
| variant_5194 | RWKKFLKIPPPKFKKVAKKF | seqid_3902 |
| variant_5195 | FWKKFLKIPPPKFKKVAKKF | seqid_3903 |
| variant_5196 | KKKLLLKIPPPKFKKVAKKF | seqid_3904 |
| variant_5197 | RKKLLLKIPPPKFKKVAKKF | seqid_3905 |
| variant_5198 | FKKLLLKIPPPKFKKVAKKF | seqid_3906 |
| variant_5199 | KWKLLLKIPPPKFKKVAKKF | seqid_3907 |
| variant_5200 | RWKLLLKIPPPKFKKVAKKF | seqid_3908 |
| variant_5201 | FWKLLLKIPPPKFKKVAKKF | seqid_3909 |
| variant_5202 | KKKKLLKIPPPKFKKVAKKF | seqid_3910 |
| variant_5203 | RKKKLLKIPPPKFKKVAKKF | seqid_3911 |
| variant_5204 | FKKKLLKIPPPKFKKVAKKF | seqid_3912 |
| variant_5205 | KWKKLLKIPPPKFKKVAKKF | seqid_3913 |
| variant_5206 | RWKKLLKIPPPKFKKVAKKF | seqid_3914 |
| variant_5207 | FWKKLLKIPPPKFKKVAKKF | seqid_3915 |
| variant_5208 | KKKLFKKIPPPKFKKVAKKF | seqid_3916 |
| variant_5209 | RKKLFKKIPPPKFKKVAKKF | seqid_3917 |
| variant_5210 | FKKLFKKIPPPKFKKVAKKF | seqid_3918 |
| variant_5211 | KWKLFKKIPPPKFKKVAKKF | seqid_3919 |
| variant_5212 | RWKLFKKIPPPKFKKVAKKF | seqid_3920 |
| variant_5213 | FWKLFKKIPPPKFKKVAKKF | seqid_3921 |
| variant_5214 | KKKKFKKIPPPKFKKVAKKF | seqid_3922 |
| variant_5215 | RKKKFKKIPPPKFKKVAKKF | seqid_3923 |
| variant_5216 | FKKKFKKIPPPKFKKVAKKF | seqid_3924 |
| variant_5217 | KWKKFKKIPPPKFKKVAKKF | seqid_3925 |
| variant_5218 | RWKKFKKIPPPKFKKVAKKF | seqid_3926 |
| variant_5219 | FWKKFKKIPPPKFKKVAKKF | seqid_3927 |
| variant_5220 | KKKLLKKIPPPKFKKVAKKF | seqid_3928 |
| variant_5221 | RKKLLKKIPPPKFKKVAKKF | seqid_3929 |
| variant_5222 | FKKLLKKIPPPKFKKVAKKF | seqid_3930 |
| variant_5223 | KWKLLKKIPPPKFKKVAKKF | seqid_3931 |
| variant_5224 | RWKLLKKIPPPKFKKVAKKF | seqid_3932 |
| variant_5225 | FWKLLKKIPPPKFKKVAKKF | seqid_3933 |
| variant_5226 | KKKKLKKIPPPKFKKVAKKF | seqid_3934 |
| variant_5227 | RKKKLKKIPPPKFKKVAKKF | seqid_3935 |
| variant_5228 | FKKKLKKIPPPKFKKVAKKF | seqid_3936 |
| variant_5229 | KWKKLKKIPPPKFKKVAKKF | seqid_3937 |
| variant_5230 | RWKKLKKIPPPKFKKVAKKF | seqid_3938 |
| variant_5231 | FWKKLKKIPPPKFKKVAKKF | seqid_3939 |
| variant_5232 | KKKLFLKIPPKFKKVAKKF | seqid_3940 |
| variant_5233 | RKKLFLKIPPKFKKVAKKF | seqid_3941 |
| variant_5234 | FKKLFLKIPPKFKKVAKKF | seqid_3942 |
| variant_5235 | KWKLFLKIPPKFKKVAKKF | seqid_3943 |
| variant_5236 | RWKLFLKIPPKFKKVAKKF | seqid_3944 |
| variant_5237 | FWKLFLKIPPKFKKVAKKF | seqid_3945 |
| variant_5238 | KKKKFLKIPPKFKKVAKKF | seqid_3946 |
| variant_5239 | RKKKFLKIPPKFKKVAKKF | seqid_3947 |
| variant_5240 | FKKKFLKIPPKFKKVAKKF | seqid_3948 |
| variant_5241 | KWKKFLKIPPKFKKVAKKF | seqid_3949 |
| variant_5242 | RWKKFLKIPPKFKKVAKKF | seqid_3950 |
| variant_5243 | FWKKFLKIPPKFKKVAKKF | seqid_3951 |
| variant_5244 | KKKLLLKIPPKFKKVAKKF | seqid_3952 |
| variant_5245 | RKKLLLKIPPKFKKVAKKF | seqid_3953 |
| variant_5246 | FKKLLLKIPPKFKKVAKKF | seqid_3954 |
| variant_5247 | KWKLLLKIPPKFKKVAKKF | seqid_3955 |
| variant_5248 | RWKLLLKIPPKFKKVAKKF | seqid_3956 |
| variant_5249 | FWKLLLKIPPKFKKVAKKF | seqid_3957 |
| variant_5250 | KKKKLLKIPPKFKKVAKKF | seqid_3958 |
| variant_5251 | RKKKLLKIPPKFKKVAKKF | seqid_3959 |
| variant_5252 | FKKKLLKIPPKFKKVAKKF | seqid_3960 |
| variant_5253 | KWKKLLKIPPKFKKVAKKF | seqid_3961 |
| variant_5254 | RWKKLLKIPPKFKKVAKKF | seqid_3962 |
| variant_5255 | FWKKLLKIPPKFKKVAKKF | seqid_3963 |
| variant_5256 | KKKLFKKIPPKFKKVAKKF | seqid_3964 |
| variant_5257 | RKKLFKKIPPKFKKVAKKF | seqid_3965 |
| variant_5258 | FKKLFKKIPPKFKKVAKKF | seqid_3966 |
| variant_5259 | KWKLFKKIPPKFKKVAKKF | seqid_3967 |
| variant_5260 | RWKLFKKIPPKFKKVAKKF | seqid_3968 |
| variant_5261 | FWKLFKKIPPKFKKVAKKF | seqid_3969 |
| variant_5262 | KKKKFKKIPPKFKKVAKKF | seqid_3970 |
| variant_5263 | RKKKFKKIPPKFKKVAKKF | seqid_3971 |
| variant_5264 | FKKKFKKIPPKFKKVAKKF | seqid_3972 |
| variant_5265 | KWKKFKKIPPKFKKVAKKF | seqid_3973 |
| variant_5266 | RWKKFKKIPPKFKKVAKKF | seqid_3974 |
| variant_5267 | FWKKFKKIPPKFKKVAKKF | seqid_3975 |
| variant_5268 | KKKLLKKIPPKFKKVAKKF | seqid_3976 |
| variant_5269 | RKKLLKKIPPKFKKVAKKF | seqid_3977 |
| variant_5270 | FKKLLKKIPPKFKKVAKKF | seqid_3978 |
| variant_5271 | KWKLLKKIPPKFKKVAKKF | seqid_3979 |
| variant_5272 | RWKLLKKIPPKFKKVAKKF | seqid_3980 |
| variant_5273 | FWKLLKKIPPKFKKVAKKF | seqid_3981 |
| variant_5274 | KKKKLKKIPPKFKKVAKKF | seqid_3982 |
| variant_5275 | RKKKLKKIPPKFKKVAKKF | seqid_3983 |
| variant_5276 | FKKKLKKIPPKFKKVAKKF | seqid_3984 |
| variant_5277 | KWKKLKKIPPKFKKVAKKF | seqid_3985 |
| variant_5278 | RWKKLKKIPPKFKKVAKKF | seqid_3986 |
| variant_5279 | FWKKLKKIPPKFKKVAKKF | seqid_3987 |
| variant_5328 | KKKLFLKIPKFKKVAKKF | seqid_3988 |
| variant_5329 | RKKLFLKIPKFKKVAKKF | seqid_3989 |
| variant_5330 | FKKLFLKIPKFKKVAKKF | seqid_3990 |
| variant_5331 | KWKLFLKIPKFKKVAKKF | seqid_3991 |
| variant_5332 | RWKLFLKIPKFKKVAKKF | seqid_3992 |
| variant_5333 | FWKLFLKIPKFKKVAKKF | seqid_3993 |
| variant_5334 | KKKKFLKIPKFKKVAKKF | seqid_3994 |
| variant_5335 | RKKKFLKIPKFKKVAKKF | seqid_3995 |
| variant_5336 | FKKKFLKIPKFKKVAKKF | seqid_3996 |
| variant_5337 | KWKKFLKIPKFKKVAKKF | seqid_3997 |
| variant_5338 | RWKKFLKIPKFKKVAKKF | seqid_3998 |
| variant_5339 | FWKKFLKIPKFKKVAKKF | seqid_3999 |
| variant_5340 | KKKLLLKIPKFKKVAKKF | seqid_4000 |
| variant_5341 | RKKLLLKIPKFKKVAKKF | seqid_4001 |
| variant_5342 | FKKLLLKIPKFKKVAKKF | seqid_4002 |
| variant_5343 | KWKLLLKIPKFKKVAKKF | seqid_4003 |
| variant_5344 | RWKLLLKIPKFKKVAKKF | seqid_4004 |
| variant_5345 | FWKLLLKIPKFKKVAKKF | seqid_4005 |
| variant_5346 | KKKKLLKIPKFKKVAKKF | seqid_4006 |
| variant_5347 | RKKKLLKIPKFKKVAKKF | seqid_4007 |
| variant_5348 | FKKKLLKIPKFKKVAKKF | seqid_4008 |
| variant_5349 | KWKKLLKIPKFKKVAKKF | seqid_4009 |
| variant_5350 | RWKKLLKIPKFKKVAKKF | seqid_4010 |
| variant_5351 | FWKKLLKIPKFKKVAKKF | seqid_4011 |
| variant_5352 | KKKLFKKIPKFKKVAKKF | seqid_4012 |
| variant_5353 | RKKLFKKIPKFKKVAKKF | seqid_4013 |
| variant_5354 | FKKLFKKIPKFKKVAKKF | seqid_4014 |
| variant_5355 | KWKLFKKIPKFKKVAKKF | seqid_4015 |
| variant_5356 | RWKLFKKIPKFKKVAKKF | seqid_4016 |
| variant_5357 | FWKLFKKIPKFKKVAKKF | seqid_4017 |
| variant_5358 | KKKKFKKIPKFKKVAKKF | seqid_4018 |
| variant_5359 | RKKKFKKIPKFKKVAKKF | seqid_4019 |
| variant_5360 | FKKKFKKIPKFKKVAKKF | seqid_4020 |
| variant_5361 | KWKKFKKIPKFKKVAKKF | seqid_4021 |
| variant_5362 | RWKKFKKIPKFKKVAKKF | seqid_4022 |
| variant_5363 | FWKKFKKIPKFKKVAKKF | seqid_4023 |
| variant_5364 | KKKLLKKIPKFKKVAKKF | seqid_4024 |
| variant_5365 | RKKLLKKIPKFKKVAKKF | seqid_4025 |
| variant_5366 | FKKLLKKIPKFKKVAKKF | seqid_4026 |
| variant_5367 | KWKLLKKIPKFKKVAKKF | seqid_4027 |
| variant_5368 | RWKLLKKIPKFKKVAKKF | seqid_4028 |
| variant_5369 | FWKLLKKIPKFKKVAKKF | seqid_4029 |
| variant_5370 | KKKKLKKIPKFKKVAKKF | seqid_4030 |
| variant_5371 | RKKKLKKIPKFKKVAKKF | seqid_4031 |
| variant_5372 | FKKKLKKIPKFKKVAKKF | seqid_4032 |
| variant_5373 | KWKKLKKIPKFKKVAKKF | seqid_4033 |
| variant_5374 | RWKKLKKIPKFKKVAKKF | seqid_4034 |
| variant_5375 | FWKKLKKIPKFKKVAKKF | seqid_4035 |
| variant_5376 | KKKLFLKIPPPKFLHSAKKF | seqid_4036 |
| variant_5377 | RKKLFLKIPPPKFLHSAKKF | seqid_4037 |
| variant_5378 | FKKLFLKIPPPKFLHSAKKF | seqid_4038 |
| variant_5379 | KWKLFLKIPPPKFLHSAKKF | seqid_4039 |
| variant_5380 | RWKLFLKIPPPKFLHSAKKF | seqid_4040 |
| variant_5381 | FWKLFLKIPPPKFLHSAKKF | seqid_4041 |
| variant_5382 | KKKKFLKIPPPKFLHSAKKF | seqid_4042 |
| variant_5383 | RKKKFLKIPPPKFLHSAKKF | seqid_4043 |
| variant_5384 | FKKKFLKIPPPKFLHSAKKF | seqid_4044 |
| variant_5385 | KWKKFLKIPPPKFLHSAKKF | seqid_4045 |
| variant_5386 | RWKKFLKIPPPKFLHSAKKF | seqid_4046 |
| variant_5387 | FWKKFLKIPPPKFLHSAKKF | seqid_4047 |
| variant_5388 | KKKLLLKIPPPKFLHSAKKF | seqid_4048 |
| variant_5389 | RKKLLLKIPPPKFLHSAKKF | seqid_4049 |
| variant_5390 | FKKLLLKIPPPKFLHSAKKF | seqid_4050 |
| variant_5391 | KWKLLLKIPPPKFLHSAKKF | seqid_4051 |
| variant_5392 | RWKLLLKIPPPKFLHSAKKF | seqid_4052 |
| variant_5393 | FWKLLLKIPPPKFLHSAKKF | seqid_4053 |
| variant_5394 | KKKKLLKIPPPKFLHSAKKF | seqid_4054 |
| variant_5395 | RKKKLLKIPPPKFLHSAKKF | seqid_4055 |
| variant_5396 | FKKKLLKIPPPKFLHSAKKF | seqid_4056 |
| variant_5397 | KWKKLLKIPPPKFLHSAKKF | seqid_4057 |
| variant_5398 | RWKKLLKIPPPKFLHSAKKF | seqid_4058 |
| variant_5399 | FWKKLLKIPPPKFLHSAKKF | seqid_4059 |
| variant_5400 | KKKLFKKIPPPKFLHSAKKF | seqid_4060 |
| variant_5401 | RKKLFKKIPPPKFLHSAKKF | seqid_4061 |
| variant_5402 | FKKLFKKIPPPKFLHSAKKF | seqid_4062 |
| variant_5403 | KWKLFKKIPPPKFLHSAKKF | seqid_4063 |
| variant_5404 | RWKLFKKIPPPKFLHSAKKF | seqid_4064 |
| variant_5405 | FWKLFKKIPPPKFLHSAKKF | seqid_4065 |
| variant_5406 | KKKKFKKIPPPKFLHSAKKF | seqid_4066 |
| variant_5407 | RKKKFKKIPPPKFLHSAKKF | seqid_4067 |
| variant_5408 | FKKKFKKIPPPKFLHSAKKF | seqid_4068 |
| variant_5409 | KWKKFKKIPPPKFLHSAKKF | seqid_4069 |
| variant_5410 | RWKKFKKIPPPKFLHSAKKF | seqid_4070 |
| variant_5411 | FWKKFKKIPPPKFLHSAKKF | seqid_4071 |
| variant_5412 | KKKLLKKIPPPKFLHSAKKF | seqid_4072 |
| variant_5413 | RKKLLKKIPPPKFLHSAKKF | seqid_4073 |
| variant_5414 | FKKLLKKIPPPKFLHSAKKF | seqid_4074 |
| variant_5415 | KWKLLKKIPPPKFLHSAKKF | seqid_4075 |
| variant_5416 | RWKLLKKIPPPKFLHSAKKF | seqid_4076 |
| variant_5417 | FWKLLKKIPPPKFLHSAKKF | seqid_4077 |
| variant_5418 | KKKKLKKIPPPKFLHSAKKF | seqid_4078 |
| variant_5419 | RKKKLKKIPPPKFLHSAKKF | seqid_4079 |
| variant_5420 | FKKKLKKIPPPKFLHSAKKF | seqid_4080 |
| variant_5421 | KWKKLKKIPPPKFLHSAKKF | seqid_4081 |
| variant_5422 | RWKKLKKIPPPKFLHSAKKF | seqid_4082 |
| variant_5423 | FWKKLKKIPPPKFLHSAKKF | seqid_4083 |
| variant_5424 | KKKLFLKIPPKFLHSAKKF | seqid_4084 |
| variant_5425 | RKKLFLKIPPKFLHSAKKF | seqid_4085 |
| variant_5426 | FKKLFLKIPPKFLHSAKKF | seqid_4086 |
| variant_5427 | KWKLFLKIPPKFLHSAKKF | seqid_4087 |
| variant_5428 | RWKLFLKIPPKFLHSAKKF | seqid_4088 |
| variant_5429 | FWKLFLKIPPKFLHSAKKF | seqid_4089 |
| variant_5430 | KKKKFLKIPPKFLHSAKKF | seqid_4090 |
| variant_5431 | RKKKFLKIPPKFLHSAKKF | seqid_4091 |
| variant_5432 | FKKKFLKIPPKFLHSAKKF | seqid_4092 |
| variant_5433 | KWKKFLKIPPKFLHSAKKF | seqid_4093 |
| variant_5434 | RWKKFLKIPPKFLHSAKKF | seqid_4094 |
| variant_5435 | FWKKFLKIPPKFLHSAKKF | seqid_4095 |
| variant_5436 | KKKLLLKIPPKFLHSAKKF | seqid_4096 |
| variant_5437 | RKKLLLKIPPKFLHSAKKF | seqid_4097 |
| variant_5438 | FKKLLLKIPPKFLHSAKKF | seqid_4098 |
| variant_5439 | KWKLLLKIPPKFLHSAKKF | seqid_4099 |
| variant_5440 | RWKLLLKIPPKFLHSAKKF | seqid_4100 |
| variant_5441 | FWKLLLKIPPKFLHSAKKF | seqid_4101 |
| variant_5442 | KKKKLLKIPPKFLHSAKKF | seqid_4102 |
| variant_5443 | RKKKLLKIPPKFLHSAKKF | seqid_4103 |
| variant_5444 | FKKKLLKIPPKFLHSAKKF | seqid_4104 |
| variant_5445 | KWKKLLKIPPKFLHSAKKF | seqid_4105 |
| variant_5446 | RWKKLLKIPPKFLHSAKKF | seqid_4106 |
| variant_5447 | FWKKLLKIPPKFLHSAKKF | seqid_4107 |
| variant_5448 | KKKLFKKIPPKFLHSAKKF | seqid_4108 |
| variant_5449 | RKKLFKKIPPKFLHSAKKF | seqid_4109 |
| variant_5450 | FKKLFKKIPPKFLHSAKKF | seqid_4110 |
| variant_5451 | KWKLFKKIPPKFLHSAKKF | seqid_4111 |
| variant_5452 | RWKLFKKIPPKFLHSAKKF | seqid_4112 |
| variant_5453 | FWKLFKKIPPKFLHSAKKF | seqid_4113 |
| variant_5454 | KKKKFKKIPPKFLHSAKKF | seqid_4114 |
| variant_5455 | RKKKFKKIPPKFLHSAKKF | seqid_4115 |
| variant_5456 | FKKKFKKIPPKFLHSAKKF | seqid_4116 |
| variant_5457 | KWKKFKKIPPKFLHSAKKF | seqid_4117 |
| variant_5458 | RWKKFKKIPPKFLHSAKKF | seqid_4118 |
| variant_5459 | FWKKFKKIPPKFLHSAKKF | seqid_4119 |
| variant_5460 | KKKLLKKIPPKFLHSAKKF | seqid_4120 |
| variant_5461 | RKKLLKKIPPKFLHSAKKF | seqid_4121 |
| variant_5462 | FKKLLKKIPPKFLHSAKKF | seqid_4122 |
| variant_5463 | KWKLLKKIPPKFLHSAKKF | seqid_4123 |
| variant_5464 | RWKLLKKIPPKFLHSAKKF | seqid_4124 |
| variant_5465 | FWKLLKKIPPKFLHSAKKF | seqid_4125 |
| variant_5466 | KKKKLKKIPPKFLHSAKKF | seqid_4126 |
| variant_5467 | RKKKLKKIPPKFLHSAKKF | seqid_4127 |
| variant_5468 | FKKKLKKIPPKFLHSAKKF | seqid_4128 |
| variant_5469 | KWKKLKKIPPKFLHSAKKF | seqid_4129 |
| variant_5470 | RWKKLKKIPPKFLHSAKKF | seqid_4130 |
| variant_5471 | FWKKLKKIPPKFLHSAKKF | seqid_4131 |
| variant_5520 | KKKLFLKIPKFLHSAKKF | seqid_4132 |
| variant_5521 | RKKLFLKIPKFLHSAKKF | seqid_4133 |
| variant_5522 | FKKLFLKIPKFLHSAKKF | seqid_4134 |
| variant_5523 | KWKLFLKIPKFLHSAKKF | seqid_4135 |
| variant_5524 | RWKLFLKIPKFLHSAKKF | seqid_4136 |
| variant_5525 | FWKLFLKIPKFLHSAKKF | seqid_4137 |
| variant_5526 | KKKKFLKIPKFLHSAKKF | seqid_4138 |
| variant_5527 | RKKKFLKIPKFLHSAKKF | seqid_4139 |
| variant_5528 | FKKKFLKIPKFLHSAKKF | seqid_4140 |
| variant_5529 | KWKKFLKIPKFLHSAKKF | seqid_4141 |
| variant_5530 | RWKKFLKIPKFLHSAKKF | seqid_4142 |
| variant_5531 | FWKKFLKIPKFLHSAKKF | seqid_4143 |
| variant_5532 | KKKLLLKIPKFLHSAKKF | seqid_4144 |
| variant_5533 | RKKLLLKIPKFLHSAKKF | seqid_4145 |
| variant_5534 | FKKLLLKIPKFLHSAKKF | seqid_4146 |
| variant_5535 | KWKLLLKIPKFLHSAKKF | seqid_4147 |
| variant_5536 | RWKLLLKIPKFLHSAKKF | seqid_4148 |
| variant_5537 | FWKLLLKIPKFLHSAKKF | seqid_4149 |
| variant_5538 | KKKKLLKIPKFLHSAKKF | seqid_4150 |
| variant_5539 | RKKKLLKIPKFLHSAKKF | seqid_4151 |
| variant_5540 | FKKKLLKIPKFLHSAKKF | seqid_4152 |
| variant_5541 | KWKKLLKIPKFLHSAKKF | seqid_4153 |
| variant_5542 | RWKKLLKIPKFLHSAKKF | seqid_4154 |
| variant_5543 | FWKKLLKIPKFLHSAKKF | seqid_4155 |
| variant_5544 | KKKLFKKIPKFLHSAKKF | seqid_4156 |
| variant_5545 | RKKLFKKIPKFLHSAKKF | seqid_4157 |
| variant_5546 | FKKLFKKIPKFLHSAKKF | seqid_4158 |
| variant_5547 | KWKLFKKIPKFLHSAKKF | seqid_4159 |
| variant_5548 | RWKLFKKIPKFLHSAKKF | seqid_4160 |
| variant_5549 | FWKLFKKIPKFLHSAKKF | seqid_4161 |
| variant_5550 | KKKKFKKIPKFLHSAKKF | seqid_4162 |
| variant_5551 | RKKKFKKIPKFLHSAKKF | seqid_4163 |
| variant_5552 | FKKKFKKIPKFLHSAKKF | seqid_4164 |
| variant_5553 | KWKKFKKIPKFLHSAKKF | seqid_4165 |
| variant_5554 | RWKKFKKIPKFLHSAKKF | seqid_4166 |
| variant_5555 | FWKKFKKIPKFLHSAKKF | seqid_4167 |
| variant_5556 | KKKLLKKIPKFLHSAKKF | seqid_4168 |
| variant_5557 | RKKLLKKIPKFLHSAKKF | seqid_4169 |
| variant_5558 | FKKLLKKIPKFLHSAKKF | seqid_4170 |
| variant_5559 | KWKLLKKIPKFLHSAKKF | seqid_4171 |
| variant_5560 | RWKLLKKIPKFLHSAKKF | seqid_4172 |
| variant_5561 | FWKLLKKIPKFLHSAKKF | seqid_4173 |
| variant_5562 | KKKKLKKIPKFLHSAKKF | seqid_4174 |
| variant_5563 | RKKKLKKIPKFLHSAKKF | seqid_4175 |
| variant_5564 | FKKKLKKIPKFLHSAKKF | seqid_4176 |
| variant_5565 | KWKKLKKIPKFLHSAKKF | seqid_4177 |
| variant_5566 | RWKKLKKIPKFLHSAKKF | seqid_4178 |
| variant_5567 | FWKKLKKIPKFLHSAKKF | seqid_4179 |
| variant_5568 | KKKLFLKIPPPKFKHSAKKF | seqid_4180 |
| variant_5569 | RKKLFLKIPPPKFKHSAKKF | seqid_4181 |
| variant_5570 | FKKLFLKIPPPKFKHSAKKF | seqid_4182 |
| variant_5571 | KWKLFLKIPPPKFKHSAKKF | seqid_4183 |
| variant_5572 | RWKLFLKIPPPKFKHSAKKF | seqid_4184 |
| variant_5573 | FWKLFLKIPPPKFKHSAKKF | seqid_4185 |
| variant_5574 | KKKKFLKIPPPKFKHSAKKF | seqid_4186 |
| variant_5575 | RKKKFLKIPPPKFKHSAKKF | seqid_4187 |
| variant_5576 | FKKKFLKIPPPKFKHSAKKF | seqid_4188 |
| variant_5577 | KWKKFLKIPPPKFKHSAKKF | seqid_4189 |
| variant_5578 | RWKKFLKIPPPKFKHSAKKF | seqid_4190 |
| variant_5579 | FWKKFLKIPPPKFKHSAKKF | seqid_4191 |
| variant_5580 | KKKLLLKIPPPKFKHSAKKF | seqid_4192 |
| variant_5581 | RKKLLLKIPPPKFKHSAKKF | seqid_4193 |
| variant_5582 | FKKLLLKIPPPKFKHSAKKF | seqid_4194 |
| variant_5583 | KWKLLLKIPPPKFKHSAKKF | seqid_4195 |
| variant_5584 | RWKLLLKIPPPKFKHSAKKF | seqid_4196 |
| variant_5585 | FWKLLLKIPPPKFKHSAKKF | seqid_4197 |
| variant_5586 | KKKKLLKIPPPKFKHSAKKF | seqid_4198 |
| variant_5587 | RKKKLLKIPPPKFKHSAKKF | seqid_4199 |
| variant_5588 | FKKKLLKIPPPKFKHSAKKF | seqid_4200 |
| variant_5589 | KWKKLLKIPPPKFKHSAKKF | seqid_4201 |
| variant_5590 | RWKKLLKIPPPKFKHSAKKF | seqid_4202 |
| variant_5591 | FWKKLLKIPPPKFKHSAKKF | seqid_4203 |
| variant_5592 | KKKLFKKIPPPKFKHSAKKF | seqid_4204 |
| variant_5593 | RKKLFKKIPPPKFKHSAKKF | seqid_4205 |
| variant_5594 | FKKLFKKIPPPKFKHSAKKF | seqid_4206 |
| variant_5595 | KWKLFKKIPPPKFKHSAKKF | seqid_4207 |
| variant_5596 | RWKLFKKIPPPKFKHSAKKF | seqid_4208 |
| variant_5597 | FWKLFKKIPPPKFKHSAKKF | seqid_4209 |
| variant_5598 | KKKKFKKIPPPKFKHSAKKF | seqid_4210 |
| variant_5599 | RKKKFKKIPPPKFKHSAKKF | seqid_4211 |
| variant_5600 | FKKKFKKIPPPKFKHSAKKF | seqid_4212 |
| variant_5601 | KWKKFKKIPPPKFKHSAKKF | seqid_4213 |
| variant_5602 | RWKKFKKIPPPKFKHSAKKF | seqid_4214 |
| variant_5603 | FWKKFKKIPPPKFKHSAKKF | seqid_4215 |
| variant_5604 | KKKLLKKIPPPKFKHSAKKF | seqid_4216 |
| variant_5605 | RKKLLKKIPPPKFKHSAKKF | seqid_4217 |
| variant_5606 | FKKLLKKIPPPKFKHSAKKF | seqid_4218 |
| variant_5607 | KWKLLKKIPPPKFKHSAKKF | seqid_4219 |
| variant_5608 | RWKLLKKIPPPKFKHSAKKF | seqid_4220 |
| variant_5609 | FWKLLKKIPPPKFKHSAKKF | seqid_4221 |
| variant_5610 | KKKKLKKIPPPKFKHSAKKF | seqid_4222 |
| variant_5611 | RKKKLKKIPPPKFKHSAKKF | seqid_4223 |
| variant_5612 | FKKKLKKIPPPKFKHSAKKF | seqid_4224 |
| variant_5613 | KWKKLKKIPPPKFKHSAKKF | seqid_4225 |
| variant_5614 | RWKKLKKIPPPKFKHSAKKF | seqid_4226 |
| variant_5615 | FWKKLKKIPPPKFKHSAKKF | seqid_4227 |
| variant_5616 | KKKLFLKIPPKFKHSAKKF | seqid_4228 |
| variant_5617 | RKKLFLKIPPKFKHSAKKF | seqid_4229 |
| variant_5618 | FKKLFLKIPPKFKHSAKKF | seqid_4230 |
| variant_5619 | KWKLFLKIPPKFKHSAKKF | seqid_4231 |
| variant_5620 | RWKLFLKIPPKFKHSAKKF | seqid_4232 |
| variant_5621 | FWKLFLKIPPKFKHSAKKF | seqid_4233 |
| variant_5622 | KKKKFLKIPPKFKHSAKKF | seqid_4234 |
| variant_5623 | RKKKFLKIPPKFKHSAKKF | seqid_4235 |
| variant_5624 | FKKKFLKIPPKFKHSAKKF | seqid_4236 |
| variant_5625 | KWKKFLKIPPKFKHSAKKF | seqid_4237 |
| variant_5626 | RWKKFLKIPPKFKHSAKKF | seqid_4238 |
| variant_5627 | FWKKFLKIPPKFKHSAKKF | seqid_4239 |
| variant_5628 | KKKLLLKIPPKFKHSAKKF | seqid_4240 |
| variant_5629 | RKKLLLKIPPKFKHSAKKF | seqid_4241 |
| variant_5630 | FKKLLLKIPPKFKHSAKKF | seqid_4242 |
| variant_5631 | KWKLLLKIPPKFKHSAKKF | seqid_4243 |
| variant_5632 | RWKLLLKIPPKFKHSAKKF | seqid_4244 |
| variant_5633 | FWKLLLKIPPKFKHSAKKF | seqid_4245 |
| variant_5634 | KKKKLLKIPPKFKHSAKKF | seqid_4246 |
| variant_5635 | RKKKLLKIPPKFKHSAKKF | seqid_4247 |
| variant_5636 | FKKKLLKIPPKFKHSAKKF | seqid_4248 |
| variant_5637 | KWKKLLKIPPKFKHSAKKF | seqid_4249 |
| variant_5638 | RWKKLLKIPPKFKHSAKKF | seqid_4250 |
| variant_5639 | FWKKLLKIPPKFKHSAKKF | seqid_4251 |
| variant_5640 | KKKLFKKIPPKFKHSAKKF | seqid_4252 |
| variant_5641 | RKKLFKKIPPKFKHSAKKF | seqid_4253 |
| variant_5642 | FKKLFKKIPPKFKHSAKKF | seqid_4254 |
| variant_5643 | KWKLFKKIPPKFKHSAKKF | seqid_4255 |
| variant_5644 | RWKLFKKIPPKFKHSAKKF | seqid_4256 |
| variant_5645 | FWKLFKKIPPKFKHSAKKF | seqid_4257 |
| variant_5646 | KKKKFKKIPPKFKHSAKKF | seqid_4258 |
| variant_5647 | RKKKFKKIPPKFKHSAKKF | seqid_4259 |
| variant_5648 | FKKKFKKIPPKFKHSAKKF | seqid_4260 |
| variant_5649 | KWKKFKKIPPKFKHSAKKF | seqid_4261 |
| variant_5650 | RWKKFKKIPPKFKHSAKKF | seqid_4262 |
| variant_5651 | FWKKFKKIPPKFKHSAKKF | seqid_4263 |
| variant_5652 | KKKLLKKIPPKFKHSAKKF | seqid_4264 |
| variant_5653 | RKKLLKKIPPKFKHSAKKF | seqid_4265 |
| variant_5654 | FKKLLKKIPPKFKHSAKKF | seqid_4266 |
| variant_5655 | KWKLLKKIPPKFKHSAKKF | seqid_4267 |
| variant_5656 | RWKLLKKIPPKFKHSAKKF | seqid_4268 |
| variant_5657 | FWKLLKKIPPKFKHSAKKF | seqid_4269 |
| variant_5658 | KKKKLKKIPPKFKHSAKKF | seqid_4270 |
| variant_5659 | RKKKLKKIPPKFKHSAKKF | seqid_4271 |
| variant_5660 | FKKKLKKIPPKFKHSAKKF | seqid_4272 |
| variant_5661 | KWKKLKKIPPKFKHSAKKF | seqid_4273 |
| variant_5662 | RWKKLKKIPPKFKHSAKKF | seqid_4274 |
| variant_5663 | FWKKLKKIPPKFKHSAKKF | seqid_4275 |
| variant_5712 | KKKLFLKIPKFKHSAKKF | seqid_4276 |
| variant_5713 | RKKLFLKIPKFKHSAKKF | seqid_4277 |
| variant_5714 | FKKLFLKIPKFKHSAKKF | seqid_4278 |
| variant_5715 | KWKLFLKIPKFKHSAKKF | seqid_4279 |
| variant_5716 | RWKLFLKIPKFKHSAKKF | seqid_4280 |
| variant_5717 | FWKLFLKIPKFKHSAKKF | seqid_4281 |
| variant_5718 | KKKKFLKIPKFKHSAKKF | seqid_4282 |
| variant_5719 | RKKKFLKIPKFKHSAKKF | seqid_4283 |
| variant_5720 | FKKKFLKIPKFKHSAKKF | seqid_4284 |
| variant_5721 | KWKKFLKIPKFKHSAKKF | seqid_4285 |
| variant_5722 | RWKKFLKIPKFKHSAKKF | seqid_4286 |
| variant_5723 | FWKKFLKIPKFKHSAKKF | seqid_4287 |
| variant_5724 | KKKLLLKIPKFKHSAKKF | seqid_4288 |
| variant_5725 | RKKLLLKIPKFKHSAKKF | seqid_4289 |
| variant_5726 | FKKLLLKIPKFKHSAKKF | seqid_4290 |
| variant_5727 | KWKLLLKIPKFKHSAKKF | seqid_4291 |
| variant_5728 | RWKLLLKIPKFKHSAKKF | seqid_4292 |
| variant_5729 | FWKLLLKIPKFKHSAKKF | seqid_4293 |
| variant_5730 | KKKKLLKIPKFKHSAKKF | seqid_4294 |
| variant_5731 | RKKKLLKIPKFKHSAKKF | seqid_4295 |
| variant_5732 | FKKKLLKIPKFKHSAKKF | seqid_4296 |
| variant_5733 | KWKKLLKIPKFKHSAKKF | seqid_4297 |
| variant_5734 | RWKKLLKIPKFKHSAKKF | seqid_4298 |
| variant_5735 | FWKKLLKIPKFKHSAKKF | seqid_4299 |
| variant_5736 | KKKLFKKIPKFKHSAKKF | seqid_4300 |
| variant_5737 | RKKLFKKIPKFKHSAKKF | seqid_4301 |
| variant_5738 | FKKLFKKIPKFKHSAKKF | seqid_4302 |
| variant_5739 | KWKLFKKIPKFKHSAKKF | seqid_4303 |
| variant_5740 | RWKLFKKIPKFKHSAKKF | seqid_4304 |
| variant_5741 | FWKLFKKIPKFKHSAKKF | seqid_4305 |
| variant_5742 | KKKKFKKIPKFKHSAKKF | seqid_4306 |
| variant_5743 | RKKKFKKIPKFKHSAKKF | seqid_4307 |
| variant_5744 | FKKKFKKIPKFKHSAKKF | seqid_4308 |
| variant_5745 | KWKKFKKIPKFKHSAKKF | seqid_4309 |
| variant_5746 | RWKKFKKIPKFKHSAKKF | seqid_4310 |
| variant_5747 | FWKKFKKIPKFKHSAKKF | seqid_4311 |
| variant_5748 | KKKLLKKIPKFKHSAKKF | seqid_4312 |
| variant_5749 | RKKLLKKIPKFKHSAKKF | seqid_4313 |
| variant_5750 | FKKLLKKIPKFKHSAKKF | seqid_4314 |
| variant_5751 | KWKLLKKIPKFKHSAKKF | seqid_4315 |
| variant_5752 | RWKLLKKIPKFKHSAKKF | seqid_4316 |
| variant_5753 | FWKLLKKIPKFKHSAKKF | seqid_4317 |
| variant_5754 | KKKKLKKIPKFKHSAKKF | seqid_4318 |
| variant_5755 | RKKKLKKIPKFKHSAKKF | seqid_4319 |
| variant_5756 | FKKKLKKIPKFKHSAKKF | seqid_4320 |
| variant_5757 | KWKKLKKIPKFKHSAKKF | seqid_4321 |
| variant_5758 | RWKKLKKIPKFKHSAKKF | seqid_4322 |
| variant_5759 | FWKKLKKIPKFKHSAKKF | seqid_4323 |
| variant_5760 | KKKLFLKIPPPKFLKSAKKF | seqid_4324 |
| variant_5761 | RKKLFLKIPPPKFLKSAKKF | seqid_4325 |
| variant_5762 | FKKLFLKIPPPKFLKSAKKF | seqid_4326 |
| variant_5763 | KWKLFLKIPPPKFLKSAKKF | seqid_4327 |
| variant_5764 | RWKLFLKIPPPKFLKSAKKF | seqid_4328 |
| variant_5765 | FWKLFLKIPPPKFLKSAKKF | seqid_4329 |
| variant_5766 | KKKKFLKIPPPKFLKSAKKF | seqid_4330 |
| variant_5767 | RKKKFLKIPPPKFLKSAKKF | seqid_4331 |
| variant_5768 | FKKKFLKIPPPKFLKSAKKF | seqid_4332 |
| variant_5769 | KWKKFLKIPPPKFLKSAKKF | seqid_4333 |
| variant_5770 | RWKKFLKIPPPKFLKSAKKF | seqid_4334 |
| variant_5771 | FWKKFLKIPPPKFLKSAKKF | seqid_4335 |
| variant_5772 | KKKLLLKIPPPKFLKSAKKF | seqid_4336 |
| variant_5773 | RKKLLLKIPPPKFLKSAKKF | seqid_4337 |
| variant_5774 | FKKLLLKIPPPKFLKSAKKF | seqid_4338 |
| variant_5775 | KWKLLLKIPPPKFLKSAKKF | seqid_4339 |
| variant_5776 | RWKLLLKIPPPKFLKSAKKF | seqid_4340 |
| variant_5777 | FWKLLLKIPPPKFLKSAKKF | seqid_4341 |
| variant_5778 | KKKKLLKIPPPKFLKSAKKF | seqid_4342 |
| variant_5779 | RKKKLLKIPPPKFLKSAKKF | seqid_4343 |
| variant_5780 | FKKKLLKIPPPKFLKSAKKF | seqid_4344 |
| variant_5781 | KWKKLLKIPPPKFLKSAKKF | seqid_4345 |
| variant_5782 | RWKKLLKIPPPKFLKSAKKF | seqid_4346 |
| variant_5783 | FWKKLLKIPPPKFLKSAKKF | seqid_4347 |
| variant_5784 | KKKLFKKIPPPKFLKSAKKF | seqid_4348 |
| variant_5785 | RKKLFKKIPPPKFLKSAKKF | seqid_4349 |
| variant_5786 | FKKLFKKIPPPKFLKSAKKF | seqid_4350 |
| variant_5787 | KWKLFKKIPPPKFLKSAKKF | seqid_4351 |
| variant_5788 | RWKLFKKIPPPKFLKSAKKF | seqid_4352 |
| variant_5789 | FWKLFKKIPPPKFLKSAKKF | seqid_4353 |
| variant_5790 | KKKKFKKIPPPKFLKSAKKF | seqid_4354 |
| variant_5791 | RKKKFKKIPPPKFLKSAKKF | seqid_4355 |
| variant_5792 | FKKKFKKIPPPKFLKSAKKF | seqid_4356 |
| variant_5793 | KWKKFKKIPPPKFLKSAKKF | seqid_4357 |
| variant_5794 | RWKKFKKIPPPKFLKSAKKF | seqid_4358 |
| variant_5795 | FWKKFKKIPPPKFLKSAKKF | seqid_4359 |
| variant_5796 | KKKLLKKIPPPKFLKSAKKF | seqid_4360 |
| variant_5797 | RKKLLKKIPPPKFLKSAKKF | seqid_4361 |
| variant_5798 | FKKLLKKIPPPKFLKSAKKF | seqid_4362 |
| variant_5799 | KWKLLKKIPPPKFLKSAKKF | seqid_4363 |
| variant_5800 | RWKLLKKIPPPKFLKSAKKF | seqid_4364 |
| variant_5801 | FWKLLKKIPPPKFLKSAKKF | seqid_4365 |
| variant_5802 | KKKKLKKIPPPKFLKSAKKF | seqid_4366 |
| variant_5803 | RKKKLKKIPPPKFLKSAKKF | seqid_4367 |
| variant_5804 | FKKKLKKIPPPKFLKSAKKF | seqid_4368 |
| variant_5805 | KWKKLKKIPPPKFLKSAKKF | seqid_4369 |
| variant_5806 | RWKKLKKIPPPKFLKSAKKF | seqid_4370 |
| variant_5807 | FWKKLKKIPPPKFLKSAKKF | seqid_4371 |
| variant_5808 | KKKLFLKIPPKFLKSAKKF | seqid_4372 |
| variant_5809 | RKKLFLKIPPKFLKSAKKF | seqid_4373 |
| variant_5810 | FKKLFLKIPPKFLKSAKKF | seqid_4374 |
| variant_5811 | KWKLFLKIPPKFLKSAKKF | seqid_4375 |
| variant_5812 | RWKLFLKIPPKFLKSAKKF | seqid_4376 |
| variant_5813 | FWKLFLKIPPKFLKSAKKF | seqid_4377 |
| variant_5814 | KKKKFLKIPPKFLKSAKKF | seqid_4378 |
| variant_5815 | RKKKFLKIPPKFLKSAKKF | seqid_4379 |
| variant_5816 | FKKKFLKIPPKFLKSAKKF | seqid_4380 |
| variant_5817 | KWKKFLKIPPKFLKSAKKF | seqid_4381 |
| variant_5818 | RWKKFLKIPPKFLKSAKKF | seqid_4382 |
| variant_5819 | FWKKFLKIPPKFLKSAKKF | seqid_4383 |
| variant_5820 | KKKLLLKIPPKFLKSAKKF | seqid_4384 |
| variant_5821 | RKKLLLKIPPKFLKSAKKF | seqid_4385 |
| variant_5822 | FKKLLLKIPPKFLKSAKKF | seqid_4386 |
| variant_5823 | KWKLLLKIPPKFLKSAKKF | seqid_4387 |
| variant_5824 | RWKLLLKIPPKFLKSAKKF | seqid_4388 |
| variant_5825 | FWKLLLKIPPKFLKSAKKF | seqid_4389 |
| variant_5826 | KKKKLLKIPPKFLKSAKKF | seqid_4390 |
| variant_5827 | RKKKLLKIPPKFLKSAKKF | seqid_4391 |
| variant_5828 | FKKKLLKIPPKFLKSAKKF | seqid_4392 |
| variant_5829 | KWKKLLKIPPKFLKSAKKF | seqid_4393 |
| variant_5830 | RWKKLLKIPPKFLKSAKKF | seqid_4394 |
| variant_5831 | FWKKLLKIPPKFLKSAKKF | seqid_4395 |
| variant_5832 | KKKLFKKIPPKFLKSAKKF | seqid_4396 |
| variant_5833 | RKKLFKKIPPKFLKSAKKF | seqid_4397 |
| variant_5834 | FKKLFKKIPPKFLKSAKKF | seqid_4398 |
| variant_5835 | KWKLFKKIPPKFLKSAKKF | seqid_4399 |
| variant_5836 | RWKLFKKIPPKFLKSAKKF | seqid_4400 |
| variant_5837 | FWKLFKKIPPKFLKSAKKF | seqid_4401 |
| variant_5838 | KKKKFKKIPPKFLKSAKKF | seqid_4402 |
| variant_5839 | RKKKFKKIPPKFLKSAKKF | seqid_4403 |
| variant_5840 | FKKKFKKIPPKFLKSAKKF | seqid_4404 |
| variant_5841 | KWKKFKKIPPKFLKSAKKF | seqid_4405 |
| variant_5842 | RWKKFKKIPPKFLKSAKKF | seqid_4406 |
| variant_5843 | FWKKFKKIPPKFLKSAKKF | seqid_4407 |
| variant_5844 | KKKLLKKIPPKFLKSAKKF | seqid_4408 |
| variant_5845 | RKKLLKKIPPKFLKSAKKF | seqid_4409 |
| variant_5846 | FKKLLKKIPPKFLKSAKKF | seqid_4410 |
| variant_5847 | KWKLLKKIPPKFLKSAKKF | seqid_4411 |
| variant_5848 | RWKLLKKIPPKFLKSAKKF | seqid_4412 |
| variant_5849 | FWKLLKKIPPKFLKSAKKF | seqid_4413 |
| variant_5850 | KKKKLKKIPPKFLKSAKKF | seqid_4414 |
| variant_5851 | RKKKLKKIPPKFLKSAKKF | seqid_4415 |
| variant_5852 | FKKKLKKIPPKFLKSAKKF | seqid_4416 |
| variant_5853 | KWKKLKKIPPKFLKSAKKF | seqid_4417 |
| variant_5854 | RWKKLKKIPPKFLKSAKKF | seqid_4418 |
| variant_5855 | FWKKLKKIPPKFLKSAKKF | seqid_4419 |
| variant_5904 | KKKLFLKIPKFLKSAKKF | seqid_4420 |
| variant_5905 | RKKLFLKIPKFLKSAKKF | seqid_4421 |
| variant_5906 | FKKLFLKIPKFLKSAKKF | seqid_4422 |
| variant_5907 | KWKLFLKIPKFLKSAKKF | seqid_4423 |
| variant_5908 | RWKLFLKIPKFLKSAKKF | seqid_4424 |
| variant_5909 | FWKLFLKIPKFLKSAKKF | seqid_4425 |
| variant_5910 | KKKKFLKIPKFLKSAKKF | seqid_4426 |
| variant_5911 | RKKKFLKIPKFLKSAKKF | seqid_4427 |
| variant_5912 | FKKKFLKIPKFLKSAKKF | seqid_4428 |
| variant_5913 | KWKKFLKIPKFLKSAKKF | seqid_4429 |
| variant_5914 | RWKKFLKIPKFLKSAKKF | seqid_4430 |
| variant_5915 | FWKKFLKIPKFLKSAKKF | seqid_4431 |
| variant_5916 | KKKLLLKIPKFLKSAKKF | seqid_4432 |
| variant_5917 | RKKLLLKIPKFLKSAKKF | seqid_4433 |
| variant_5918 | FKKLLLKIPKFLKSAKKF | seqid_4434 |
| variant_5919 | KWKLLLKIPKFLKSAKKF | seqid_4435 |
| variant_5920 | RWKLLLKIPKFLKSAKKF | seqid_4436 |
| variant_5921 | FWKLLLKIPKFLKSAKKF | seqid_4437 |
| variant_5922 | KKKKLLKIPKFLKSAKKF | seqid_4438 |
| variant_5923 | RKKKLLKIPKFLKSAKKF | seqid_4439 |
| variant_5924 | FKKKLLKIPKFLKSAKKF | seqid_4440 |
| variant_5925 | KWKKLLKIPKFLKSAKKF | seqid_4441 |
| variant_5926 | RWKKLLKIPKFLKSAKKF | seqid_4442 |
| variant_5927 | FWKKLLKIPKFLKSAKKF | seqid_4443 |
| variant_5928 | KKKLFKKIPKFLKSAKKF | seqid_4444 |
| variant_5929 | RKKLFKKIPKFLKSAKKF | seqid_4445 |
| variant_5930 | FKKLFKKIPKFLKSAKKF | seqid_4446 |
| variant_5931 | KWKLFKKIPKFLKSAKKF | seqid_4447 |
| variant_5932 | RWKLFKKIPKFLKSAKKF | seqid_4448 |
| variant_5933 | FWKLFKKIPKFLKSAKKF | seqid_4449 |
| variant_5934 | KKKKFKKIPKFLKSAKKF | seqid_4450 |
| variant_5935 | RKKKFKKIPKFLKSAKKF | seqid_4451 |
| variant_5936 | FKKKFKKIPKFLKSAKKF | seqid_4452 |
| variant_5937 | KWKKFKKIPKFLKSAKKF | seqid_4453 |
| variant_5938 | RWKKFKKIPKFLKSAKKF | seqid_4454 |
| variant_5939 | FWKKFKKIPKFLKSAKKF | seqid_4455 |
| variant_5940 | KKKLLKKIPKFLKSAKKF | seqid_4456 |
| variant_5941 | RKKLLKKIPKFLKSAKKF | seqid_4457 |
| variant_5942 | FKKLLKKIPKFLKSAKKF | seqid_4458 |
| variant_5943 | KWKLLKKIPKFLKSAKKF | seqid_4459 |
| variant_5944 | RWKLLKKIPKFLKSAKKF | seqid_4460 |
| variant_5945 | FWKLLKKIPKFLKSAKKF | seqid_4461 |
| variant_5946 | KKKKLKKIPKFLKSAKKF | seqid_4462 |
| variant_5947 | RKKKLKKIPKFLKSAKKF | seqid_4463 |
| variant_5948 | FKKKLKKIPKFLKSAKKF | seqid_4464 |
| variant_5949 | KWKKLKKIPKFLKSAKKF | seqid_4465 |
| variant_5950 | RWKKLKKIPKFLKSAKKF | seqid_4466 |
| variant_5951 | FWKKLKKIPKFLKSAKKF | seqid_4467 |
| variant_5952 | KKKLFLKIPPPKFKKSAKKF | seqid_4468 |
| variant_5953 | RKKLFLKIPPPKFKKSAKKF | seqid_4469 |
| variant_5954 | FKKLFLKIPPPKFKKSAKKF | seqid_4470 |
| variant_5955 | KWKLFLKIPPPKFKKSAKKF | seqid_4471 |
| variant_5956 | RWKLFLKIPPPKFKKSAKKF | seqid_4472 |
| variant_5957 | FWKLFLKIPPPKFKKSAKKF | seqid_4473 |
| variant_5958 | KKKKFLKIPPPKFKKSAKKF | seqid_4474 |
| variant_5959 | RKKKFLKIPPPKFKKSAKKF | seqid_4475 |
| variant_5960 | FKKKFLKIPPPKFKKSAKKF | seqid_4476 |
| variant_5961 | KWKKFLKIPPPKFKKSAKKF | seqid_4477 |
| variant_5962 | RWKKFLKIPPPKFKKSAKKF | seqid_4478 |
| variant_5963 | FWKKFLKIPPPKFKKSAKKF | seqid_4479 |
| variant_5964 | KKKLLLKIPPPKFKKSAKKF | seqid_4480 |
| variant_5965 | RKKLLLKIPPPKFKKSAKKF | seqid_4481 |
| variant_5966 | FKKLLLKIPPPKFKKSAKKF | seqid_4482 |
| variant_5967 | KWKLLLKIPPPKFKKSAKKF | seqid_4483 |
| variant_5968 | RWKLLLKIPPPKFKKSAKKF | seqid_4484 |
| variant_5969 | FWKLLLKIPPPKFKKSAKKF | seqid_4485 |
| variant_5970 | KKKKLLKIPPPKFKKSAKKF | seqid_4486 |
| variant_5971 | RKKKLLKIPPPKFKKSAKKF | seqid_4487 |
| variant_5972 | FKKKLLKIPPPKFKKSAKKF | seqid_4488 |
| variant_5973 | KWKKLLKIPPPKFKKSAKKF | seqid_4489 |
| variant_5974 | RWKKLLKIPPPKFKKSAKKF | seqid_4490 |
| variant_5975 | FWKKLLKIPPPKFKKSAKKF | seqid_4491 |
| variant_5976 | KKKLFKKIPPPKFKKSAKKF | seqid_4492 |
| variant_5977 | RKKLFKKIPPPKFKKSAKKF | seqid_4493 |
| variant_5978 | FKKLFKKIPPPKFKKSAKKF | seqid_4494 |
| variant_5979 | KWKLFKKIPPPKFKKSAKKF | seqid_4495 |
| variant_5980 | RWKLFKKIPPPKFKKSAKKF | seqid_4496 |
| variant_5981 | FWKLFKKIPPPKFKKSAKKF | seqid_4497 |
| variant_5982 | KKKKFKKIPPPKFKKSAKKF | seqid_4498 |
| variant_5983 | RKKKFKKIPPPKFKKSAKKF | seqid_4499 |
| variant_5984 | FKKKFKKIPPPKFKKSAKKF | seqid_4500 |
| variant_5985 | KWKKFKKIPPPKFKKSAKKF | seqid_4501 |
| variant_5986 | RWKKFKKIPPPKFKKSAKKF | seqid_4502 |
| variant_5987 | FWKKFKKIPPPKFKKSAKKF | seqid_4503 |
| variant_5988 | KKKLLKKIPPPKFKKSAKKF | seqid_4504 |
| variant_5989 | RKKLLKKIPPPKFKKSAKKF | seqid_4505 |
| variant_5990 | FKKLLKKIPPPKFKKSAKKF | seqid_4506 |
| variant_5991 | KWKLLKKIPPPKFKKSAKKF | seqid_4507 |
| variant_5992 | RWKLLKKIPPPKFKKSAKKF | seqid_4508 |
| variant_5993 | FWKLLKKIPPPKFKKSAKKF | seqid_4509 |
| variant_5994 | KKKKLKKIPPPKFKKSAKKF | seqid_4510 |
| variant_5995 | RKKKLKKIPPPKFKKSAKKF | seqid_4511 |
| variant_5996 | FKKKLKKIPPPKFKKSAKKF | seqid_4512 |
| variant_5997 | KWKKLKKIPPPKFKKSAKKF | seqid_4513 |
| variant_5998 | RWKKLKKIPPPKFKKSAKKF | seqid_4514 |
| variant_5999 | FWKKLKKIPPPKFKKSAKKF | seqid_4515 |
| variant_6000 | KKKLFLKIPPKFKKSAKKF | seqid_4516 |
| variant_6001 | RKKLFLKIPPKFKKSAKKF | seqid_4517 |
| variant_6002 | FKKLFLKIPPKFKKSAKKF | seqid_4518 |
| variant_6003 | KWKLFLKIPPKFKKSAKKF | seqid_4519 |
| variant_6004 | RWKLFLKIPPKFKKSAKKF | seqid_4520 |
| variant_6005 | FWKLFLKIPPKFKKSAKKF | seqid_4521 |
| variant_6006 | KKKKFLKIPPKFKKSAKKF | seqid_4522 |
| variant_6007 | RKKKFLKIPPKFKKSAKKF | seqid_4523 |
| variant_6008 | FKKKFLKIPPKFKKSAKKF | seqid_4524 |
| variant_6009 | KWKKFLKIPPKFKKSAKKF | seqid_4525 |
| variant_6010 | RWKKFLKIPPKFKKSAKKF | seqid_4526 |
| variant_6011 | FWKKFLKIPPKFKKSAKKF | seqid_4527 |
| variant_6012 | KKKLLLKIPPKFKKSAKKF | seqid_4528 |
| variant_6013 | RKKLLLKIPPKFKKSAKKF | seqid_4529 |
| variant_6014 | FKKLLLKIPPKFKKSAKKF | seqid_4530 |
| variant_6015 | KWKLLLKIPPKFKKSAKKF | seqid_4531 |
| variant_6016 | RWKLLLKIPPKFKKSAKKF | seqid_4532 |
| variant_6017 | FWKLLLKIPPKFKKSAKKF | seqid_4533 |
| variant_6018 | KKKKLLKIPPKFKKSAKKF | seqid_4534 |
| variant_6019 | RKKKLLKIPPKFKKSAKKF | seqid_4535 |
| variant_6020 | FKKKLLKIPPKFKKSAKKF | seqid_4536 |
| variant_6021 | KWKKLLKIPPKFKKSAKKF | seqid_4537 |
| variant_6022 | RWKKLLKIPPKFKKSAKKF | seqid_4538 |
| variant_6023 | FWKKLLKIPPKFKKSAKKF | seqid_4539 |
| variant_6024 | KKKLFKKIPPKFKKSAKKF | seqid_4540 |
| variant_6025 | RKKLFKKIPPKFKKSAKKF | seqid_4541 |
| variant_6026 | FKKLFKKIPPKFKKSAKKF | seqid_4542 |
| variant_6027 | KWKLFKKIPPKFKKSAKKF | seqid_4543 |
| variant_6028 | RWKLFKKIPPKFKKSAKKF | seqid_4544 |
| variant_6029 | FWKLFKKIPPKFKKSAKKF | seqid_4545 |
| variant_6030 | KKKKFKKIPPKFKKSAKKF | seqid_4546 |
| variant_6031 | RKKKFKKIPPKFKKSAKKF | seqid_4547 |
| variant_6032 | FKKKFKKIPPKFKKSAKKF | seqid_4548 |
| variant_6033 | KWKKFKKIPPKFKKSAKKF | seqid_4549 |
| variant_6034 | RWKKFKKIPPKFKKSAKKF | seqid_4550 |
| variant_6035 | FWKKFKKIPPKFKKSAKKF | seqid_4551 |
| variant_6036 | KKKLLKKIPPKFKKSAKKF | seqid_4552 |
| variant_6037 | RKKLLKKIPPKFKKSAKKF | seqid_4553 |
| variant_6038 | FKKLLKKIPPKFKKSAKKF | seqid_4554 |
| variant_6039 | KWKLLKKIPPKFKKSAKKF | seqid_4555 |
| variant_6040 | RWKLLKKIPPKFKKSAKKF | seqid_4556 |
| variant_6041 | FWKLLKKIPPKFKKSAKKF | seqid_4557 |
| variant_6042 | KKKKLKKIPPKFKKSAKKF | seqid_4558 |
| variant_6043 | RKKKLKKIPPKFKKSAKKF | seqid_4559 |
| variant_6044 | FKKKLKKIPPKFKKSAKKF | seqid_4560 |
| variant_6045 | KWKKLKKIPPKFKKSAKKF | seqid_4561 |
| variant_6046 | RWKKLKKIPPKFKKSAKKF | seqid_4562 |
| variant_6047 | FWKKLKKIPPKFKKSAKKF | seqid_4563 |
| variant_6096 | KKKLFLKIPKFKKSAKKF | seqid_4564 |
| variant_6097 | RKKLFLKIPKFKKSAKKF | seqid_4565 |
| variant_6098 | FKKLFLKIPKFKKSAKKF | seqid_4566 |
| variant_6099 | KWKLFLKIPKFKKSAKKF | seqid_4567 |
| variant_6100 | RWKLFLKIPKFKKSAKKF | seqid_4568 |
| variant_6101 | FWKLFLKIPKFKKSAKKF | seqid_4569 |
| variant_6102 | KKKKFLKIPKFKKSAKKF | seqid_4570 |
| variant_6103 | RKKKFLKIPKFKKSAKKF | seqid_4571 |
| variant_6104 | FKKKFLKIPKFKKSAKKF | seqid_4572 |
| variant_6105 | KWKKFLKIPKFKKSAKKF | seqid_4573 |
| variant_6106 | RWKKFLKIPKFKKSAKKF | seqid_4574 |
| variant_6107 | FWKKFLKIPKFKKSAKKF | seqid_4575 |
| variant_6108 | KKKLLLKIPKFKKSAKKF | seqid_4576 |
| variant_6109 | RKKLLLKIPKFKKSAKKF | seqid_4577 |
| variant_6110 | FKKLLLKIPKFKKSAKKF | seqid_4578 |
| variant_6111 | KWKLLLKIPKFKKSAKKF | seqid_4579 |
| variant_6112 | RWKLLLKIPKFKKSAKKF | seqid_4580 |
| variant_6113 | FWKLLLKIPKFKKSAKKF | seqid_4581 |
| variant_6114 | KKKKLLKIPKFKKSAKKF | seqid_4582 |
| variant_6115 | RKKKLLKIPKFKKSAKKF | seqid_4583 |
| variant_6116 | FKKKLLKIPKFKKSAKKF | seqid_4584 |
| variant_6117 | KWKKLLKIPKFKKSAKKF | seqid_4585 |
| variant_6118 | RWKKLLKIPKFKKSAKKF | seqid_4586 |
| variant_6119 | FWKKLLKIPKFKKSAKKF | seqid_4587 |
| variant_6120 | KKKLFKKIPKFKKSAKKF | seqid_4588 |
| variant_6121 | RKKLFKKIPKFKKSAKKF | seqid_4589 |
| variant_6122 | FKKLFKKIPKFKKSAKKF | seqid_4590 |
| variant_6123 | KWKLFKKIPKFKKSAKKF | seqid_4591 |
| variant_6124 | RWKLFKKIPKFKKSAKKF | seqid_4592 |
| variant_6125 | FWKLFKKIPKFKKSAKKF | seqid_4593 |
| variant_6126 | KKKKFKKIPKFKKSAKKF | seqid_4594 |
| variant_6127 | RKKKFKKIPKFKKSAKKF | seqid_4595 |
| variant_6128 | FKKKFKKIPKFKKSAKKF | seqid_4596 |
| variant_6129 | KWKKFKKIPKFKKSAKKF | seqid_4597 |
| variant_6130 | RWKKFKKIPKFKKSAKKF | seqid_4598 |
| variant_6131 | FWKKFKKIPKFKKSAKKF | seqid_4599 |
| variant_6132 | KKKLLKKIPKFKKSAKKF | seqid_4600 |
| variant_6133 | RKKLLKKIPKFKKSAKKF | seqid_4601 |
| variant_6134 | FKKLLKKIPKFKKSAKKF | seqid_4602 |
| variant_6135 | KWKLLKKIPKFKKSAKKF | seqid_4603 |
| variant_6136 | RWKLLKKIPKFKKSAKKF | seqid_4604 |
| variant_6137 | FWKLLKKIPKFKKSAKKF | seqid_4605 |
| variant_6138 | KKKKLKKIPKFKKSAKKF | seqid_4606 |
| variant_6139 | RKKKLKKIPKFKKSAKKF | seqid_4607 |
| variant_6140 | FKKKLKKIPKFKKSAKKF | seqid_4608 |
| variant_6141 | KWKKLKKIPKFKKSAKKF | seqid_4609 |
| variant_6142 | RWKKLKKIPKFKKSAKKF | seqid_4610 |
| variant_6143 | FWKKLKKIPKFKKSAKKF | seqid_4611 |

### 3.1.3 Sequence Motif Hel1-HB-Hel2

| | | |
|---|---|---|
| Variant_1 | KWKLFKKIGPKFLHLAKKF- | SEQ ID NO: 4709 |
| | NH₂ | |
| Variant_2 | KWKLFKKGPGKFLHSAKKF- | SEQ ID NO: 4710 |
| | NH₂ | |
| Variant_3 | KWKLFKKIEKVGQGPGKFLHSA | SEQ ID NO: 4711 |
| | KKFG-NH₂ | |
| Variant_4 | WKLFKKIPKFLHLAKKF-NH₂ | SEQ ID NO: 4712 |
| Variant_5 | FKLFLLIPKFLHLAKKF-NH₂ | SEQ ID NO: 4713 |
| Variant_6 | KWFKKIPKFLHLAKKF-NH₂ | SEQ ID NO: 4714 |
| Variant_7 | WFKKIPKFLHLAKKF-NH₂ | SEQ ID NO: 4715 |
| Variant_8 | KWKKIPKFLHLLKKF-NH₂ | SEQ ID NO: 4716 |
| Variant_9 | WFKKIPKFLHLLKKF-NH₂ | SEQ ID NO: 4717 |
| Variant_10 | KWKLFKKIPFLHLAKKF-NH₂ | SEQ ID NO: 4718 |
| Variant_11 | KWKLFKKIPKFLHLAKK-NH₂ | SEQ ID NO: 4719 |
| Variant_12 | KWKLFKKIPLHLAKKF-NH₂ | SEQ ID NO: 4720 |
| Variant_13 | KWKLFKKIPKFLHLAK-NH₂ | SEQ ID NO: 4721 |
| Variant_14 | KWKLFKKIPHLAKKF-NH₂ | SEQ ID NO: 4722 |
| Variant_15 | KWKLFKKIPKFLHLA-NH₂ | SEQ ID NO: 4723 |
| Variant_16 | KWKLFKKIPLAKKF-NH₂ | SEQ ID NO: 4724 |
| Variant_17 | KWKLFKKIPKFLHL-NH₂ | SEQ ID NO: 4725 |
| Variant_18 | FKKALHLFKPIKKFLKWK-NH₂ | SEQ ID NO: 4726 |
| Variant_19 | KFLHLAKKFPKWKLFKKI-NH₂ | SEQ ID NO: 4727 |
| Variant_20 | KWKKLLKKPLLKKLLKKL-NH₂ | SEQ ID NO: 4728 |
| Variant_21 | KWKLKPLLKKLLKKL-NH₂ | SEQ ID NO: 4729 |
| Variant_22 | KWKKLLKKPLKLKL-NH₂ | SEQ ID NO: 4730 |
| Variant_23 | KLLKKPLKLKL-NH₂ | SEQ ID NO: 4731 |

Likewise included are the above sequences except that the C-terminal end has not been amidated, and hence especially those sequences which are terminated by a carboxyl group (in salt or acid form).

### 3.1.4 Modifications of SEQ ID NO: 3

The peptide according to SEQ ID NO: 3 is extended at the N- and/or C-terminal end by any one or more amino acid residues. Nonlimiting examples of additional residues comprise Asp, Pro, Asn, Gly. In the case of simultaneous extension of N and C terminus, the additional N-terminal residue is preferably Pro or Gly, and the residue assigned to the C terminus is preferably Asp or Asn.

| | |
|---|---|
| PKWKLFKKIPKFLHLAKKF-NH₂ | (SEQ ID NO: 4732) |
| KWKLFKKIPKFLHLAKKFD-NH₂ | (SEQ ID NO: 4733) |
| PKWKLFKKIPKFLHLAKKFD-NH₂ | (SEQ ID NO: 4734) |
| GKWKLFKKIPKFLHLAKKF-NH₂ | (SEQ ID NO: 4735) |
| KWKLFKKIPKFLHLAKKFN-NH₂ | (SEQ ID NO: 4736) |
| GKWKLFKKIPKFLHLAKKFN-NH₂ | (SEQ ID NO: 4737) |
| PKWKLFKKIPKFLHLAKKFN-NH₂ | (SEQ ID NO: 4738) |

Likewise included are the above sequences except that the C-terminal end has not been amidated, and hence especially those sequences which are terminated by a carboxyl group (in salt or acid form).

### 3.2 Further Modifications of Inventive Peptides

In addition to the peptide sequences shown above, preference is also given to functional equivalents, functional derivatives and salts of this sequence.

“Functional equivalents” are understood in the course of the invention to mean especially mutants which have, in at least one sequence position of the abovementioned amino acid sequences, an amino acid other than that specified, but nevertheless have the property of prevention, inhibition and treatment of dandruff. “Functional equivalents” thus comprise the mutants obtainable by one or more amino acid additions, substitutions, deletions and/or inversions, where the changes mentioned may occur in any sequence position provided that they lead to a mutant with the inventive profile of properties. Functional equivalence exists especially also when the reactivity patterns between mutant and unchanged polypeptide correspond in qualitative terms.

“Functional equivalents” in the above sense are also “precursors” of the polypeptides described, and “functional derivatives” and “salts” of the polypeptides.

“Precursors” are natural or synthetic precursors of the polypeptides with or without the desired biological activity.

Examples of suitable amino acid substitutions can be found in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The expression “salts” is understood to mean both salts of carboxyl groups and acid addition salts of amino groups of the inventive peptide molecules. Salts of carboxyl groups can be prepared in a manner known per se and comprise inorganic salts, for example sodium, calcium, ammonium, iron and zinc salts, and salts with organic bases, for example amines such as triethanolamine, arginine, lysine, piperidine and the like. Acid addition salts, for example salts with mineral acids such as hydrochloric acid and sulfuric acid, and salts with organic acids such as acetic acid and oxalic acid, likewise form part of the subject matter of the invention.

“Functional derivatives” (or “derivatives”) of inventive polypeptides can likewise be prepared on functional amino acid side groups or on the N- or C-terminal end thereof with the aid of known techniques. Such derivatives comprise, for example, aliphatic esters of carboxylic acid groups, amides of carboxylic acid groups, obtainable by reaction with ammonia or with a primary or secondary amine; N-acyl derivatives of free amino groups, prepared by reaction with acyl groups; or O-acyl derivatives of free hydroxyl groups, prepared by reaction with acyl groups. Furthermore, it is additionally possible for any 1 to 5, for example 2, 3 or 4, D- or L-amino acid residues to be bonded covalently (peptidically) at the N- and/or C-terminal end; or it is possible for 1 to 5, for example 1, 2, 3 or 4 in each case, residues to be absent at the N- and/or C-terminal end.

Nonlimiting examples of additional N- and/or C-terminal residues comprise Asp, Pro, Asn, Gly. In the case of simultaneous extension of N and C terminus, the additional N-terminal residue is preferably Pro or Gly, and the residue assigned to the C terminus is preferably Asp or Asn.

By variation of the amino acid sequence of the antimicrobial peptides described or fusion with additional protein or peptide sequences, it is possible to generate structures which specifically recognize particular surfaces, for example skin, nails, hair, or are recognized and bound by these surfaces or the receptors present.

This makes it possible to more effectively bring the antimicrobial peptides described to the desired site of action, or to improve the uptake thereof. By coupling or fusion of binding proteins to the antimicrobial peptides described, protein-peptide fusion products originating therefrom would be directed in a more controlled manner to appropriate sites of action, for example microorganism surfaces or body compartments, or would reside longer at these sites, which results in a prolonged and improved peptide effect. Furthermore, it is possible by variation of the amino acid sequence of the antimicrobial peptides described or fusion with additional protein or peptide sequences to direct the peptides in a controlled manner to desired sites of action, in order thus to achieve, for example, higher peptide specificity, lower peptide consumption or peptide dose, and faster or stronger peptide action.

### 3.3 Nucleic Acids, Expression Constructs, Vectors and Microorganisms for Preparation of the Antimicrobial Peptides

### Nucleic Acids:

The invention further comprises the nucleic acid molecules which code for the peptides and fusion peptides used in accordance with the invention.

All nucleic acid sequences mentioned herein (single- and double-strand DNA and RNA sequences, for example cDNA and mRNA) can be prepared in a manner known per se by chemical synthesis from the nucleotide units, for example by fragment condensation of individual overlapping, complementary nucleic acid units of the double helix. The chemical synthesis of oligonucleotides can be effected, for example, in a known manner, by the phosphoramidite method (Voet, Voet, 2^{nd} edition, Wiley Press New York, pages 896-897). The addition of synthetic oligonucleotides and filling of gaps with the aid of the Klenow fragment of DNA polymerase and ligation reactions, and also general cloning methods, are described in Sambrook et al. (1989), Molecular Cloning: A laboratory manual, Cold Spring Harbor Laboratory Press.

The invention relates both to isolated nucleic acid molecules which code for inventive polypeptides or proteins or biologically active sections thereof, and to nucleic acid fragments which can be used, for example, as hybridization probes or primers for identification or amplification of inventive coding nucleic acids.

The inventive nucleic acid molecules may additionally comprise untranslated sequences from the 3′ and/or 5′ end of the coding gene region.

An “isolated” nucleic acid molecule is separated from other nucleic acid molecules present in the natural source of the nucleic acid, and may moreover be essentially free of other cellular material or culture medium when it is produced by recombinant techniques, or free of chemical precursors or other chemicals when it is synthesized chemically.

An inventive nucleic acid molecule can be isolated by means of standard molecular biological techniques and the sequence information provided in accordance with the invention. For example, cDNA can be isolated from a suitable cDNA library, by using one of the specifically disclosed complete sequences or a section thereof as a hybridization probe, and standard hybridization techniques (as described, for example, in Sambrook, J., Fritsch, E. F. and Maniatis, T. Molecular Cloning: A Laboratory Manual. 2^{nd} edition, Cold Spring Harbor Laboratory, Cold Spring Harbor Laboratory Press, Cold Spring Harbor, N.Y., 1989). Moreover, it is possible to isolate a nucleic acid molecule comprising one of the sequences disclosed or a section thereof by polymerase chain reaction, using the oligonucleotide primers which have been established on the basis of this sequence. The nucleic acid thus amplified can be cloned into a suitable vector and characterized by DNA sequence analysis. The inventive oligonucleotides can also be prepared by standard synthesis methods, for example with an automatic DNA synthesis system.

The invention further comprises the nucleic acid molecules complementary to the specifically described nucleotide sequences, or a section thereof.

The inventive nucleotide sequences enable the production of probes and primers which can be used for identification and/or cloning of homologous sequences in other cell types and organisms. Such probes or primers usually comprise a nucleotide sequence region which, under stringent conditions, hybridizes at least about 12, preferably at least about 25, for example about 40, 50 or 75, consecutive nucleotides of a sense strand of an inventive nucleic acid sequence or of a corresponding antisense strand.

The invention also comprises those nucleic acid sequences which comprise what are called silent mutations or have been altered in accordance with the codon usage of a specific original or host organism as compared with a specified sequence, and likewise naturally occurring variants, for example splice variants or allele variants, thereof. Likewise provided are sequences obtainable by conservative nucleotide substitutions (i.e. the amino acid in question is replaced by an amino acid of the same charge, size, polarity and/or solubility).

The invention also provides the molecules derived from the specifically disclosed nucleic acids by sequence polymorphisms. These genetic polymorphisms may exist between individuals within a population on the basis of natural variation. These natural variations typically cause a variance of 1 to 5% in the nucleotide sequence of a gene.

In addition, the invention also comprises nucleic acid sequences which hybridize with or are complementary to the abovementioned coding sequences. These polynucleotides can be found when searching through genomic or cDNA libraries and can optionally be amplified therefrom with suitable primers by means of PCR and then isolated, for example, with suitable probes. A further option is that of transforming suitable microorganisms with inventive polynucleotides or vectors, to propagate the microorganisms and hence the polynucleotides, and then to isolate them. In addition, it is also possible to synthesize inventive polynucleotides by a chemical route.

The property of being able to “hybridize” onto polynucleotides is understood to mean the ability of a poly- or oligonucleotide to bind to a virtually complementary sequence under stringent conditions, while there are no unspecific bindings between noncomplementary partners under these conditions. For this purpose, the sequences should be 70-100% complementary, preferably 90-100%. The property of complementary sequences being able to bind specifically to one another is utilized, for example, in the Northern or Southern blot technique, or in primer binding in PCR or RT-PCR. Typically, oligonucleotides are used for this purpose from a length of 30 base pairs. Stringent conditions are understood, for example, in the Northern blot technique to mean the use of a wash solution at 50-70° C., preferably 60-65° C., for example 0.1×SSC buffer with 0.1% SDS (20×SSC: 3 M NaCl, 0.3 M Na citrate, pH 7.0), for elution of unspecifically hybridized cDNA probes or oligonucleotides. As mentioned above, only nucleic acids which are complementary to a high degree remain bound to one another. The establishment of stringent conditions is known to those skilled in the art and is described, for example, in Ausubel et al., Current Protocols in Molecular Biology, John Wiley & Sons, N.Y. (1989), 6.3.1-6.3.6.

### Expression Constructs and Vectors:

The invention also provides expression constructs comprising, under the genetic control of regulatory nucleic acid sequences, a nucleic acid sequence coding for an inventive polypeptide, and vectors comprising at least one of these expression constructs. Such inventive constructs preferably comprise a promoter 5′ upstream from the particular coding sequence, and a terminator sequence 3′ downstream, and optionally further customary regulatory elements, each operatively linked to the coding sequence. An “operative linkage” is understood to mean the sequential arrangement of promoter, coding sequence, terminator and optionally further regulatory elements in such a way that each of the regulatory elements can fulfill its function as intended in the expression of the coding sequence. Examples of operatively linkable sequences are targeting sequences, and enhancers, polyadenylation signals and the like. Further regulatory elements comprise selectable markers, amplification signals, origins of replication and the like. Suitable regulatory sequences are described, for example, in Goeddel, Gene Expression Technology: Methods in Enzymology 185, Academic Press, San Diego, Calif. (1990).

In addition to the artificial regulation sequences, the natural regulation sequence may still be present in front of the actual structure gene. By genetic variation, this natural regulation can optionally be switched off and the expression of the genes can be increased or reduced. The gene construct may, however, also be of simpler construction, which means that no additional regulation signals are inserted in front of the structure gene and the natural promoter with its regulation is not deleted. Instead, the natural regulation sequence is mutated in such a way that there is no longer any regulation and the gene expression is enhanced or reduced. The nucleic acid sequences may be present in one or more copies in the gene construct.

Examples of usable promoters are: cos, tac, trp, tet, trp-tet, lpp, lac, lpp-lac, laclq, T7, T5, T3, gal, trc, ara, SP6, lambda-PR or lambda-PL promoter, which advantageously find use in Gram-negative bacteria; and the Gram-positive promoters amy and SPO2, the yeast promoters ADC1, MFa, AC, P-60, CYC1, GAPDH, or the plant promoters CaMV/355, SSU, OCS, lib4, usp, STLS1, B33, not, or the ubiquitin or phaseolin promoter. Particular preference is given to the use of inducible promoters, for example light- and especially temperature-inducible promoters, such as the PᵣPₗ promoter. In principle, it is possible to use all natural promoters with their regulation sequences. In addition, it is also advantageously possible to use synthetic promoters.

The regulatory sequences mentioned are intended to enable the controlled expression of the nucleic acid sequences and protein expression. According to the host organism, this can mean, for example, that the gene is expressed or overexpressed only after induction, or that it is expressed and/or overexpressed immediately.

The regulatory sequences or factors can preferably positively influence expression, and increase or lower it as a result. For instance, the regulatory elements can advantageously be enhanced at the transcription level, by using strong transcription signals such as promoters and/or “enhancers”. In addition, however, it is also possible to enhance translation, for example by improving the stability of the mRNA.

An expression cassette is produced by fusing a suitable promoter with a suitable coding nucleotide sequence and a terminator signal or polyadenylation signal. For this purpose, standard recombination and cloning techniques are used, as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which enables optimal expression of the genes in the host. Vectors are well-known to those skilled in the art and can be found, for example, in “Cloning Vectors” (Pouwels P. H. et al., eds., Elsevier, Amsterdam-New York-Oxford, 1985). Vectors, apart from plasmids, are also all other vectors known to those skilled in the art, for example phages, viruses such as SV40, CMV, baculovirus and adenovirus, transposons, IS elements, phasmids, cosmids, and linear or circular DNA. These vectors can be replicated autonomously in the host organism or replicated chromosomally.

### Examples of Suitable Expression Vectors May Include:

Customary fusion expression vectors such as pGEX (Pharmacia Biotech Inc; Smith, D. B. and Johnson, K. S. (1988) Gene 67:31-40), pMAL (New England Biolabs, Beverly, Mass.) and pRIT 5 (Pharmacia, Piscataway, N.J.), in the case of which, respectively, glutathione S-transferase (GST), maltose E-binding protein and protein A are fused to the recombinant target protein.

Non-fusion protein expression vectors such as pTrc (Amann et al., (1988) Gene 69:301-315) and pET 11d (Studier et al. Gene Expression Technology: Methods in Enzymology 185, Academic Press, San Diego, Calif. (1990) 60-89).

Yeast expression vector for expression in the yeast S. cerevisiae, such as pYepSec1 (Baldari et al., (1987) Embo J. 6:229-234), pMFa (Kurjan and Herskowitz (1982) Cell 30:933-943), pJRY88 (Schultz et al., (1987) Gene 54:113-123) and pYES2 (Invitrogen Corporation, San Diego, Calif.). Vectors and methods for construction of vectors suitable for use in other fungi such as filamentous fungi comprise those described in detail in: van den Hondel, C. A. M. J. J. & Punt, P. J. (1991) “Gene transfer systems and vector development for filamentous fungi, in: Applied Molecular Genetics of Fungi, J. F. Peberdy et al., eds., p. 1-28, Cambridge University Press: Cambridge.

Baculovirus vectors available for expression of proteins in cultured insect cells (for example Sf9 cells) comprise the pAc series (Smith et al., (1983) Mol. Cell. Biol. 3:2156-2165) and the pVL series (Lucklow and Summers, (1989) Virology 170:31-39).

Plant expression vectors such as those described in detail in: Becker, D., Kemper, E., Schell, J. and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721.

Mammalian expression vectors such as pCDM8 (Seed, B. (1987) Nature 329:840) and pMT2PC (Kaufman et al. (1987) EMBO J. 6:187-195).

Further suitable expression systems for prokaryotic and eukaryotic cells are described in chapters 16 and 17 of Sambrook, J., Fritsch, E. F. and Maniatis, T., Molecular cloning: A Laboratory Manual, 2^{nd} edition, Cold Spring Harbor Laboratory, Cold Spring Harbor Laboratory Press, Cold Spring Harbor, N.Y., 1989.

### Recombinant Microorganisms:

The inventive vectors can be used to produce recombinant microorganisms which have been transformed, for example, with at least one inventive vector and can be used for production of the inventive polypeptides. Advantageously, the above-described inventive recombinant constructs are introduced into and expressed in a suitable host system. Preference is given to using familiar cloning and transfection methods known to those skilled in the art, for example coprecipitation, protoplast fusion, electroporation, retroviral transfection and the like in order to bring about expression of the nucleic acids mentioned in the particular expression system. Suitable systems are described, for example, in Current Protocols in Molecular Biology, F. Ausubel et al., eds., Wiley Interscience, New York 1997, or Sambrook et al., Molecular Cloning: A Laboratory Manual. 2^{nd} ed., Cold Spring Harbor Laboratory, Cold Spring Harbor Laboratory Press, Cold Spring Harbor, N.Y., 1989.

According to the invention, it is also possible to produce homologously recombined microorganisms. For this purpose, a vector comprising at least a section of an inventive gene or of a coding sequence is produced, in which at least one amino acid deletion, addition or substitution has optionally been introduced, in order to alter the inventive sequence, for example to functionally disrupt it (“knockout” vector). The sequence introduced may, for example, also be a homolog from a related microorganism or be derived from a mammalian, yeast or insect source. The vector used for homologous recombination may alternatively be configured such that the endogenous gene has been mutated or altered in some other way on homologous recombination, but still encodes the functional protein (for example, the upstream regulatory region may be altered in such a way that this alters the expression of the endogenous protein). The altered section of the inventive gene is in the homologous recombination vector. The construction of suitable vectors for homologous recombination is described, for example, in Thomas, K. R. and Capecchi, M. R. (1987) Cell 51:503.

Suitable host organisms are in principle all organisms which enable expression of the inventive nucleic acids, allele variants thereof, or functional equivalents or derivatives thereof. Host organisms are understood to mean, for example, bacteria, fungi, yeasts, or plant or animal cells. Preferred organisms are bacteria, such as those of the genera Escherichia, for example Escherichia coli, Streptomyces, Bacillus or Pseudomonas, eukaryotic microorganisms such as Saccharomyces cerevisiae, Aspergillus, higher eukaryotic cells from animals or plants, for example Sf9 or CHO cells.

Successfully transformed organisms can be selected by means of marker genes likewise present in the vector or in the expression cassette. Examples of such marker genes are genes for antibiotic resistance and for enzymes which catalyze a coloring reaction, which causes staining of the transformed cell. These can then be selected by means of automatic cell sorting. Microorganisms which have been successfully transformed with a vector and bear a corresponding antibiotic resistance gene (for example G418 or hygromycin) can be selected by means of appropriate antibiotic-comprising media or nutrient media. Marker proteins which are presented on the cell surface can be utilized for selection by means of affinity chromatography.

As alternative production methods for inventive sequences, reference is also made to chemical synthesis methods known per se, such as solid phase synthesis or liquid phase synthesis.

### 3.4 Recombinant Production of the Polypeptides:

The peptides used in accordance with the invention can be produced recombinantly in a manner known per se, by cultivating a microorganism which produces polypeptides, optionally inducing the expression of the polypeptides and isolating them from the culture. The polypeptides can thus also be produced on the industrial scale if desired.

The recombinant microorganism can be cultivated and fermented by known processes. Bacteria can be multiplied, for example, in TB or LB medium and at a temperature of 20 to 40° C. and a pH of 6 to 9. Details of suitable cultivation conditions are described, for example in T. Maniatis, E. F. Fritsch and J. Sambrook, Molecular Cloning: A Laboratory Manual, Cold Spring Harbor Laboratory, Cold Spring Harbor, N.Y. (1989).

If the polypeptides are not secreted into the culture medium, the cells are then disrupted and the product is obtained from the lysate by known protein isolation methods. Alternative methods of disrupting the cells are by high-frequency ultrasound, by high pressure, for example in a French pressure cell, by osmolysis, by the action of detergents, lytic enzymes or organic solvents, by homogenizers or by combination of a plurality of the methods mentioned.

Purification of the polypeptides can be achieved by known chromatographic methods, such as molecular sieve chromatography (gel filtration), such as Q-Sepharose chromatography, ion exchange chromatography and hydrophobic chromatography, and by other customary methods such as ultrafiltration, crystallization, salting out, dialysis and native gel electrophoresis. Suitable methods are described, for example in Cooper, T. G., Biochemische Arbeitsmethoden [The Tools of Biochemistry], Walter de Gruyter publishers, Berlin, New York, or in Scopes, R., Protein Purification, Springer Verlag, New York, Heidelberg, Berlin.

It is particularly advantageous to isolate the recombinant protein using vector systems or oligonucleotides which extend the cDNA with particular nucleotide sequences, and hence code for altered polypeptides or fusion proteins which serve, for example, for simpler purification. Suitable modifications of this kind are, for example, “tags” which function as anchors, for example the modification known as the hexa-histidine anchor, or epitopes which can be recognized as antigens by antibodies (described, for example, in Harlow, E. and Lane, D., 1988, Antibodies: A Laboratory Manual. Cold Spring Harbor (N.Y.) Press). These anchors can serve to attach the proteins to a solid support, for example a polymer matrix, which can be introduced, for example, into a chromatography column, or can be used on a microtiter plate or another support.

At the same time, these anchors can also be used for recognition of the proteins. The protein can also be recognized using customary markers, such as fluorescent dyes, enzyme markers which form a detectable reaction product after reaction with a substrate, or radioactive labels, alone or in combination with the anchors for derivatization of the proteins.

The compositions and medicaments comprising inventive antimicrobial peptides have a broad field of use in human and veterinary therapy, especially for treatment of mycoses, preferably of dermatomycoses, and also for treatment of bacterial infections, especially external infections, for example infections of the skin, nails, hair and mucous membranes.

Suitable excipients and additives for the production of formulations are familiar to those skilled in the art. The excipients and additives are preferably cosmetically and/or pharmaceutically acceptable excipients. Pharmaceutically acceptable excipients are the excipients which are known to be usable in the sectors of pharmacy and of food technology and adjoining fields, especially those listed in relevant pharmacopeias (e.g. DAB, Ph. Eur., BP, NF), and other excipients whose properties do not oppose physiological use.

Suitable excipients may be: lubricants, wetting agents, emulsifiers and suspension media, preservatives, antioxidants, antiirritants, chelating agents, emulsion stabilizers, film formers, gel formers, odor masking agents, hydrocolloids solvents, solubilizers, neutralizers, permeation accelerators, pigments, quaternary ammonium compounds, refatting and superfatting agents, ointment, cream or oil bases, silicone derivatives, stabilizers, sterilants, propellents, desiccants, opacifiers, thickeners, waxes, softeners, white oil. A configuration in this regard is based on specialist knowledge, as detailed, for example in Fiedler, H. P. Lexikon der Hilfsstoffe für Pharmazie, Kosmetik and angrenzende Gebiete [Lexicon of Excipients for Pharmacy, Cosmetics and Adjoining Fields], 4^{th} ed., Aulendorf: ECV-Editio-Kantor-Verlag, 1996.

It is possible with preference to use nonionic surfactants. Preferentially suitable examples are zwitterionic surfactants such as cocamidopropylbetaine, positively charged surfactants such as hexadecyltrimethylammonium bromide (CTAB), and uncharged surfactants such as block polymers and glucosides.

Suitable anionic surfactants are, for example, alkyl sulfates, alkyl ether sulfates, alkyl sulfonates, alkyl aryl sulfonates, alkyl succinates, alkyl sulfosuccinates, N-alkyl sarcosinates, acyl taurates, acyl isethionates, alkyl phosphates, alkyl ether phosphates, alkyl ether carboxylates, alpha-olefinsulfonates, especially the alkali metal and alkaline earth metal salts, e.g. sodium, potassium, magnesium, calcium, and ammonium and triethanolamine salts. The alkyl ether sulfates, alkyl ether phosphates and alkyl ether carboxylates may have between 1 and 10 ethylene oxide or propylene oxide units, preferably 1 to 3 ethylene oxide units, in the molecule.

Suitable examples are sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkyl betaines, alkylamido propylbetaines, alkyl sulfobetaines, alkyl glycinates, alkyl carboxy glycinates, alkyl amphoacetates or propionates, alkyl amphodiacetates or dipropionates.

For example, it is possible to use cocodimethyl sulfopropyl betaine, lauryl betaine, cocamidopropyl betaine or sodium cocamphopropionate.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is approx. 6 to 60 mols for one mole of alcohol. Also suitable are alkylamine oxides, mono- or dialkylalkanolamides, fatty acid esters of polyethylene glycols, alkyl polyglycosides or sorbitan ether esters.

The invention will now be illustrated further with reference to the nonlimiting examples which follow.

### EXPERIMENTAL

The studies described hereinafter were conducted on the Malassezia furfur strain DSM 6170.

### Example 1

### Long-Term Stability of P18 Tested on Malassezia Furfur

Since storage may be necessary over a prolonged period, a 1 mM P18 peptide solution (P18 sequence H-KWKLFKKIPKFLHLAKKF-NH₂) was subsequently stored at 37° C. over 12 weeks, and the antifungal activity of the stored solution on Malassezia furfur was compared with the activity of a freshly made up 1 mM P18 peptide solution. This was done using a growth test, which was conducted as follows:

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 100 μl per well of M472 Pityrosporum medium and inoculated with M. furfur suspension from the overnight culture. The M. furfur suspension was adjusted at the start of the experiment to an optical density, measured at 600 nm, of 0.02. The concentrates of the inhibitor solutions were 1 mM in water.

The following growth was compared:
M. furfur suspension without addition of an inhibitor
M. furfur suspension and addition of an aqueous fresh P18 solution with a final concentration of 50 μM
M. furfur suspension and addition of an aqueous fresh P18 solution with a final concentration of 25 μM
M. furfur suspension and addition of a P18 solution which had been stored at 37° C. for 12 weeks with a final concentration of 50 μM
M. furfur suspension and addition of a P18 solution which had been stored at 37° C. for 12 weeks with a final concentration of 25 μM

The microtiter plate was incubated with shaking at 30° C.

The growth was observed by measuring the optical density over 24 hours. Subsequently, the colony forming units (CFU) were determined by streaking 1 μl and 5 μl of each of the suspensions and, after incubation over 6 days, counting the colonies. The CFU was determined in order to rule out any influence of the biphasic medium, and also the growth form of M. furfur, on the optical density. The experiments were conducted at least in triple determinations.

TABLE 1
Counting of the colony forming units
(the figures shown are means and standard deviations)

| | | | | | Addition |
|---|---|---|---|---|---|
| | | | | Addition | of |
| | | | Addition of | of aqueous | aqueous |
| | | Addition of | aqueous | P18 | stored P18 |
| | | aqueous | stored P18 | solution | solution |
| Incubation | | P18 solution | solution | [final | [final |
| time | No | [final conc. | [final conc. | conc. | conc. 25 |
| [h] | inhib. | 50 μM] | 50 μM] | 25 μM] | μM] |
| 16-18 | >1000 | 0 ± 0 | 0 ± 0 | 5 ± 8 | 6 ± 7 |
| 24 | >1000 | 1 ± 1 | 1 ± 1 | 1 ± 2 | 5 ± 5 |

The results of the growth test show that the growth of M. furfur measured as colony forming unit has been effectively inhibited by the P18 peptide solution stored for 12 weeks and the fresh P18 peptide solution. This means that the storage of the 1 mM P18 peptide solution has not affected the activity of the solution; the solution was consequently storable over this period.

### Example 2

### Formulability of P18

The formulability of the peptide P18 was tested in three different shampoo base formulations. For this purpose, the formulations with the following compositions were first produced:

TABLE 2
Composition of the base formulations

| | Formulation | Formulation | Formulation |
|---|---|---|---|
| Brand name (INCI) | 31-1 | 31-2 | 31-3 |
| Texapon NSO (Sodium | 40% | 30% | 20% |
| Laureth Sulfate) | | | |
| Tego Betain L7 | 10% | 10% | 20% |
| (Cocamidopropyl | | | |
| Betaine) | | | |

The components were mixed and dissolved. NaOH was used to adjust the pH to pH 6-7. Thereafter, two 100 mM solutions of peptide P18 (P18 sequence H-KWKLFKKIPKFLHLAKKF-NH₂) were prepared. DMSO was the solvent for one solution; it was water for the other solution. The appropriate volume of the 100 mM P18 peptide solution was added to each of the formulations, such that the final concentration in formulations 31-1 and 31-2 was 10 mM, and the final concentration of peptide P18 in formulation 31-3 was 5 mM. The formulations thus obtained were clear and homogenous.

### Example 3

### Effect of Shampoo Base Formulations with P18 as an Ingredient

The aim of the experiment was to study the effect of a shampoo base formulation with the P18 peptide ingredient (P18 sequence H-KWKLFKKIPKFLHLAKKF-NH₂). For this purpose, in this experiment P18 peptide was added directly to the formulation. First of all, the formulations were produced with the following compositions:

TABLE 3
Composition of the shampoo base formulation and
shampoo base formulation with P18 as an ingredient

| | | Shampoo base |
|---|---|---|
| | Shampoo | formulation |
| | base | 31-3 |
| | formulation | with P18 as an |
| Brand name (INCI) | 31-3 | ingredient |
| Texapon NSO (Sodium Laureth Sulfate) | 20% | 20% |
| Tego Betain L7 (Cocamidopropyl Betaine) | 20% | 20% |
| P18 Peptide | — | 5 mM (~1%) |
| (H-KWKLFKKIPKFLHLAKKF-NH₂) | | |

The components Texapon NSO and Tego Betain L7 were mixed and dissolved. NaOH was used to adjust the pH to pH 6-7. Thereafter, a 100 mM aqueous solution of peptide P18 (P18 sequence H-KWKLFKKIPKFLHLAKKF-NH₂) was prepared. The appropriate volume of the 100 mM P18 peptide solution was added to the formulation in each case, such that the final concentration of peptide P18 in formulation 31-3 was 5 mM. As already described above, the formulation thus obtained was clear and homogeneous. The effect of the formulations against the fungus Malassezia furfur was now compared with the shampoo base formulation which did not comprise any P18 peptide.

In summary, the test was conducted as follows.

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 170 μl per well of M472 Pityrosporum medium and inoculated with 10 μl of M. furfur suspension from an overnight culture. This corresponded to an optical density of 0.02-0.1 measured at 620 nm. To this mixture were added 20 μl of shampoo base formulation or 20 μl of shampoo base formulation 31-3 with P18 as an ingredient.

Accordingly, in this experiment, in summary, the following mixtures were compared with one another:
M. furfur suspension
M. furfur suspension and addition of 20 μl of shampoo base formulation 31-3
M. furfur suspension and addition of 20 μl of shampoo base formulation 31-3 with P18 as an ingredient

The microtiter plate was incubated with shaking at 30° C.

After incubation for 24 hours, the colony forming units (CFU) were determined by resuspending 1 μl of each of the suspensions in 10 μl of medium and then streaking them. After incubation over 6 days, the colonies were counted on the plate. The CFU was determined in order to rule out any influence of the biphasic medium, and also the growth form of M. furfur, on the optical density. The experiments were each conducted in double determination, at least in two independent experiments.

TABLE 4
Counting of colony forming units after incubation time of 24 hours

| | | | M. furfur |
|---|---|---|---|
| | | | suspension and |
| | | M. furfur | addition of |
| | | suspension and | shampoo base |
| | | addition of | formulation 31-3 |
| | M. furfur | shampoo base | with P18 as an |
| | suspension | formulation 31-3 | ingredient |
| 24 h | >2000 | 300 ± 199 | 0 ± 0 |

The results show that the shampoo base formulation 31-3 already has a measurable growth-inhibiting effect against Malassezia furfur. However, shampoo base formulation 31-3 with P18 as an ingredient has the effect that no growth of M. furfur is detectable any longer. This shows that the antifungal action of the P18 peptide is maintained in this formulation. Since no further growth of M. furfur has been found, it can even be assumed that even relatively small concentrations of the P18 peptide ingredient or of comparable peptides, and also other comparable formulations, are suitable for growth inhibition of Malassezia furfur and other Malassezia ssp.

### Example 4

### Growth Inhibition of Malassezia Furfur at Equal Concentrations of P18 Peptide, Zinc Pyrithione, Climbazole and Ketoconazole Based on Percentages by Weight (% (Weight/Weight))

The molar masses of P18 peptide (P18 peptide sequence H-KWKLFKKIPKFLHLAKKF-NH₂) and of the currently commercial ingredients of antidandruff shampoos for growth inhibition of the fungus Malassezia furfur differ significantly. The molar mass of P18 peptide is 2300 g/mol, that of zinc pyrithione 317 g/mol, that of ketoconazole 531 g/mol and climbazole has a molar mass of 292 g/mol. Since the experiments regarding growth inhibition of M. furfur in the preceding examples had always been performed with comparable molarities so far, the growth inhibition of Malassezia furfur in these examples was studied with use of equal concentrations of P18 peptide, zinc pyrithione, climbazole and ketoconazole based on the percentages by weight (% (weight/weight)). For this purpose, the procedure was as follows:

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 100 μl per well of M472 Pityrosporum medium and inoculated with M. furfur suspension from the overnight culture. The M. furfur suspension was adjusted at the start of the experiment to an optical density, measured at 600 nm, of 0.02. The concentrates of the inhibitor solutions were 1 mM in DMSO for P18 peptide and 10 mM in DMSO for zinc pyrithione, ketoconazole and climbazole. The final DMSO concentration was kept the same in all experiments. This means that, in the case of higher concentrations of the concentrates of zinc pyrithione, ketoconazole and climbazole, an appropriate volume of DMSO was added to the mixture so as to give comparability to the mixtures containing P18 peptide.

The following growth was compared by measuring the optical density:
M. furfur suspension and addition of the P18 solution with a final concentration of 50 μM (corresponds to 0.0115% (w/w))
M. furfur suspension and addition of the zinc pyrithione (ZPT) solution with a final concentration of 362 μM (comparison to the mixture containing 50 μM P18 peptide)
M. furfur suspension and addition of the ketoconazole solution with a final concentration of 216 μM (comparison to the mixture containing 50 μM P18 peptide)
M. furfur suspension and addition of the climbazole solution with a final concentration of 390 μM (comparison to the mixture containing 50 μM P18 peptide)

The microtiter plate was incubated with shaking at 30° C.

After incubation for 24 hours, the colony forming units (CFU) were determined by streaking 10 μl of medium from each of the suspensions onto agar plates. After incubation over the course of 6 days, the colonies on the plate were counted. The CFU was determined in order to rule out any influence of the biphasic medium, and also the growth form of M. furfur, on the optical density. The experiments were each conducted in double determination, at least in two independent experiments.

TABLE 5
Counting of colony forming units (CFU)
(the figures shown are means and standard deviations of the experiments)

| | Addition of | Addition of | Addition of | Addition |
|---|---|---|---|---|
| | P18 peptide | zinc pyrithione | climbazole | of ketoconazole |
| Incubation | solution | solution | solution | solution |
| time | [final conc. | [final conc. | [final conc. | [final conc. |
| [h] | 50 μM] | 362 μM] | 390 μM] | 216 μM] |
| 24 | 1 ± 1 | 60 ± 40 | 137 ± 33 | 84 ± 76 |
| 40 | 0 ± 0 | 26 ± 7 | 35 ± 15 | 9 ± 5 |

It was observed that the addition of the P18 peptide solution over the test period reduced the CFU and consequently the growth of Malassezia furfur to a greater degree than the comparative substances zinc pyrithione (ZPT), climbazole and ketoconazole.

These results show that P18 peptide at equal concentrations based on the percentages by weight (% (w/w)) leads to effective, at least comparable inhibition of growth of Malassezia furfur as compared with zinc pyrithione, climbazole and ketoconazole.

### Example 5

### Incubation Times with P18 Peptide Between 5 Minutes and 1 Hour

Since the growth inhibition of M. furfur by P18 peptide (P18 peptide sequence H-KWKLFKKIPKFLHLAKKF-NH₂) had at first been studied only over incubation times greater than one hour, the effect of P18 peptide was now tested within the first few minutes (5 minutes, 10 minutes and 20 minutes) of incubation time until the first hour after addition to an M. furfur overnight culture, and compared with zinc pyrithione (Sigma Aldrich) as a control substance. For this purpose, the concentrations 100 μM, 200 μM and 500 μM of P18 peptide and of zinc pyrithione as a control substance were used in the experiment. The experiments were conducted as follows:

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 100 μl per well of M472 Pityrosporum medium and inoculated with M. furfur suspension from the overnight culture. The M. furfur suspension was adjusted at the start of the experiment to an optical density, measured at 600 nm, of 0.1.

The following growth was compared:
M. furfur suspension and addition of P18 peptide with a final concentration of 100 μM, plating after 5 minutes, 10 minutes, 20 minutes and 60 minutes
M. furfur suspension and addition of P18 peptide with a final concentration of 200 μM, plating after 5 minutes, 10 minutes, 20 minutes and 60 minutes
M. furfur suspension and addition of P18 peptide with a final concentration of 500 μM, plating after 5 minutes, 10 minutes, 20 minutes and 60 minutes
M. furfur suspension and addition of zinc pyrithione with a final concentration of 100 μM, plating after 5 minutes, 10 minutes, 20 minutes and 60 minutes
M. furfur suspension and addition of zinc pyrithione with a final concentration of 200 μM, plating after 5 minutes, 10 minutes, 20 minutes and 60 minutes
M. furfur suspension and addition of zinc pyrithione with a final concentration of 500 μM, plating after 5 minutes, 10 minutes, 20 minutes and 60 minutes

The microtiter plate was incubated with shaking at 30°.

After the incubation times specified, the colony forming units (CFU) were determined by streaking 50 μl of each of the suspensions and, after incubation over the course of 6 days, counting the colonies. The CFU was determined in order to rule out any influence of the biphasic medium, and also the growth form of M. furfur, on the optical density. The experiments were repeated independently of one another. The figures shown are the colony forming units which, in all experiments, show fewer than 1000 colonies, i.e. a distinct growth-inhibiting effect.

TABLE 6
Counting of the colony forming units after the incubation time specified

| | Final | | | | |
|---|---|---|---|---|---|
| | concentration | Colony forming units after incubation time | | | |
| | in | specified | | | |
| | the experiment | 5 | 10 | 20 | 60 |
| Substance | [μM] | minutes | minutes | minutes | minutes |
| P18 | 100 | >1000 | >1000 | 604 | 149 ± 60 |
| | 200 | >1000 | >1000 | >1000 | 52 ± 16 |
| | 500 | >1000 | 398 ± 290 | 26 ± 6 | 1 ± 1 |
| ZPT (zinc | 100 | >1000 | >1000 | >1000 | >1000 |
| pyrithione) | 200 | >1000 | >1000 | >1000 | >1000 |
| | 500 | >1000 | >1000 | >1000 | >1000 |

The results show that the Malassezia furfur living cell count was already distinctly reduced within the first 10 minutes of incubation with the P18 peptide as compared with the incubation with zinc pyrithione. After incubation for 60 minutes, the colony forming units were already distinctly reduced at lower concentrations of P18 peptide as compared with shorter incubation times. This means that the mechanism of action of P18 peptide differs significantly from that of zinc pyrithione, and P18 peptide already exhibits action against M. furfur after a short incubation time.

### Example 6

### Effect of P18 Variants

The inhibitory effect of the following P18 variants on M. furfur was studied:

| | |
|---|---|
| H-KWKLFKKIPKFLHLAKKF-NH₂ | (P18; carboxyl |
| | terminus amidated) |
| H-KWKLFKKIPKFLHLAKKF-OH | (P18-OH; carboxyl |
| | terminus not modified) |
| H-PKWKLFKKIPKFLHLAKKFD-OH | (P18AC-OH; carboxyl |
| | terminus not modified) |
| H-PKWKLFKKIPKFLHLAKKFN-NH₂ | (P18AC-NH₂; carboxyl |
| | terminus amidated) |

The experiments were conducted as follows:

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 100 μl per well of M472 Pityrosporum medium and inoculated with M. furfur suspension from the overnight culture. The M. furfur suspension was adjusted at the start of the experiment to an optical density, measured at 600 nm, of 0.1.

The peptide variants were dissolved with a final concentration of 1 mM in dimethyl sulfoxide (DMSO). It is also possible to dispense with DMSO and to use a purely aqueous peptide solution. 5 μl of this solution were added to 100 μl of M. furfur suspension (final concentration of the P18 peptide 50 μM). In a control mixture, the same amount of DMSO without P18 peptide was added.

The microtiter plate was incubated with shaking at 30°.

After 24 h, the colony forming units (CFU) were determined by streaking 10 μl of each of the suspensions and, after incubation over the course of 6 days, counting the colonies. Two independent experiments each with three identical mixtures were conducted, and the number of colony forming units was averaged.

TABLE 7
Averaged number of colony forming units (CFU) after incubation of
M. furfur with 50 μM of different P18 variants

| Mixtures | CFU |
|---|---|
| M. furfur suspension without additions | 1377 |
| Suspension + DMSO | 150 |
| Suspension + DMSO + P18 | 0 |
| Suspension + DMSO + P18-OH | 3 |
| Suspension + DMSO + P18AC-OH | 37 |

The experiment shows that the DMSO solvent already causes a reduction in the CFU. In addition, however, all P18 variants exhibit enhanced inhibition of M. furfur as compared with the control with DMSO. The efficacy increases in the sequence P18AC-OH; P18-OH; P18-NH2. The number of negatively charged carboxyl groups in the peptide molecule decreases in the same sequence. It can therefore be concluded that peptide variants with low negative charge exhibit the best effect.

### Example 7

### Kinetics of the Effect of P18 Peptide on M. Furfur in the Presence of a Shampoo Base Formulation

The effect of the P18 peptide (H-KWKLFKKIPKFLHLAKKF-NH₂; carboxyl terminus amidated) as compared with zinc pyrithione and climbazole with different contact times was tested in the presence of the shampoo base formulation. The experiments were conducted as follows:

The following shampoo base formulation was made up:

TABLE 8
Composition of the shampoo base formulation

| | Shampoo base |
|---|---|
| Brand name (INCI) | formulation 31-3 |
| Texapon NSO (Sodium Laureth | 20% |
| Sulfate) | |
| Tego Betain L7 | 20% |
| (Cocamidopropyl | |
| Betaine) | |

The Texapon NSO and Tego Betain L7 components were mixed and dissolved. NaOH was used to adjust the pH to pH 6-7.

The effect of P18, ZPT and climbazole on M. furfur was studied as follows:

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 100 μl per well of M472 Pityrosporum medium and inoculated with M. furfur suspension from the overnight culture. The M. furfur suspension was adjusted at the start of the experiment to an optical density, measured at 600 nm, of 0.1. 10% (v/v) of shampoo base formulation 31-3 was added to the M. furfur suspension.

P18 peptide was dissolved with a concentration of 230 g/l in water. The zinc pyrithione and climbazole actives were dissolved with a concentration of 230 g/l in DMSO, which left some of the actives suspended because they were insoluble.

The peptide or active solutions were added to the M. furfur suspension with shampoo base formulation with final concentrations of 2.3 g/l; 1.15 g/l; 0.46 g/l and 0.23 g/l.

The microtiter plate was incubated with shaking at 30°.

After incubation of the mixtures for 5 min; 10 min; 20 min; 60 min and 24 h, the colony forming units (CFU) were determined by streaking 1 μl of each of the suspensions and, after incubation over the course of 6 days, counting the colonies.

It is found that P18 in this test has better properties than zinc pyrithione or climbazole.

### Example 8

### Long-Term Stability of P18 Peptide in Shampoo Base Formulations, Tested on M. Furfur

The long-term stability of P18 peptide (H-KWKLFKKIPKFLHLAKKF-NH₂; carboxyl terminus amidated) in shampoo base formulations was tested.

The following shampoo base formulations were made up:

TABLE 9
Composition of the formulations

| | Formulation | Formulation | Formulation |
|---|---|---|---|
| Brand name (INCI) | 31-1-10 | 31-3-5 | 31-3-2 |
| Texapon NSO (Sodium | 40% | 20% | 20% |
| Laureth Sulfate) | | | |
| Tego Betain L7 | 10% | 20% | 20% |
| (Cocamidopropyl | | | |
| Betaine) | | | |
| P18 peptide | 10 mM | 5 mM | 2 mM |

The Texapon NSO and Tego Betain L7 components were mixed and dissolved. NaOH was used to adjust the pH to pH 6-7. Thereafter, a 100 mM aqueous solution of peptide P18 was prepared. The appropriate volume of the 100 mM P18 peptide solution was added to each of the formulations, such that the final concentrations of peptide P18 listed in Table 15 were obtained.

The formulations were stored at 40° C.

After 0; 12 and 22 days, the effect of the formulations on M. furfur was studied.

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 100 μl per well of M472 Pityrosporum medium and inoculated with M. furfur suspension from the overnight culture. The M. furfur suspension was adjusted at the start of the experiment to an optical density, measured at 600 nm, of 0.1.

10% (v/v) of the stored formulations 31-1-10; 31-3-5; 31-3-2 (Table 9) was added to the M. furfur suspension.

The microtiter plate was incubated with shaking at 30° C.

After 24 hours, the colony forming units (CFU) were determined. For this purpose, 1 μl and 10 μl of each of the suspensions were streaked and, after incubation over the course of 6 days, the colonies were counted.

It is found that P18 has stable properties under the conditions tested.

### Example 9

### Determination of the Minimum Inhibitory Concentration (MIC) of Peptide P18 in the Presence of a Shampoo Base Formulation

The minimum inhibitory concentration (MIC) of peptide P18 (H-KWKLFKKIPKFLHLAKKF-NH₂; carboxyl terminus amidated) in the presence of shampoo base formulation 31-3 (Table 10) was tested in the following manner:

The following shampoo base formulation was made up:

TABLE 10
Composition of the shampoo base formulation

| | Shampoo base |
|---|---|
| | formulation 31-3 |
| | with P18 as an |
| Brand name (INCI) | ingredient |
| Texapon NSO (Sodium Laureth | 20% |
| Sulfate) | |
| Tego Betain L7 | 20% |
| (Cocamidopropyl | |
| Betaine) | |

To prepare the shampoo base formulation, the Texapon NSO and Tego Betain L7 components were mixed and dissolved. NaOH was used to adjust the pH to pH 6-7.

The effect of the peptide on M. furfur was studied as follows:

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 170 μl per well of M472 Pityrosporum medium and inoculated with 10 μl of M. furfur suspension from an overnight culture. This corresponded to an optical density of 0.02-0.1, measured at 620 nm. 20 μl of shampoo base formulation 31-3 were added to this mixture. Peptide P18 was dissolved with a concentration of 10 mM in DMSO. Appropriate amounts of the P18 solution were added in order to obtain the final concentrations listed in Table 11.

The microtiter plate was incubated with shaking at 30° C.

After incubation for 24 hours, the colony forming units (CFU) were determined by resuspending 1 μl of each of the suspensions in 10 μl of medium, and then streaking them. After incubation over the course of 6 days, the colonies on the plate were counted. The experiment was conducted in double determination.

TABLE 11
Counting of colony forming units (CFU) after incubation time of 24 hours

| P18 concentration | CFU |
|---|---|
| 0 μM | 538 |
| 25 μM | 80 |
| 50 μM | 32 |
| 100 μM | 0 |
| 200 μM | 0 |
| 300 μM | 0 |
| 400 μM | 0 |
| 500 μM | 0 |

The results show that the growth of M. furfur is fully inhibited from a concentration of 100 μM P18. The minimum inhibitory concentration in the presence of shampoo base formulation 31-3 is thus between 50 μM and 100 μM. This shows that the antifungal effect of P18 peptide in this formulation is preserved, although the activity is reduced somewhat by the shampoo base formulation used as compared with mixtures without shampoo base formulation (cf. Example 1).

### Example 10

### Combination of Peptide P18 with Conventional Fungicidal Actives

The effect of active combinations consisting of a proportion of the conventional fungicidal active zinc pyrithione or ketoconazole or climbazole and peptide P18 (H-KWKLFKKIPKFLHLAKKF-NH₂; carboxyl terminus amidated) was tested in aqueous solution and in the presence of shampoo base formulation 31-3 in the following manner:

The following shampoo base formulation was made up:

TABLE 12
Composition of the shampoo base formulation

| | Shampoo base |
|---|---|
| Brand name (INCI) | formulation 31-3 |
| Texapon NSO (Sodium Laureth | 20% |
| Sulfate) | |
| Tego Betain L7 | 20% |
| (Cocamidopropyl | |
| Betaine) | |

To produce the shampoo base formulation, the Texapon NSO and Tego Betain L7 components were mixed and dissolved. NaOH was used to adjust the pH to pH 6-7.

The effect of the peptide P18 and of ZPT on M. furfur was studied as follows:

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 170 μl per well of M472 Pityrosporum medium and inoculated with 10 μl of M. furfur suspension from an overnight culture. This corresponded to an optical density of 0.02-0.1 measured at 620 nm. To this mixture were added either 20 μl of shampoo base formulation 31-3 or water. Peptide P18 was dissolved with a concentration of 10 mM in water. The conventional fungicidal active was dissolved with a concentration of 10 mM in DMSO. Appropriate amounts of the P18 solution and of the solution of the conventional fungicidal active were added to the mixtures to obtain the final concentrations listed in Table 13.

TABLE 13
Concentrations of the conventional fungicidal actives and P18

| | Concentration of | |
|---|---|---|
| Mixture | conventional fungicidal | P18 concentration |
| No. | active [μM] | [μM] |
| 1 | 500 | 1000 |
| 2 | 500 | 200 |
| 3 | 500 | 50 |
| 4 | 500 | 0 |
| 5 | 100 | 1000 |
| 6 | 100 | 200 |
| 7 | 100 | 50 |
| 8 | 100 | 0 |
| 9 | 20 | 1000 |
| 10 | 20 | 200 |
| 11 | 20 | 50 |
| 12 | 20 | 0 |
| 13 | 0 | 1000 |
| 14 | 0 | 200 |
| 15 | 0 | 50 |
| 16 | 0 | 0 |

The microtiter plate was incubated with shaking at 30° C.

After incubation for 24 hours, the colony forming units (CFU) were determined by resuspending 1 μl of each of the suspensions in 10 μl of medium and then streaking them. After incubation over the course of 6 days, the colonies on the plate were counted.

It was found that combinations of P18 with conventional fungicidal actives have better properties than the conventional fungicidal actives alone.

### Example 11

### Effect of P18 in the Presence of Nonionic and Zwitterionic Surfactants

The aim of the experiment was to study the effect of nonionic and zwitterionic surfactants on the activity of P18 peptide (P18 sequence H-KWKLFKKIPKFLHLAKKF-NH₂). For this purpose, the following surfactants were used:

Pluracare F 68 (a polyoxyethylene-polyoxypropylene-polyoxyethylene block copolymer)
Plantacare 818 (a glucoside)
Tego Betain L7 (cocamidopropyl betaine)

The test was conducted as follows.

Growth medium: M472 Pityrosporum medium according to DSMZ
40 g/1 malt extract
20 g/l ox bile

### 10 g/l Tween 40

The components were sterilized at 121° C., 1 bar gauge for 20 minutes.

2 g/l olive oil (was sterile-filtered and added after the autoclaving of the other components)

Since the medium was a biphasic medium, the complete medium was treated with ultrasound in order to enlarge the phase boundary.

For agar plates, 150 g/l agar-agar were optionally added to the medium.

The growth test was effected as follows: a shake culture containing M472 Pityrosporum medium was inoculated with M. furfur and incubated with shaking at 30° C. and 200 rpm overnight.

A 96-well microtiter plate was filled with 85 μl per well of an M. furfur suspension in M472 Pityrosporum medium which had an optical density of 0.1 measured at 620 nm. 10 μl of detergent solution and 5 μl of peptide solution were added to the culture. The concentrations of the detergent solutions were adjusted such that the final concentration of the particular detergent in the mixture was 2%; 4% or 6%. The concentrations of the P18 peptide solutions were adjusted such that the final concentration of the P18 peptide in the mixture was 0 μM; 50 μM; 100 μM; 250 μM; or 500 μM.

The microtiter plate was incubated with shaking at 30° C.

After incubation for 24 hours, the colony forming units (CFU) were determined by resuspending 1 μl of each of the suspensions in 10 μl of medium and then streaking them. After incubation over the course of 6 days, the colonies on the plate were counted. The CFU was determined in order to rule out any influence of the biphasic medium, and also the growth form of M. furfur, on the optical density. The experiments were each conducted in triplicate determination at least in two independent experiments.

TABLE 14
Means of the colony forming units (CFU) counted per μl after
incubation time 24 hours. The concentration figures are based
on the final concentration in the assay.

| | P18 conc. | Detergent concentration | | |
|---|---|---|---|---|
| | [μM] | 2% | 4% | 6% |
| Pluracare F68 | 0 | >1000 | >1000 | n.d. |
| | 50 | 4 | 2 | n.d. |
| | 100 | 1 | 0 | n.d. |
| | 250 | 0 | 0 | n.d. |
| | 500 | 0 | 0 | n.d. |
| Plantacare 818 | 0 | >1000 | 975 | 706 |
| | 50 | 6 | 39 | 106 |
| | 100 | 0 | 5 | 10 |
| | 250 | 0 | 0 | 0 |
| | 500 | 0 | 0 | 0 |
| Tego Betain L7 | 0 | 748 | 694 | 302 |
| | 50 | 0 | 3 | 0 |
| | 100 | 0 | 0 | 0 |
| | 250 | 0 | 0 | 0 |
| | 500 | 0 | 0 | 0 |

n.d. = not determined.

The results show that P18 in the presence of nonionic or zwitterionic detergents retains its effect against Malassezia furfur.

### Formulation Example 12

Inventive formulations comprising peptide P18 are described hereinafter. Peptide P18 is used in the examples which follow to represent all other peptides described above. It is obvious to the person skilled in the art that all other inventive peptides specified herein can also be used in the formulations specified below.

In the formulations, peptide 18 can be used as the sole active or in combination with other antifungal or antibacterial actives (see description).

### Example

Gel

| | |
|---|---|
| Hydroxyethylcellulose | 1.0% (w/w) |
| Propylene glycol | 10.0% (w/w) |
| Peptide P18 | 1.0% (w/w) |
| make up with distilled water to | 100.0% (w/w) |

### Example

Ointment

| | |
|---|---|
| Peptide P18 | 1.0% (w/w) |
| make up with a mixture of liquid paraffin/ | 100.0% (w/w) |
| white soft paraffin (ratio 1:4) to | |

### Example

Oil in water emulsion

| | |
|---|---|
| Peptide P18 | 1.0% (w/w) |
| Liquid paraffin | 6.0% (w/w) |
| Cetostearyl alcohol | 7.2% (w/w) |
| White soft paraffin | 15.0% (w/w) |
| Glycerol | 5.0% (w/w) |
| make up with distilled water to | 100.0% (w/w) |

### Example

Ointment

| | |
|---|---|
| Peptide P18 | 1.0% (w/w) |
| Sorbitan monopalmitate | 2.0% (w/w) |
| White soft paraffin | 97.0% (w/w) |

### Example 13

### Effect of P18 on Trichophyton Rubrum

Effect of peptide P18 (P18 sequence H-KWKLFKKIPKFLHLAKKF-NH₂, (Bachem AG, Switzerland)) on Trichophyton rubrum (DSM 21146)

MEP growth medium according to DSMZ (German Collection of Microorganisms and Cell Cultures, Braunschweig, Germany):

30 g of malt extract (Becton, Dickinson and Company, Sparks, USA)
3 g of soya peptone (Becton, Dickinson and Company, Sparks, USA)
make up to 1 liter, set pH 5.6

The components were sterilized at 121° C., 1 bar gauge for 20 minutes.

For agar plates, 15 g/l agar-agar were added to the medium.

The growth test was effected as follows: an agar plate on which the Trichophyton rubrum culture had grown was rinsed with 5 ml of MEP medium. 100 μl of the T. rubrum suspension obtained were added to 10 ml of medium.

A 96-well microtiter plate was filled with 190 μl per well of the T. rubrum suspension. Then a dilution series of the peptide with a final concentration in the wells between 0 and 1000 ppm was added. For this purpose, 10 μl per well of peptide solution with a concentration of 0 ppm to 20 000 ppm were added to the T. rubrum suspension, such that final peptide concentrations in the wells in the range between 0 ppm and 1000 ppm were obtained.

The microtiter plate was incubated at 30° C. The fungal growth was assessed by measuring the optical density at 620 nm and observed over 7 days. The experiments were conducted at least in double determination and were independently repeated at least once.

From a concentration of 125 ppm to 250 ppm and concentrations rising to 1000 ppm in the dilution series of the peptide, no further growth of T. rubrum was found, and so no significant turbidity was measured. A sterile control likewise remained without turbidity, while the suspension without addition of peptide had distinct turbidity.

The results of the growth tests therefore exhibited an antifungal effect of peptide P18 on T. rubrum at a minimum inhibitory concentration of 125 ppm-250 ppm.

### Example 14

### Minimum Inhibitory Concentration of P18 on Malassezia Furfur

Minimum inhibitory concentration of peptide P18 (P18 sequence H-KWKLFKKIPKFLHLAKKF-NH₂ (Bachem AG, Switzerland)) based on Malassezia furfur (DSM 6170)

Growth medium M472 Pityrosporum medium according to DSMZ:

40 g of malt extract (Becton, Dickinson and Company, Sparks, USA)
20 g of ox bile (Merck, Darmstadt, Germany)

### 10 g of Tween 40 (Sigma Aldrich Chemie GmbH, Steinheim, Germany)

make up to 1 liter.

The components were sterilized at 121° C., 1 bar gauge for 20 minutes.

Subsequently, 2 ml of olive oil (sterile-filtered) were added.

For agar plates, 15 g/l agar-agar were added to the medium.

A shake culture containing M472 Pityrosporum medium was inoculated with M. furfur and incubated with shaking at 30° C. and 200 rpm overnight.

The suspension was adjusted to an optical density measured at 600 nm of 0.1. A 96-well microtiter plate was filled with 95 μl per well of the M. furfur suspension. Then a dilution series of the peptide with a final concentration in the wells between 0 and 1000 ppm was added. To this end, 5 μl per well of peptide solution with a concentration in the range from 0 ppm to 20 000 ppm were added to the M. furfur suspension, such that final concentrations of the peptide in the wells in the range between 0 ppm and 1000 ppm were obtained.

The microtiter plate was incubated at 30° C., 600 rpm. The fungal growth was assessed by measuring the optical density at 620 nm and observed over 72 hours. The experiments were conducted at least in double determination and independently repeated at least once.

From a concentration of 125 ppm and concentrations rising to 1000 ppm of the dilution series, no further growth of M. furfur was found, and so no significant turbidity was measured. A sterile control likewise remained without turbidity, while the suspension without addition of peptide had distinct turbidity after 24 hours of incubation.

The results of the growth experiments therefore exhibited an antifungal effect of peptide P18 on M. furfur at a minimum inhibitory concentration of 125 ppm.

### Example 15

### Effect of P18 on Klebsiella Pneumoniae

Effect of peptide P18 (P18 sequence H-KWKLFKKIPKFLHLAKKF-NH₂ (Bachem AG, Switzerland)) on Klebsiella pneumoniae (DSM 681) to study.

TSBY growth medium (Becton, Dickinson and Company, Sparks, USA)
17 g of pancreatin-degraded caseine
3 g of pancreatin-degraded soya
2.5 g of dextrose
5 g of sodium chloride
2.5 g of dipotassium phosphate
3 g of yeast extract (Becton, Dickinson and Company, Sparks, USA)
make up to 1 liter, set pH 7

The components were sterilized at 121° C., 1 bar gauge for 20 minutes.

For agar plates, 15 g/l agar-agar were added to the medium.

A shake culture containing TSBY medium was inoculated with K. pneumoniae and incubated with shaking at 37° C. and 200 rpm overnight.

The suspension was adjusted to an optical density measured at 600 nm of 0.1. A 96-well microtiter plate was filled with 95 μl per well of the K. pneumoniae suspension. Then a dilution series of the peptide with a final concentration in the wells between 0 and 1000 ppm was added. To this end, 5 μl per well of peptide solution with a concentration in the range from 0 ppm to 20 000 ppm were added to the K. pneumoniae suspension, so as to obtain final concentrations of the peptide in the wells in the range between 0 ppm and 1000 ppm.

The microtiter plate was incubated at 37° C., 600 rpm. The bacterial growth was assessed by measuring the optical density at 620 nm and observing it over 24 hours. The experiments were conducted at least in double determination and were independently repeated at least once.

From a concentration of 125 ppm and concentrations rising to 1000 ppm of the dilution series, no further growth of K. pneumoniae was found, and so no significant turbidity was measured. A sterile control likewise remained without turbidity, while the K. pneumoniae suspension without addition of peptide had distinct turbidity after 24 hours of incubation.

The results of the growth experiments therefore exhibited an antibacterial effect of peptide P18 on K. pneumoniae at a minimum inhibitory concentration of 125 ppm.

### Example 16

### Short-Term Effect of P18 on Candida albicans

An effect of P18 on Candida albicans after incubation over several hours is known (Lee, D. G., Hahm, K. S., Shin, S. Y. Structure and fungicidal activity of a synthetic antimicrobial peptide, P18, and its truncated peptides, Biotechnology Letters, 2004, 26: 337-341). The aim of this experiment was to study the effect of peptide P18 (P18 sequence H-KWKLFKKIPKFLHLAKKF-NH₂ (Bachem AG, Switzerland)) against Candida albicans (DSM 11948) in the course of brief incubation within the first hour and to compare it with the effect of ZPT (zinc pyrithione). For this purpose, the procedure was as follows.

YM growth medium (Becton, Dickinson and Company, Sparks, USA)
3 g of yeast extract
3 g of malt extract
5 g of peptone
10 g of dextrose
make up to 1 liter, set pH 6.2

The components were sterilized at 121° C., 1 bar gauge for 20 minutes.

For agar plates, 15 g/l agar-agar were added to the medium.

The growth test was effected as follows: 5 ml of YM medium were inoculated with C. albicans and incubated at 30° C. and 200 rpm overnight.

1.5 ml reaction vessels were filled with 1 ml each of YM medium and inoculated with the C. albicans suspension of the overnight culture. The resulting C. albicans suspension was set at the start of the experiment to an optical density, measured at 600 nm, of 0.1. The concentration of the peptide solution in water or ZPT solution (zinc pyrithione, >96%, Sigma Aldrich) dissolved in DMSO was 2% (w/w).

The following growth was compared:
C. albicans suspension and addition of P18 peptide with a final concentration in the 1.5 ml reaction vessel of 50 ppm after 5 minutes, 10 minutes, 20 minutes and 60 minutes
C. albicans suspension and addition of P18 peptide with a final concentration in the 1.5 ml reaction vessel of 100 ppm after 5 minutes, 10 minutes, 20 minutes and 60 minutes
C. albicans suspension and addition of P18 peptide with a final concentration in the 1.5 ml reaction vessel of 200 ppm after 5 minutes, 10 minutes, 20 minutes and 60 minutes
C. albicans suspension and addition of P18 peptide with a final concentration in the 1.5 ml reaction vessel of 500 ppm after 5 minutes, 10 minutes, 20 minutes and 60 minutes
C. albicans suspension and addition of P18 peptide with a final concentration in the 1.5 ml reaction vessel of 1000 ppm after 5 minutes, 10 minutes, 20 minutes and 60 minutes
C. albicans suspension and addition of ZPT with a final concentration in the 1.5 ml reaction vessel of 50 ppm after 5 minutes, 10 minutes, 20 minutes and 60 minutes
C. albicans suspension and addition of ZPT with a final concentration in the 1.5 ml reaction vessel of 100 ppm after 5 minutes, 10 minutes, 20 minutes and 60 minutes
C. albicans suspension and addition of ZPT with a final concentration in the 1.5 ml reaction vessel of 200 ppm after 5 minutes, 10 minutes, 20 minutes and 60 minutes
C. albicans suspension and addition of ZPT with a final concentration in the 1.5 ml reaction vessel of 500 ppm after 5 minutes, 10 minutes, 20 minutes and 60 minutes
C. albicans suspension and addition of ZPT with a final concentration in the 1.5 ml reaction vessel of 1000 ppm after 5 minutes, 10 minutes, 20 minutes and 60 minutes

The 1.5 ml reaction vessels were incubated at room temperature.

After the incubation times specified, the colony forming units (CFU) were determined by streaking 1 μl of each of the suspensions diluted in 20 μl of YM medium and, after incubation for 2 days, counting the colonies.

TABLE 15
Counting of colony forming units (CFU) (the figures shown are means and
standard deviations of the experiments)

| | | Colony forming units after the incubation time | | | |
|---|---|---|---|---|---|
| | Final peptide | specified | | | |
| | concentration in | 5 | 10 | 20 | 60 |
| Substance | the experiment | minutes | minutes | minutes | minutes |
| — | 0 ppm | 1080 ± 107 | 1040 ± 136 | 1048 ± 6 | 1051 ± 160 |
| P18 | 50 ppm | 986 ± 280 | 944 ± 209 | 954 ± 14 | 518 ± 82 |
| | 100 ppm | 718 ± 116 | 628 ± 112 | 454 ± 127 | 25 ± 0 |
| | 200 ppm | 242 ± 122 | 72 ± 2 | 29 ± 2 | 1 ± 1 |
| | 500 ppm | 39 ± 1 | 5 ± 6 | 1 ± 1 | 0 ± 0 |
| | 1000 ppm | 10 ± 2 | 1 ± 2 | 0 ± 0 | 0 ± 0 |
| ZPT | 50 ppm | 830 ± 190 | 1096 ± 51 | 986 ± 20 | 982 ± 93 |
| | 100 ppm | 809 ± 100 | 1048 ± 74 | 806 ± 105 | 762 ± 212 |
| | 200 ppm | 676 ± 204 | 906 ± 331 | 882 ± 190 | 711 ± 125 |
| | 500 ppm | 863 ± 35 | 832 ± 192 | 710 ± 71 | 617 ± 134 |
| | 1000 ppm | 806 ± 20 | 700 ± 153 | 550 ± 88 | 482 ± 69 |

The results show that the C. albicans living cell count in the concentration range studied was already drastically reduced within the first 20 minutes by the effect of P18. For ZPT, no significant inhibition was observed within the concentration range studied over the period observed. The results indicate a different mechanism of action of peptide P18 compared to ZPT. The causes of this may be different points of attack of the two antimicrobial substances. While ZPT possibly inhibits membrane transport (Chandler et al. 1978 Antimicrobial Agents and Chemotherapy, 14: 60-68), P18 possible interacts with the fungal membrane and dissolves it, as already described for the effect of antimicrobial peptides on bacteria. On the other hand, binding to relevant regions of DNA or to relevant proteins might also be a possible mode of action of P18, or else a combination of the effect on several points of attack.

In addition, the different effect can be explained by detoxification of the ZPT by C. albicans.

### Example 17

### Short-Term Effect of P18 on Trichophyton Rubrum

The aim of this experiment was to study the effect of peptide P18 (P18 sequence H-KWKLFKKIPKFLHLAKKF-NH₂ (Bachem AG, Switzerland)) against Trichophyton rubrum (DSM21146) in the course of brief incubation within the first hour, and to compare it with the effect of ZPT (zinc pyrithione). For this purpose, the procedure was as follows.

MEP growth medium according to DSMZ:

30 g of malt extract (Becton, Dickinson and Company, Sparks, USA)
3 g of soya peptone (Becton, Dickinson and Company, Sparks, USA)
make up to 1 liter, set pH 5.6

The components were sterilized at 121° C., 1 bar gauge for 20 minutes.

For agar plates, 15 g/l agar-agar was added to the medium.

The growth test was effected as follows: an agar plate on which the Trichophyton rubrum culture had grown was rinsed with 5 ml of MEP medium.

1.5 ml reaction vessels were each filled with 1 ml of MEP medium and inoculated with 10 μl of the T. rubrum suspension. The concentration of the peptide solution in water or ZPT solution (zinc pyrithione, >96%, Sigma Aldrich) dissolved in DMSO was 2% (w/w).

The following growth was compared:
T. rubrum suspension and addition of P18 peptide with a final concentration in the 1.5 ml reaction vessel of 50 ppm after 5 minutes, 10 minutes, 20 minutes and 60 minutes
T. rubrum suspension and addition of P18 peptide with a final concentration in the 1.5 ml reaction vessel of 100 ppm after 5 minutes, 10 minutes, 20 minutes and 60 minutes
T. rubrum suspension and addition of P18 peptide with a final concentration in the 1.5 ml reaction vessel of 200 ppm after 5 minutes, 10 minutes, 20 minutes and 60 minutes
T. rubrum suspension and addition of P18 peptide with a final concentration in the 1.5 ml reaction vessel of 500 ppm after 5 minutes, 10 minutes, 20 minutes and 60 minutes
T. rubrum suspension and addition of P18 peptide with a final concentration in the 1.5 ml reaction vessel of 1000 ppm after 5 minutes, 10 minutes, 20 minutes and 60 minutes
T. rubrum suspension and addition of ZPT with a final concentration in the 1.5 ml reaction vessel of 50 ppm after 5 minutes, 10 minutes, 20 minutes and 60 minutes
T. rubrum suspension and addition of ZPT with a final concentration in the 1.5 ml reaction vessel of 100 ppm after 5 minutes, 10 minutes, 20 minutes and 60 minutes
T. rubrum suspension and addition of ZPT with a final concentration in the 1.5 ml reaction vessel of 200 ppm after 5 minutes, 10 minutes, 20 minutes and 60 minutes
T. rubrum suspension and addition of ZPT with a final concentration in the 1.5 ml reaction vessel of 500 ppm after 5 minutes, 10 minutes, 20 minutes and 60 minutes
T. rubrum suspension and addition of ZPT with a final concentration in the 1.5 ml reaction vessel of 1000 ppm after 5 minutes, 10 minutes, 20 minutes and 60 minutes

The 1.5 ml reaction vessels were incubated at room temperature.

After the incubation times specified, the colony forming units (CFU) were determined by streaking 1 μl of each of the suspensions diluted with 20 μl of MEP medium and, after incubation for 2 days, counting the colonies. By way of example, the CFUs of an experiment which was conducted in double determination are shown.

TABLE 16
Counting of colony forming units (CFU) (the figures shown are
means of the experiments)

| | | Colony forming units after the | | | |
|---|---|---|---|---|---|
| | Final peptide | incubation time specified | | | |
| | concentration in | 5 | 10 | 20 | 60 |
| Substance | the experiment | minutes | minutes | minutes | minutes |
| — | 0 ppm | 58 | 48 | 64 | 48 |
| P18 | 50 ppm | 59 | 55 | 35 | 23 |
| | 100 ppm | 41 | 22 | 36 | 18 |
| | 200 ppm | 32 | 39 | 24 | 5 |
| | 500 ppm | 30 | 18 | 9 | 1 |
| | 1000 ppm | 17 | 18 | 10 | 2 |
| ZPT | 50 ppm | 44 | 51 | 51 | 55 |
| | 100 ppm | 38 | 54 | 50 | 48 |
| | 200 ppm | 44 | 51 | 56 | 49 |
| | 500 ppm | 43 | 47 | 51 | 44 |
| | 1000 ppm | 52 | 62 | 47 | 48 |

The results show that the T. rubrum living cell count within the concentration range studied is reduced by a maximum factor of 20 by the effect of P18 as early as in the first 60 minutes. In the case of a cell count 10 times higher, the living cell count was reduced at most by the factor of 2 (data not shown). These results show that peptide P18 causes inhibition of the growth of T. rubrum.

Since the effect depends on the cell count, the peptide itself appears to be consumed by its effect. These results indicate a mechanism of action in which peptide P18 does not catalyze the growth-inhibiting effect, but rather is involved irreversibly in the reaction. The reason for this might accordingly be an irreversible interaction of the peptide with the fungal membrane or with DNA.

In addition, the results indicate different mechanisms of action of ZPT and P18, since no significant inhibition has been detected for ZPT within the concentration range studied over the period observed. The causes of this might be the same as discussed in the comparable example for C. albicans (Example 16).

### Example 18

### Short-Term Effect of P18 on Escherichia coli

An effect of P18 on Escherichia coli after incubation over several hours is known (Shin et al. 1999 Journal of Peptide Research, 53: 82-90). The aim of this experiment was to study the effect of peptide P18 (P18 sequence H-KWKLFKKIPKFLHLAKKF-NH₂ (Bachem AG, Switzerland)) on Escherichia coli (Stratagene, BLR) in the course of brief incubation within the first hour, and to compare it with the effect of ZPT (zinc pyrithione). For this purpose, the procedure was as follows.

LB growth medium (Becton, Dickinson and Company, Sparks, USA)
3 g of yeast extract
3 g of malt extract
5 g of peptone
10 g of dextrose
make up to 1 liter, set pH 7

The components were sterilized at 121° C., 1 bar gauge for 20 minutes.

For agar plates, 15 g/l agar-agar was added to the medium.

The growth test was effected as follows: 10 ml of LB medium were inoculated with E. coli and incubated at 37° C. and 200 rpm overnight.

1.5 ml reaction vessels were each filled with 1 ml of LB medium and inoculated with the E. coli suspension from the overnight culture. The E. coli suspension was adjusted at the start of the experiment to an optical density, measured at 600 nm, of 0.1. The concentration of the peptide solution in water or ZPT solution (zinc pyrithione, >96%, Sigma Aldrich) dissolved in DMSO was 2% (w/w).

The following growth was compared:
E. coli suspension and addition of P18 peptide with a final concentration in the 1.5 ml reaction vessel of 50 ppm after 5 minutes, 10 minutes, 20 minutes and 60 minutes
E. coli suspension and addition of P18 peptide with a final concentration in the 1.5 ml reaction vessel of 100 ppm after 5 minutes, 10 minutes, 20 minutes and 60 minutes
E. coli suspension and addition of P18 peptide with a final concentration in the 1.5 ml reaction vessel of 200 ppm after 5 minutes, 10 minutes, 20 minutes and 60 minutes
E. coli suspension and addition of P18 peptide with a final concentration in the 1.5 ml reaction vessel of 500 ppm after 5 minutes, 10 minutes, 20 minutes and 60 minutes
E. coli suspension and addition of P18 peptide with a final concentration in the 1.5 ml reaction vessel of 1000 ppm after 5 minutes, 10 minutes, 20 minutes and 60 minutes
E. coli suspension and addition of ZPT with a final concentration in the 1.5 ml reaction vessel of 50 ppm after 5 minutes, 10 minutes, 20 minutes and 60 minutes
E. coli suspension and addition of ZPT with a final concentration in the 1.5 ml reaction vessel of 100 ppm after 5 minutes, 10 minutes, 20 minutes and 60 minutes
E. coli suspension and addition of ZPT with a final concentration in the 1.5 ml reaction vessel of 200 ppm after 5 minutes, 10 minutes, 20 minutes and 60 minutes
E. coli suspension and addition of ZPT with a final concentration in the 1.5 ml reaction vessel of 500 ppm after 5 minutes, 10 minutes, 20 minutes and 60 minutes
E. coli suspension and addition of ZPT with a final concentration in the 1.5 ml reaction vessel of 1000 ppm after 5 minutes, 10 minutes, 20 minutes and 60 minutes

The 1.5 ml reaction vessels were incubated at room temperature.

After the incubation times specified, the colony forming units (CFU) were determined by streaking 1 μl of each of the suspensions diluted in 20 μl of LB medium and, after incubation for 2 days, counting the colonies.

TABLE 17
Counting of colony forming units (CFU) (the figures shown
are means and standard deviations of the experiments)

| | Final peptide | Colony forming units after the incubation time | | | |
|---|---|---|---|---|---|
| | concentration in | specified | | | |
| Substance | the experiment | 5 minutes | 10 minutes | 20 minutes | 60 minutes |
| — | 0 ppm | 2000 ± 0 | 2000 ± 0 | 2000 ± 0 | 2000 ± 0 |
| P18 | 50 ppm | 2000 ± 0 | 2000 ± 0 | 2000 ± 0 | 1208 ± 532 |
| | 100 ppm | 2000 ± 0 | 2000 ± 0 | 1836 ± 108 | 726 ± 88 |
| | 200 ppm | 2000 ± 0 | 2000 ± 0 | 1310 ± 144 | 192 ± 56 |
| | 500 ppm | 2000 ± 0 | 2000 ± 0 | 816 ± 96 | 24 ± 16 |
| | 1000 ppm | 2000 ± 0 | 2000 ± 0 | 369 ± 383 | 6 ± 2 |
| ZPT | 50 ppm | 2000 ± 0 | 2000 ± 0 | 2000 ± 0 | 2000 ± 0 |
| | 100 ppm | 2000 ± 0 | 2000 ± 0 | 2000 ± 0 | 2000 ± 0 |
| | 200 ppm | 2000 ± 0 | 2000 ± 0 | 2000 ± 0 | 2000 ± 0 |
| | 500 ppm | 2000 ± 0 | 2000 ± 0 | 2000 ± 0 | 2000 ± 0 |
| | 1000 ppm | 2000 ± 0 | 2000 ± 0 | 2000 ± 0 | 2000 ± 0 |

The results show that the E. coli living cell count within the concentration range studied is drastically reduced by the effect of P18 as early as in the first 60 minutes.

For ZPT, no significant inhibition was observed within the concentration range studied over the period observed.

Causes of the different efficacy of P18 and ZPT may be different points of attack of the two antimicrobial substances. While ZPT possibly inhibits membrane transport (Chandler et al. 1978 Antimicrobial Agents and Chemotherapy, 14: 60-68), P18 possibly interacts with the bacterial membrane and dissolves it, as already described for the effect of antimicrobial peptides on bacteria. On the other hand, binding to relevant regions of DNA or to relevant proteins might also be a possible mode of action of P18, or else a combination of several mechanisms of action. In addition, the different effect can be explained by detoxification of the ZPT by E. coli.

### Example 19

### Short-Term Effect of P18 on Staphylococcus Epidermidis

An effect of P18 on Staphylococcus epidermidis after incubation over several hours is known (Shin et al., 2002, Biochemical and Biophysical Research Communications, 290: 558-562). The aim of this experiment was to study the effect of peptide P18 (P18 sequence H-KWKLFKKIPKFLHLAKKF-NH₂ (Sachem AG, Switzerland)) on Staphylococcus epidermidis (DSM 1798) in the course of brief incubation within the first hour, and to compare it with the effect of ZPT (zinc pyrithione). For this purpose, the procedure was as follows.

TSBY growth medium:
Ready-made TSB medium (Becton, Dickinson and Company, Sparks, USA);
17 g of pancreatin-degraded casein
3 g of pancreatin-degraded soya
2.5 g of dextrose
5 g of sodium chloride
2.5 g of dipotassium phosphate
3 g of yeast extract (Becton, Dickinson and Company, Sparks, USA)
make up to 1 liter, set pH 7

The components were sterilized at 121° C., 1 bar gauge for 20 minutes.

For agar plates, 15 g/l agar-agar were added to the medium.

The growth test was effected as follows: 5 ml of TSBY medium were inoculated with S. epidermidis and incubated at 37° C. and 200 rpm overnight.

1.5 ml reaction vessels were each filled with 1 ml of TSBY medium and inoculated with S. epidermidis suspension from the overnight culture. The S. epidermidis suspension was adjusted at the start of the experiment to an optical density, measured at 600 nm, of 0.1. The concentration of the peptide solution in water or ZPT solution (zinc pyrithione, >96%, Sigma Aldrich) dissolved in DMSO was 2% (w/w).

The following growth was compared:
S. epidermidis suspension and addition of P18 peptide with a final concentration in the 1.5 ml reaction vessel of 50 ppm after 5 minutes, 10 minutes, 20 minutes and 60 minutes
S. epidermidis suspension and addition of P18 peptide with a final concentration in the 1.5 ml reaction vessel of 100 ppm after 5 minutes, 10 minutes, 20 minutes and 60 minutes
S. epidermidis suspension and addition of P18 peptide with a final concentration in the 1.5 ml reaction vessel of 200 ppm after 5 minutes, 10 minutes, 20 minutes and 60 minutes
S. epidermidis suspension and addition of P18 peptide with a final concentration in the 1.5 ml reaction vessel of 500 ppm after 5 minutes, 10 minutes, 20 minutes and 60 minutes
S. epidermidis suspension and addition of P18 peptide with a final concentration in the 1.5 ml reaction vessel of 1000 ppm after 5 minutes, 10 minutes, 20 minutes and 60 minutes
S. epidermidis suspension and addition of ZPT with a final concentration in the 1.5 ml reaction vessel of 50 ppm after 5 minutes, 10 minutes, 20 minutes and 60 minutes
S. epidermidis suspension and addition of ZPT with a final concentration in the 1.5 ml reaction vessel of 100 ppm after 5 minutes, 10 minutes, 20 minutes and 60 minutes
S. epidermidis suspension and addition of ZPT with a final concentration in the 1.5 ml reaction vessel of 200 ppm after 5 minutes, 10 minutes, 20 minutes and 60 minutes
S. epidermidis suspension and addition of ZPT with a final concentration in the 1.5 ml reaction vessel of 500 ppm after 5 minutes, 10 minutes, 20 minutes and 60 minutes
S. epidermidis suspension and addition of ZPT with a final concentration in the 1.5 ml reaction vessel of 1000 ppm after 5 minutes, 10 minutes, 20 minutes and 60 minutes

The 1.5 ml reaction vessels were incubated at room temperature.

After the incubation times specified, the colony forming units (CFU) were determined by streaking 1 μl of each of the suspensions diluted in 20 μl of TSBY medium and, after incubation for 2 days, counting the colonies.

TABLE 18
Counting of the colony forming units (CFU) (the figures shown are means and
standard deviations of the experiments)

| | Final peptide | Colony forming units after the incubation time | | | |
|---|---|---|---|---|---|
| | concentration in | specified | | | |
| Substance | the experiment | 5 minutes | 10 minutes | 20 minutes | 60 minutes |
| — | 0 ppm | 2000 ± 0 | 2000 ± 0 | 2000 ± 0 | 2000 ± 0 |
| P18 | 50 ppm | 221 ± 5 | 19 ± 3 | 4.75 ± 0.4 | 0.75 ± 0.4 |
| | 100 ppm | 171 ± 219 | 6.5 ± 4.2 | 0.5 ± 0 | 0 ± 0 |
| | 200 ppm | 14 ± 18 | 5.75 ± 4.6 | 0.25 ± 0.4 | 0 ± 0 |
| | 500 ppm | 19 ± 22 | 0.25 ± 0.4 | 0.75 ± 1.1 | 0 ± 0 |
| | 1000 ppm | 1.25 ± 0.4 | 0 ± 0 | 0 ± 0 | 0 ± 0 |
| ZPT | 50 ppm | 2000 ± 0 | 2000 ± 0 | 1033 ± 1368 | 522 ± 721 |
| | 100 ppm | 2000 ± 0 | 2000 ± 0 | 1200 ± 1131 | 707 ± 698 |
| | 200 ppm | 2000 ± 0 | 2000 ± 0 | 2000 ± 0 | 2000 ± 0 |
| | 500 ppm | 2000 ± 0 | 2000 ± 0 | 2000 ± 0 | 2000 ± 0 |
| | 1000 ppm | 2000 ± 0 | 2000 ± 0 | 2000 ± 0 | 2000 ± 0 |

The results show that the S. epidermidis living cell count within the concentration range studied was reduced drastically by the effect of P18 as early as in the first 5 minutes. For ZPT, low inhibition was observed at 50 ppm and 100 ppm within the concentration range studied over the period observed. For higher concentrations, no significant inhibition was observed for ZPT.

### Example 20

### P18 in Formulation Against Candida albicans

The aim of the experiment was to study the effect of a pharmaceutical base formulation with the P18 peptide ingredient (P18 sequence H-KWKLFKKIPKFLHLAKKF-NH₂ (Bachem AG, Switzerland)). For this purpose, the following base formulation was used:

1% hydroxycellulose
10% propylene glycol
made up with water.

Based on the base formulation, formulations with rising P18 concentrations (500 ppm to 10 000 ppm) were prepared from a 20% concentrate solution.

The effect of the formulations on C. albicans (DSM 11948) was studied.

For this purpose, the procedure was as follows.

YM growth medium (Becton, Dickinson and Company, Sparks, USA)
3 g of yeast extract
3 g of malt extract
5 g of peptone
10 g of dextrose
make up to 1 liter, set pH 6.2

The components were sterilized at 121° C., 1 bar gauge for 20 minutes.

For agar-agar plates, 15 g/l agar-agar was added to the medium.

The growth test was effected as follows: 5 ml of YM medium were inoculated with C. albicans and incubated at 30° C. and 200 rpm overnight.

1.5 ml reaction vessels were each filled with 1 ml of YM medium and inoculated with the C. albicans suspension from the overnight culture such that an optical density—measured at 600 nm—of 0.1 was obtained at the start of the experiment. The resulting C. albicans suspension was mixed in a ratio of 1:9 (formulation: C. albicans suspension) with the formulations. 1 μl of the culture was diluted with 20 μl of YM medium after 5 minutes, 10 minutes, 20 minutes and 60 minutes, and then plated out. After incubation over 24 hours, the colonies on the plates were counted.

TABLE 19
Counting of the colony forming units (CFU) (the figures shown are means and
standard deviations of the experiments)

| | Peptide | Colony forming units after the incubation time | | | |
|---|---|---|---|---|---|
| | concentration in | specified | | | |
| Substance | the formulation | 5 minutes | 10 minutes | 20 minutes | 60 minutes |
| — | C. albicans | 1434 ± 76 | 1452 ± 45 | 1506 ± 235 | 1404 ± 141 |
| | suspension | | | | |
| | without | | | | |
| | formulation | | | | |
| — | 0 ppm | 1505 ± 38 | 1352 ± 91 | 1526 ± 26 | 1441 ± 216 |
| P18 | 500 ppm | 1484 ± 28 | 1445 ± 38 | 1262 ± 354 | 962 ± 738 |
| | 1000 ppm | 863 ± 553 | 616 ± 76 | 251 ± 109 | 3.25 ± 1.8 |
| | 2000 ppm | 29 ± 23 | 3.5 ± 3.5 | 0.25 ± 0.4 | 0 ± 0 |
| | 5000 ppm | 1 ± 0.7 | 0 ± 0 | 0 ± 0 | 0 ± 0 |
| | 10 000 ppm | 0.5 ± 0 | 0 ± 0 | 0 ± 0 | 0 ± 0 |

The results show that the formulation itself (0 ppm) does not have any growth-inhibiting effect. After incubation for 60 minutes, significant inhibition of growth was observed for a formulation containing 1000 ppm of P18. After a treatment time of only 5 minutes, an effect was obtained with a formulation which comprised 5000 ppm of P18 peptide.

### Example 21

### P18 in Formulation Against Escherischia Coli

The aim of the experiment was to study the effect of a pharmaceutical base formulation containing the P18 peptide ingredient (P18 sequence H-KWKLFKKIPKFLHLAKKF-NH₂ (Bachem AG, Switzerland)). For this purpose, the following base formulation was used:

1% hydroxycellulose
10% propylene glycol
made up with water.

Based on the base formulation, formulations with rising P18 concentrations (500 ppm to 10 000 ppm) were prepared from a 20% concentrate solution.

The effect of the formulations on E. coli (Stratagene, BLR) was studied.

For this purpose, the procedure was as follows.

LB growth medium (Becton, Dickinson and Company, Sparks, USA)
3 g of yeast extract
3 g of malt extract
5 g of peptone
10 g of dextrose
make up to 1 liter, set pH 7

The components were sterilized at 121° C., 1 bar gauge for 20 minutes.

For agar plates, 15 g/l agar-agar was added to the medium.

The growth test was effected as follows: 5 ml of LB medium were inoculated with E. coli and incubated at 37° C. and 200 rpm overnight.

1.5 ml reaction vessels were each filled with 1 ml of LB medium and inoculated with the E. coli suspension from the overnight culture such that an optical density, measured at 600 nm, of 0.1 was obtained at the start of the experiment. The resulting E. coli suspension was mixed in a ratio of 1:9 (formulation: E. coli suspension) with the formulations. 1 μl of the culture was diluted with 20 μl of LB medium after 5 minutes, 10 minutes, 20 minutes and 60 minutes, and then plated out. After incubation over 24 hours, the colonies on the plates were counted.

TABLE 20
Counting of the colony forming units (CFU) (the figures shown are means and
standard deviations of the experiments)

| | Peptide | Colony forming units after the incubation time | | | |
|---|---|---|---|---|---|
| | concentration in | specified | | | |
| Substance | the formulation | 5 minutes | 10 minutes | 20 minutes | 60 minutes |
| | | 2000 ± 0 | 2000 ± 0 | 2000 ± 0 | 2000 ± 0 |
| — | 0 ppm | 2000 ± 0 | 2000 ± 0 | 2000 ± 0 | 2000 ± 0 |
| P18 | 500 ppm | 2000 ± 0 | 2000 ± 0 | 2000 ± 0 | 709 ± 451 |
| | 1000 ppm | 2000 ± 0 | 2000 ± 0 | 1619 ± 534 | 332 ± 236 |
| | 2000 ppm | 2000 ± 0 | 2000 ± 0 | 733 ± 349 | 93 ± 127 |
| | 5000 ppm | 2000 ± 0 | 1225 ± 339 | 411 ± 516 | 29 ± 36 |
| | 10 000 ppm | 1778 ± 314 | 901 ± 120 | 226 ± 286 | 7.75 ± 9.5 |

The results show that the formulation itself (0 ppm) does not have any growth-inhibiting effect. After incubation for 60 minutes, a significant inhibition of growth is observed for a formulation containing 10 000 ppm of P18.

### Example 22

### P18 in Formulation Against Further Fungi and Bacteria

The aim of the experiment was to study the effect of a pharmaceutical base formulation containing the P18 peptide ingredient (P18 sequence H-KWKLFKKIPKFLHLAKKF-NH₂ (Bachem AG, Switzerland)) on further fungi and bacteria. For this purpose, the following base formulation was used:

1% hydroxycellulose
10% propylene glycol
made up with water.

Based on the base formulation, formulations containing 10 000 ppm of P18 peptide were prepared from a 20% concentrate solution.

The effect of the formulations on S. epidermidis (DSM 1798), M. furfur (DSM 6170) and T. rubrum (DSM 21146) was studied.

For this purpose, the procedure was as follows.

TSBY growth medium for S. epidermidis
Ready-made TSB medium (Becton, Dickinson and Company, Sparks, USA)
17 g of pancreatin-degraded casein
3 g of pancreatin-degraded soya
2.5 g of dextrose
5 g of sodium chloride
2.5 g of dipotassium phosphate
3 g of yeast extract (Becton, Dickinson and Company, Sparks, USA)
make up to 1 liter, set pH 7

The components were sterilized at 121° C., 1 bar gauge for 20 minutes.

M472 growth medium according to DSMZ for M. furfur
40 g of malt extract (Becton, Dickinson and Company, Sparks, USA)
20 g of ox bile (Merck, Darmstadt, Germany)

### 10 g of Tween 40 (Sigma-Aldrich Chemie GmbH, Steinheim, Germany)

make up to 1 liter.

The components were sterilized at 121° C., 1 bar gauge for 20 minutes.

Subsequently, 2 ml of olive oil (sterile-filtered) were added.

MEP growth medium according to DSMZ for T. rubrum
30 g of malt extract (Becton, Dickinson and Company, Sparks, USA)
3 g of soya peptone (Becton, Dickinson and Company, Sparks, USA)
make up to 1 liter, set pH 5.6.

The components were sterilized at 121° C., 1 bar gauge for 20 minutes.

For agar plates, 15 g/l agar-agar were added to the medium.

The growth test was effected as follows:

For S. aureus and S. epidermidis, 5 ml of medium were inoculated with the microorganism and incubated at 37° C. and 200 rpm overnight.

For M. furfur, 5 ml of medium were inoculated with the microorganism and incubated at 30° C. and 200 rpm overnight.

For T. rubrum, an agar plate on which it had grown was rinsed with 5 ml of medium. 10 μl of the resulting suspension were added to 1 ml of MEP medium and used directly in the experiment.

1.5 ml reaction vessels were each filled with 1 ml of medium and inoculated with the fungal or bacterial suspension from the overnight culture such that the resulting suspensions at the start of the experiment had been adjusted to an optical density, measured at 600 nm, of 0.1. The resulting suspension was mixed with the formulations in a ratio of 1:9 (formulation: microbial suspension). 1 μl of the culture was diluted with 20 μl of medium after 5 minutes and 60 minutes and then plated out. After incubation over 24 hours, the colonies on the plates were counted.

TABLE 21
Counting of the colony forming units (CFU) (the figures shown
are means and standard deviations of the experiments)

| | Peptide | Colony forming units after | |
|---|---|---|---|
| | concentration in | the incubation time specified | |
| Organism | the formulation | 5 minutes | 60 minutes |
| S. epidermidis | 0 ppm | 2000 ± 0 | 2000 ± 0 |
| S. epidermidis | 10 000 ppm | 790 ± 393 | 513 ± 20 |
| M. furfur | 0 ppm | 886 ± 45 | 729 ± 11 |
| M. furfur | 10 000 ppm | 671 ± 13 | 2 ± 2 |
| T. rubrum | 0 ppm | 67 ± 16 | 52 ± 10 |
| T. rubrum | 10 000 ppm | 25 ± 8 | 6 ± 1 |

The formulation itself (0 ppm) did not have any growth-inhibiting effect (data not shown).

The results show a growth-inhibiting effect of the formulation containing P18. A better effect was observed for the fungi studied than for the S. epidermidis bacterium tested.

In summary, a treatment with a formulation which comprised P18 caused an up to >300-fold reduction in the cell count.

Consequently, the results show a broad antimicrobial effect of peptide P18. Differences are observed for different microorganisms. The results additionally show that the mechanisms of action of P18 and ZPT differ distinctly, especially since no significant effect of ZPT was detected over only a short incubation period.

Peptide P18 possibly acts on the fungal or bacterial membrane, the DNA or at both action sites. The effect which can be observed after only brief incubation indicates a central action site which is essential for the survival of the microorganisms, irrespective of the growth of the microorganisms.

During the effect, the peptide itself is inactivated, possible by irreversible binding to membrane constituents or relevant regions of the DNA.

### Example 23

### Comparison of the Efficacy of P18 and AFPP on the Growth of Malassezia Furfur

WO 00/32220 describes the effect of the antifungal polypeptide AFPP from Aspergillus giganteus on the growth of Malassezia furfur.

In order to compare the effect of P18 (SEQ ID NO. 3; sequence: KWKLFKKIPKFLHLAKKF-NH₂ (Bachem. AG, Switzerland)) with the effect of AFPP, AFPP was provided from the culture supernatant of the A. giganteus strain CBS 526.65 (Organobalance, Berlin). The purification was effected according to Theis et al. (Theis T., Wedde M., Meyer V., Stahl U. (2003) The antifungal protein from Aspergillus giganteus causes membrane permeabilization. Antimicrob. Agents Chemother. 47:588-593; Theis T., Marx F., Salvenmoser W., Stahl U., Meyer V. (2005) New insights into the target site and mode of action of the antifungal protein (AFP) of Aspergillus giganteus. Res Microbiol. 156:47-56.)

After replacing the buffer and concentration, a 2% (w/w) AFPP solution in phosphate buffer (10 mM sodium phosphate, pH 7.5, 100 mM NaCl) was obtained. The purification was confirmed by N-terminal sequencing; HPLC analysis showed a purity of the AFPP solution of greater than 99%.

The P18 concentrate was also present as a 2% (w/w) solution in phosphate buffer.

The experiments for comparison of the two peptides were conducted as follows:

Growth medium: M472 Pityrosporum medium according to DSMZ (German Collection of Microorganisms and Cell Cultures, Braunschweig, Germany)
40 g of malt extract (Becton, Dickinson and Company, Sparks, USA)
20 g of ox bile (Merck, Darmstadt, Germany)

### 10 g of Tween 40 (Sigma-Aldrich Chemie GmbH, Steinheim, Germany)

The components were sterilized at 121° C., 1 bar gauge for 20 minutes.

2 g/l of olive oil (was sterile-filtered and added after the other components had been autoclaved)

For agar plates, 15 g/l agar-agar was added to the medium.

The growth test was effected as follows: a shake culture containing M472 Pityrosporum medium was inoculated with M. furfur DSM 6170 (DSMZ) and incubated with shaking at 30° C. and 200 rpm overnight.

For each test mixture, a 1.5 ml reaction vessel was filled with Pityrosporum medium and inoculated with the M. furfur overnight culture to give a start OD of 0.1, measured at 600 nm.

The growth of the following test mixtures was monitored:
M. furfur suspension and addition of 30 μl of phosphate buffer (in order to rule out any influence of the phosphate buffer on the effect of the active peptides at a final concentration of 0.1%)
M. furfur suspension and addition of 60 μl of phosphate buffer (in order to rule out any influence of the phosphate buffer on the effect of the active peptides at a final concentration of 0.2%)
M. furfur suspension and addition of 90 μl of phosphate buffer (in order to rule out any influence of the phosphate buffer on the effect of the active peptides at a final concentration of 0.3%)
M. furfur suspension with a final P18 concentration of 0.1% in the reaction volume
M. furfur suspension with a final P18 concentration of 0.2% in the reaction volume
M. furfur suspension with a final P18 concentration of 0.3% in the reaction volume
M. furfur suspension with a final AFPP concentration of 0.1% in the reaction volume
M. furfur suspension with a final AFPP concentration of 0.2% in the reaction volume
M. furfur suspension with a final AFPP concentration of 0.3% in the reaction volume

The reaction volume was 600 μl in each case. The growth of M. furfur in the test mixtures was observed over a period of 24 hours. For this purpose, the mixtures were diluted 1:10 in M472 medium after 5 minutes, 20 minutes and 24 hours, and then 10 μl were plated out. The CFU (colony forming units) were determined by counting after incubation at 30° C. for 6 days.

The experiments were effected in double determination and were independently repeated at least once.

No significant influence of the phosphate buffer on growth was observed.

TABLE 22
Counting of colony forming units (CFU) (the figures shown
are means and standard deviations of the experiments)

| | Final peptide | | | |
|---|---|---|---|---|
| | concentration | Colony forming units after the incubation | | |
| | in the | time specified | | |
| Substance | experiment | 5 minutes | 20 minutes | 24 hours |
| — | 0.0% | 390 ± 13 | 496 ± 178 | >1000 |
| P18 | 0.1% | 205 ± 192 | 48 ± 56 | 0 |
| | 0.2% | 56 ± 52 | 5 ± 4 | 0 |
| | 0.3% | 18 ± 19 | 1 ± 0 | 0 |
| AFPP | 0.1% | 442 ± 161 | 424 ± 22 | >1000 |
| | 0.2% | 448 ± 147 | 424 ± 62 | >1000 |
| | 0.3% | 423 ± 166 | 618 ± 116 | >1000 |

The results show that the addition of P18 reduced the CFU to a much greater degree than comparable concentrations of AFPP within the period observed. This shows that the addition of P18 achieves better inhibition of growth of M. furfur compared to AFPP, and P18 is therefore more effective.

### Example 24

### Comparison of the Efficacy of P18 and AFPP on the Growth of Malassezia furfur in a Shampoo Formulation

In order to study the efficacy of AFPP compared to P18 (SEQ ID NO. 3; sequence: KWKLFKKIPKFLHLAKKF-NH₂ (Bachem AG, Switzerland)) in a shampoo formulation, the experiment described in Example 24 was repeated with a shampoo formulation.

The procedure was as follows:

Both peptides were present as 2% (w/w) solutions in phosphate buffer (10 mM sodium phosphate, pH 7.5, 100 mM NaCl).

The following formulation was used:

TABLE 23
Composition of the shampoo formulation used

| | | |
|---|---|---|
| Trade name | INCI | % |

Phase A

| | | |
|---|---|---|
| Plantacare 818 UP | Coco Glucoside | 15.00 |
| Tween 20 | Polysorbate 20 | 5.00 |
| Plantacare 2000 | Decyl Glucoside | 5.00 |
| Genapol L3 | Laureth-3 | 2.00 |
| Millipore water | Aqua | 69.50 |
| citric acid (50% solution) | Sodium Chloride | q.s. |

Phase B

| | | |
|---|---|---|
| Stepan PEG 6000 DS | PEG-150 | 3.00 |
| | Distearate | |
| | | liquid |

### Production:

Weigh in and dissolve the components of phase A.

### Adjust the pH to 6-7.

Add phase B and heat to approx. 50° C.
Cool to room temperature while stirring.

Peptide solution with a final concentration of 0.1% and 0.2% was added to the shampoo formulation. The resulting formulations were stirred over 16 hours in order to obtain homogeneous solutions. The same procedure was repeated with equivalent volumes of the phosphate buffer in order to rule out any influence of the phosphate buffer on the test results.

The subsequent procedure was as follows:

Growth medium: M472 Pityrosporum medium according to DSMZ:
40 g of malt extract (Becton, Dickinson and Company, Sparks, USA)
20 g of ox bile (Merck, Darmstadt, Germany)

### 10 g of Tween 40 (Sigma-Aldrich Chemie GmbH, Steinheim, Germany)

The components were sterilized at 121° C., 1 bar gauge for 20 minutes.

2 g/l olive oil (was sterile-filtered and added after the other components had been autoclaved)

For agar plates, 15 g/l agar-agar was added to the medium.

The growth test was effected as follows: a shake culture containing M472 Pityrosporum medium was inoculated with M. furfur DSM 6170 (DSMZ) and incubated with shaking at 30° C. and 200 rpm overnight.

For each test mixture, a 1.5 ml reaction vessel was filled with Pityrosporum medium and inoculated with the M. furfur overnight culture to give a start OD of 0.1, measured at 600 nm.

The growth of the following test mixtures was monitored:
M. furfur suspension and addition of phosphate buffer in shampoo formulation in an equivalent amount to the volumes which were added with peptide-shampoo solution (in order to rule out any influence of the phosphate buffer on the effect of the active peptides at concentrations in the shampoo formulation of 0.1% or 0.2%)
M. furfur suspension and shampoo formulation with a P18 concentration of 0.1%
M. furfur suspension and shampoo formulation with a P18 concentration of 0.2%
M. furfur suspension and shampoo formulation with an AFPP concentration of 0.1%
M. furfur suspension and shampoo formulation with a final AFPP concentration of 0.2%

The ratio of the shampoo formulation to the M. furfur culture medium was 1:10 in all test mixtures (shampoo formulation: M. furfur culture medium). The reaction volume was 1 ml.

The growth of M. furfur in the test mixtures was observed over a period of 20 minutes. For this purpose, the mixtures were diluted in M472 medium in a ratio of 1:10 after 10 minutes and 20 minutes, and then 10 μl were plated out. The CFU (colony forming units) were determined by counting after incubation at 30° C. for 6 days.

The experiments were effected in double determination and were independently repeated at least once.

No significant influence of the phosphate buffer on the growth of M. furfur was observed. The addition of the formulation without active peptide already caused a reduction in the CFU.

TABLE 24
Counting of the colony forming units (CFU) (the figures shown
are means and standard deviations of the experiments)

| | |
|---|---|
| | Colony forming units after the |
| Concentration in | incubation time specified |

| Substance | the formulation | 10 minutes | 20 minutes |
|---|---|---|---|
| — | 0.0% | 369 ± 39 | 557 ± 99 |
| P18 | 0.1% | 80 ± 10 | 38 ± 21 |
| | 0.2% | 21 ± 17 | 3 ± 1 |
| AFPP | 0.1% | 370 ± 149 | 403 ± 140 |
| | 0.2% | 531 ± 175 | 342 ± 52 |

The results of the experiments show that the addition of shampoo formulations which comprised rising P18 concentrations reduced the CFU to a much greater degree than comparable formulations with rising AFPP concentrations within the period observed. This shows that the addition of P18 achieves better inhibition of M. furfur growth compared to AFPP, and P18 is therefore more effective.

### Example 25

### Comparison of the Efficacy of Further Inventive Peptides and AFPP on the Growth of Malassezia Furfur

In order to compare the effect of other inventive peptides with the effect of the antifungal polypeptide AFPP from Aspergillus giganteus on the growth of Malassezia furfur, the procedure was as follows:

As a comparison, the peptide variants with SEQ ID NO. 4726 (sequence: FKKALHLFKPIKKFLKWK-NH₂ (Bachem AG, Switzerland)) and SEQ ID NO. 4727 (sequence: KFLHLAKKFPKWKLFKKI-NH₂ (Bachem AG, Switzerland)) were selected. The peptide and AFPP concentrates were in the form of 2% (w/w) solutions in phosphate buffer (10 mM sodium phosphate, pH 7.5, 100 mM NaCl).

The experiments for comparison of the peptides were conducted as follows:

Growth medium: M472 Pityrosporum medium according to DSMZ
40 g of malt extract (Becton, Dickinson and Company, Sparks, USA)
20 g of ox bile (Merck, Darmstadt, Germany)

### 10 g of Tween 40 (Sigma-Aldrich Chemie GmbH, Steinheim, Germany)

The components were sterilized at 121° C., 1 bar gauge for 20 minutes.

2 g/l olive oil (was sterile-filtered and added after the other components had been autoclaved)

For agar plates, 15 g/l agar-agar was added to the medium.

The growth test was effected as follows: a shake culture containing M472 Pityrosporum medium was inoculated with M. furfur DSM 6170 (DSMZ) and incubated with shaking at 30° C. and 200 rpm overnight.

For each test mixture, a 1.5 ml reaction vessel was filled with Pityrosporum medium and inoculated with the M. furfur overnight culture to give a start OD of 0.1, measured at 600 nm.

The growth of the following test mixtures was monitored:
M. furfur suspension and addition of 90 μl of phosphate buffer (in order to rule out any influence of the phosphate buffer on the effect of the active peptides at a final concentration of 0.3%)
M. furfur suspension with a final concentration of the inventive peptide variant with SEQ ID NO. 4726 of 0.3% in the reaction volume
M. furfur suspension with a final concentration of the inventive peptide variant with SEQ ID NO. 4727 of 0.3% in the reaction volume
M. furfur suspension with a final AFPP concentration of 0.3% in the reaction volume.

The reaction volume was 600 μl in each case. The growth of M. furfur in the test mixtures was observed over a period of 10 minutes. For this purpose, the mixtures were diluted 1:10 in M472 medium after incubation for 10 minutes, and then 10 μl were plated out. The CFU (colony forming units) were determined by counting after incubation at 30° C. for 6 days.

The experiments were effected in double determination and were repeated independently.

No significant influence of the phosphate buffer on the growth was observed.

TABLE 25
Counting of colony forming units (CFU) (the figures shown
are means and standard deviations of the experiments)

| | Final peptide | Colony forming units after the |
|---|---|---|
| | concentration in | incubation time specified |
| Substance | the experiment | 10 minutes |
| — | 0.0% | 887 ± 116 |
| Peptide | 0.3% | 3 ± 1 |
| variant SEQ | | |
| ID NO. 4726 | | |
| Peptide | 0.3% | 59 ± 46 |
| variant SEQ | | |
| ID NO. 4727 | | |
| AFPP | 0.3% | 688 ± 116 |

The results show that the addition of the inventive peptide variants reduced the CFU to a much greater degree than comparable concentrations of AFPP within the period observed. This shows that the addition of the inventive peptide variants achieves better inhibition of growth of M. furfur compared to AFPP, and the inventive peptide variants are therefore more effective.

1-20. (canceled)

21. A pharmaceutical composition comprising, in a pharmaceutical carrier, a peptide comprising at least one sequence motif of the following general formula I
Hel1-HB-Hel2 (I)
in which
HB comprises 1 to 5 consecutive amino acid residues and represents a subsequence motif with the function of a helix breaker, and
Hel1 and Hel2 are identical or different subsequence motifs each comprising 5 to 15 consecutive amino acid residues which are selected essentially from hydrophilic residues and hydrophobic residues other than proline, and are each capable of forming an alpha-helix arm, at least one of the helix arms in the axial projection thereof having an incomplete separation into a hydrophobic and hydrophilic helix half.

22. A pharmaceutical composition comprising, in a pharmaceutical carrier, a peptide comprising at least one sequence motif of the following general formula I
Hel1-HB-Hel2 (I)
in which
HB comprises 1 to 5 consecutive amino acid residues and represents a subsequence motif with the function of a helix breaker, and
Hel1 and Hel2 are identical or different subsequence motifs each comprising 5 to 15 consecutive amino acid residues which are selected essentially from hydrophilic residues and hydrophobic residues other than proline, and are each capable of forming an alpha-helix arm,
the peptide having a percentage alpha-helicity value of about 7 to 98%, in 50% (v/v) trifluoroethanol, pH 7.0, or about 8 to 60% in 30 mM SDS, pH 7.0, in each case determined by CD spectrometry.

23. A pharmaceutical composition comprising, in a pharmaceutical carrier, at least one peptide with a sequence or a repetitive sequence motif according to SEQ ID NO: 1:
(SEQ ID NO: 1)
X1 X2K X3 X4 X5KIP X10 KFX6X7 X8 AX9KF
in which
X10 is a peptide bond or any one or two basic or hydrophobic amino acid residues or one or two proline residues and
X1 to X9 are any basic or hydrophobic amino acid residues other than proline;
where the repetitive sequence motifs may be the same or different;
and/or mutants or derivatives thereof.

24. The composition according to claim 21, comprising at least one peptide with a sequence or a repetitive sequence motif according to SEQ ID NO: 2:
(SEQ ID NO: 2)
X1 X2K X3 X4 X5KIP X11 X12 KFX6X7 X8 AX9KF
in which
X1 is lysine, arginine or phenylalanine,
X2 is lysine or tryptophan,
X3 is leucine or lysine,
X4 is phenylalanine or leucine,
X5 is leucine or lysine,
X6 is leucine or lysine,
X7 is histidine or lysine,
X8 is alanine, leucine, valine or serine,
X9 is leucine or lysine,
X11 is proline or a chemical bond, and
X12 is proline or a chemical bond,
where the repetitive sequence motifs are the same or different;
and/or mutants or derivatives thereof.

25. The composition according to claim 24, comprising a peptide with a sequence or a repetitive sequence motif according to SEQ ID NO; 3
(SEQ ID NO: 3)
KWKLFKKIPKFLHLAKKF
and/or a mutant or derivative thereof.

26. The composition according to claim 21, comprising a peptide with a repetitive sequence motif, with a multitude of peptides of the general formula I or according to SEQ ID NO: 1, 2 or 3 or variants or derivatives thereof peptide-bonded to one another via linker groups.

27. The composition according to claim 26, wherein the linkers comprise 1 to 10 consecutive identical or different amino acid residues, preferably selected from alanine, glycine, threonine and serine.

28. The composition according to claim 21, wherein the C-terminal carboxyl group of the peptide has been amidated.

29. The composition according to claim 21, comprising at least one peptide as defined above which has a minimum inhibitory concentration with respect to Malassezia furfur in the range from about 1500 to 0.1 μM, determined under standard conditions.

30. A composition comprising a fusion product of at least one pharmaceutical excipient or active and at least one peptide according to claim 21.

31. The composition according to claim 21, additionally comprising at least one further pharmaceutical active.

32. The composition according to claim 21, additionally comprising at least one anti-inflammatory active.

33. The composition according to claim 21, additionally comprising an antimicrobial active for inhibition of the growth and/or activity of unwanted bacteria.

34. The composition according to claim 21, additionally comprising a sebum-regulating active.

35. The composition according to claim 21, wherein the peptide is present in a proportion of 0.0001 to 50% by weight, based on the total weight of the finished composition.

36. A process for producing the pharmaceutical composition according to claim 21, wherein the peptide as defined in claim 21 is formulated to the desired administration form together with at least one customary pharmaceutical excipient and optionally further pharmaceutical actives.


SEQUENCE LISTING (12 sequences)

SEQ ID NO: 1 - protein, 19 aa, Artificial Sequence
  Sequence Motif 1
XXKXXXKIPXKFXXXAXKF

SEQ ID NO: 2 - protein, 20 aa, Artificial Sequence
  Sequence Motif 2
XXKXXXKIPXXKFXXXAXKF

SEQ ID NO: 3 - protein, 18 aa, Artificial Sequence
  P18 Peptide
KWKLFKKIPKFLHLAKKF

SEQ ID NO: 4 - protein, 18 aa, Artificial Sequence
  RP18 Peptide
RWKLFKKIPKFLHLAKKF

SEQ ID NO: 5 - protein, 18 aa, Artificial Sequence
  KKFP18 Peptide
FKKLFKKIPKFLHAAKKF

SEQ ID NO: 6 - protein, 18 aa, Artificial Sequence
  KKLP18 Peptide
KWKLLKKIPKFKKLALKF

SEQ ID NO: 7 - protein, 18 aa, Artificial Sequence
  AP18 Peptide
KWKLFKKIPKFLHAAKKF

SEQ ID NO: 8 - protein, 18 aa, Artificial Sequence
  KFLP18 Peptide
KWKKFLKIPKFLHAAKKF

SEQ ID NO: 9 - protein, 18 aa, Artificial Sequence
  KLLP18 Peptide
KWKKLLKIPKFLHAAKKF

SEQ ID NO: 10 - protein, 13 aa, Artificial Sequence
  KLsyn Peptide
KWKKLPKKLLKLL

SEQ ID NO: 11 - protein, 18 aa, Artificial Sequence
  Rev-1 Peptide
FKKALHLFKPIKKFLKWK

SEQ ID NO: 12 - protein, 18 aa, Artificial Sequence
  Rev-2 Peptide
KFLHLAKKFPKWKLFKKI